SEQUENCE LISTING (24 sequences)

SEQ ID NO: 1 - protein, 1946 aa, synthetic construct
  REGION 1..1946: note = Synthesized
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 883 aa, synthetic construct
  REGION 1..883: note = Synthesized
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 1965 aa, synthetic construct
  REGION 1..1965: note = Synthesized
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 5898 bp, synthetic construct
  misc_feature 1..5898: note = Synthesized
  source 1..5898: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggaacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagcctcgagcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 5 - protein, 1646 aa, synthetic construct
  REGION 1..1646: note = Synthesized
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - DNA, 4941 bp, synthetic construct
  misc_feature 1..4941: note = Synthesized
  source 1..4941: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggtacatcagagagcgccacccctgaaagtggtcccgggagcgagccagccacatctgggtcggaaacgccaggcacatccgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgtctgacaagaacactggtgattattacgaggacagttatgaagatatttcagcatacttgctgagtaaaaacaatgccattgaaccaagaagcttctctgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 7 - protein, 1945 aa, synthetic construct
  REGION 1..1945: note = Synthesized
TRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 9 - protein, 288 aa, synthetic construct
  REGION 1..288: note = Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 10 - protein, 46 aa, synthetic construct
  REGION 1..46: note = Synthesized
TSTEEGASISDKNTGDYYEDSYEDISAYLLSKNNAIEPRSFSDKTH

SEQ ID NO: 11 - protein, 45 aa, synthetic construct
  REGION 1..45: note = Synthesized
TSTEEGASSDKNTGDYYEDSYEDISAYLLSKNNAIEPRSFSDKTH

SEQ ID NO: 12 - protein, 1947 aa, synthetic construct
  REGION 1..1947: note = Synthesized
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 288 aa, synthetic construct
  REGION 1..288: note = Synthesized
GSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 14 - protein, 144 aa, synthetic construct
  REGION 1..144: note = Synthesized
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 15 - protein, 32 aa, synthetic construct
  REGION 1..32: note = Synthesized
DKNTGDYYEDSYEDISAYLLSKNNAIEPRSFS

SEQ ID NO: 16 - protein, 19 aa, Homo sapiens
MQIELSTCFFLCLLRFCFS

SEQ ID NO: 17 - protein, 745 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQN

SEQ ID NO: 18 - protein, 684 aa, Homo sapiens
EITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - protein, 22 aa, Homo sapiens
MIPARFAGVLLALALILPGTLC

SEQ ID NO: 20 - protein, 741 aa, Homo sapiens
AEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKR

SEQ ID NO: 21 - protein, 103 aa, synthetic construct
  REGION 1..103: note = Synthesized
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDAT

SEQ ID NO: 22 - protein, 374 aa, synthetic construct
  REGION 1..374: note = Synthesized
CSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEP

SEQ ID NO: 23 - protein, 226 aa, synthetic construct
  REGION 1..226: note = Synthesized
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 291 aa, synthetic construct
  REGION 1..291: note = Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASS

(19) United States
(12) Patent Application Publication
(10) Pub. No.: US 2025/0092117 A1
(43) Pub. Date: Mar. 20, 2025

(54) METHODS OF TREATING HEMOPHILIA A

(71) Applicant: BIOVERATIV THERAPEUTICS INC., Waltham, MA (US)

(72) Inventors: Craig BENSON, Waltham, MA (US); Pronabesh DASMAHAPATRA, Waltham, MA (US); Suresh KATRAGADDA, Waltham, MA (US); Yunzhi LIN, Sandy, UT (US); Annemieke WILLEMZE, Waltham, MA (US); Amanda WILSON, Waltham, MA (US)

(21) Appl. No.: 18/819,571

(22) Filed: Aug. 29, 2024

Related U.S. Application Data
(63) Continuation of application No. PCT/US2023/063828, filed on Mar. 7, 2023.
(60) Provisional application No. 63/483,391, filed on Feb. 6, 2023.
(60) Provisional application No. 63/481,418, filed on Jan. 25, 2023.
(60) Provisional application No. 63/480,328, filed on Jan. 18, 2023.
(60) Provisional application No. 63/383,764, filed on Nov. 15, 2022.
(60) Provisional application No. 63/380,857, filed on Oct. 25, 2022.
(60) Provisional application No. 63/371,327, filed on Aug. 12, 2022.
(60) Provisional application No. 63/369,893, filed on Jul. 29, 2022.
(60) Provisional application No. 63/359,704, filed on Jul. 8, 2022.
(60) Provisional application No. 63/335,970, filed on Apr. 28, 2022.
(60) Provisional application No. 63/318,361, filed on Mar. 9, 2022.
(60) Provisional application No. 63/317,957, filed on Mar. 8, 2022.

(51) Int. Cl. C07K 14/755; A61K 9/00; A61K 38/00; A61P 7/04; A61P 29/02; C07K 14/00

(52) U.S. Cl. CPC C07K 14/755; A61K 9/0019; A61P 7/04; A61P 29/02; C07K 14/001; A61K 38/00; C07K 2319/30; C07K 2319/50

(57) ABSTRACT

The present disclosure provides a method of treating hemophilia A in a human subject in need thereof comprising administering to the subject a chimeric protein comprising (i) a factor VIII (FVIII) protein and (ii) a von Willebrand factor (VWF) fragment comprising a D′ domain of VWF and a D3 domain of VWF.

### RELATED APPLICATIONS

This application is a continuation of International Patent Application No. PCT/US2023/063828, filed Mar. 7, 2023, which claims the benefit of U.S. Provisional Patent Application Serial Nos. 63/317,957, filed Mar. 8, 2022; 63/318,361, filed Mar. 9, 2022; 63/335,970, filed Apr. 28, 2022; 63/359,704, filed Jul. 8, 2022; 63/369,893, filed Jul. 29, 2022; 63/371,327, filed Aug. 12, 2022; 63/380,857, filed Oct. 25, 2022; 63/383,764, filed Nov. 15, 2022; 63/480,328, filed Jan. 18, 2023; 63/481,418, filed Jan. 25, 2023; and 63/483,391, filed Feb. 6, 2023; all of which are incorporated by reference in their entirety for all purposes.

### REFERENCE TO SEQUENCE LISTING SUBMITTED ELECTRONICALLY

The content of the electronically submitted sequence listing in XML file (Name: 756173_SA9-487PCCON_ST26.txt; Size: 55,821 bytes; Date of Creation: Aug. 28, 2024) is incorporated herein by reference in its entirety.

### BACKGROUND OF THE DISCLOSURE

Hemophilia A is an X chromosome-linked bleeding disorder that occurs predominantly in males, and is characterized by deficiency of functional clotting Factor VIII (FVIII). The worldwide prevalence of hemophilia A is estimated to be 1 in 10,000 and worldwide incidence is approximately 1 in 5000 male births. The severity of disease is characterized by the endogenous level of FVIII measured in the plasma. Severe hemophilia A (<1% endogenous FVIII activity level; or <1 IU/dL) accounts for approximately 30% to 50% of all cases of hemophilia A (Aznar et al. Haemophilia. 2009; 15(3): 665-75.)

Individuals with severe hemophilia experience frequent bleeding episodes into major joints, soft tissue, and muscle, either spontaneously or following minor trauma. The disease can be acutely life-threatening. Repeated bleeding can lead to debilitating long-term complications, including hemophilic arthropathy from bleeding into the joints (Roosendall & Lafeber. Semin Thromb Hemost. 2003; 29(1): 37-42). Another severe complication is the development of target joints from inflammation due to prior bleeding. Intracranial hemorrhage can result in disability and death and is the leading cause of hemorrhagic death in individuals with hemophilia (Witmer et al. Br J Haematol. 2011. 152(2): 211-6.). Significant effects on physical and psychosocial well-being and quality of life and substantial financial burden have been reported in patients with severe hemophilia (Lee et al. J Korean Med Sci. 2016; 31(1): 33-8).

The current recommended standard of care involves the regular administration (routine prophylaxis) of FVIII to minimize the number of bleeding episodes. Routine prophylaxis has been associated with improvements in long-term outcomes, but is a demanding regimen limited by the need for frequent intravenous (IV) administration. See Manco-Johnson et al., N Engl J Med. 357(6):535-44 (2007). Extended half-life FVIII products have reduced the frequency of FVIII administration for prophylaxis; however, all interact with von Willebrand factor (VWF) and have comparable circulating half-lives, consistent with an upper limit on the half-life of rFVIII variants due to the half-life of endogenous VWF. See, e.g., Pipe et al., Blood. 128(16):2007-16 (2016). Prophylactic dosing for these FVIII products is every 3 to 5 days.

In addition to the patient burden that results from frequent administration, it is well established that the currently accepted FVIII activity trough level (1% to 3%) is not adequate to protect patients from all bleeds and the resulting morbidity associated with such bleeding episodes. Joint bleeds still occur at these levels, leaving patients susceptible to long-term morbidity (Soucie et al. Blood Adv. 2018; V.2: 2136-2144; Manco-Johnson et al. Blood. 2017; 2368-2374. The ability to increase patient protection by achieving higher sustained levels of factor activity has been remains a critical need for patients and follows recommendations from the World Federation of Hemophilia (Skinner M W. Haemophilia. 2012;18(Suppl 4):1-12).

There remains a need for improved clotting factor replacement products with extended half-life for use in the treatment of hemophilia patients. Next-generation extended half-life FVIII products that prevent and control bleeding episodes for longer periods of time, resulting in less frequent administration, would potentially address the challenges of adherence to demanding prophylactic regimens, which in turn could improve the quality of life for hemophilia patients.

### SUMMARY OF THE DISCLOSURE

Efanesoctocog alfa, also known as “BIW001”, “efanesoctocogum alfa”, “efa”, and “rFVIIIFc-VWF-XTEN”, is the first recombinant FVIII (rFVIII) engineered to be independent of VWF, extending its half-life and capacity to prevent and control bleeding episodes for longer periods of time. This reduces the burden of frequent intravenous (IV) administration for individuals with hemophilia A, and in turn improves adherence and outcomes, including quality of life.

Treatment of hemophilia A with efanesoctocog alfa has the potential to achieve and maintain higher factor activity levels in patients and with less frequent administration than currently available therapies.

Certain aspects of the present disclosure are directed to a method of treating hemophilia in a human subject in need thereof comprising administering to the subject multiple doses of a chimeric protein comprising (i) a factor VIII (FVIII) protein and (ii) a von Willebrand factor (VWF) fragment comprising a D′ domain of VWF and a D3 domain of VWF at a dosing interval, wherein at least one of the multiple doses is, e.g., from about 45 IU/kg to about 70 IU/kg and the dosing interval is at least about every 7 days. In some embodiments, the hemophilia is severe hemophilia A. In some embodiments, at least one of the multiple doses is about 50 IU/kg. In some embodiments each of the multiple doses is about 50 IU/kg.

In some embodiments, the treatment of hemophilia A comprises controlling or decreasing the incidence or frequency of bleeding episodes (such as treated bleeding episodes, e.g., treated bleeding episodes that are spontaneous bleeding episodes or traumatic bleeding episodes, as well as untreated bleeding episodes other than normal bleeding episodes) in a human subject in need thereof. In some embodiments, the treatment of hemophilia A comprises preventing or treating a bleeding episode (such as a treated bleeding episode, e.g., a treated bleeding episode that is a spontaneous bleeding episode or a traumatic bleeding episode, as well as an untreated bleeding episode other than a normal bleeding episode) in a human subject in need thereof.

In some embodiments, the dosing frequency is at least once every week. In some embodiments, the dosing frequency is once per week.

In some embodiments, the chimeric protein is administered as prophylactic treatment.

In some embodiments, the chimeric protein is administered as on-demand treatment.

Certain aspects of the present disclosure are directed to a method of achieving an annualized bleeding rate (ABR) of 2 or less, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

Certain aspects of the present disclosure are directed to a method of reducing an annualized bleeding rate (ABR) compared to treatment with a Factor VIII (FVIII) replacement protein that is capable of being bound by endogenous von Willebrand Factor (VWF), comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a VWF fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of reducing an annualized bleeding rate (ABR) compared to prophylactic treatment with a complex, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide sequence, (c) a thrombin-cleavable linker comprising at least a portion of the a2 region of FVIII, and (d) a second Fc region, wherein the first Fc region and the second Fc region are covalently attached to each other by at least one disulfide bond and wherein the complex comprises a FVIII protein and a wild-type VWF protein or a portion thereof, wherein the FVIII protein and the VWF protein or portion thereof are not directly or indirectly covalently attached to each other.

Certain aspects of the present disclosure are directed to a method of reducing an annualized bleeding rate (ABR) compared to prophylactic treatment with emicizumab, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide sequence, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of reducing an amount of pain medication needed to decrease or manage pain associated with hemophilia A, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

Certain aspects of the present disclosure are directed to A method of maintaining a FVIII activity level of at least 2%, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide sequence, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of reducing the risk of a bleeding episode that requires more than one supplemental on-demand administration of a FVIII replacement protein during the prophylactic treatment of hemophilia A, wherein the prophylactic treatment of hemophilia A comprises administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of resolving a bleeding episode, comprising administering to a human subject in need thereof a single on-demand dose of a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of prophylactically treating hemophilia A, the method comprising administering to a subject in need thereof a therapeutically effective amount of a chimeric protein, wherein a total of less than 3500 IU/kg/year, less than 3400 IU/kg/year, less than 3300 IU/kg/year, less than 3200 IU/kg/year, less than 3100 IU/kg/year, less than 3000 IU/kg/year, less than 2900 IU/kg/year, less than 2800 IU/kg/year, less than 2700 IU/kg/year, or about 2600 IU/kg/year of the chimeric protein is administered to the subject, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of prophylactically treating hemophilia A, comprising administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, wherein the subject does not administer any on-demand treatment for hemophilia A before engaging in strenuous activity.

Certain aspects of the present disclosure are directed to A method of improving quality of life with the prophylactic treatment of hemophilia A, comprising administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of improving one or more joint outcomes, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method of on-demand treatment of a minor bleed, a moderate bleed, or a major bleed, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a method for the perioperative management of bleeding, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

Certain aspects of the present disclosure are directed to a method of achieving an ABR of 2 or less, comprising administering to a human subject who has hemophilia A in need thereof 50 IU/kg of efanesoctocog alfa once weekly.

Certain aspects of the present disclosure are directed to a method of reducing an ABR compared to treatment with a FVIII replacement protein that is capable of being bound by endogenous VWF, comprising administering to a human subject who has hemophilia A in need thereof 50 IU/kg of efanesoctocog alfa once weekly.

Certain aspects of the present disclosure are directed to a method of reducing an ABR compared to prophylactic treatment with a complex, comprising administering to a human subject who has hemophilia A in need thereof a therapeutically effective amount of 50 IU/kg of efanesoctocog alfa once weekly, wherein the complex comprises a FVIII protein and a wild-type VWF protein or a portion thereof, wherein the FVIII protein and the VWF protein or portion thereof are not directly or indirectly covalently attached to each other.

Certain aspects of the present disclosure are directed to a method of reducing an ABR compared to prophylactic treatment with emicizumab, comprising administering to a human subject who has hemophilia A in need thereof 50 IU/kg of efanesoctocog alfa once weekly.

Certain aspects of the present disclosure are directed to a method of reducing an amount of pain medication needed to decrease or manage pain associated with hemophilia A, comprising administering to a human subject who has hemophilia A in need thereof 50 IU/kg of efanesoctocog alfa once weekly.

Certain aspects of the present disclosure are directed to A method of maintaining a FVIII activity level of at least 2%, comprising administering to a human subject who has hemophilia A in need thereof 50 IU/kg of efanesoctocog alfa once weekly.

Certain aspects of the present disclosure are directed to a method of reducing the risk of a bleeding episode that requires more than one supplemental on-demand administration of a FVIII replacement protein during the prophylactic treatment of hemophilia A, wherein the prophylactic treatment of hemophilia A comprises administering to a human subject in need thereof 50 IU/kg of efanesoctocog alfa once weekly.

Certain aspects of the present disclosure are directed to a method of resolving a bleeding episode, comprising administering to a human subject in need thereof a single on-demand dose of 50 IU/kg of efanesoctocog alfa.

Certain aspects of the present disclosure are directed to a method of prophylactically treating hemophilia A, the method comprising administering to a subject in need thereof a therapeutically effective amount of efanesoctocog alfa, wherein a total of less than 3500 IU/kg/year, less than 3400 IU/kg/year, less than 3300 IU/kg/year, less than 3200 IU/kg/year, less than 3100 IU/kg/year, less than 3000 IU/kg/year, less than 2900 IU/kg/year, less than 2800 IU/kg/year, less than 2700 IU/kg/year, or about 2600 IU/kg/year of efanesoctocog alfa is administered to the subject.

Certain aspects of the present disclosure are directed to a method of prophylactically treating hemophilia A, comprising administering to a human subject in need thereof 50 IU/kg of efanesoctocog alfa once weekly, wherein the subject does not administer any on-demand treatment for hemophilia A before engaging in strenuous activity.

Certain aspects of the present disclosure are directed to A method of improving quality of life with the prophylactic treatment of hemophilia A, comprising administering to a human subject in need thereof 50 IU/kg of efanesoctocog alfa once weekly.

Certain aspects of the present disclosure are directed to a method of improving one or more joint outcomes, comprising administering to a human subject who has hemophilia A in need thereof 50 IU/kg of efanesoctocog alfa once weekly.

Certain aspects of the present disclosure are directed to a method of on-demand treatment of a minor bleed, a moderate bleed, or a major bleed, comprising administering to a human subject who has hemophilia A in need thereof 50 IU/kg of efanesoctocog alfa.

Certain aspects of the present disclosure are directed to a method for the perioperative management of bleeding, comprising administering to a human subject who has hemophilia A in need thereof 50 IU/kg of efanesoctocog alfa.

Certain aspects of the present disclosure are directed to kits comprising a pharmaceutical label and a chimeric protein, wherein the pharmaceutical label comprises a recommendation or instruction for practicing a method disclosed herein, and wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to kits comprising a pharmaceutical label and a chimeric protein, wherein the pharmaceutical label comprises information that is sufficient to enable a human subject, a caregiver, or a medical practitioner to practice the method disclosed herein and wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in achieving an annualized bleeding rate (ABR) of 2 or less, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in reducing an annualized bleeding rate (ABR) compared to treatment with a Factor VIII (FVIII) replacement protein that is capable of being bound by endogenous VWF, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in reducing an annualized bleeding rate (ABR) compared to prophylactic treatment with a complex, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and wherein the complex comprises a FVIII protein and a wild-type VWF protein or a portion thereof, wherein the FVIII protein and the VWF protein or portion thereof are not directly or indirectly covalently attached to each other.

Certain aspects of the present disclosure are directed to a chimeric protein for use in reducing an annualized bleeding rate (ABR) compared to prophylactic treatment with emicizumab, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the disclosure are directed to a chimeric protein for use in reducing an amount of pain medication needed to decrease or manage pain associated with hemophilia A, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

Certain aspects of the disclosure are directed to a chimeric protein for use in maintaining a FVIII activity level of at least 2%, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in reducing the risk of a bleeding episode that requires more than one supplemental on-demand administration of a FVIII replacement protein during the prophylactic treatment of hemophilia A, wherein the prophylactic treatment of hemophilia A comprises administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in resolving a bleeding episode, comprising administering to a human subject in need thereof a single on-demand dose of a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in prophylactically treating hemophilia A, the method comprising administering to a human subject in need thereof a therapeutically effective amount of the chimeric protein, wherein a total of less than 3500 IU/kg/year, less than 3400 IU/kg/year, less than 3300 IU/kg/year, less than 3200 IU/kg/year, less than 3100 IU/kg/year, less than 3000 IU/kg/year, less than 2900 IU/kg/year, less than 2800 IU/kg/year, less than 2700 IU/kg/year, or about 2600 IU/kg/year of a chimeric protein is administered to the subject, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and wherein the chimeric protein is administered to the at a dose of 45 IU/kg to 70 IU/kg of a chimeric protein at a dosing interval of about 6 days to about 14 days.

Certain aspects of the present disclosure are directed to a chimeric protein for use in reducing the need to administer on-demand prior to strenuous activity in the prophylactic treatment of hemophilia A, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in improving quality of life with the prophylactic treatment of hemophilia A, wherein a therapeutically effective amount of the chimeric protein is administered to a human subject, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in improving one or more joint outcomes, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to a chimeric protein for use in the on-demand treatment of a minor bleed, a moderate bleed, or a major bleed, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

Certain aspects of the present disclosure are directed to chimeric protein for use in the perioperative management of bleeding, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

### BRIEF DESCRIPTION OF THE DRAWINGS/FIGURES

FIG. 1 is a non-limiting schematic representation of an exemplary FVIII-ELNN-Fc/D′D3-ELNN-Fc heterodimer. Abbreviations are FVIII: factor VIII; VWF: von Willebrand Factor; A1, A2, A3, C1, C2: domains of FVIII; D′D3: domains of VWF; Fc: Fc region of immunoglobulin constant region.

FIG. 2 is a graphical representation of the design of the Phase 3 clinical study disclosed in Example 1.

FIG. 3 is a graphical representation showing that efanesoctocog alfa weekly prophylaxis provided highly effective prevention against bleeds in the Phase 3 study described in Example 1. The ABR is for treated bleeding episodes.

FIG. 4 is a graphical representation showing that efanesoctocog alfa prophylaxis in the Phase 3 study described in Example 1 provided superior bleed protection compared to pre-study prophylaxis (242HA201/OBS16221) (n=78). The ABRs are for treated bleeding episodes.

FIG. 5 is a graphical representation of the distribution of annualized bleeding rate (ABR) and annualized joint bleeding rate (AjBR) for study subjects in Arm A of Example 1. The ABRs and AjBRs are for treated bleeding episodes.

FIG. 6 is a graphical representation of the bleeding event rate per study week in Arm A. The analysis includes treated bleeds with non-missing bleed dates (n=79) up to Week 52 in Arm A. Bleeding episodes are mapped to each week based on event date and time. Bleeding event rates are calculated as number of bleeds per participant week. A linear regression model was used to determine the slope and 95% confidence interval (CI) of the slope.

FIG. 7 is a graphical representation of the time after the last prophylaxis dose to spontaneous bleeding episode (A), and traumatic bleeding episode (B) in Arm A. For traumatic bleeding episodes, one traumatic bleeding episode (not shown) occurred 13 days after the last prophylaxis dose, and one traumatic bleeding episode (not shown) occurred during screening and thus had no prior prophylaxis dose.

FIG. 8 is a graphical representation of the intra-participant comparison of weekly injection frequency (A) and weekly FVIII consumption (B) for subjects receiving conventional standard-of-care FVIII prophylaxis (pre-study) or efanesoctocog alfa prophylaxis (on-study).

FIG. 9 is a graphical representation of the observed mean ABR, showing that efanesoctocog alfa prophylaxis reduced bleeding events after switching from efanesoctocog alfa on-demand treatment in Arm B of the Phase 3 study described in Example 1. The ABRs are for treated bleeding episodes.

FIG. 10 is a graphical representation of measurements of physical health according to Haem-A-QoL score, pain assessment according to PROMIS 3a Q1, and joint health according to Hemophilia Joint Health Score (HJHS). Efanesoctocog alfa prophylaxis demonstrated significant improvement in physical health, pain, and joint health.

FIG. 11 is a graphical representation of the LS mean change from baseline to Week 52 in Haem-A-QoL domains and Total score in Arm A. Nominal p-values except for the Physical Health domain score. ** p:0.0001, * p<0.05. For physical health, pre-specified endpoint included in the multiplicity procedure; all other domains were analyzed post hoc. Lower scores denote better HRQoL. Abbreviations: CI, confidence interval; Haem-A-QoL: Haemophilia Quality of Life Questionnaire for Adults; HRQoL: health-related quality of life; LS: least squares.

FIG. 12 is a graphical representation of the observed mean change from baseline to Week 52 in Haem-A-QoL domains and Total score in Arm B. Lower scores denote better health related quality of life. All domains and Total score were exploratory endpoints. Abbreviations: Haem-A-QoL: Haemophilia Quality of Life Questionnaire for Adults; HRQoL: health-related quality of life; SD: standard deviation.

FIG. 13 is a graphical representation of the proportion of patients with improvements, no change, or worsening from baseline to Week 52 in EuroQol 5-dimension 5-level (EQ-5D-5L) dimensions in Arm A. Each dimension was scored by patients as one of five levels of perceived problems: 1=‘no problems’, 2=‘slight problems’, 3=‘moderate problems’, 4=‘severe problems’ and 5=‘extreme problems or unable’. For each EQ-5D-5L dimension a patient was considered to have improved if they improved at least 1 category; a patient was considered to have worsened if they worsened at least 1 category.

FIG. 14 is a visual representation of the endpoints met in the Phase 3 study outlined in Example 1.

FIG. 15 is a line graph displaying the mean Factor VIII (FVIII) activity level (IU/dL, %) over 14 days following a dose of efanesoctocog alfa at week 1 (circle line) or at week 26 (triangle line). Once weekly efanesoctocog alfa provided mean factor VIII activity levels >40% for up to 4 days and >10% at Day 7.

FIG. 16 is a visual representation of the timelines of perioperative management for each major surgery in the study. Abbreviations: TKP, Total knee prosthesis. Only factor activity measurements from central laboratory are shown. On the day of surgery, in all instances, the surgery followed factor activity measurement and efanesoctocog alfa dosing. Footnotes: (a) Orthopedic surgery. (b) Two surgeries in the same patient.

FIG. 17 is a graphical representation of the design of the Phase 1 study disclosed in Example 2. Samples for FVIII inhibitor testing were obtained before administering each rFVIII product and at 14 and 28 days after efanesoctocog alfa was administered.

FIG. 18 is a line graph displaying the mean (±SD) plasma FVIII activity level (IU/dl, %) after administration of a single dose of octocog alfa (Advate®) at 50 IU/kg (circles), rurioctocog alfa (Adynovi®) at 50 IU/kg (squares), or efanesoctocog alfa at 50 IU/kg (triangles), as outlined in Example 2.

FIG. 19 includes a graphical representation showing that efanesoctocog alfa provides high sustained FVIII levels throughout the weekly dosing interval, as assessed in the study of Example 1.

FIG. 20 is a graph showing the cumulative Distribution Function of Change from Baseline in Haem-A-QoL Physical Health Score at Week 52 in Arm A

FIG. 21 is a graph showing the cumulative distribution function of change from baseline in pain for PROMIS Pain intensity 3a first item (at its worst) scores at Week 52 for Arm A.

FIG. 22 is a graph showing shift analyses for PROMIS Pain intensity first item 3a (pain at its worst) for Arm A. Only patients who provided a score at each visit were considered in this graph.

FIG. 23 is a graph showing pain medication use at baseline and at week 52 of the study of Example 1. Depicted are the number of days pain medication related to hemophilia was administered within 2 weeks of the visit (n=159). Percentages were based on the number of participants in the safety analysis. Assessments during major surgical periods were excluded. The maximal day of taking pain medication related to hemophilia was counted if there were more than 1 pain medication reported.

FIGS. 24A-24C. FIG. 24A is a graph and additional disclosures showing that efanesoctocog alfa prophylaxis in the study of Example 1 provided highly effective protection against bleeds, superior to prior FVIII therapy. ABR, annualized bleed rate; CI, confidence interval; FVIII, factor VIII; IQR, interquartile range. Footnotes: (a) The CI of the mean ABR was estimated using a negative-binomial model with the total number of treated bleeding episodes during the efficacy period as the response variable and log-transformed efficacy period duration (in years) as an offset variable. (b) Estimated using a negative binomial regression model with treatment (efanesoctocog alfa prophylaxis versus prestudy FVIII prophylaxis) as covariate. (c) P value relates to the null hypothesis that the rate ratio of efanesoctocog alfa prophylaxis/prestudy prophylaxis is equal to 1. Abbreviations: ABR, annualized bleed rate; CI, confidence interval; FVIII, factor VIII; IQR, interquartile range. FIGS. 24B-C are graphs showing that low bleed rates were observed after a switch to efanesoctocog alfa prophylaxis in both study arms of the study of Example 1. ABR, annualized bleed rate; SD, standard deviation. Footnotes: (a) Based on treated bleeds. (b) Mean (SD) estimated number of bleeds in the 12 months prior to the study was 3.2 (5.4). (c) Mean (SD) estimated number of bleeds in the 12 months prior to the study was 35.7 (22.2).

FIG. 25 is a line graph displaying the mean Factor VIII (FVIII) activity level (IU/dL) over 14 days (168 hr) following a dose of efanesoctocog alfa in all participants <12 years of age.

FIG. 26 is a graph displaying the change in Hemophilia Joint Health Score (HJHS) total score from baseline measurement to Week 52 in participants in Arm A (weekly prophylaxis) of the Phase 3 open-label, multicenter study, separated by age. LS mean (95% CI), and P value estimated by mixed-effect model with repeated measures with visit as a fixed effect, and baseline HJHS as covariate. Includes only patients with HJHS measurements at both timepoints.

FIGS. 27A-27B are graphs displaying the change in HJHS score from baseline measurement to Week 52 in Arm A (FIG. 27A) and Arm B (FIG. 27B) of the Phase 3 open-label, multicenter study, separated by HJHS domain. HJHS assessments within 2 weeks after a joint or muscle bleed were excluded. Joint scores post joint surgeries were replaced using the last observation carried forward method. Assessments during other major surgical periods were excluded. HJHS domains are assessed on the following ranges of scores: swelling (0-3); duration of swelling (0 or 1); muscle atrophy (0-2); crepitus on motion (0-2); flexion loss (0-3); extension loss (0-3); joint pain (0-2); strength (0-4). The domain score was calculated if all 6 joints for each domain were present.

FIG. 28 is a graph showing the estimated mean annualized bleed rates (ABR) for all participants (n=74) in the completed study disclosed in Example 3. Data shown is for all participants (overall) and for each of the indicated age groups. ABR, annualized bleed rate; CI, confidence interval.

FIG. 29 is a graphical representation of the distribution of annualized bleeding rate (ABR), annualized joint bleeding rate (AjBR), and annualized spontaneous bleeding rate (AsBR) for all study participants (n=74) in the completed study disclosed in Example 3.

FIG. 30 is a line graph displaying the mean Factor VIII (FVIII) activity level (IU/dL, %) over 7 days following the first dose of efanesoctocog alfa for participants under 6 years old (circle line) or 6 to 12 years old (triangle line). Data shown is from the PK Analysis Set, n=19 (<6 years old) and 18 (6-<12 years old).

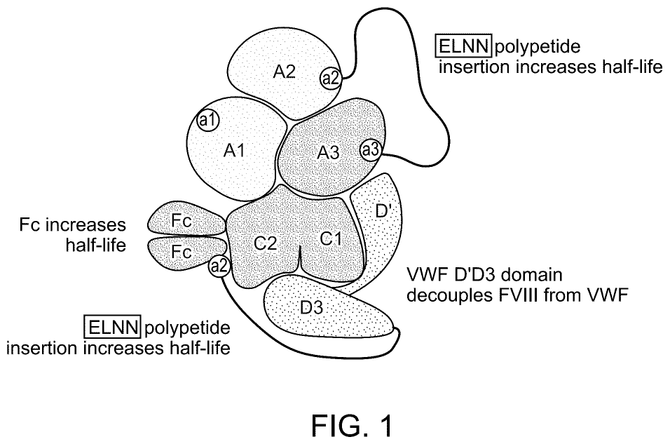

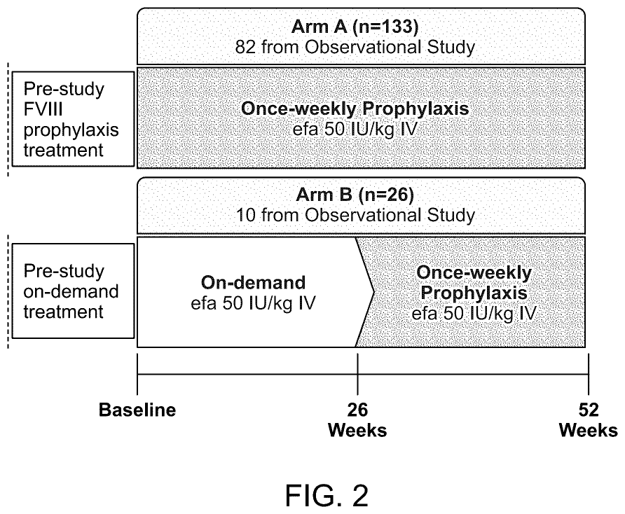

### DETAILED DESCRIPTION

The present disclosure is directed to methods of treating hemophilia A. In some embodiments, these methods comprise use of a chimeric protein such as efanesoctocog alfa, which comprises two polypeptides, i.e., a first polypeptide comprising a FVIII polypeptide comprising a first ELNN polypeptide sequence insert fused to a first Fc region, and a second polypeptide comprising a VWF fragment (e.g., a VWF fragment comprising mutations as disclosed herein) fused to a second Ig constant region by a second ELNN polypeptide sequence, wherein the first ELNN polypeptide sequence contains about 280 to 300 amino acids (e.g., about 288 amino acids) and the second ELNN polypeptide sequence contains about 140 to 150 amino acids (e.g., about 144 amino acids), and the first Ig constant region and the second Ig constant region are covalently linked together by disulfide bonds.

In some embodiments, the present disclosure is directed to methods of treating hemophilia A with chimeric protein comprising (i) a factor VIII (FVIII) polypeptide and (ii) a von Willebrand factor (VWF) fragment comprising a D′ domain of VWF and a D3 domain of VWF (e.g., a D′D3 region that may be mutated to substitute cysteines involved in dimerization). Also disclosed are pharmaceutical compositions comprising the chimeric protein. Also disclosed are uses of the chimeric protein or pharmaceutical composition disclosed herein for treating hemophilia A.

In some embodiments, the chimeric protein is efanesoctocog alfa.

### I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, useful descriptions relating to certain terms in the field of hemophilia A may be found in Blanchette et al. J Thromb Haemost. 2014; 12(11):1935-9, the entire contents of which are incorporated herein by reference.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

In some embodiments, a polynucleotide is an isolated nucleic acid molecule or construct, e.g., messenger RNA (mRNA) or plasmid DNA (pDNA). Depending on context, a “polynucleotide” may be any one or more nucleic acid segments, e.g., DNA or RNA fragments, present in a larger polynucleotide, such as a genome or vector. By “isolated” nucleic acid or polynucleotide is intended a nucleic acid molecule, DNA or RNA, which is not in its natural milieu (e.g., it is not within a wild-type cell or virus). For example, a recombinant polynucleotide encoding a Factor VIII protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or full-length polypeptide to produce a secreted or mature form of the polypeptide. In some embodiments, a native signal peptide or a functional derivative of that sequence that retains the ability to direct the secretion of the polypeptide that is operably associated with it. It will be understood that, in instances where a polypeptide sequence is disclosed with a signal peptide, the form of the polypeptide sequence without the signal peptide is also disclosed.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, but does not refer to any particular amino acid length. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms, depending on context. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions, or additions.

The term “VWF fragment” includes any VWF fragment that interacts with FVIII and retains at least one or more properties that are normally provided to FVIII by full-length VWF, e.g., preventing or reducing premature activation to FVIlla, preventing or reducing premature proteolysis, preventing or reducing clearance, preventing or reducing association with phospholipid membranes that could lead to premature clearance, preventing or reducing binding to FVIII clearance receptors that can bind naked FVIII but not VWF-bound FVIII, and/or stabilizing the FVIII heavy chain and light chain interactions. A VWF fragment referred to herein is a VWF polypeptide that is less than the full-length VWF protein, wherein the VWF fragment retains the ability to interact with and/or bind to FVIII. In some embodiments, a VWF fragment is a fragment (which may be mutated) of full-length VWF that binds to a FVIII polypeptide such that the FVIII polypeptide has reduced binding to, or does not bind, full length VWF (e.g., endogenous VWF in a subject). In some embodiments, the VWF fragment is a human VWF fragment, or a mutant VWF fragment.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In some embodiments, a string of amino acids can be conservatively replaced with a structurally similar string that differs in order and/or composition of side chain family members.

As known in the art, “sequence identity” between two polypeptides is determined by comparing the amino acid sequence of one polypeptide to the sequence of a second polypeptide. Similarly, “sequence identity” between two polynucleotides is determined by comparing the nucleotide sequence of one polynucleotide to the sequence of a second polynucleotide. The terms “% identical”, “% identity” or similar terms are intended to refer, in particular, to the percentage of nucleotides or amino acids (as applicable) which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. For example, the optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g, at http://blast.ncbi.nlm.nih.gov/Blast.cgi). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment. When discussed herein, whether any particular polypeptide is, e.g., at least about 50%, 60%, 70%, 75%, 80%, 85%, 90%, 95%, 99%, or 100% identical to another polypeptide, can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, WI 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present disclosure, the parameters are set such that the percentage of identity is calculated over the full-length of the reference polypeptide sequence and that gaps in homology of up to 5% of the total number of amino acids in the reference sequence are allowed.

As used herein, an “amino acid corresponding to” or an “equivalent amino acid” in a VWF sequence or a FVIII sequence is identified by alignment to maximize the identity or similarity between a first VWF or FVIII sequence and a second VWF or FVIII sequence. The number used to identify an equivalent amino acid in a second VWF or FVIII sequence is based on the number used to identify the corresponding amino acid in the first VWF or FVIII sequence.

As used herein, the term “insertion site” refers to a position in a FVIII polypeptide, or fragment, variant, or derivative thereof, which is immediately downstream of the position at which a half-life extending moiety or heterologous moiety can be inserted. An “insertion site” is specified as a number, the number being the number of the amino acid in mature native (wild type) human FVIII (SEQ ID NO: 8) to which the insertion site corresponds, which is immediately C-terminal to the position of the insertion. For example, the phrase “comprises an ELNN polypeptide at an insertion site which corresponds to amino acid 1656 of SEQ ID NO: 8” indicates that the heterologous moiety is inserted immediately after the amino acid residue corresponding to amino acid residue 1656 of SEQ ID NO: 8 (without requiring amino acid residue 1657 of SEQ ID NO: 8 to be present).

The terms “inserted,” “is inserted,” “inserted into” or grammatically related terms, as used herein with respect to insertions of ELNN polypeptide into FVIII refers to the position of an ELNN polypeptide in a chimeric protein relative to the analogous position in native mature human FVIII (SEQ ID NO: 8). As used herein the terms refer to the characteristics of the recombinant FVIII polypeptide relative to native mature human FVIII, and do not indicate, imply or infer any methods or process by which the chimeric protein was made. For example, in reference to a chimeric protein provided herein, the phrase “an ELNN polypeptide is inserted immediately downstream of residue 745 of the FVIII polypeptide” means that the chimeric protein comprises an ELNN polypeptide immediately downstream of an amino acid residue which corresponds to amino acid residue 745 in native mature human FVIII, e.g., bounded by amino acids corresponding to amino acid residues 745 and 746 of native mature human FVIII (without requiring the presence of an amino acid residue corresponding to 746 of native mature human FVIII), and does not connote an order or method of production for which the chimeric protein was constructed.

As used herein, the terms “ELNN polypeptide” and “ELNN” are synonymous, and refer to extended length polypeptides comprising non-naturally occurring, substantially non-repetitive sequences (e.g., polypeptide motifs) that are composed mainly of small hydrophilic amino acids, with the sequence having a low degree or no secondary or tertiary structure under physiologic conditions. Such extended length polypeptides include unstructured hydrophilic polypeptides comprising repeating motifs of 6 natural amino acids (G, A, P, E, S, and/or T). In some embodiments, an ELNN polypeptide comprises multiple motifs of 6 natural amino acids (G, A, P, E, S, T), wherein the motifs are the same or comprise a combination of different motifs. ELNN polypeptides can confer certain desirable pharmacokinetic, physicochemical and pharmaceutical properties when linked to a VWF fragment or a FVIII sequence of the disclosure to create a chimeric protein. Such desirable properties include but are not limited to enhanced pharmacokinetic parameters and solubility characteristics. ELNN polypeptides are known in the art, and non-limiting descriptions relating to and examples of ELNN polypeptides known as XTEN® polypeptides are available in Schellenberger et al., (2009) Nat Biotechnol 27(12):1186-90; Brandl et al., (2020) Journal of Controlled Release 327:186-197; and Radon et al., (2021) Advanced Functional Materials 31, 2101633 (pages 1-33), the entire contents of each of which are incorporated herein by reference.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an immunoglobulin Fc domain. A fusion protein is created, for example, by chemical synthesis, or by creating and translating a polynucleotide in which the peptide regions are encoded in the desired relationship. A chimeric protein can further comprise a second amino acid sequence associated with the first amino acid sequence by a covalent, non-peptide bond or a non-covalent bond.

With respect to sequences, the term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. Depending on context, the term “linked” means not only a fusion of a first amino acid sequence to a second amino acid sequence at the C-terminus or the N-terminus, but also includes insertion of the whole first amino acid sequence (or the second amino acid sequence) at an insertion site of the second amino acid sequence (or the first amino acid sequence, respectively). In some embodiments, the first amino acid sequence can be linked to a second amino acid sequence by a peptide bond or a linker. In some embodiments, a first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” may also be indicated by a hyphen (-).

With respect to two polypeptides, the term “associated with” refers to one or more covalent or non-covalent bonds formed between a first polypeptide and a second polypeptide. In some embodiments, the term “associated with” means a covalent, non-peptide bond or a non-covalent bond. This association can be indicated by a colon, i.e., (:). In some embodiments, it means a covalent bond except a peptide bond. For example, the amino acid cysteine comprises a thiol group that can form a disulfide bond or bridge with a thiol group on a second cysteine residue. In many naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity. In some embodiments, the one or more covalent bonds between the first amino acid chain and the second amino acid chain is two disulfide bonds. In some embodiments, the one or more covalent bonds between the first amino acid chain and the second amino acid chain is two disulfide bonds between a first Fc portion on the first amino acid chain and a second Fc portion on the second amino acid chain, wherein the two disulfide bonds occur in the hinge region of the two Fc portions.

In some embodiments, a polypeptide has an enzymatic cleavage site cleaved by an enzyme that is activated during the clotting cascade, such that cleavage of such sites occurs at the site of clot formation. Exemplary such sites include, e.g., those recognized by thrombin, Factor Xla or Factor Xa. Other enzymatic cleavage sites are known in the art and described in elsewhere herein. In constructs that include more than one processing or cleavage site, it will be understood that such sites can be the same or different.

In some embodiments, half-life can be represented by the time required for half the quantity administered to a subject to be cleared from the circulation and/or other tissues in the animal. In some embodiments, when a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, FVIII and chimeric proteins comprising FVIII are monophasic, and thus do not have an alpha phase, but just the single beta phase. Therefore, in some embodiments, the term half-life as used herein refers to the half-life of the polypeptide in the β-phase. In some embodiments, the half-life is expressed as the half-life of the terminal phase.

The composition, e.g., the chimeric protein, can be administered to a subject using methods known in the art. In some embodiments, the administration is intravenous. In some embodiments, the administration is subcutaneous. In some embodiments, the administration is self-administration. In some embodiments, a parent administers the chimeric protein to a child. In some embodiments, the chimeric protein is administered to a subject by a healthcare practitioner, such as a medical doctor, a medic, or a nurse.

In some embodiments, a single dose is administered to a subject. In some embodiments, multiple doses are administered to a subject. A single dose can be administered all at once, e.g., as a bolus, or over a period of time, e.g., via an intravenous infusion.

In some embodiments, the composition, e.g., the chimeric protein, is delivered via a slow push IV injection of 8±2 minutes. In some embodiments, the composition is delivered at a rate of administration determined by the subject's comfort level. In some embodiments, the composition is delivered via a slow push IV injection at a rate of administration determined by the subject's comfort level and according to the following vial injection rate recommendations: For subjects weighing 555 kg, the minimum injection duration per vial is 2 minutes per vial; for subjects weighing >55 kg, the minimum injection duration per vial is 1 minute per vial.

When referring to co-administration of more than one composition, a dose of composition A can be administered concurrently with a dose of composition B. Alternatively, a dose of composition A can be administered before or after a dose of composition B. In some embodiments, composition A and composition B are combined into a single formulation.

As used herein, the term “interval” or “dosing interval” refers to the amount of time that elapses between a first dose of composition A and a subsequent dose of the same composition administered to a subject. A dosing interval can refer to the time that elapses between multiple doses.

The term “dosing frequency” as used herein refers to the number of doses administered per a specific dosing interval. For example, a dosing frequency can be written as once a week, once every two weeks, etc.

As used herein in the context of hemophilia A, the term “prophylactic treatment” refers to the administration of a therapy for the treatment of hemophilia A, where such treatment is intended to prevent or reduce the severity of one or more symptoms of hemophilia A, e.g., bleeding episodes, such as one or more spontaneous bleeding episodes, and/or joint damage. To prevent or reduce the severity of such symptoms, e.g., bleeding episodes and the progression of joint disease, hemophilia A patients may receive regular infusions of clotting factor as part of a prophylactic treatment regimen.

A “prophylactic” treatment can also refer to the preemptive administration of the composition described herein, e.g., a protein (such as a chimeric protein), to a subject in order to control, manage, prevent, or reduce the occurrence or severity of one or more symptoms of hemophilia A, e.g., bleeding episodes. In some embodiments, prophylactic treatment with a clotting factor, e.g., FVIII, is used to treat subjects with severe hemophilia A. In some embodiments, prophylactic treatment refers to administering a composition disclosed herein to a subject in need thereof to reduce the occurrence of one or more symptoms of hemophilia A.

The term “on-demand treatment” or “episodic treatment” refers to the “as needed” administration of a FVIII replacement therapy (such as a chimeric protein) in response to symptoms of hemophilia A, e.g., a bleeding episode (such as a spontaneous bleeding episode or a traumatic bleeding episode), or before an activity that can cause bleeding. In some embodiments, the on-demand treatment can be given to a subject when bleeding starts, such as after an injury, or when bleeding is expected, such as before surgery. In some embodiments, the on-demand treatment can be given prior to activities that increase the risk of bleeding, such as contact sports. In some embodiments, on-demand treatment can be administered to a subject who is receiving prophylactic treatment, e.g., if supplemental FVIII replacement protein doses are administered to treat a bleeding episode or before strenuous activity. In some embodiments, the on-demand treatment is given as a single dose. In some embodiments, the on-demand treatment is given as a first dose, followed by one or more additional doses. In some embodiments, when the chimeric protein disclosed herein is administered on-demand, the one or more additional doses can be administered at least about 12 hours, at least about 24 hours, at least about 36 hours, at least about 48 hours, at least about 60 hours, at least about 72 hours, at least about 84 hours, at least about 96 hours, at least about 108 hours, or at least about 120 hours after the first dose. It should be noted, however, that the dosing interval associated with on-demand treatment is not the same as the dosing interval used for prophylactic treatment.

“Treat”, “treatment”, “treating”, as used herein in the context of hemophilia A includes, e.g., the reduction in severity of hemophilia A; the amelioration of one or more symptoms associated with hemophilia A; the provision of beneficial effects to a subject with hemophilia A, without necessarily curing the hemophilia A; and/or the prophylaxis of one or more symptoms associated with hemophilia A.

A “treated bleeding episode” is defined as a bleeding episode that requires administration of an on-demand dose of FVIII replacement treatment, and starts from the first sign of bleeding and ends no more than 72 hours after the last dose to treat the bleeding episode. Any subsequent bleeding at the same location and doses administered 572 hours from the previous dose are considered as the same bleeding episode. Multiple bleeding locations treated with a given dose are considered part of the same bleeding episode. Any dose to treat a bleeding episode that is administered >72 hours after the preceding dose for a bleeding episode for a given location or locations is considered the first dose to treat a new bleeding episode in the same location or locations, as applicable. Any dose used to treat new bleeding at a different location (or set of locations) is considered a dose to treat a separate bleeding episode, regardless of the time from the last dose to treat an existing bleeding episode.

If a bleeding episode occurs on the same day as a regularly scheduled prophylaxis dose, then: (1) the dose that would have been considered a prophylaxis dose is instead considered an on-demand dose (as are any additional doses given on the same day) for the bleeding episode for the purpose of assessing the number and/or duration of treated bleeding episodes, if the bleeding episode would require FVIII replacement treatment to stop the bleeding; and (2) if the bleeding episode would not require FVIII replacement treatment to stop the bleeding, the bleeding episode is considered as an untreated bleeding episode. In this case the prophylaxis dose administered as scheduled is not considered an on-demand dose.

Bleeding episodes or hemorrhages are classified as either spontaneous or traumatic. “Spontaneous bleeding episodes” are bleeding episodes that occur when there is no known contributing factor, such as a definite trauma or antecedent strenuous activity. “Traumatic bleeding episodes” are bleeding episodes that occur when there is a known or believed reason for the bleed. For example, if a participant exercised strenuously and then had a bleeding episode in the absence of any obvious injury, the bleeding episode is still recorded as traumatic. Target joint bleeding episodes can be traumatic if a known action led to bleeding into the joint.

A “spontaneous treated bleeding episode” is a bleeding episode that is both a spontaneous bleeding episode and a treated bleeding episode. A “traumatic treated bleeding episode” is a bleeding episode that is both a traumatic bleeding episode and a treated bleeding episode.

An “untreated spontaneous bleeding episode” is a bleeding episode that does not require an on-demand dose and occurs when there is no known contributing factor, such as a definite trauma or antecedent strenuous activity. A “untreated traumatic bleeding episode” is a bleeding episode that is untreated and occurs when there is a known or believed reason for the bleed.

As used herein, both “spontaneous” and “traumatic” bleeding excludes bleeding during or resulting from surgery.

“Normal bleeds” are instances of bleeding that would be expected to occur as a result of an injury in a subject who does not have a bleeding disorder. Non-limiting examples of typical cause bleeds include wounds, cuts, and lacerations.

In the context of treating hemophilia A, a “therapeutically effective amount” is an amount that, when administered to a subject for the treatment of hemophilia A, is sufficient to achieve a desired therapeutic effect.

A “pharmaceutical label” is “a display of written matter upon a container or printed medium that comprises statements about the administration, efficacy, and/or safety of a therapeutic agent such as a chimeric protein or a pharmaceutical composition comprising a chimeric protein.”

### II. Chimeric Proteins

In an aspect, the present disclosure is directed to methods of treating hemophilia A with a chimeric protein comprising a first polypeptide which comprises a Factor VIII (“FVIII”) protein or a portion thereof and a first immunoglobulin (“Ig”) constant region or a portion thereof, and a second polypeptide which comprises a von Willebrand Factor (“VWF”) fragment and a second Ig constant region or a portion thereof. In some embodiments, the chimeric protein comprises (i) a first polypeptide comprising a FVIII polypeptide, an ELNN polypeptide inserted within the B domain of the FVIII protein (e.g. replacing a portion of the B domain, such as the majority of the B domain), and a first Fc region; and (ii) a second polypeptide comprising a VWF fragment, a second ELNN polypeptide sequence, an a2 linker, and a second Fc region. In some embodiments, the chimeric protein disclosed herein is a FVIII-ELNN-Fc/D′D3-ELNN-Fc heterodimer.

In some embodiments, the chimeric protein is efanesoctocog alfa. Efanesoctocog alfa, also known as “BIVV001”, “efanesoctocogum alfa”, “efa”, and “rFVIIIFc-VWF-XTEN”, is described in Chhabra et al. Blood 2020; 135(17): 1484-1496, Konkle et al., N Eng J Med 2020; 383:1018-1027, and the International Nonproprietary Names for Pharmaceutical Substances (INN) WHO Drug Information, 2019, Vol. 33, No. 4, p.828-30, the entire contents of each of which are hereby incorporated by reference in their entireties. Efanesoctocog alfa is an exemplary FVIII-ELNN-Fc/D′D3-ELNN-Fc heterodimer. Efanesoctocog alfa temporarily replaces the missing FVIII needed for effective hemostasis in patients with a deficiency of FVIII. Efanesoctocog alfa is the first FVIII therapy that is engineered to circulate independently from VWF, thereby uncoupling FVIII from VWF clearance and extending half-life.

In some embodiments, the chimeric protein is a FVIII-ELNN-Fc/D′D3-ELNN-Fc heterodimer comprising (i) a first polypeptide comprising the amino acid sequence of SEQ ID NO: 1 and (ii) a second polypeptide comprising the amino acid sequence of SEQ ID NO: 2. In some embodiments, the chimeric protein comprises (i) a first polypeptide and (ii) a second polypeptide that are covalently linked via one or more disulfide bonds (e.g., two disulfide bonds). In some embodiments, the chimeric protein comprises a FVIII polypeptide encoded by the nucleic acid sequence of SEQ ID NO: 4. In some embodiments, the chimeric protein comprises a VWF fragment encoded by the nucleic acid sequence of SEQ ID NO: 6.

In some embodiments, the chimeric protein is efanesoctocog alfa. In some embodiments, the efanesoctocog alfa has a FVIII activity of at least 1600 IU/mg. In some embodiments, the efanesoctocog alfa has an activity of at least 1700 IU/mg. In some embodiments, the efanesoctocog alfa has an activity of at least 1800 IU/mg. In some embodiments, the efanesoctocog alfa has an activity of at least 1900 IU/mg. In some embodiments, the efanesoctocog alfa has an activity of 1600 IU/mg to 2000 IU/mg.

In some embodiments, the chimeric protein comprises a FVIII polypeptide comprising the amino acid sequence of SEQ ID NO: 1. In some embodiments, the chimeric protein comprises a FVIII polypeptide comprising one or more disulfide bridges at one or more of the following locations: residues 153-179, 248-329, 528-554, 630-711, 1220-1246, 1287-1291, 1409-1557, 1562-1714, 1761-1821, and/or 1867-1925 of SEQ ID NO: 1. In some embodiments, the chimeric protein comprises a FVIII polypeptide comprising one or more disulfide bridges at each of the following locations: residues 153-179, 248-329, 528-554, 630-711, 1220-1246, 1287-1291, 1409-1557, 1562-1714, 1761-1821, and 1867-1925 of SEQ ID NO: 1. In some embodiments, the chimeric protein comprises a FVIII polypeptide comprising one or more Cys-SH residues at residues 310, 692, and/or 1388 of SEQ ID NO: 1. In some embodiments, the chimeric protein comprises a FVIII polypeptide comprising a Cys-SH residues at each of residues 310, 692, and/or 1388 of SEQ ID NO: 1.

In some embodiments, the chimeric protein comprises a FVIII polypeptide comprises one or more N-glycosylation sites at residues N41, N239, N1198, N1506, and/or N1797 of SEQ ID NO: 1. In some embodiments, the chimeric protein comprises a FVIII polypeptide comprises one or more O-glycosylation sites at residues 746-1036 of SEQ ID NO: 1 and/or the Ser and Thr residues in the linker peptides. In some embodiments, the chimeric protein comprises a FVIII polypeptide comprises one or more Tyr-sulfation sites at residues 346, 718, 719, 723, 729, 1052, and/or 1068 of SEQ ID NO: 1.

In some embodiments, the chimeric protein comprises the VWF fragment comprising the amino acid sequence of the D′D3 sequence within the amino acid sequence of SEQ ID NO: 2. In some embodiments, the chimeric protein comprises a VWF fragment comprising one or more disulfide bridges at one or more of the following locations: residues 4-45, 13-41, 25-36, 29-64, 47-58, 66-88, 83-100, 86-95, 104-233,126-268, 135-230, 151-158, 283-326, 297-321, 308-348, 328-334, 338-363, 367-410, 386-406, 390-402, 394-433, 414-427, 436-464, 459-474, 462-471, 698-758, and/or 804-862 of SEQ ID NO: 2. In some embodiments, the chimeric protein comprises a VWF fragment comprising one or more disulfide bridges at each of the following locations: residues 4-45, 13-41, 25-36, 29-64, 47-58, 66-88, 83-100, 86-95, 104-233, 126-268, 135-230, 151-158, 283-326, 297-321, 308-348, 328-334, 338-363, 367-410, 386-406, 390-402, 394-433, 414-427, 436-464, 459-474, 462-471, 698-758, and/or 804-862 of SEQ ID NO: 2.

In some embodiments, the chimeric protein comprises a VWF fragment that comprises one or more N-glycosylation sites at residues N94, N384, N734 of SEQ ID NO: 2. In some embodiments, the chimeric protein comprises a VWF fragment comprising one or more O-glycosylation sites at residues 478-625 of SEQ ID NO: 2 and/or the Ser and Thr residues in the linker peptides. In some embodiments, the chimeric protein comprises a VWF fragment comprising one or more Tyr-sulfation sites at residues 632, 633, 637, and/or 643 of SEQ ID NO: 2.

In some embodiments, a chimeric protein comprises a polypeptide chain comprising a D′ domain of VWF, a D3 domain of VWF, an ELNN polypeptide sequence comprising the amino acid sequence, an a2 linker, and a Fc region. In some embodiments, the chimeric protein comprises a polypeptide comprising a D′ domain of VWF comprising the amino acid sequence of SEQ ID NO: 21, a D3 domain of VWF comprising the amino acid sequence of SEQ ID NO: 22, an ELNN polypeptide sequence comprising the amino acid sequence of SEQ ID NO: 14 (AE144_5A), an a2 linker comprising the amino acid sequence of SEQ ID NO: 15, and/or a Fc region comprising the amino acid sequence of SEQ ID NO: 23.

Polynucleotides encoding chimeric proteins and portions thereof are also included herein. In some embodiments, a polynucleotide sequence encodes a VWF fragment comprising a D1, D2, D′, and/or D3 domain of VWF. In some embodiments, a polynucleotide encodes aVWF fragment comprises a D1D2 region of VWF comprising the amino acid sequence of SEQ ID NO: 20. In some embodiments, a polynucleotide encodes a VWF fragment that further comprises a VWF signal peptide sequence. In some embodiments, the VWF signal peptide comprises the amino acid sequence of SEQ ID NO: 19. In some embodiments, a polynucleotide encodes a VWF fragment comprising a VWF signal peptide comprising the amino acid sequence of SEQ ID NO: 19, a D1D2 region of VWF comprising the amino acid sequence of SEQ ID NO: 20, a D′ domain of VWF comprising the amino acid sequence of SEQ ID NO: 21, a D3 domain of VWF comprising the amino acid sequence of SEQ ID NO: 22, an ELNN polypeptide sequence comprising the amino acid sequence of SEQ ID NO: 14 (AE144_5A), an a2 linker comprising the amino acid sequence of SEQ ID NO: 15, and/or a Fc region comprising the amino acid sequence of SEQ ID NO: 23.

In some embodiments, the chimeric protein of the present disclosure comprises: (i) a first polypeptide comprising a FVIII polypeptide with a first ELNN polypeptide sequence inserted into it, and a first Fc region; and (ii) a second polypeptide comprising a VWF fragment comprising a D′ domain of VWF and a D3 domain of VWF, a second ELNN polypeptide sequence, an a2 linker of FVIII, and a second Fc region; wherein: the FVIII polypeptide has a deletion of amino acids 746 to 1648 corresponding to mature FVIII; the first ELNN polypeptide sequence is inserted within the FVIII polypeptide immediately downstream of amino acid 745 corresponding to mature FVIII (SEQ ID NO: 8); the first ELNN polypeptide sequence comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of AE288 (SEQ ID NO: 9); the first Fc region is fused to the C-terminus of the FVIII polypeptide; the second ELNN polypeptide sequence is fused to the C-terminus of the VWF fragment; the second ELNN polypeptide sequence comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of AE144_5A (SEQ ID NO: 14); the a2 linker is fused to the C-terminus of the ELNN polypeptide; the a2 linker comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of SEQ ID NO: 15; the second Fc region is fused to the C-terminus of the a2 linker; and the first Fc region is covalently linked to the second Fc region by a disulfide bond (e.g., two disulfide bonds).

In some embodiments, the chimeric protein of the disclosure comprises two polypeptide sequences, a first polypeptide sequence comprising an amino acid sequence at least about 80%, 90%, 95%, 99%, or 100% identical to the amino acid sequence set forth in SEQ ID NO: 1; and a second polypeptide sequence comprising a VWF fragment comprising a D′ domain of VWF and a D3 domain of VWF and an Fc region. In some embodiments, the chimeric protein of the disclosure comprises two polypeptide sequences, a first polypeptide sequence comprising FVIII polypeptide and an Fc region; and a second polypeptide sequence comprising an amino acid sequence at least about 80%, 90%, 95%, 99%, or 100% identical to the amino acid sequence set forth in SEQ ID NO: 2. In some embodiments, the chimeric protein of the disclosure comprises two polypeptide sequences, a first polypeptide sequence comprising an amino acid sequence at least about 80%, 90%, 95%, 99%, or 100% identical to the amino acid sequence set forth in SEQ ID NO: 1 and a second polypeptide sequence comprising an amino acid sequence at least about 80%, 90%, 95%, 99%, or 100% identical to the amino acid sequence set forth in SEQ ID NO: 2. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence and the second polypeptide sequence are linked to each other by a disulfide bond. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence and the second polypeptide sequence are linked to each other by two disulfide bonds. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence comprises a first Fc portion, wherein the second polypeptide sequence comprises a second Fc portion, wherein the first Fc portion and the second Fc portion are linked to each other by two disulfide bonds in the hinge region.

In some embodiments, provided herein is a combination of polynucleotides, that encode a first and a second polypeptide, comprising a polynucleotide that encodes an amino acid sequence at least about 80%, 90%, 95%, 99%, or 100% identical to SEQ ID NO: 1, SEQ ID NO: 3, or SEQ ID NO: 7; and wherein a polypeptide that encodes an amino acid sequence at least about 80%, 90%, 95%, 99%, or 100% identical to SEQ ID NO: 2 or SEQ ID NO: 5.

In some embodiments, the chimeric protein of the disclosure comprises: (i) a first polypeptide comprising a FVIII polypeptide comprising a first FVIII polypeptide fragment comprising the amino acid sequence of SEQ ID NO: 17; a first ELNN polypeptide sequence comprising the amino acid sequence of SEQ ID NO: 9 (AE288); a second FVIII polypeptide fragment comprising the amino acid sequence of SEQ ID NO: 18; and a first Fc region comprising the amino acid sequence of SEQ ID NO: 23; and (ii) a second polypeptide comprising a VWF fragment comprising: a D′ domain of VWF comprising the amino acid sequence of SEQ ID NO: 21; a D3 domain of VWF comprising the amino acid sequence of SEQ ID NO: 22; a second ELNN polypeptide sequence comprising the amino acid sequence of SEQ ID NO: 14 (AE144_5A); an a2 linker comprising the amino acid sequence of SEQ ID NO: 15; and a second Fc region comprising the amino acid sequence of SEQ ID NO: 23, and wherein the first Fc region is covalently linked to the second Fc region by a disulfide bond (e.g., two disulfide bonds).

In some embodiments, the chimeric protein of the disclosure comprises a FVIII polypeptide comprising a first ELNN polypeptide sequence, a first Fc region, and a VWF fragment comprising a D′ domain of VWF, a D3 domain of VWF, a second ELNN polypeptide sequence, an a2 linker of FVIII and a second Fc region, wherein the FVIII polypeptide comprises the amino acid sequence of SEQ ID NO: 17, the first ELNN polypeptide sequence comprises the amino acid sequence of AE288 (SEQ ID NO: 9) and is fused to the C-terminus of SEQ ID NO: 17, the FVIII polypeptide further comprises the amino acid sequence of SEQ ID NO: 18, the first Fc region comprises the amino acid sequence of SEQ ID NO: 23 and is fused to the C-terminus of SEQ ID NO: 18; the D′ domain of VWF comprises the amino acid sequence of SEQ ID NO: 21; the D3 domain of VWF comprises the amino acid sequence of SEQ ID NO: 214, the second ELNN polypeptide sequence comprises the amino acid sequence of AE144_5A (SEQ ID NO: 14) and is fused to the C-terminus of the D3 domain of VWF; the a2 linker comprises the amino acid sequence of SEQ ID NO: 15 and is fused to the C-terminus of the second ELNN polypeptide sequence; the second Fc region comprises the amino acid sequence of SEQ ID NO: 23 and is fused to the C-terminus of the a2 linker; and wherein the first Fc region is covalently linked to the second Fc region by a disulfide bond.

In some embodiments, a chimeric protein comprises a first polypeptide and a second polypeptide. Included herein are polynucleotides (e.g. sets of polynucleotides) that encode the first polypeptide and the second polypeptide of a chimeric protein. In some embodiments, a polynucleotide encodes a polypeptide of a chimeric protein that comprises a FVIII polypeptide comprising a FVIII signal peptide comprising the amino acid sequence of SEQ ID NO: 16. In some embodiments, a polynucleotide encodes a polypeptide of a chimeric protein that comprises a VWF fragment comprising a VWF signal peptide comprising the amino acid sequence of SEQ ID NO: 19. In some embodiments, a polynucleotide encodes a polypeptide of a chimeric protein that comprises a VWF fragment comprising a D1D2 domain of VWF comprising the amino acid sequence of SEQ ID NO: 20.

In some embodiments, provided herein is combination of polynucleotides that encode a first and a second polypeptide, wherein the first polypeptide comprises the amino acid sequence of SEQ ID NO: 3; and wherein the second polypeptide comprises the amino acid sequence of SEQ ID NO: 2. In some embodiments, provided herein is combination of polynucleotides that encode a first and a second polypeptide, wherein the first polypeptide comprises the amino acid sequence of SEQ ID NO: 5; and wherein the second polypeptide comprises the amino acid sequence of SEQ ID NO: 2. In some embodiments, provided herein is combination of polynucleotides that encode a first and a second polypeptide, wherein the first polypeptide comprises the amino acid sequence of SEQ ID NO: 3; and wherein the second polypeptide comprises the amino acid sequence of SEQ ID NO: 7. In some embodiments, provided herein is combination of polynucleotides that encode a first and a second polypeptide, wherein the first polypeptide comprises the amino acid sequence of SEQ ID NO: 5; and wherein the second polypeptide comprises the amino acid sequence of SEQ ID NO: 2.

In some embodiments, the chimeric protein comprises a first polypeptide comprising the amino acid sequence of SEQ ID NO: 1 and a second polypeptide comprising the amino acid sequence of SEQ ID NO: 2. In some embodiments, the chimeric protein consists essentially of a first polypeptide having the amino acid sequence of SEQ ID NO: 1 and a second polypeptide having the amino acid sequence of SEQ ID NO: 2.

In some embodiments, the chimeric protein comprises one or more disulfide bridges between the first polypeptide and the second polypeptide. In some embodiments, the chimeric protein comprises two disulfide bridges between the first polypeptide and the second polypeptide. In some embodiments, the chimeric protein comprises a first polypeptide comprising the amino acid sequence of SEQ ID NO: 1 and a second polypeptide comprising the amino acid sequence of SEQ ID NO: 2, wherein the chimeric protein comprises a disulfide bridge between residue 1726 of SEQ ID NO: 1 and residue 663 of SEQ ID NO: 2, and a disulfide bridge between residue 1729 of SEQ ID NO: 1 and residue 666 of SEQ ID NO: 2.

### III. Methods of Treating Hemophilia A

Certain aspects of the present disclosure are directed to methods of treating hemophilia A in a subject in need thereof, comprising administering to the subject a chimeric protein comprising a FVIII polypeptide and a VWF fragment at a dosing interval. In some embodiments, the method comprises administering to the subject multiple doses of a chimeric protein, e.g., efanesoctocog alfa, comprising (i) a FVIII polypeptide and (ii) a VWF fragment comprising a D′ domain of VWF and a D3 domain of VWF at a dosing interval. In some aspects, the present disclosure is directed to methods of treating a hemophilia A, in a subject in need thereof, comprising administering to the subject multiple doses of a FVIII polypeptide and multiple doses of a VWF fragment at a dosing interval. In some embodiments, the hemophilia A is severe hemophilia A.

In some embodiments, disclosed herein are methods of treating hemophilia A with a chimeric protein for routine prophylaxis to reduce the frequency of bleeding episodes. In some embodiments, disclosed herein are methods of treating hemophilia A with a chimeric protein for on-demand treatment and control of bleeding episodes. In some embodiments, disclosed herein are methods of treating hemophilia A with a chimeric protein for perioperative management of bleeding.

Also disclosed herein are uses of a chimeric protein according to the methods of treatment disclosed herein. Also disclosed herein are uses of a chimeric protein to treat hemophilia A in a subject. In some embodiments, the chimeric protein is efanesoctocog alfa.

The chimeric proteins described herein can be administered by any means known in the art. In some embodiments, the chimeric protein is administered by intravenous injection, intravenous infusion, or subcutaneous administration. In some embodiments, the chimeric protein is administered intravenously. In some embodiments, the chimeric protein is administered subcutaneously.

In some embodiments, the subject is less than 12 years old and is intravenously administered 50 IU/kg once per week.

### A. Improvement in Bleeding Episode Outcomes

In some embodiments, the treatment of hemophilia A comprises preventing a bleeding episode or reducing the number of bleeding episodes in a human subject in need thereof. In some embodiments, the treatment of hemophilia A comprises treating a bleeding episode in a human subject in need thereof. In some embodiments, the treatment of hemophilia A comprises controlling the incidence or frequency of a bleeding episode in a human subject in need thereof. In some embodiments, the treatment of hemophilia A comprises decreasing the incidence or frequency of a bleeding episode in a human subject in need thereof.

In some embodiments, disclosed herein are prophylactic treatment methods for preventing, reducing or controlling spontaneous bleeds, traumatic bleeds, and normal bleeds. In an aspect, disclosed herein are methods for reducing annualized bleeding rate (ABR) resulting from hemophilia A in a subject in need thereof. In some embodiments, the ABR is the ABR of spontaneous bleeding episodes (the “spontaneous ABR”). In some embodiments, the ABR is the ABR of traumatic bleeding episodes (the “traumatic ABR”). In some embodiments, the ABR is the ABR of spontaneous and traumatic bleeding episodes (the “total ABR”). In some embodiments, the ABR is the ABR of spontaneous and traumatic bleeding episodes other than normal bleeds (the “total hemophilia ABR”). In some embodiments, the ABR is the ABR of spontaneous treated bleeding episodes (the “spontaneous treated ABR”). In some embodiments, the ABR is the ABR of traumatic treated bleeding episodes (the “traumatic treated ABR”). In some embodiments, the ABR is the ABR of spontaneous treated bleeding episodes and traumatic treated bleeding episodes (the “total treated ABR”). In some embodiments, the methods reduce the ABR of the subject to less than one. In some embodiments, the ABR of the subject is from 0 to about 0.25. In some embodiments, the ABR of the subject is from 0.25 to about 0.50. In some embodiments, the ABR of the subject is from 0.50 to about 0.75. In some embodiments, the ABR of the subject is from 0.75 to about 1. In some embodiments, the methods reduce the ABR of the subject to nearly zero. In some embodiments, the methods reduce the ABR of the subject to zero. In some embodiments, the ABR is the spontaneous ABR. In some embodiments, the ABR is the traumatic ABR. In some embodiments, the ABR is the total ABR. In some embodiments, the ABR is the total hemophilia ABR. In some embodiments, the ABR is the spontaneous treated ABR. In some embodiments, the ABR is the traumatic treated ABR. In some embodiments, the ABR is the total treated ABR.

In some embodiments, the ABR is measured by the number of spontaneous bleeding episodes in a subject. In some embodiments, disclosed herein are methods for reducing spontaneous bleeding episodes in a subject in need thereof. In some embodiments, the ABR is measured by both the number of spontaneous bleeding episodes and traumatic bleeding episodes in a subject. In some embodiments, disclosed herein are methods for reducing both the number of spontaneous bleeding episodes and traumatic bleeding episodes in a subject.

Also disclosed herein are methods for reducing ABR resulting from hemophilia A in a subject, wherein the subject is administered less than 3500 IU/kg/year of a chimeric protein to the subject in need thereof. In some embodiments, the method comprises administering less than 3500 IU/kg/year of the chimeric protein reduce the ABR of the subject to less than 3, less than 2, less than 1, or zero. In some embodiments, the method comprises administering less than 3400 IU/kg/year, less than 3300 IU/kg/year, less than 3200 IU/kg/year, less than 3100 IU/kg/year, less than 3000 IU/kg/year, less than 2900 IU/kg/year, less than 2800 IU/kg/year, less than 2700 IU/kg/year, or less than 2600 IU/kg/year of the chimeric protein. In some embodiments, the method comprises administering less than 3000 IU/kg/year. In some embodiments, the method comprises administering less than 2900 IU/kg/year. In some embodiments, the method comprises administering less than 2800 IU/kg/year. In some embodiments, the method comprises administering less than 2700 IU/kg/year. In some embodiments, the method comprises administering about 2600 IU/kg/year. In some embodiments, the ABR is less than 3 and the method comprises administering less than 3000 IU/kg/year. In some embodiments, the ABR is less than 3 and the method comprises administering less than 2900 IU/kg/year. In some embodiments, the ABR is less than 3 and the method comprises administering less than 2800 IU/kg/year. In some embodiments, the ABR is less than 3 and the method comprises administering less than 2700 IU/kg/year. In some embodiments, the ABR is less than 3 and the method comprises administering about 2600 IU/kg/year. In some embodiments, the ABR is less than 2 and the method comprises administering less than 3000 IU/kg/year. In some embodiments, the ABR is less than 2 and the method comprises administering less than 2900 IU/kg/year. In some embodiments, the ABR is less than 2 and the method comprises administering less than 2800 IU/kg/year. In some embodiments, the ABR is less than 2 and the method comprises administering less than 2700 IU/kg/year. In some embodiments, the ABR is less than 2 and the method comprises administering about 2600 IU/kg/year. In some embodiments, the ABR is less than 1 and the method comprises administering less than 3000 IU/kg/year. In some embodiments, the ABR is less than 1 and the method comprises administering less than 2900 IU/kg/year. In some embodiments, the ABR is less than 1 and the method comprises administering less than 2800 IU/kg/year. In some embodiments, the ABR is less than 1 and the method comprises administering less than 2700 IU/kg/year. In some embodiments, the ABR is less than 1 and the method comprises administering about 2600 IU/kg/year. In some embodiments, the ABR is between 1 and 0.5 and the method comprises administering less than 2700 IU/kg/year. In some embodiments, the ABR is between 1 and 0.5 and the method comprises administering about 2600 IU/kg/year. In some embodiments, the ABR is less than 1 and the method comprises administering about 2600 IU/kg/year. In some embodiments, the ABR is between 0.5 and 0 and the method comprises administering less than 2700 IU/kg/year. In some embodiments, the ABR is between 0.5 and 0 and the method comprises administering about 2600 IU/kg/year. In some embodiments, the ABR is less than 0.5 and the method comprises administering less than 2700 IU/kg/year. In some embodiments, the ABR is less than 0.5 and the method comprises administering about 2600 IU/kg/year. In some embodiments, the ABR is 0 and the method comprises administering about 2600 IU/kg/year. In some embodiments, the chimeric protein is efanesoctocog alfa.

In some embodiments, disclosed herein are methods for reducing or controlling traumatic bleeding episodes in a subject in need thereof. In some embodiments, the methods for reducing or controlling traumatic bleeding episodes are prophylactic treatment methods. In some embodiments, the prophylactic treatment methods are able to reduce or control traumatic bleeding episodes without requiring administration of supplemental on-demand FVIII replacement treatment.

Also disclosed herein are prophylactic treatment methods for reducing the number of bleeding episodes that require two or more administrations of supplemental on-demand FVIII treatments to control bleeding in a subject. In some embodiments, at least about 50%, at least about 70%, at least about 75%, at least about 77%, at least about 80%, at least about 85%, at least about 90%, at least about 95%, at least about 99%, or at least about 100% of the bleeding episodes in a subject are controlled by a single supplemental on-demand FVIII replacement treatment dose. In some embodiments, at least 99% of the bleeding episodes in a subject are controlled by one or two supplemental on-demand FVIII replacement treatment doses. In some embodiments, from about 70% to about 99%, from about 70% to about 80%, from about 75% to about 80%, from about 75% to about 85%, from about 70% to about 90%, or from about 80% to about 95% of the bleeding episodes in a subject are controlled by a single supplemental on-demand FVIII replacement treatment dose. In some embodiments, the bleeding episodes are traumatic bleeding episodes. In some embodiments, the bleeding episodes are spontaneous bleeding episodes.

In some embodiments, disclosed herein are methods for reducing ABR resulting from hemophilia A in a specific body part of a subject in need thereof. In some embodiments, the methods reduce ABR in a joint of the subject. In some embodiments, the methods reduce ABR in a joint of the subject to less than 3, less than 2, less than 1, or zero. In some embodiments, the methods reduce ABR in a muscle of the subject. In some embodiments, the methods reduce ABR in a muscle of the subject to less than 3, less than 2, less than 1, or zero. In some embodiments, the methods reduce ABR in the skin or mucosa of the subject. In some embodiments, the methods reduce ABR in the skin or mucosa of the subject to less than 3, less than 2, less than 1, or zero. In some embodiments, the ABR is the ABR of spontaneous bleeds. In some embodiments, the ABR is the ABR of traumatic bleeds other than normal bleeds.

In some embodiments, the methods disclosed herein result in reduced healthcare resource utilization (HRU) for medical encounters attributable to hemophilia A for the subject. In some embodiments, the number of medical care encounters (e.g., outpatient, urgent-care, emergency room) is reduced for the subject. In some embodiments, the duration of hospitalization (e.g. total days or length of stay, including duration by wards, such as the intensive care unit) is reduced for the subject. In some embodiments, the reduction over a given length of time during treatment with a chimeric protein compared to the same amount of time immediately before treatment with the chimeric protein began. In some embodiments, the length of time is at least 1, 3, 6, 9, or 12 months. In some embodiments, the length of time is about 1, 3, 6, 9, or 12 months.

In some embodiments, the methods disclosed herein produce a greater ABR reduction in a subject with hemophilia A than treatment with another clotting factor replacement product. Non-limiting examples of other clotting factor replacement products include Afstyla®, Advate®, Adynovate®, Eloctate®, Jivi®, Nuwiq©, Xyntha®, Recombinate®, Helixate®, Kogenate®, Hemofil M®, Koate-DVI®, Monarc-M®, Monoclate-P®, Alphanate®, Humate-P®, Wilate®, Kovaltry®, Novoeight®, and Voncento®. Provided herein is a method for reducing ABR compared to lonoctocog alfa, octocog alfa, rurioctocog alfa pegol, damoctocog alfa pegol, efmoroctocog alfa, simoctocog alfa, moroctocog alfa, a plasma derived FVIII, beroctocog alfa, an FVIII/VWF complex, and/or turoctocog alfa, comprising administering to a subject in need thereof a chimeric protein disclosed herein (such as efanesoctocog alfa) once per week at a dose of about 50 IU/kg. In some embodiments, the method comprises reducing ABR compared to lonoctocog alfa, octocog alfa, rurioctocog alfa pegol, damoctocog alfa pegol, efmoroctocog alfa, simoctocog alfa, moroctocog alfa, plasma derived FVIII, beroctocog alfa, FVIII/VWF complex, and/or turoctocog alfa when administered according to a label approved by the United States Food and Drug Administration or the European Medicines Agency, comprising administering to a subject in need thereof a chimeric protein disclosed herein (such as efanesoctocog alfa) once per week at a dose of about 50 IU/kg. In some embodiments, the method comprises reducing ABR compared to lonoctocog alfa, octocog alfa, rurioctocog alfa pegol, damoctocog alfa pegol, efmoroctocog alfa, simoctocog alfa, moroctocog alfa, plasma derived FVIII, beroctocog alfa, FVIII/VWF complex, and/or turoctocog alfa when administered at 2 to 3 times per week, comprising administering to a subject in need thereof a chimeric protein disclosed herein (such as efanesoctocog alfa) once per week at a dose of about 50 IU/kg. In some embodiments, the ABR is reduced by at least 50%. In some embodiments, the ABR is reduced by at least 60%. In some embodiments, the ABR is reduced by at least 70%. In some embodiments, the ABR is reduced by at least 75%. In some embodiments, the ABR is reduced by at least 77%. In some embodiments, the ABR is reduced by at least 80%. In some embodiments, the ABR is reduced by at least 85%. In some embodiments, the ABR is reduced by at least 90%. In some embodiments, the ABR is reduced by at least 95%. In some embodiments, the ABR is reduced by 100%. In some embodiments, the ABR is reduced by at least 50% to 75%. In some embodiments, the ABR is reduced by at least 70% to 90%. In some embodiments, the ABR is reduced by at least 75% to 80%. In some embodiments, the ABR is reduced by at least 75% to 85%. In some embodiments, the ABR is reduced by at least 75% to 90%. In some embodiments, the ABR is reduced by at least 75% to 90%. In some embodiments, the ABR is reduced in comparison to lonoctocog alfa, (Afstyla®), wherein the Ionoctocog alfa was previously administered at a dose of 20 to 50 IU/kg 2 to 3 times weekly. In some embodiments, the ABR is reduced in comparison to lonoctocog alfa, (Afstyla®), wherein the Ionoctocog alfa was previously administered at a dose of 30 to 50 IU/kg 2 to 3 times weekly in children less than 12 years of age.

In some embodiments, the ABR is reduced in comparison to octocog alfa (Advate®), wherein the octocog alfa was previously administered at a dose of 20 to 40 IU/kg every other day or 3 to 4 times weekly. In some embodiments, the ABR is reduced in comparison to octocog alfa (Kogenate®), wherein the octocog alfa was previously administered at a dose of 25 IU/kg 3 times a week. In some embodiments, the ABR is reduced in comparison to octocog alfa (Kogenate®), wherein the octocog alfa was previously administered at a dose of 25 IU/kg every other day.

In some embodiments, the ABR is reduced in comparison to rurioctocog alfa pegol (Adynovate®/Adynovi), wherein the rurioctocog alfa pegol was previously administered at a dose of 40 to 50 IU/kg twice weekly. In some embodiments, the ABR is reduced in comparison to rurioctocog alfa pegol (Adynovate®/Adynovi), wherein the rurioctocog alfa pegol was previously administered at a dose of 55 IU/kg twice weekly in children less than 12 years of age. n some embodiments, the ABR is reduced in comparison to rurioctocog alfa pegol (Adynovate®/Adynovi), wherein the rurioctocog alfa pegol was previously administered at a dose of no more than 70 IU/kg twice weekly in children less than 12 years of age.

In some embodiments, the ABR is reduced in comparison to damoctocog alfa pegol (Jivi®) wherein the damoctocog alfa pegol was previously administered at a dose of 30 to 40 IU/kg twice weekly. In some embodiments, the ABR is reduced in comparison to damoctocog alfa pegol (Jivi®), wherein the damoctocog alfa pegol was previously administered at a dose of 45 to 60 IU/kg every 5 days.

In some embodiments, the ABR is reduced in comparison to efmoroctocog alfa (Eloctate®), wherein the efmoroctocog alfa was previously administered at a dose of 50 IU/kg every 4 days. In some embodiments, the ABR is reduced in comparison to efmoroctocog alfa (Eloctate®), wherein the efmoroctocog alfa was previously administered at a dose of 25-65 IU/kg every 3 to 5 days. In some embodiments, the ABR is reduced in comparison to efmoroctocog alfa (Eloctate®), wherein the efmoroctocog alfa was previously administered at a dose of 50 IU/kg twice weekly in children less than 6 years of age. In some embodiments, the ABR is reduced in comparison to efmoroctocog alfa (Eloctate®), wherein the efmoroctocog alfa was previously administered at a dose of 80 IU/kg twice weekly in children less than 6 years of age.

In some embodiments, the ABR is reduced in comparison to simoctocog alfa (Nuwiq®), wherein the simoctocog alfa was previously administered at a dose of 30 to 40 IU/kg every other day.

In some embodiments, the ABR is reduced in comparison to moroctocog alfa, wherein the moroctocog alfa was previously administered at a dose of 30 IU/kg 3 times weekly.

In some embodiments, the ABR is reduced in comparison to an FVIII/VWF complex (Alphanate®), wherein the FVIII/VWF complex, was previously administered at a dose of 30 IU/kg 3 times weekly.

In some embodiments, the ABR is reduced compared to a bispecific antibody that binds to FIX or FIXa and FX. In some embodiments, the bispecific antibody is emicizumab. In some embodiments, better bleed protection is achieved with the chimeric protein than is typically achieved with Q1W administration of emicizumab. In some embodiments, better bleed protection is achieved with the chimeric protein than is typically achieved with Q4W administration of emicizumab. In some embodiments, better hemostasis is achieved with the chimeric protein than is typically achieved with Q1W administration of emicizumab. In some embodiments, better hemostasis is achieved with the chimeric protein than is typically achieved with Q4W administration of emicizumab.

In some embodiments, the ABR is the spontaneous ABR. In some embodiments, the ABR is the traumatic ABR. In some embodiments, the ABR is the total ABR. In some embodiments, the ABR is the total hemophilia ABR. In some embodiments, the ABR is the spontaneous treated ABR. In some embodiments, the ABR is the traumatic treated ABR. In some embodiments, the ABR is the total treated ABR.

In some embodiments, disclosed herein are methods for reducing annualized joint bleeding rate (AJBR) resulting from hemophilia A in a subject in need thereof. In some embodiments, AJBR is measured and assessed in the same manner as ABR, described above. In some embodiments the AJBR or joint bleeding episodes that require or prompt treatment is less than 2, or 1. In some embodiments the AJBR or joint bleeding episodes that require or prompt treatment is less than 1. In some embodiments the AJBR or joint bleeding episodes that require or prompt treatment is between 1 and 0.5. In some embodiments the AJBR or joint bleeding episodes that require or prompt treatment is between 0.5 and 0. In some embodiments the AJBR or joint bleeding episodes that require or prompt treatment is 0. In some embodiments, treatment with the chimeric protein results in an annualized joint bleeding rate (AjBR) of less than about 1. In some embodiments, treatment with the chimeric protein results in an annualized joint bleeding rate (AjBR) of about 0. In some embodiments, treatment with the chimeric protein results in an annualized joint bleeding rate (AjBR) of about 0.25 to 0.75. In some embodiments, treatment with the chimeric protein results in an annualized joint bleeding rate (AjBR) of about 0.75 to about 1.

Joints with several consecutive bleeds within a 6-month period are often referred to as “target joints,” and these joints often progress to hemophilic arthropathy. Resolution of a target joint is classified as 2 or fewer spontaneous bleeding episodes during a consecutive 12-month period. In some embodiments, disclosed herein are methods for resolving a target joint in a subject. In some embodiments, administration of a chimeric protein results in complete resolution of a target joint. In some embodiments, a subject has at least 1 target joint. In some embodiments, a subject has at least 2 target joints. In some embodiments, a subject has at least 3 target joints. In some embodiments, a subject has at least 4 target joints. In some embodiments, a subject has at least 5 target joints. In some embodiments, a subject has at least 6 target joints. In some embodiments, a subject has at least 1-6 target joints. In some embodiments, a subject has at least 1-3 target joints. In some embodiments, a subject has at least 3-6 target joints. In some embodiments, a subject has at least 1, at least 2, at least 3, at least 4, at least 5, or at least 6 target joints. In some embodiments, treatment with the chimeric protein resolves all the subject's target joint(s). In some embodiments, the subject has more than one target joint when the subject is first administered the chimeric protein, and all of the target joints are resolved within 12 months of treatment with the chimeric protein.

In some embodiments, a FVIII activity level (e.g., about 2%, about 5%, about 10%, about 15%, or about 20%) is maintained for the entire duration of a method in which a therapeutically effective amount (a dose of about 45 IU/kg to about 70 IU/kg of a chimeric protein at a dosing interval of about 6 days to about 14 days) of the chimeric protein is administered. In some embodiments, a FVIII activity level of at least 10% is maintained for the entire duration of a method in which a dose of about 50 IU/kg of the chimeric protein is administered about every 7 days. In some embodiments, a FVIII activity level of at least 12.5% is maintained for the entire duration of a method in which a dose of about 50 IU/kg of the chimeric protein is administered about every 7 days. In some embodiments, a FVIII activity level of at least 15% is maintained for the entire duration of a method in which a dose of about 50 IU/kg of the chimeric protein is administered about every 7 days. In some embodiments, a FVIII activity level of from about 10% to about 15% is maintained for the entire duration of a method in which a dose of about 50 IU/kg of the chimeric protein is administered about every 7 days. In some embodiments, a FVIII activity level of from about 10% to about 15% is maintained for the entire duration of a method in which a dose of about 50 IU/kg of the chimeric protein is administered about every 7 days. In some embodiments, the FVIII activity level is as assessed by a one-stage activated partial thromboplastin time (aPTT) assay. In some embodiments, the aPTT assay uses Actin FSL as a reagent. In some embodiments, the aPTT assay does not use Actin FS as a reagent.

Also provided herein are methods of prophylactically treating hemophilia A, comprising administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, wherein the subject is not administered any on-demand treatment for hemophilia A before engaging in strenuous activity.

In some embodiments, strenuous activity is an activity that raises the subject's heart rate. In some embodiments, strenuous activity is an activity that raises the subject's heart rate to at least 115 beats per minute. In some embodiments, strenuous activity is activity that raises a subject's heart rate to at least about 115, at least about 120, or at least about 125 beats per minute. In some embodiments, strenuous activity is activity that raises a subject's heart rate to at least about 115 beats per minute. In some embodiments, strenuous activity is activity that raises a subject's heart rate to at least about 120 beats per minute. In some embodiments, strenuous activity is activity that raises a subject's heart rate to at least about 125 beats per minute. In some embodiments, strenuous activity is activity that raises a subject's heart rate to at least about 115 to about 120 beats per minute, to at least about 115 to about 125 beats per minute, to at least about 115 to about 130 beats per minute, or to at least about 120 to about 130 beats per minute. In some embodiments, strenuous activity is activity that raises a subject's heart rate to at least about 115 to about 120 beats per minute. In some embodiments, strenuous activity is activity that raises a subject's heart rate to at least about 115 to about 125 beats per minute. In some embodiments, strenuous activity is activity that raises a subject's heart rate to at least about 115 to about 130 beats per minute. In some embodiments, strenuous activity is activity that raises a subject's heart rate to at least about 120 to about 130 beats per minute.

In some embodiments, the subject has increased confidence when engaging in physical activity, such as strenuous activity. In some embodiments, the subject has increased confidence that no bleed will occur when engaging in physical activity, such as strenuous activity. In some embodiments, the increase is compared to when the subject has received treatment with an FVIII replacement therapy other than the chimeric protein (e.g., a therapy that is currently approved by the European Union Medicines Agency, the Japanese Pharmaceuticals and Medical Devices Agency, the People's Republic of China National Medical Products Administration, the Republic of Korea Ministry of Food and Drug Safety, and/or the United States Food and Drug Administration) or other treatment for hemophilia A such as emicizumab. In some embodiments, the subject experiences reduced anxiety that a bleed will occur when engaging in physical activity. In some embodiments, the subject has reduced anxiety that an injury will occur when engaging in physical activity. In some embodiments, the reduction is compared to when the subject has received treatment with an FVIII replacement therapy other than the chimeric protein (e.g., a therapy that is currently approved by the European Union Medicines Agency, the Japanese Pharmaceuticals and Medical Devices Agency, the People's Republic of China National Medical Products Administration, the Republic of Korea Ministry of Food and Drug Safety, and/or the United States Food and Drug Administration) or other treatment for hemophilia A such as emicizumab.

In some embodiments, the subject has an increased ability to engage in physical activity when being treated with the chimeric protein compared to before treatment with the chimeric protein. For example, the subject can engage in more strenuous activity without an increased risk of a bleeding episode when being treated with the chimeric protein compared to before treatment with the chimeric protein. In some embodiments, the subject's level of fitness improves when the subject is being treated with the chimeric protein compared to before treatment with the chimeric protein. Physical activity measures include, for example, raw acceleration, activity counts, steps taken, physical activity intensity, activity bouts, sedentary bouts, body position, and/or other biometric measures. In some embodiments, the assessment of physical activity is performed using a triaxial medical grade accelerometer, such as the ActiGraph Activity Monitor. In some embodiments, the subject's body mass index improves when the subject is being treated with the chimeric protein compared to before the treatment begins. In some embodiments, the subject's body mass index decreases from above 25 (e.g., 25-29.9 or 30 or more) to from 18.5 to 24.9. In some embodiments, the subject's body mass index increases from below 18.5 to from 18.5 to 24.9.

In some embodiments, administration of the chimeric protein reduces the risk of a bleeding episode that requires more than one supplemental on-demand administration of a FVIII replacement protein during the prophylactic treatment of hemophilia A, wherein the risk is reduced by at least about 70%, at least about 75%, at least about 77%, at least about 80%, at least about 85%, at least about 90%, at least about 95%, or by 100%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least about 70%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least about 75%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least about 80%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least about 85%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least about 90%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least about 95%. In some embodiments, administration of the chimeric protein reduces the risk of a bleeding episode that requires more than one supplemental on-demand administration of a FVIII replacement protein during the prophylactic treatment of hemophilia A, wherein the risk is reduced by 100%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least from about 70% to 99%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least from about 70% to 80%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least from about 75% to 80%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least from about 75% to 85%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least from about 70% to 90%. In some embodiments, administration of the chimeric protein reduces the risk of a bleeding episode that requires more than one supplemental on-demand administration of a FVIII replacement protein during the prophylactic treatment of hemophilia A, wherein the risk is reduced by at least from about 80% to 95%.

### B. Improvement in Joint Health Outcomes

In an aspect, disclosed herein are methods for improving one or more joint health outcomes in a subject with hemophilia A. One example of a joint health outcome is the International Society on Thrombosis and Haemostasis (ISTH) 4-point assessment scale disclosed in Blanchette et al. J Thromb Haemost. 2014; 12(11):1935-9. The ISTH 4-point scale is depicted in Table 1. In some embodiments, the methods disclosed herein result in an ISTH 4-point assessment outcome of excellent for at least 75%, 80%, 90%, 95%, 99%, or 100% of bleeds. In some embodiments, the methods disclosed herein result in an ISTH 4-point assessment outcome of excellent for at least 95% of bleeds. In some embodiments, the methods disclosed herein result in an ISTH 4-point assessment outcome of good or excellent for at least 75%, 80%, 90%, 95%, 99%, or 100% of bleeds. In some embodiments, the bleeds are spontaneous bleeds. In some embodiments, the bleeds are traumatic bleeds. In some embodiments, the bleeds are spontaneous and traumatic bleeds.

TABLE 1
ISTH assessment of treatment of acute bleeds

| | |
|---|---|
| Excellent | Complete pain relief within 8 h and/or complete resolution |
| | of signs of bleeding after the initial injection and not |
| | requiring any further replacement therapy for relief of |
| | persistent symptoms and signs in the same joint within 72 h |
| Good | Significant pain relief and/or improvement in signs of |
| | bleeding within approximately 8 h after a single injection, |
| | but requiring more than one dose of replacement therapy |
| | within 72 h for complete resolution |
| Moderate | Modest pain relief and/or improvement in signs of bleeding |
| | within approximately 8 h after the initial injection and |
| | requiring more than one injection within 72 h but without |
| | complete resolution |
| None | No or minimal improvement, or condition worsens, within |
| | approximately 8 h after the initial injection |

Another example of a joint health outcome in a subject is based on the Physician's Global Assessment (PGA), which is a physician's assessment of the subject's response to treatment using a 4-point response scale (see Table 2). In some embodiments, the methods disclosed herein result in a PGA outcome of excellent. In some embodiments, the methods disclosed herein result in achieving an excellent outcome for at least 95% of joint bleeds as assessed using the PGA scale. In some embodiments, the methods disclosed herein result in a PGA of outcome effective for at least 75%, 80%, 90%, 95%, 99%, or 100% of bleeds. In some embodiments, the bleeds are spontaneous bleeds. In some embodiments, the bleeds are traumatic bleeds. In some embodiments, the methods disclosed herein result in a PGA outcome of excellent or effective, for at least 75%, 80%, 90%, 95%, 99%, or 100% of bleeds. In some embodiments, the bleeds are spontaneous bleeds. In some embodiments, the bleeds are traumatic bleeds

TABLE 2
PGA 4-point response scale.

| | |
|---|---|
| Excellent | Bleeding episodes responded to fewer than or the usual |
| | number of injections or less than or the usual dose of |
| | FVIII, OR |
| | The rate of breakthrough bleeding during prophylaxis |
| | was less than or equal to that usually observed |
| Effective | Most bleeding episodes responded to the same number of |
| | injections and dose, but some required more injections |
| | or higher doses, OR |
| | There was a minor increase in the rate of breakthrough |
| | bleeding. |
| Partially | Bleeding episodes most often required more injections |
| Effective | and/or higher doses than expected, OR |
| | Adequate breakthrough bleeding prevention during |
| | prophylaxis required more frequent injections and/or |
| | doses. |
| Ineffective | Routine failure to control hemostasis or hemostatic |
| | control required additional agents |

Another example of a joint health outcome in a subject is based on the Hemophilia Joint Health Score (HJHS) (Hilliard et al. (2006) Haemophilia. 12(5):518-25; Feldman et al. (2011) Arthritis Care Res. 63(2):223-30; both incorporated herein by reference). Joint function assessed by physical joint examination performed by an experienced health care professional is often used as the primary outcome for hemophilic arthropathy. HJHS is a validated 11-item scoring tool developed for the assessment of joint health in subjects with hemophilia. Following domains were assessed for elbows, knee and ankle joints: swelling (score 0=no swelling to 3=severe), duration of swelling (score 0=no swelling and 1=>=6 months), muscle atrophy (score 0=none to 2=severe), crepitus on motion (score 0=none to 2=severe), flexion loss (score 0=<5″ to 3=>20″), extension loss (score 0=<5″ to 3=>20″), joint pain (score 0=no pain through active range of motion to 2=pain through active range) and strength (score 0=holds test position with maximum resistance to 4=trace/no muscle contraction), in each item 0=none and higher score=severe damage. Global gait score is an assessment of walking, stairs, running, and hopping on 1 leg, and is scored from 0-4. The assessment is administered by a healthcare professional trained in the use of anthropometric measures.

In some embodiments, disclosed herein are methods for improving the total HJHS score in subject. In some embodiments, disclosed herein are methods for improving one or more HJHS domain scores in a subject. In some embodiments, disclosed herein are methods for improving the swelling and/or strength HJHS domain score in a subject. In some embodiments, the HJHS score of a subject improves by at least 1 compared to the HJHS score of the subject before the subject first received prophylactic treatment with the chimeric protein described herein. In some embodiments, the HJHS score of a subject improves by at least 2 compared to the HJHS score of the subject before the subject first received prophylactic treatment with the chimeric protein described herein. In some embodiments, the HJHS score of a subject improves by at least 3 compared to the HJHS score of the subject before the subject first received prophylactic treatment with the chimeric protein described herein. In some embodiments, the HJHS score of a subject improves by at least 4 compared to the HJHS score of the subject before the subject first received prophylactic treatment with the chimeric protein described herein. In some embodiments, the HJHS score of a subject improves by at least 5 compared to the HJHS score of the subject before the subject first received prophylactic treatment with the chimeric protein described herein. In some embodiments, the HJHS score of a subject improves by at least 1 to 3 compared to the HJHS score of the subject before the subject first received prophylactic treatment with the chimeric protein described herein. In some embodiments, the HJHS score of a subject improves by at least 1 to 4 compared to the HJHS score of the subject before the subject first received prophylactic treatment with the chimeric protein described herein. In some embodiments, the HJHS score of a subject improves by at least 1 to 5 compared to the HJHS score of the subject before the subject first received prophylactic treatment with the chimeric protein described herein.

In some embodiments, improving one or more joint outcomes comprises increased joint flexion ability, increased joint extension, increased range of movement, increased muscle strength, decreased swelling, decreased duration of swelling, decreased crepitus, decreased pain, or decreased muscle atrophy of one or more joints. In some embodiments, improving one or more joint outcomes comprises improved walking, improved ability to use stairs, improved running ability, or improved ability to hop on one leg.

In some embodiments, the HJHS score of a subject improves by at least 5% compared to the HJHS score of the subject before the subject first received prophylactic treatment with the chimeric protein described herein. In some embodiments, the HJHS score of a subject improves by at least 10% compared to the HJHS score of the subject before the subject first received prophylactic treatment with the chimeric protein described herein. In some embodiments, the HJHS score of a subject improves by at least 12.5% compared to the HJHS score of the subject before the subject first received prophylactic treatment with the chimeric protein described herein. In some embodiments, the HJHS score of a subject improves by at least 15% compared to the HJHS score of the subject before the subject first received prophylactic treatment with the chimeric protein described herein. In some embodiments, the HJHS score of a subject improves by at least 20% compared to the HJHS score of the subject before the subject first received prophylactic treatment with the chimeric protein described herein. In some embodiments, the HJHS score of a subject improves by at least 10% to 12.5% compared to the HJHS score of the subject before the subject first received prophylactic treatment with the chimeric protein described herein. In some embodiments, the HJHS score of a subject improves by at least 5% to 10% compared to the HJHS score of the subject before the subject first received prophylactic treatment with the chimeric protein described herein. In some embodiments, the HJHS score of a subject improves by at least 12.5% to 15% compared to the HJHS score of the subject before the subject first received prophylactic treatment with the chimeric protein described herein. In some embodiments, the HJHS score of a subject improves by at least 10% to 15% compared to the HJHS score of the subject before the subject first received prophylactic treatment with the chimeric protein described herein. In some embodiments, the HJHS score of a subject improves by at least 15% to 20% compared to the HJHS score of the subject before the subject first received prophylactic treatment with the chimeric protein described herein.

In some embodiments, subjects treated with the chimeric protein described herein show an improvement in joint health as assessed by ultrasound. In some embodiments, the improvement in joint health is assessed using the US joint images from the Joint Tissue Activity and Damage Examination (JADE) Protocol in Musculoskeletal Ultrasound (MSKUS) and/or Hemophilia Early Arthropathy Detection with Ultrasound (HEAD-US). In some embodiments, joint health is assessed using the HEAD-US. The HEAD-US scoring system (alternatively, HEAD-US protocol) is based on 3 parameters (synovitis [score, 0-2], cartilage [0-4], and subchondral bone [0-2]) for 6 joints (left and right knees, elbows, and ankles). Decreased scores indicate joint health improvements. In some embodiments, subjects show an improvement in synovial hypertrophy. In some embodiments, subjects show an improvement in cartilage thickness. In some embodiments, subjects show an improvement in synovial hyperaemia, as observed by power Doppler (JADE) or power Doppler Ultrasound (PDUS).

### C. Improvement in Quality of Life Outcomes

In an aspect, disclosed herein are methods for improving one or more quality of life (QoL) outcomes in a subject with hemophilia A.

One example of a QoL outcome is based on the Haemophilia Quality of Life Questionnaire (Haem-A-QoL), which measures health-related QoL in subjects with hemophilia. (see, e.g., Wyrwich et al. Haemophilia. 2015; 21(5):578-84; von Mackensen et al., Haemophilia. 2017; 23(3):383-391). In some embodiments, disclosed herein are methods for improving the total Haem-A-QoL score in subject. In some embodiments, disclosed herein are methods for improving one or more Haem-A-QoL domain scores in subject. In some embodiments, disclosed herein are methods for improving the Haem-A-QoL view of yourself domain score in a subject. In some embodiments, disclosed herein are methods for improving the Haem-A-QoL work and school domain score in a subject. In some embodiments, disclosed herein are methods for improving the Haem-A-QoL sports and leisure domain score in a subject. In some embodiments, disclosed herein are methods for improving the Haem-A-QoL treatment domain score in a subject. In some embodiments, disclosed herein are methods for improving the Haem-A-QoL feeling domain score in a subject. In some embodiments, disclosed herein are methods for improving the Haem-A-QoL partnership and sexuality domain score in a subject. In some embodiments, disclosed herein are methods for improving the Haem-A-QoL physical health score in a subject. In some embodiments, the Haem-A-QoL score of the subject improves by at least 5% compared to the Haem-A-QoL score of the subject before the subject first received prophylactic treatment with the chimeric protein. In some embodiments, the Haem-A-QoL score of the subject improves by at least 10% compared to the Haem-A-QoL score of the subject before the subject first received prophylactic treatment with the chimeric protein. In some embodiments, the Haem-A-QoL score of the subject improves by at least 12.5% compared to the Haem-A-QoL score of the subject before the subject first received prophylactic treatment with the chimeric protein. In some embodiments, the Haem-A-QoL score of the subject improves by at least 15% compared to the Haem-A-QoL score of the subject before the subject first received prophylactic treatment with the chimeric protein. In some embodiments, the Haem-A-QoL score of the subject improves by at least 20% compared to the Haem-A-QoL score of the subject before the subject first received prophylactic treatment with the chimeric protein. In some embodiments, the Haem-A-QoL score of the subject improves by at least 5% to 10% compared to the Haem-A-QoL score of the subject before the subject first received prophylactic treatment with the chimeric protein. In some embodiments, the Haem-A-QoL score of the subject improves by at least 10% to 12.5% compared to the Haem-A-QoL score of the subject before the subject first received prophylactic treatment with the chimeric protein. In some embodiments, the Haem-A-QoL score of the subject improves by at least 12.5% to 15% compared to the Haem-A-QoL score of the subject before the subject first received prophylactic treatment with the chimeric protein. In some embodiments, the Haem-A-QoL score of the subject improves by at least 10% to 15% compared to the Haem-A-QoL score of the subject before the subject first received prophylactic treatment with the chimeric protein. In some embodiments, the Haem-A-QoL score of the subject improves by at least 15% to 20% compared to the Haem-A-QoL score of the subject before the subject first received prophylactic treatment with the chimeric protein.

In some embodiments, the Haem-A-QoL score of the subject improves by at least 5 compared to the Haem-A-QoL score of the subject before the subject first received prophylactic treatment with the chimeric protein. In some embodiments, the Haem-A-QoL score of the subject improves by at least 6 compared to the Haem-A-QoL score of the subject before the subject first received prophylactic treatment with the chimeric protein. In some embodiments, the Haem-A-QoL score of the subject improves by at least 7 compared to the Haem-A-QoL score of the subject before the subject first received prophylactic treatment with the chimeric protein. In some embodiments, the Haem-A-QoL score of the subject improves by at least 8 compared to the Haem-A-QoL score of the subject before the subject first received prophylactic treatment with the chimeric protein. In some embodiments, the Haem-A-QoL score of the subject improves by at least 9 compared to the Haem-A-QoL score of the subject before the subject first received prophylactic treatment with the chimeric protein. In some embodiments, the Haem-A-QoL score of the subject improves by at least 10 compared to the Haem-A-QoL score of the subject before the subject first received prophylactic treatment with the chimeric protein. In some embodiments, the Haem-A-QoL score of the subject improves by at least 5, at least 6, at least 7, at least 8, at least 9, or at least 10 compared to the Haem-A-QoL score of the subject before the subject first received prophylactic treatment with the chimeric protein. In some embodiments, the Haem-A-QoL score of the subject improves by at least 5 to 7 compared to the Haem-A-QoL score of the subject before the subject first received prophylactic treatment with the chimeric protein. In some embodiments, the Haem-A-QoL score of the subject improves by at least 5 to 8 compared to the Haem-A-QoL score of the subject before the subject first received prophylactic treatment with the chimeric protein. In some embodiments, the Haem-A-QoL score of the subject improves by at least 5 to 9 compared to the Haem-A-QoL score of the subject before the subject first received prophylactic treatment with the chimeric protein. In some embodiments, the Haem-A-QoL score of the subject improves by at least 5 to 10 compared to the Haem-A-QoL score of the subject before the subject first received prophylactic treatment with the chimeric protein.

Another example of QoL outcomes are based on the Hemophilia Activities List (HAL) and pediatric HAL (pedHAL) questionnaires. These questionnaires assess the participant's functional ability to perform activities of daily living. In general, the HAL is assessed in patients >18 years of age, and the pedHAL is assessed in patients <18 years of age. Additional details regarding the HAL/pedHAL can be found in Van Genderen FR, et al. Haemophilia 2006 January, 12(1):36-46. In some embodiments, a subject is at least 18 years old and the subject's HAL score increases by at least 5% upon treatment with the chimeric protein. In some embodiments, a subject is at least 18 years old and the subject's HAL score increases by at least 10%. In some embodiments, a subject is less than 18 years old and the subject's pedHAL score increases by at least 5%. In some embodiments, a subject is less than 18 years old and the subject's pedHAL score increases by at least 10%.

Another example of QoL outcomes are based on the EuroQoL 5-dimension 5-level (EQ-5D-5L), which assesses 5 dimensions of health outcome (mobility, self-care, usual activities, pain/discomfort, anxiety/depression) from a wide variety of interventions on a common scale, for purposes of converting into a single summary score of health state. EQ-5D is a generic quality of life measure that is not specific to hemophilia, and as such, is not as sensitive to changes (improvements) in quality of life as hemophilia-specific measures. The EQ-5D-5L comprises a descriptive system and a visual analog system (VAS). The EQ-5D index score can also be measured, which is the value attached to an EQ-5D profile according to a set of weights that reflect, on average, people's preferences about how good or bad the state is. Additional details regarding the EQ-5D-5L can be found in Janssen et al. Qual Life Res 2013; 22(7):1717-27, and Devlin et al. Appl Health Econ Health Policy 2017; 15:127-137. In some embodiments, the subject's EQ-5D-5L score increases by at least 5% upon treatment with the chimeric protein. In some embodiments, subject's EQ-5D-5L score increases by at least 10%.

In some embodiments, treatment with a chimeric protein disclosed herein reduces pain associated with hemophilia A (e.g., pain resulting from bleeding or inflammation or tissue damage from bleeds). In some embodiments, the pain is chronic pain. In some embodiments, the pain is joint pain. In some embodiments, the pain is chronic joint pain.

Another example of a QoL outcome is based on the Patient-Reported Outcomes Measurement Information System (PROMIS). PROMIS is a system of reliable and precise measures of participant-reported heath status (Cella et al. Medical Care. 2007; 45(5 Suppl 1): S3-11). The PROMIS initiative is part of the National Institute of Health (NIH) goal to develop systems to support NIH-funded research across all its institutes and centers. PROMIS measures cover physical, mental and social health and can be used for many chronic conditions. These questionnaires use a 5-point Likert response scale. The PROMIS-SF (short form) v 1.0 Pain Intensity 3a tool consists of 3 questions on the patient's pain over the past 7 days, to which they are asked to respond: ‘had no pain,’ ‘mild,’ ‘moderate,’ ‘severe,’ and ‘very severe.’ (Cook et al. J Clin Epidemiol 2016; 73:89-102, the entire content of which is incorporated herein by reference).

The PROMIS-SF v2.0 Physical Function 6b tool consists of 2-items from item-improved Health Assessment Questionnaire (HAQ) and 4-items from item-improved Physical Function-10 (PF-10) instruments. Both of these instruments assess a subject's present abilities and has 5-response options: HAQ: 1=without any difficulty, 2=with little difficulty, 3=with some difficulty, 4=with much difficulty, 5=unable to do. PF-10: 1=not at all, 2=very little, 3=somewhat, 4=quite a lot, 5=cannot do. Total score can be determined from the average scores of component items, which range from 0 (no disability) to 100 (worst disability). In some embodiments, a T-score is calculated, which rescales raw scale score (sum of scores from all questions answered) into a standardized score with a mean of 50 and standard deviation of 10, based on scoring tables provided in PROMIS Scoring Manuals (lower score=better outcome). In some embodiments, the subject's T-score improves when the subject is being treated with the chimeric protein compared to before treatment with the chimeric protein.

In some embodiments, disclosed herein are methods for improving the PROMIS pain intensity score in subject. In some embodiments, disclosed herein are methods for improving the PROMIS physical function score in a subject. In some embodiments, treatment with a chimeric protein disclosed herein reduces chronic pain. In some embodiments, improving the quality of life of the subject comprises reducing the subject's self-reported pain intensity over a period of time, such as over the previous 7 days. In some embodiments, treatment with a chimeric protein disclosed herein reduces joint pain. In some embodiments, the subject's self-reported pain intensity improves (i.e., decreases or is reduced) by at least 10%.

In some embodiments, the subject's self-reported pain intensity is chosen from the choices of “very severe”, “severe”, “moderate”, “mild”, or “had no pain”, and wherein the intensity improves from “very severe” to “severe”, “moderate”, “mild”, or “had no pain”. In some embodiments, the subject's self-reported pain intensity is chosen from the choices of “very severe”, “severe”, “moderate”, “mild”, or “had no pain”, and wherein the intensity improves from “severe” to “moderate”, “mild”, or “had no pain”. In some embodiments, the subject's self-reported pain intensity is chosen from the choices of “very severe”, “severe”, “moderate”, “mild”, or “had no pain”, and wherein the intensity improves from “moderate” to “mild” or “had no pain”. In some embodiments, the subject's self-reported pain intensity is chosen from the choices of “very severe”, “severe”, “moderate”, “mild”, or “had no pain”, and wherein the intensity improves from “mild” to “had no pain”.

In some embodiments, improving the quality of life of the subject comprises reducing the subject's self-reported pain intensity, wherein the subject's self-reported pain intensity changes from severe to moderate, mild, or no pain.

In some embodiments, the subject's self-reported pain intensity is scored on a scale of 1 to 5. In some embodiments, the subject's self-reported pain intensity is scored on a scale of 1 to 5 with 1=no pain, 2=mild pain, 3=moderate pain, 4=severe pain, and 5=very severe pain. In some embodiments, the score improves from 5 to 4. In some embodiments, the score improves from 5 to less than 4. In some embodiments, the score improves from 5 to less than 3. In some embodiments, the score improves from 5 to 2 or less. In some embodiments, the score improves from 4 to 3. In some embodiments, the score improves from 4 to less than 3. In some embodiments, the score improves from 4 to 2 or less. In some embodiments the score improves from 3 to 2 or 1. In some embodiments, the score improves from 2 to 1.

In some embodiments, the subject's self-reported pain intensity is scored on a scale of 1 to 10.

In some embodiments, treatment with a chimeric protein disclosed herein reduces the amount of pain medication needed to decrease or manage pain associated with hemophilia A. In some embodiments, the amount of pain medication is decreased compared to the amount needed when the subject is treated for hemophilia A with (i) a FVIII replacement protein that is capable of being bound by endogenous von Willebrand Factor (VWF); (ii) a complex, wherein the complex comprises a FVIII protein and a wild-type VWF protein or a portion thereof, wherein the FVIII protein and the VWF protein or portion thereof are not directly or indirectly covalently attached to each other; or (iii) emicizumab. In some embodiments, the amount of pain medication is decreased (i) compared to the amount needed when the subject is treated for hemophilia A with prophylactic treatment with emicizumab, (ii) compared to the amount needed when the subject is treated for hemophilia A with prophylactic treatment with a FVIII replacement protein other than the chimeric protein, wherein the FVIII replacement protein is capable of being bound by endogenous VWF, (iii) compared to the amount a corresponding subject who receives prophylactic treatment with emicizumab needs, or (iv) compared to the amount a corresponding subject who receives prophylactic treatment for hemophilia A that comprises administration of an FVIII replacement protein other than the chimeric protein needs, wherein the FVIII replacement protein is capable of being bound by endogenous VWF. In some embodiments, the number of doses of the pain medication is reduced. In some embodiments, the number of doses of the pain medication over the course of a day or a week is reduced. In some embodiments, a subject is administered a chimeric protein disclosed herein followed by the reduced dose of the pain medication. In some embodiments, the pain medication comprises a nonsteroidal anti-inflammatory drug (NSAID), an opioid, or a nonopioid pain reliever. In some embodiments, the pain medication comprises a nonopioid pain reliever. In some embodiments, the pain medication comprises an NSAID. In some embodiments, the pain medication comprises fentanyl, hydromorphone, morphine, oxycodone, oxymorphone, tramadol, oxycodone, oxycodone, acetaminophen (also known as paracetamol), ibuprofen, naproxen sodium, metamizole sodium, celecoxib, ketorolac, diclofenac, diclofenac potassium, diclofenac epolamine, lidocaine, or aspirin. In some embodiments, the pain medication is acetaminophen. In some embodiments, the pain medication is celecoxib.

In some embodiments, treatment with a chimeric protein disclosed herein reduces the amount of pain medication needed to decrease or manage pain associated with hemophilia A before, during, or within 1, 3, or 6 hours after the strenuous activity (i) compared to the amount needed when the subject is treated for hemophilia A with prophylactic treatment with emicizumab, (ii) compared to the amount needed when the subject is treated for hemophilia A with prophylactic treatment with a FVIII replacement protein other than the chimeric protein, wherein the FVIII replacement protein is capable of being bound by endogenous VWF, (iii) compared to the amount a corresponding subject who receives prophylactic treatment with emicizumab needs, or (iv) compared to the amount a corresponding subject who receives prophylactic treatment for hemophilia A that comprises administration of an FVIII replacement protein other than the chimeric protein, wherein the FVIII replacement protein is capable of being bound by endogenous VWF. In some embodiments, the subject does not need pain medication to decrease or manage pain associated with hemophilia A before, during, or within 1, 3, or 6 hours after the strenuous activity.

In some embodiments, the strenuous activity is an activity that raises the subject's heart rate to at least 115 beats per minute. In some embodiments, the strenuous activity is an activity that raises the subject's heart rate to at least 115 beats per minute. Non-limiting examples of a strenuous activity include parkour, running, swimming, bicycling, rock climbing, mountain climbing, other climbing, and hiking, In some embodiments, the strenuous activity is a sport. In some embodiments, the strenuous activity is a contact sport. Non-limiting examples of a strenuous activity include soccer, tennis, skiing, snowboarding, skateboarding, basketball, football, hockey, rugby, boxing, wrestling, or a martial art.

The methods of the present disclosure can also be directed to treating a bleeding episode or a symptom resulting from hemophilia A in a subject in need thereof. Examples of bleeding episodes and symptoms include hemarthrosis, muscle bleeds, oral bleeds, hemorrhage, hemorrhage into muscles, oral hemorrhage, bleeding from trauma, trauma capitis, gastrointestinal bleeding, intracranial hemorrhage, intra-abdominal hemorrhage, intrathoracic hemorrhage, bleeding from a bone fracture, central nervous system bleeding, bleeding in the retropharyngeal space, bleeding in the retroperitoneal space, bleeding in the iliopsoas sheath, or any combination thereof.

In some embodiments, the methods of the present disclosure are directed to improving a subject's quality of life is improved compared to (i) prophylactic treatment (e.g., previous prophylactic treatment) with emicizumab, (ii) prophylactic treatment (e.g., previous prophylactic treatment) with a FVIII replacement protein other than the chimeric protein, wherein the FVIII replacement protein is capable of being bound by endogenous VWF, (iii) to the quality of life of a corresponding subject who receives prophylactic treatment with emicizumab, or (iv) the quality of life of a corresponding subject who receives prophylactic treatment for hemophilia A that comprises administration of an FVIII replacement protein other than the chimeric protein, wherein the FVIII replacement protein is capable of being bound by endogenous VWF.

In some embodiments, a subject's quality of life is improved compared to prophylactic treatment (e.g., previous prophylactic treatment) with emicizumab. In some embodiments, a subject's quality of life is improved compared to prophylactic treatment (e.g., previous prophylactic treatment) with a FVIII replacement protein other than the chimeric protein, wherein the FVIII replacement protein is capable of being bound by endogenous VWF. In some embodiments, a subject's quality of life is improved compared to the quality of life of a corresponding subject who receives prophylactic treatment with emicizumab. In some embodiments, a subject's quality of life is improved compared to the quality of life of a corresponding subject who receives prophylactic treatment for hemophilia A that comprises administration of an FVIII replacement protein other than the chimeric protein, wherein the FVIII replacement protein is capable of being bound by endogenous VWF.

In some embodiments, to a subject has a reduction in the number of bleeds and bleed-related complications when treated with the chimeric protein compared to previous treatment. In some embodiments, a subject experiences a reduction in pain when treated with the chimeric protein compared to previous treatment. In some embodiments, to a subject has improved joint mobility when treated with the chimeric protein compared to previous treatment. In some embodiments, there is a shorter time of administration of the chimeric protein compared to a previous treatment. In some embodiments, a subject feels better protected when treated with the chimeric protein compared to previous treatment. In some embodiments, a subject has greater confidence that a bleeding episode will not occur (e.g., when engaging in physical activity such as strenuous activity) when treated with the chimeric protein compared to previous treatment. In some embodiments, a subject has reduced fatigue when treated with the chimeric protein compared to before treatment with the chimeric protein started. In some embodiments, a subject has reduced fatigue when treated with the chimeric protein compared to previous treatment. In some embodiments, a subject has an increased sense of well-being when treated with the chimeric protein compared to previous treatment. In some embodiments, a subject has improved mobility. In some embodiments, a subject has an improved ability to use fine hand movements. In some embodiments, the subject has an improved ability to close buttons, knit, and/or type.

In some embodiments, the quality of life of a subject is improved compared to the quality of life that would be expected for a corresponding subject who does not receive treatment with the chimeric protein. In some embodiments, the corresponding subject is a subject with hemophilia A who has similar health, weight, body mass index, and age. In some embodiments, the corresponding subject is a hypothetical subject who is the same as the subject in every way except the type of hemophilia A treatment he or she receives.

### D. Perioperative Management

Also provided herein are methods for the perioperative management of bleeding, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (c) a second Fc region, and wherein the first Fc region and the second Fc region are covalently attached to each other by at least one disulfide bond.

In some embodiments, the subject is expected to receive surgery within 72 hours. In some embodiments, the subject is receiving surgery. In some embodiments, the subject has received surgery within the last week. In some embodiments, the surgery is major surgery. In some embodiments, the surgery is minor surgery. In some embodiments, the surgery is hip, knee, or ankle replacement surgery. In some embodiments, the surgery is organ transplant surgery. In some embodiments, the organ transplant surgery is a lung, liver, stem cell, cornea, heart, kidney, or pancreas transplant.

In some embodiments, the therapeutically effective amount achieves an excellent hemostatic response as assessed by a surgeon.

In some embodiments, the therapeutically acceptable amount is a dose from about from 30 IU/kg to 50 IU/kg. In some embodiments, the therapeutically effective amount is a dose of 30 IU/kg. In some embodiments, the therapeutically effective amount is a dose of 50 IU/kg.

In some embodiments, a total of about 30 to about 50 IU/kg of the chimeric protein is administered on the day of surgery or in the 24 hours prior to surgery. In some embodiments, a total of about 30 to about 50 IU/kg of the chimeric protein is administered in the 72 hours following surgery. In some embodiments, a total of about 75 to about 125 IU/kg of the chimeric protein is administered from day 4 after surgery to day 14 after surgery. In some embodiments, a total of about 150 to about 200 IU/kg of the chimeric protein is administered from 24 hours before surgery through two weeks after surgery.

In some embodiments, a dose is administered before the surgery begins. In some embodiments, a dose is administered before and after the surgery. In some embodiments, a dose is administered once. In some embodiments, a dose is administered every 2 or 3 days after surgery until bleeding from the surgery has ended. In some embodiments, a dose of from 30 IU/kg to 50 IU/kg is administered less than 24 hours before the surgery. In some embodiments, a dose of from 30 IU/kg to 50 IU/kg is administered less than 24 hours before the surgery, and then at least one additional dose of from 30 IU/kg to 50 IU/kg is administered after the surgery until bleeding from the surgery stops. In some embodiments, a dose of from 30 IU/kg to 50 IU/kg is administered within 24 hours before the surgery begins, and then an additional dose of from 30 IU/kg to 50 IU/kg is administered from about 2 to about 3 days after the surgery has ended. In some embodiments, the dose is administered 2 to 3 days or less after a prophylactic dose.

In some embodiments, the therapeutically effective amount comprises a preoperative loading dose of 50 IU/kg, followed by one or more doses of 30 or 50 IU/kg every 2 to 3 days. In some embodiments, the therapeutically effective amount comprises a preoperative loading dose of 50 IU/kg, followed by one or more doses of 30 or 50 IU/kg every 2 to 3 days as needed to reduce or control bleeding associated with the surgery. In some embodiments, the therapeutically effective amount comprises a preoperative loading dose of 50 IU/kg, followed by one or more doses of 30 or 50 IU/kg every 2 to 3 days for up to one week. In some embodiments, the therapeutically effective amount comprises a preoperative loading dose of 50 IU/kg, followed by one or more doses of 30 or 50 IU/kg every 2 to 3 days for at least about one week. In some embodiments, the preoperative loading dose is administered within 3 days of the surgery. In some embodiments, the preoperative loading dose is administered within 2 days of the surgery.

In some embodiments, the risk that the subject will require a blood transfusion as a result of the surgery (e.g., during or after the surgery) is reduced when the subject is treated with the chimeric protein compared to when the subject or a corresponding subject is administered a FVIII replacement protein that is capable of being bound by endogenous VWF.

### E. Dosing

In some embodiments, the chimeric protein, e.g., efanesoctocog alfa, is administered as a single dose or as multiple doses. In some embodiments, the amounts of each of the multiple doses are the same. In some embodiments, one or more of the multiple doses is different from one or more of the other multiple doses. In some embodiments, at least one of the multiple doses of the chimeric protein is from about 45 IU/kg to about 70 IU/kg. In some embodiments, each of the multiple doses of the chimeric protein is from about 45 IU/kg to about 70 IU/kg. In some embodiments, the chimeric protein is administered once per week at a dose of 50 IU/kg.

In some embodiments, each dose of the multiple doses is about 45 IU/kg. In some embodiments, each dose of the multiple doses is about 50 IU/kg. In some embodiments, each dose of the multiple doses is about 55 IU/kg.

In some embodiments, the chimeric protein, e.g., efanesoctocog alfa, is administered prophylactically. In some embodiments, at least one of the multiple doses can be from about 45 IU/kg to about 70 IU/kg. In some embodiments, at least one of the multiple doses administered prophylactically is about 50 IU/kg. In some embodiments, each of the multiple doses administered prophylactically is about 50 IU/kg.

In some embodiments, the chimeric protein, e.g., efanesoctocog alfa, is administered on-demand. When administered on-demand, the chimeric protein can be administered as a single dose or as multiple doses. In some embodiments, the chimeric protein is administered as one or more doses of from about 45 IU/kg to about 70 IU/kg. In some embodiments, at least one of the multiple doses administered prophylactically is about 50 IU/kg. In some embodiments, each of the multiple doses administered prophylactically is about 50 IU/kg. In some embodiments, a single on-demand dose is about 50 IU/kg.

In certain aspects, the disclosure relates to a method of resolving a bleeding episode. In some embodiments, the method of resolving a bleeding episode comprises administering to a human subject in need thereof a single on-demand dose of a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

In some embodiments, every dose of the chimeric protein that is administered to the subject is about the same for at least 1 month. In some embodiments, every prophylactic dose of the chimeric protein that is administered to the subject is about the same for at least 1 month. In some embodiments, every dose of the chimeric protein that is administered to the subject is about the same for at least 3 months. In some embodiments, every prophylactic dose of the chimeric protein that is administered to the subject is about the same for at least 3 months. In some embodiments, every dose of the chimeric protein that is administered to the subject is about the same for at least 6 months. In some embodiments, every prophylactic dose of the chimeric protein that is administered to the subject is about the same for at least 6 months. In some embodiments, every dose of the chimeric protein that is administered to the subject is about the same for at least 9 months. In some embodiments, every prophylactic dose of the chimeric protein that is administered to the subject is about the same for at least 9 months. In some embodiments, every dose of the chimeric protein that is administered to the subject is about the same for at least 12 months. In some embodiments, every prophylactic dose of the chimeric protein that is administered to the subject is about the same for at least 12 months.

In some embodiments, the subject was previously treated for hemophilia A with a FVIII replacement protein other than a chimeric protein disclosed herein (such as efanesoctocog alfa), and the subject is more compliant with a dose regimen of the chimeric protein (e.g., adheres to the dose regimen, such as a once-weekly dose of 50 IU/kg) than the subject was with the dose regimen of the previous treatment. In some embodiments, the subject misses fewer doses of the chimeric protein compared to doses missed of the previous treatment.

In some embodiments, the subject is previously treated for hemophilia A with a FVIII replacement protein other than a chimeric protein disclosed herein (such as efanesoctocog alfa) via intravenous administration, before switching to treatment with the chimeric protein via intravenous injection. In some embodiments, the health of one or more injection sites that are used for intravenous injections improves after the subject switches. In some embodiments, the improved health at the one or more injection sites includes reduced swelling, reduced redness, or reduced itching.

In some embodiments, the subject has received on-demand treatment for at least 1 to 7 bleeding episodes within the last year (e.g., within a year before the first administration of the chimeric protein or before the initiation of prophylactic treatment with the chimeric protein). In some embodiments, the subject has received on-demand treatment for at least 2 to 5 bleeding episodes within the last year. In some embodiments, the subject has received on-demand treatment for at least 3 to 5 bleeding episodes within the last year. In some embodiments, the subject has received on-demand treatment for at least 3 to 6 bleeding episodes within the last year. In some embodiments, the subject has received on-demand treatment for at least 4 to 7 bleeding episodes within the last year. In some embodiments, the subject has received on-demand treatment for at least 1, at least 2, at least 3, at least 4, at least 5, at least 6, or at least 7 bleeding episodes within the last year. In some embodiments, the subject has received on-demand treatment for at least 1 bleeding episode within the last year. In some embodiments, the subject has received on-demand treatment for at least 2 bleeding episodes within the last year. In some embodiments, the subject has received on-demand treatment for at least 3 bleeding episodes within the last year. In some embodiments, the subject has received on-demand treatment for at least 4 bleeding episodes within the last year. In some embodiments, the subject has received on-demand treatment for at least 5 bleeding episodes within the last year. In some embodiments, the subject has received on-demand treatment for at least 6 bleeding episodes within the last year. In some embodiments, the subject has received on-demand treatment for at least 7 bleeding episodes within the last year.

### F. Dosing Interval

In certain embodiments, especially for prophylactic treatment, the chimeric protein, e.g., efanesoctocog alfa, is administered as multiple doses at a dosing interval. In some embodiments, the dosing interval is about 7 days. In some embodiments, the dosing interval is about 8 days. In some embodiments, the dosing interval is about 9 days. In some embodiments, the dosing interval is about 10 days. In some embodiments, the dosing interval is about 11 days. In some embodiments, the dosing interval is about 12 days. In some embodiments, the dosing interval is about 13 days. In some embodiments, the dosing interval is about 14 days. In some embodiments, the dosing interval is about 6 days to about 8 days. In some embodiments, the dosing interval is from 7 to 14 days. In some embodiments, 50 IU/kg of the chimeric protein (e.g., efanesoctocog alfa) is administered once weekly.

In some embodiments, the dosing frequency, e.g., for prophylactic treatment of hemophilia A, is at least once every week or at least once every 2 weeks. In some embodiments, the dosing interval, e.g., for prophylactic treatment of hemophilia A, is once per week. In some embodiments, the dosing interval, e.g., for prophylactic treatment of hemophilia A, is once every 2 weeks.

In some embodiments, the chimeric protein is administered for prophylactic treatment of hemophilia A. Prophylactic treatment of hemophilia A includes alleviating or reducing the severity of the symptoms of hemophilia A on a continuous or nearly continuous basis. In some embodiments, the prophylactic treatment is administered before the occurrence of a symptom of hemophilia A, e.g., before a bleeding incident. In some embodiments, the prophylactic treatment is administered on a regular basis, e.g., at a dosing interval described herein, to prevent the onset of or to lessen the severity of a symptom prior to the onset of the symptom.

In some embodiments, the chimeric protein is administered before an activity that can bring on one or more symptom of hemophilia A, e.g., as an on-demand treatment. For example, a chimeric protein of the present disclosure can be administered to a subject suffering from hemophilia A before the subject engages in an activity that otherwise increases the risk of physical trauma and/or a bleeding incident. When administered on-demand, the chimeric protein can be administered as a single dose or as multiple doses. In some embodiments, the on-demand treatment comprises administering multiple doses of the chimeric protein at a dosing interval of at least about 12 hours, at least about 24 hours, at least about 36 hours, at least about 48 hours, at least about 60 hours, at least about 72 hours, at least about 84 hours, at least about 96 hours, at least about 108 hours or at least about 120 hours. In certain embodiments, the on-demand treatment comprises administering at least 2 doses, at least 3 doses, at least 4 doses or at least 5 doses of the chimeric protein.

In some embodiments, the subject has previously been treated with one or more FVIII replacement therapy. In some embodiments, the subject failed to respond to a previous FVIII replacement therapy. In some embodiments, the FVIII replacement therapy is ELOCTATE® or ADVATE®.

In some embodiments, the subject is an adult. In some embodiments, the subject is an adult male. In some embodiments, the subject is an adult female. In some embodiments, the subject is a child, e.g., less than or equal to about 12 years old, less than or equal to or equal to about 11 years old, less than or equal to about 10 years old, less than or equal to about 9 years old, less than or equal to about 8 years old, less than or equal to about 7 years old, less than or equal to about 6 years old, less than or equal to about 5 years old, less than or equal to about 4 years old, less than or equal to about 3 years old, less than or equal to about 2 years old, or less than or equal to about 1 year old). In some embodiments, the subject is a female. In some embodiments, the subject is a male. In some embodiments, the subject is a female less than or equal to about 12 years old. In some embodiments, the subject is a female less than or equal to about 11 years old. In some embodiments, the subject is a female less than or equal to about 10 years old. In some embodiments, the subject is a male less than or equal to about 12 years old. In some embodiments, the subject is a male less than or equal to about 11 years old. In some embodiments, the subject is a male less than or equal to about 10 years old. In some embodiments, the subject is at least 12 years old. In some embodiments, the subject is at least 18 years old. In some embodiments, the subject has received previous treatment for hemophilia A (prophylaxis or on-demand) with a recombinant and/or plasma-derived FVIII, or cryoprecipitate for at least 150 exposure days (EDs). In some embodiments, the subject has received previous treatment for hemophilia A (prophylaxis or on-demand) with a recombinant and/or plasma-derived FVIII, or cryoprecipitate for less than 150 EDs. In some embodiments, the subject has not received previous treatment for hemophilia A (prophylaxis or on-demand) with a recombinant and/or plasma-derived FVIII, or cryoprecipitate.

A chimeric protein described herein can be administered by any means known in the art. In some embodiments, the chimeric protein is administered by intravenous injection, intravenous infusion, or subcutaneous administration. In some embodiments, the chimeric protein is administered intravenously. In some embodiments, the chimeric protein is administered subcutaneously.

In some embodiments, administration of the chimeric protein, e.g., efanesoctocog alfa, once per week results in a minimum (trough) FVIII plasma activity level between doses of at least about 5%, at least about 6%, at least about 7%, at least about 8%, at least about 9%, at least about 10%, or at least about 15%. In some embodiments, the minimum FVIII plasma activity level is at least about 5%. In some embodiments, the minimum FVIII plasma activity level is at least about 4%. In some embodiments, the minimum FVIII plasma activity level is at least about 5%. In some embodiments, the minimum FVIII plasma activity level is at least about 5.6%. In some embodiments, the minimum FVIII plasma activity level is at least about 7%. In some embodiments, the minimum FVIII plasma activity level is at least about 10%. In some embodiments, the minimum FVIII plasma activity level is at least about 12%. In some embodiments, the minimum FVIII plasma activity level is at least about 15%. In some embodiments, the minimum FVIII plasma activity level is at least about 20%. As used herein, plasma activity level is expressed as a percent (%). Alternatively, plasma activity can be expressed in IU/dL units, wherein 1% is equal to 1 IU/dL. In some embodiments, the FVIII activity level is as assessed by an aPTT assay. In some embodiments, the aPTT assay uses Actin FSL as a reagent. In some embodiments, the aPTT assay does not use Actin FS as a reagent.

In some embodiments, administration of the chimeric protein (e.g., efanesoctocog alfa) once per week results in a high sustained FVIII activity in the normal to near-normal range for a longer duration than currently marketed hemophilia A clotting factor replacement therapies (e.g., lonoctocog alfa, octocog alfa, rurioctocog alfa pegol, damoctocog alfa pegol, efmoroctocog alfa, simoctocog alfa, moroctocog alfa, plasma derived FVIII, beroctocog alfa, FVIII/VWF complex, and/or turoctocog alfa). In some embodiments, administration of the chimeric protein, e.g., efanesoctocog alfa, once per week results in a high sustained FVIII activity of at least >40% for a longer duration than currently marketed hemophilia A clotting factor replacement therapies (e.g., lonoctocog alfa, octocog alfa, rurioctocog alfa pegol, damoctocog alfa pegol, efmoroctocog alfa, simoctocog alfa, moroctocog alfa, plasma derived FVIII, beroctocog alfa, FVIII/VWF complex, and/or turoctocog alfa). In some embodiments, administration of the chimeric protein (e.g., efanesoctocog alfa) to a subject with hemophilia A achieves health equity with a corresponding subject who does not have hemophilia A. In some embodiments, administration of the chimeric protein (e.g., efanesoctocog alfa) to a subject with hemophilia A achieves a functional cure of the subject's hemophilia A. In some embodiments, the FVIII activity level is as assessed by an aPTT assay. In some embodiments, the aPTT assay uses Actin FSL as a reagent. In some embodiments, the aPTT assay does not use Actin FS as a reagent.

In some embodiments, the FVIII plasma activity level is at least about 40% at least about 4 days after the administration of the chimeric protein, e.g., at a dose of 50 IU/kg. In some embodiments, the FVIII plasma activity level is at least about 25% at least about 5 days after the administration of the chimeric protein, e.g., at a dose of 50 IU/kg. In some embodiments, the FVIII plasma activity level is at least about 30% at least about 5 days after the administration of the chimeric protein, e.g., at a dose of 50 IU/kg. In some embodiments, the FVIII plasma activity level is at least about 35% at least about 5 days after the administration of the chimeric protein, e.g., at a dose of 50 IU/kg. In some embodiments, the FVIII plasma activity level is at least about 5% at least about 7 days after the administration of the chimeric protein, e.g., at a dose of 50 IU/kg. In some embodiments, the FVIII plasma activity level is at least about 7.5% at least about 7 days after the administration of the chimeric protein, e.g., at a dose of 50 IU/kg. In some embodiments, the FVIII plasma activity level is at least about 10% at least about 7 days after the administration of the chimeric protein, e.g., at a dose of 50 IU/kg. In some embodiments, the FVIII plasma activity level is at least about 15% at least about 7 days after the administration of the chimeric protein, e.g., at a dose of 50 IU/kg. In some embodiments, the FVIII plasma activity level is at least about 2% at least about 14 days after the administration of the chimeric protein, e.g., at a dose of 50 IU/kg. In some embodiments, the FVIII plasma activity level is at least about 1% at least about 14 days after the administration of the chimeric protein, e.g., at a dose of 50 IU/kg. In some embodiments, the FVIII activity level is as assessed by an aPTT assay.

### IV. Pharmaceutical Compositions

In an aspect, the present disclosure is directed to pharmaceutical compositions of a chimeric protein, e.g., efanesoctocog alfa.

In some embodiments, the present disclosure is directed to a pharmaceutical composition comprising: efanesoctocog alfa, L-histidine, L-arginine hydrochloride, sucrose, calcium chloride, and polysorbate 80. In some embodiments, the pharmaceutical composition comprises 250, 500, 1000, 2000, 3000 or 4000 IU of efanesoctocog alfa. In some embodiments, the pharmaceutical composition comprises 1000 IU of efanesoctocog alfa. In some embodiments, the pharmaceutical composition comprises efanesoctocog alfa and a buffer comprising 10 mM L-histidine, 250 mM Arginine-HCl, 5 mM CaCl₂), 5% (w/v) sucrose, 0.05% (w/v) polysorbate 80. In some embodiments, the pharmaceutical composition has a pH of about 7.0. In some embodiments, the pharmaceutical composition has a pH of 6.8 to 7.2. In some embodiments, the pharmaceutical composition has a pH of 6.8. In some embodiments, the pharmaceutical composition has a pH of 7.0.

In some embodiments, the chimeric protein is in a pharmaceutical composition that comprises about 250 IU, 500 IU, 1000 IU, 2000 IU, 3000 IU, or 4,000 IU of the chimeric protein. In some embodiments, the chimeric protein is efanesoctocog alfa. In some embodiments, the efanesoctocog alfa is in a pharmaceutical composition that comprises: (a) 45 mg/mL to 60 mg/mL sucrose; (b) 1.5 mg/mL to 2.0 mg/mL L-histidine; (c) 40 mg/mL to 60 mg/mL L-arginine; (d) 0.5 mg/mL to 0.9 mg/mL calcium chloride; and (e) 0.4 mg/mL to 0.7 mg/mL polysorbate 80. In some embodiments, the efanesoctocog alfa is in a pharmaceutical composition that comprises: (a) about 56.12 mg/ml sucrose; (b) about 1.74 mg/ml L-histidine; (c) about 59.11 mg/ml L-arginine-HCl; (d) about 0.62 mg/ml calcium chloride; and (e) about 0.56 mg/ml polysorbate 80. In some embodiments, the efanesoctocog alfa is in a pharmaceutical composition that comprises: (a) 5% (w/v) to 7.5% (w/v) sucrose; (b) 7.5 mM to 12.5 mM histidine; (c) 225 mM to about 300 mM arginine; (d) 5 mM to 6 mM calcium chloride; and (e) 0.01% (w/v) to about 0.075% (w/v) polysorbate 80.

In some embodiments, the lyophilized pharmaceutical composition is in a lyophilized cake. In some embodiments, the lyophilized cake is white. In some embodiments, the lyophilized cake is less than Y4 in the European Pharmacopoeia color scale. See Degree of Coloration of Liquids (Method 2.2.2), European Pharmacopoeia, 10^{th} Ed. (2021).

In some embodiments, the pharmaceutical composition disclosed herein is a lyophilized pharmaceutical composition (e.g., powder). In some embodiments, the pharmaceutical composition is supplied in a sterile vial that requires reconstitution with sterile water.

In some embodiments, the lyophilized pharmaceutical composition and sterile water are combined to produce an injectable solution. In some embodiments, the lyophilized pharmaceutical composition is combined with about 2 mL to about 5 mL of sterile water. In some embodiments, the lyophilized pharmaceutical composition is combined with about 3 mL of sterile water. In some embodiments, the lyophilized pharmaceutical composition is combined with 3 mL of sterile water. In some embodiments, the sterile water is USP grade sterile water. In some embodiments, the sterile water is USP grade sterile water for injection. In some embodiments, the sterile water is pyrogen-free or nonpyrogenic. In some embodiments, the sterile water does not contain a bacteriostatic or antimicrobial agent. In some embodiments, the sterile water contains a bacteriostatic or antimicrobial agent. In some embodiments, the sterile water is sterilized using a filter. In some embodiments, the sterile water is sterilized using a 0.1 μm filter. In some embodiments, the sterile water is distilled water. In some embodiments, the sterile water is sterile, nonpyrogenic, distilled water, hypotonic, with an osmolarity of zero mOsmol/L, and does not contain a bacteriostatic or antimicrobial agent.

Also disclosed herein is a pharmaceutical kit for treatment of hemophilia A comprising a label and a chimeric protein. In some embodiments, the label comprises instructions for administering the chimeric protein. In some embodiments, the label comprises instructions for administering the chimeric protein once per week at a dose of 50 IU/kg. In some embodiments, the pharmaceutical label comprises an instruction or recommendation for practicing a method disclosed herein.

In some embodiments, the chimeric protein in the pharmaceutical kit is in a lyophilized pharmaceutical composition. In some embodiments, the kit further comprises a diluent for reconstituting the lyophilized pharmaceutical composition. In some embodiments, the diluent is sterile water. In some embodiments, the diluent is in a pre-filled syringe.

In some embodiments, for a dose of 50 IU/kg, the expected in vivo peak increase in Factor VIII level expressed as IU/dL (or % of normal) is estimated using the following formula: Estimated Increment of Factor VIII (IU/dL or % of normal)=50 IU/kg×2 (IU/dL per IU/kg). In some embodiments, potency is determined using an aPTT-based one-stage clotting assay. In some embodiments, the aPTT assay uses Actin FSL as a reagent. In some embodiments, the aPTT assay does not use Actin FS as a reagent.

In some embodiments, a chimeric protein (such as efanesoctocog alfa) is administered in a routine prophylaxis dose regimen. In some embodiments, the recommended dose for routine prophylaxis is 50 IU/kg administered every 7 days.

In some embodiments, the chimeric protein is provided as part of a kit with a pharmaceutical label. wherein the pharmaceutical label comprises information that is sufficient to enable a human subject, a caregiver, or a medical practitioner to practice a method of administering a chimeric protein as described herein.

In some embodiments, a chimeric protein (such as efanesoctocog alfa) is administered on-demand and/or for the control of a bleeding episode. In some embodiments, the dosing for the on-demand treatment and control of bleeding episodes is as described in Table 3. Examples of minor bleeds include, but are not limited to, mild epistaxis, early signs of joint bleeding, and mild soft tissue bleeds. Examples of moderate bleeds include, but are not limited to, epistaxis with heavy blood flow, muscle bleeding, gastrointestinal/oral mucosal bleeds, gum bleeding after dental extraction, hematuria, and hemarthrosis. Examples of major to life-threatening bleeds include, but are not limited to, epistaxis with very heavy blood flow, retropharyngeal and pharyngeal bleeding, abdominal and retroperitoneal bleeding, post-surgical bleeding, and CNS bleeding. Additional examples of minor, moderate, and major bleeds are shown in Table 3.

TABLE 3
Exemplary dosing based on bleed type.

| | Recommended | |
|---|---|---|
| Type of Bleeding | dose (IU/kg) | Additional information |
| Minor and Moderate | 50 IU/kg | A single dose should be sufficient for |
| For example: uncomplicated joint | | minor and moderate bleeds. |
| bleeds, minor muscular bleeds, | | For minor and moderate bleeding |
| mucosal or subcutaneous bleeds | | episodes occurring within 2 to 3 days |
| | | after a recent prophylactic dose, |
| | | an initial 30 IU/kg dose may be used. |
| | | Additional doses of 30 or 50 IU/kg |
| | | every 2 to 3 days may be considered. |
| Major | 50 IU/kg | Additional doses of 30 or 50 IU/kg |
| For example: Intracranial, | | every 2 to 3 days can be given. |
| retroperitoneal, iliopsoas and | | |
| neck bleeds, muscle bleeds with | | |
| compartment syndrome and | | |
| bleeds associated with a | | |
| significant decrease in the | | |
| hemoglobin level | | |

In some embodiments, for resumption of prophylaxis (if applicable) after treatment of a bleed, it is recommended to allow an interval of at least 72 hours between the last 50 IU/kg dose for treatment of a bleed and resuming prophylaxis dosing. In some embodiments, thereafter, prophylaxis can be continued as usual on the subject's regular schedule.

Having now described the present disclosure in detail, the same will be more clearly understood by reference to the following examples, which are included herewith for purposes of illustration only and are not intended to be limiting of the disclosure. All patents, publications, and articles referred to herein are expressly and specifically incorporated herein by reference.

### EXAMPLES

### Example 1: A Phase 3 Open-Label, Multicenter Study of the Safety, Efficacy, and Pharmacokinetics of Intravenous Efanesoctocog Alfa in Previously Treated Patients ≥12 Years of Age with Severe Hemophilia a

Efanesoctocog alfa, also known as “BIW001”, “efanesoctocogum alfa”, “Efa”, and “rFVIIIFc-VWF-XTEN”, is engineered to have an extended half-life that is independent of von Willebrand factor (VWF). Preclinical and clinical experience indicate that Efa has an extended half-life, irrespective of VWF level, and maintains high sustained FVIII activity levels for longer than currently marketed FVIII replacement treatments.

The object of the present study was to determine the safety, efficacy, and pharmacokinetics (PK) of efanesoctocog alfa administered as every week (QW) prophylaxis or on-demand treatment at a dose of 50 IU/kg IV. The study enrolls previously treated patients (PTPs) with severe hemophilia A aged 12 years or older. The study design takes into consideration the published guidelines for the clinical investigation of recombinant and human plasma-derived factor VIII products per the European Medicines Agency (EMA) Guideline on the clinical investigation of recombinant and human plasma-derived factor VIII products (Jul. 26, 2018).

Based on current treatment patterns for hemophilia A, the current study design allows for inclusion of hemophilia patients treated with either prophylactic therapy (Arm A) or on-demand therapy (Arm B).

### A. Study Design

This is a Phase 3, open-label, multinational, multicenter study of the safety, efficacy and PK of IV efanesoctocog alfa in previously treated patients PTPs >12 years of age with severe hemophilia A (defined as <1 IU/dL [<1%] endogenous FVIII). Participants currently on a prophylaxis treatment regimen with FVIII enter Arm A and receive efanesoctocog alfa at a dose of 50 IU/kg IV once-weekly (QW) on a prophylaxis treatment regimen for 52 weeks. A number of participants of Arm A have at least 6 months of participation in observational study 242HA201/OBS16221 prior to baseline.

A number of participants currently on an on-demand treatment regimen enter Arm B and receive efanesoctocog alfa at a dose of 50 IU/kg IV on an on-demand basis for 26 weeks, then receive efanesoctocog alfa at a dose of 50 IU/kg IV QW on a prophylaxis treatment regimen for 26 weeks. The switch design of Arm B enables an intra-subject comparison of on-demand treatment vs prophylactic treatment. Finally, Arm B also supports evaluation of efficacy of efanesoctocog alfa for on-demand treatment of bleeding episodes and surgical management.

Participants (except those in the sequential PK subgroup of Arm A) undergo abbreviated PK sampling after the first dose of efanesoctocog alfa (Baseline). Participants in the Sequential PK Subgroup of Arm A undergo more extensive PK sampling at Baseline and again at Week 26.

### Screening

Participants undergo a washout period of at least 48 hours prior to the Screening inhibitor test to obtain interpretable test results. Washout prior to efanesoctocog alfa Day 1 dose administration (Baseline Visit) is at least 96 hours (4 days) if the participant is receiving a conventional FVIII product and at least 120 hours (5 days) if current treatment is with an extended half-life (EHL) FVIII product. The washout period prior to the Baseline Visit for subjects in abbreviated PK sampling may be modified based on individual PK data and clinical phenotype.

### Enrollment and Baseline (Day 1)

Participants are enrolled after all screening assessments have been successfully completed and after the participant is determined to be eligible to participate in the study (see inclusion/exclusion criteria). A number of participants are enrolled into Arm A: Prophylaxis Treatment regimen. A number of participants are enrolled in Arm B: On-Demand Switch to Prophylaxis Treatment regimen. Some participants are enrolled in the sequential PK subgroup of Arm A.

Participants who enter Arm A receive efanesoctocog alfa as weekly prophylaxis for 52 weeks. Additional doses are given as necessary to treat breakthrough bleeding episodes. Doses are given in clinic according to the Schedule of Activities (SoA) (Table 4). Other doses are administered by the participant or caregiver at home, or in clinic.

Participants who enter Arm B receive efanesoctocog alfa to treat bleeds during the 26-week on-demand period. At Week 26, participants in Arm B switch to prophylaxis dosing, and dose efanesoctocog alfa at 50 IU/kg once weekly, until Week 52. Additional doses are given as necessary to treat breakthrough bleeding episodes during the 26-week prophylaxis period. Doses are given in clinic according to the SoA (Table 4). Other doses are administered by participant or caregiver at home, or in clinic.

Participants are supplied with an electronic patient diary (ePD) at the Baseline visit to record all bleeding episodes and doses of efanesoctocog alfa administered after the Baseline visit. Entries are made in a timely manner and it is preferred that details of doses are entered immediately upon administration or within 7 days. Participants are prompted to enter bleeding location, type (spontaneous or traumatic), and reasons for dosing (prophylaxis or treatment of a bleeding episode). An ePD training is completed at the study site at the Baseline visit.

Participants with pre-planned surgery participating in the sequential PK subgroup do not enter the surgery subgroup until repeat PK sampling has been completed 26 weeks following the efanesoctocog alfa Day 1 PK profiling.

Participants from any arm who undergo major surgery during the study are included in the surgery subgroup.

All participants have an initial dose of efanesoctocog alfa on Day 1 and have subsequent PK assessments.

### Study Clinic Visits

All enrolled participants come in to scheduled visits at the following time points: Baseline (Day 1), Week 4, Week 13, Week 26, Week 39, and Week 52. Details of each visit are provided in Table 4.

For participants in Arm A: each scheduled study visit subsequent to efanesoctocog alfa Day 1 is arranged 7 days (±1 day) after the preceding prophylaxis dose. The participant should withhold the weekly prophylaxis dose on the day of the study visit, so that it can be administered at the study visit.

A subgroup of participants in Arm A (Sequential PK subgroup) undergo Baseline efanesoctocog alfa PK sampling and repeat efanesoctocog alfa PK sampling at the Week 26 Visit (to occur 7 (+2) days after preceding prophylaxis dose).

For participants in Arm B: each scheduled study visit subsequent to Week 26 is to be arranged when a scheduled dose is due 7 days (±1 day) after the preceding prophylaxis dose. The participant should withhold the weekly prophylaxis dose, so that it can be administered at the study visit.

Participants undergo efficacy and safety assessments throughout their participation in the study. Safety assessments include testing for inhibitor development to FVIII. A Follow-up Safety Visit or Telephone Call occurs 2 to 3 weeks after the last dose of efanesoctocog alfa, unless the participant enrolls in the open-label extension study. Participants complete their End of Study (EOS) Visit 52 weeks (±7 days) post-Baseline, or earlier if EOS is been declared.

### Statistical Analyses

Data for bleeding episodes are collected by each participant via an electronic diary or, in the case of bleeding episodes occurring and/or treated at the study site, on the electronic case report form and include type of bleed, location of bleed, and treatment dates, if applicable. This information is used to derive the primary and related secondary efficacy endpoints.

All analyses of bleeding endpoints is based on treated bleeding episodes, except for a summary of ABR for all bleeding episodes which includes both treated and untreated bleeding episodes.

All primary analyses of bleeding endpoints is based on treated bleeding episodes consistent with ISTH criteria (Blanchette 2014) using a standardized definition. A bleeding episode starts from the first sign of bleeding and ends no more than 72 hours after the last injection to treat the bleeding episode. Any subsequent bleeding at the same location and injections administered less than or equal to 72 hours from the previous injection will be considered the same bleeding episode. Multiple bleeding locations treated with a single injection will also be considered a single bleeding episode. Any injection to treat the bleeding episode that is administered more than 72 hours after the preceding one is considered the first injection to treat a new bleeding episode in the same location. Any injection used to treat subsequent bleeding at a different location is considered a separate bleeding episode, regardless of the time from the last injection to treat a bleeding episode. If a bleeding episode occurs on the same day as a regularly scheduled prophylaxis dose, participants are instructed to record in their diary treated and untreated bleeds and the timing of prophylactic and bleed treatments.

In addition, an analysis of all bleeding episodes (including untreated bleeds) is performed. The definition of all bleeds follows the ISTH criteria, except that untreated bleeds are counted based on the date and time of the bleed itself.

The following hemophilia history variables are summarized by study arm and treatment regimen, including the surgery subgroup, and overall using descriptive statistics: age at diagnosis of severe hemophilia (years); family inhibitor history (Yes, No); blood type (A, B, AB, O); Rh factor (Positive, Negative); lowest documented historical FVIII level (<1%, >1%); FVIII genotype; Human immunodeficiency virus (HIV) status; Hepatitis C (HCV) status; Hepatitis B (HBV) status; vaccination history in past year (yes, no); types of FVIII product previously administered at study entry; age at start of first prophylaxis regimen (years; <6, 6 to <10, 10 to <18, and >18 years); number of prior exposure days to FVIII (<150, 2150); number of bleeds in the past 12 months; number of joint bleeds in the past 12 months; and target joint at baseline.

In addition, the most recent pre-study FVIII regimen and other prior FVIII regimens used in the past 12 months are collected and summarized including: categories for most recent pre-study FVIII regimen (prophylaxis, on-demand); time on pre-study regimen (>12 months, 6-12 months, <6 months); and frequency of injections for those participants who indicated prophylaxis as their most recent pre-study regimen.

TABLE 4
Schedule of Activities (SoA)

| | Screening^{a} | Baseline^{b} | | | |
|---|---|---|---|---|---|
| | (Week −8 | BIVV001 | Week 4^{c} | Week 13^{c} | Week 26^{c, d} |
| Tests and assessments | to Day 1) | Day 1 | ±7 days | ±7 days | ±7 days |
| Informed consent^{g} | X | | | | |
| Assessment of eligibility | X | X | | | |
| Demographics^{h} | X | | | | |
| Weight | X | X | X | X | X |
| Height [symbol] | X | | | | |
| Medical, surgical, and | X | | | | |
| hemophilia historyⁱ | | | | | |
| FVIII activity | X | | | | |
| (1-stage aPIT assay)^{k} | | | | | |
| Genotype [symbol] | | X | | | |
| In-clinic BIVV001 | | X | X | X | X |
| dosing [symbol] | | (applicable | (applicable | (applicable | |
| | | to both Arm | to Arm A only) | to Arm A only) | |
| | | A and Arm B) | | | |

Safety

| | | | | | |
|---|---|---|---|---|---|
| Physical exam [symbol] | X | X | X | | X |
| Vital signs^{m} | X | X | X | | X |
| Pregnancy testingⁿ | X | X | | X | X |
| HIV, HBV, and HCV | X | | | | |
| status^{o} | | | | | |
| CD4 count, viral load^{p} | X | | | | |
| Hematology^{q} | X | X | X | X | X |
| Coagulation parameters^{q} | | X | | | X |
| Clinical chemistry^{r} | X | X | X | X | X |
| von Willebrand | X | X | X | X | X |
| Comprehensive Panel^{s} | | | | | |
| Nijmegen-modified | X | X | X | X | X |
| Bethesda assay | | | | | |
| (inhibitor assay)^{k} | | | | | |
| Anti-rFVIIIFc-VWF-XTEN | X | X | X | X | X |
| Antibody (ADA)^{s} | | | | | |

| | |
|---|---|
| Adverse event/serious | <<ongoing monitor and record at all visits>> |
| adverse event recording^{t} | |
| Prior and concomitant | <<ongoing monitor and record at all visits>> |
| medications and concomitant | |
| therapies and procedures^{u} | |
| Monthly telephone call^{v} | <<ongoing once per month starting at Week 4>> |

Efficacy

| HJHS joint assessment^{w} | | X | | | X |
|---|---|---|---|---|---|
| Investigator's target | | X | | | |
| joint assessment | | | | | |
| Physician's global | | | X | X | X |
| assessment of response | | | | | |
| to treatment (PGA) | | | | | |
| Investigator's assessment of | | <<ongoing>> | | | |
| participant's response to | | | | | |
| treatment of bleeding episodes | | | | | |
| treated at the study site^{x} | | | | | |
| Ultrasound joint | | X | | | |
| assessments^{y} | | | | | |
| Serum and plasma | X | X | X | X | X |
| samples^{z} (optional) | | | | | |

| | | | |
|---|---|---|---|
| Electronic patient diary (ePD) | X [symbol] | X | <<ongoing>> |
| training/administration/review | | | |

| | |
|---|---|
| Participants completion of | <<ongoing>> |
| ePD including at-home dosing, | |
| bleeding episodes, and | |
| assessment of response to | |
| treatment of bleeding | |
| episodes^{aa} | |

Pharmacokinetics

| | | | | |
|---|---|---|---|---|
| Abbreviated pharmacokinetic | X | | | |
| Sampling^{bb} | | | | |
| Sequential pharmacokinetic | X | | | X |
| sampling^{cc} | | | | |
| FVIII peak and trough | | X | X | X |
| sampling^{dd} | | Arm A only | Arm A only | |

Participant-Reported Outcomes

| | | | | | |
|---|---|---|---|---|---|
| PROMIS Assessments^{ee} | | X | | | X |
| [symbol] -A-QoL (≥17 years old) | | X | | | X |
| or [symbol] QoL (<17 years old) | | | | | |
| HAL (≥18 years old) or | | X | | | X |
| pedHAL (<18 years old) | | | | | |
| TSQM-9 | | X | | | X |
| Patient Global Impression | | X | | | X |
| of Severity (activity and | | | | | |
| joint)^{ff} | | | | | |
| Patient Global Impression | | | | | X |
| of Change | | | | | |
| EQ-50-5L | | X | | | X |
| Treatment Preference Survey^{gg} | | | | | |
| Physical Activity Monitor | X | X | X | X | X |
| (Act/Graph Activity Monitor)^{hh} | | | | | |
| Exit interior^{kk} | | | | | |
| Healthcare Resource Utilization | | X | | | X |

| | | Week 52/ | | Safet [symbol] |
|---|---|---|---|---|
| | Week 39^{c} | EOS/ET^{c} | Unscheduled | follow- [symbol] |
| Tests and assessments | ±7 days | ±7 days | visit^{e} | call or v [symbol] |
| Informed consent^{g} | | | | |
| Assessment of eligibility | | | | |
| Demographics^{h} | | | | |
| Weight | X | X | | |
| Height [symbol] | | | | |
| Medical, surgical, and | | | | |
| hemophilia historyⁱ | | | | |
| FVIII activity | | | | |
| (1-stage aPIT assay)^{k} | | | | |
| Genotype [symbol] | | | | |
| In-clinic BIVV001 | X | X | | |
| dosing [symbol] | | | | |
| Safety | | | | |
| Physical exam [symbol] | | X | X | |
| Vital signs^{m} | | X | X | |
| Pregnancy testingⁿ | X | X | | |
| HIV, HBV, and HCV | | | | |
| status^{o} | | | | |
| CD4 count, viral load^{p} | | | | |
| Hematology^{q} | X | X | | |
| Coagulation parameters^{q} | | X | | |
| Clinical chemistry^{r} | X | X | | |
| von Willebrand | X | X | | |
| Comprehensive Panel^{s} | | | | |
| Nijmegen-modified | X | X | | |
| Bethesda assay | | | | |
| (inhibitor assay)^{k} | | | | |
| Anti-rFVIIIFc-VWF-XTEN | X | X | | |
| Antibody (ADA)^{s} | | | | |
| Adverse event/serious | <<ongoing monitor and record at all visits>> | | | |
| adverse event recording^{t} | | | | |
| Prior and concomitant | <<ongoing monitor and record at all visits>> | | | |
| medications and concomitant | | | | |
| therapies and procedures^{u} | | | | |
| Monthly telephone call^{v} | <<ongoing once per month starting at Week 4>> | | | |
| Efficacy | | | | |
| HJHS joint assessment^{w} | | X | | |
| Investigator's target | | | | |
| joint assessment | | | | |
| Physician's global | X | X | | |
| assessment of response | | | | |
| to treatment (PGA) | | | | |
| Investigator's assessment of | <<ongoing>> | | | |
| participant's response to | | | | |
| treatment of bleeding episodes | | | | |
| treated at the study site^{x} | | | | |
| Ultrasound joint | | X | | |
| assessments^{y} | | | | |
| Serum and plasma | X | X | | |
| samples^{z} (optional) | | | | |
| Electronic patient diary (ePD) | <<ongoing>> | | | |
| training/administration/review | | | | |
| Participants completion of | <<ongoing>> | | | |
| ePD including at-home dosing, | | | | |
| bleeding episodes, and | | | | |
| assessment of response to | | | | |
| treatment of bleeding | | | | |
| episodes^{aa} | | | | |
| | Pharmacokinetics | | | |
| Abbreviated pharmacokinetic | | | | |
| Sampling^{bb} | | | | |
| Sequential pharmacokinetic | | | | |
| sampling^{cc} | | | | |
| FVIII peak and trough | X | X | | |
| sampling^{dd} | | | | |
| | Participant-Reported Outcomes | | | |
| PROMIS Assessments^{ee} | | X | | |
| [symbol] -A-QoL (≥17 years old) | | X | | |
| or [symbol] QoL (<17 years old) | | | | |
| HAL (≥18 years old) or | | X | | |
| pedHAL (<18 years old) | | | | |
| TSQM-9 | | X | | |
| Patient Global Impression | | X | | |
| of Severity (activity and | | | | |
| joint)^{ff} | | | | |
| Patient Global Impression | | X | | |
| of Change | | | | |
| EQ-50-5L | | X | | |
| Treatment Preference Survey^{gg} | | X | | |
| Physical Activity Monitor | X | X | | |
| (Act/Graph Activity Monitor)^{hh} | | | | |
| Exit interior^{kk} | | X | | |
| Healthcare Resource Utilization | | X | | |

Efanesoctocog alfa = BIVV001 = rFVIIIFc-VWF-XTEN (recombinant coagulation factor VIII Fc - von Willebrand factor - ELNN fusion protein), BU = Bethesda units, FVIII = Factor VIII, HBV = hepatitis B virus, HCV = hepatitis C virus, HIV = human immunodeficiency virus, HJHS = Hemophilia Joint Health Score HAL = Hemophilia Activities List, pedHAL = Pediatric Hemophilia Activities List, TSQM = Treatment Satisfaction Questionnaire for Medications, PGIC = Patient Global Impression of Change, PGIS = Patient Global Impression of Severity
^{a}Screening is accomplished over the course of more than 1 study visit if needed. The Screening Period is up to 8 weeks before Baseline. Washout prior to the Screening inhibitor test is at least 48 hours to obtain interpretable test results.
^{b}Washout prior to efanesoctocog alfa Day 1 dose administration is at least 96 hours (4 days) if the participant is receiving a conventional FVIII product and at least 120 hours (5 days) if current treatment is with an EHL FVIII product. The washout period before the Baseline Visit for subjects in abbreviated PK sampling may be modified at the discretion of the Investigator based on individual PK data and clinical phenotype in consultation with medical monitor.
^{c}Participants schedule their study visits to be 7 ± 1 day after the previous prophylactic dose of efanesoctocog alfa.
^{d}At this visit, participants in Arm B (on-demand treatment) switch to prophylaxis. Participants in the Sequential PK arm should schedule their Week 26 study visit to be 7 (+2) days after the previous prophylactic dose of efanesoctocog alfa.
^{e}Unscheduled visits may be necessary during the study to repeat any blood sampling if required, or at the discretion of the Investigator. If a participant has an unscheduled visit, the Investigator will record data as appropriate based on the purpose of the visit.
^{f}This call or visit occurs 14 (+7) days after the last dose of efanesoctocog alfa, unless the participant enrolls in the open-label extension study.
^{g}Informed consent from the participant or the participant's legal guardian is obtained.
^{h}Demographics include sex, race, ethnicity, and date of birth (year of birth only), as permitted by local regulations.
ⁱIncludes hemophilia history assessment of disease severity, blood type, and Rh factor if not previously documented. For Repeat Screening Visit, update with any changes since original Screening Visit.
^{j}HLA genotype will not be needed if previously documented.
^{k}Washout prior to the Screening inhibitor test is at least 48 hours to obtain interpretable test results. Washout prior to efanesoctocog alfa Day 1 dose administration is at least 96 hours (4 days) if the participant is receiving a conventional FVIII product and at least 120 hours (5 days) if current treatment is with an EHL FVIII product. The washout period prior to efanesoctocog alfa Day 1 dose administration for subjects in abbreviated PK sampling is modified based on individual PK data and clinical phenotype. Washout prior to all other scheduled inhibitor tests must be at least 7 ± 1 days. Inhibitor and ADA samples are collected prior to efanesoctocog alfa dosing. Separate samples for anti-rFVIIIFc-VWF-XTEN antibody (ADA) testing are collected at the same timepoint when any samples are collected for inhibitor testing (including samples for suspected inhibitor and confirmatory inhibitor tests). All inhibitor assays, including the assay for the Screening Visit and any confirmatory assays, are performed by the central laboratory using the Nijmegen-modified Bethesda assay. If a Nijmegen-modified Bethesda assay result returns as ≥0.6 BU/mL, a separate sample must be collected and tested for confirmation of inhibitor development within 2 to 4 weeks of the original sample. Testing for potential ADA formation is performed using a validated rFVIIIFc-VWF-XTEN-specific ADA assay. Confirmed positive samples are further characterized for antibodies specific to FVIII, Fc, D′D3, or ELNN polypeptide.
^{l}Participants in Arm A have efanesoctocog alfa doses given at each applicable scheduled visit. Participants in Arm B have efanesoctocog alfa doses at visits Day 1, Weeks 26, 39, and 52. During the Scheduled study visits, efanesoctocog alfa is delivered via a slow push IV injection of 8 ± 2 minutes, at a rate of administration determined by the participant's comfort level. For all efanesoctocog alfa injections performed at home efanesoctocog alfa is delivered via a slow push IV injection at a rate of administration determined by the participant's comfort level according to the vial and injection rate recommendations. Injection start and stop time is recorded. Other doses may be self/caregiver administered at home (or in clinic).
^{m}Vital signs include blood pressure, pulse rate, respiratory rate, and temperature (° C.). Vital signs should be taken after the participant has been resting supine for 5 minutes. Vital signs will be measured pre-injection and 30 (±15 minutes) from the start of injection at clinic visits. Vital signs should also be taken at any unscheduled visit.
ⁿApplicable to female participants only. A serum pregnancy test should be performed at screening. For all other time points, the choice of the pregnancy test (urine or serum) is at the discretion of the Investigator. If a urine test cannot be confirmed as negative (e.g., an ambiguous result), a serum pregnancy test is required. In such cases, the participant is excluded from further participation if the serum pregnancy result is positive.
^{o}For participants who have been historically negative, viral testing is performed at a central laboratory. Human immunodeficiency virus (HIV) tests include HIV-1 antibodies, HIV-2 antibodies and HIV-1 p24 antigen. Hepatitis B virus (HBV) tests include HBV surface antigen, anti-HBV surface antibody and anti-HBV core antibody. Hepatitis C virus (HCV) tests include anti-HCV antibodies.
^{p}For participants known to be HIV antibody positive, CD4 count and viral load tests are performed at the central laboratory if results are not available from within 26 weeks prior to screening.
^{q}Hematology parameters include red blood cell (RBC) count, white blood cell (WBC) count and differential, platelet count, hemoglobin (Hgb), and hematocrit (Hct). Blood samples for hematology analysis are collected prior to efanesoctocog alfa dosing.
^{r}Clinical chemistry parameters include alanine aminotransferase (ALT), aspartate aminotransferase (AST), alkaline phosphatase (ALP), gamma glutamyl transferase (GGT), bilirubin, blood urea nitrogen (BUN), creatinine, glucose, total protein, sodium, potassium, and chloride. Blood samples for clinical chemistry analysis are collected prior to efanesoctocog alfa dosing.
^{s}The Von Willebrand comprehensive panel (includes assessments of von Willebrand Factor [VWF] ristocetin cofactor activity and VWF antigen). Blood samples for analysis of Von Willebrand comprehensive panel are collected prior to any efanesoctocog alfa dosing.
^{t}Adverse events (AEs) and serious adverse events (SAEs) occurring after signing of the informed consent form (ICF) through the Safety Follow-Up Call or Visit are recorded.
^{u}Prior and concomitant medications from up to 30 days prior to Screening and concomitant therapies and procedures from signing of ICF through the Safety Follow-Up Call or Visit are recorded. Pain medication related to Hemophilia and administered within 2 weeks prior to the visit are recorded on the CRF.
^{v}In addition to scheduled clinic visits, telephone calls are planned approximately once a month for the site staff to check on each participant's status. During the monthly telephone call, assessments of “spontaneous” and “traumatic” bleeding episodes will be noted.
^{w}Each participant's joints is examined per the Haemophilia Joint Health Score (HJHS). At baseline, the presence of any target joints is assessed according to ISTH criteria.
^{x}For bleeding episodes treated at the study sites, the participant is contacted approximately 72 hours from the time the efanesoctocog alfa injection was administered to treat the bleeding episode and participant's assessment of response to efanesoctocog alfa treatment is recorded using a 4-point bleeding response scale. For bleeding episodes that are treated at home, participants will record response to bleeding episodes in the ePD.
^{y}Anatomical structural joint-health assessment via ultrasound imaging per Joint Tissue Activity and Damage Examination (JADE) protocol and/or Hemophilia Early Arthropathy Detection with Ultrasound (HEAD-US) in a subpopulation of participants from Arm A at selected sites. The Ultrasound can be performed at the Baseline visit or up to 2 weeks after the Baseline Visit.
^{z}Samples are collected prior to any efanesoctocog alfa dosing and are optionally archived.
^{aa}Participants record all bleeding episodes beginning at Baseline. Assessment of response to bleeding episodes using a 4-point bleeding response scale. The participant should record response approximately 72 hours from the time the first efanesoctocog alfa injection was administered to treat the bleeding episode, unless treatment of the bleed was administered in the clinic. In that case, the investigator reports the participant's response to treatment.
^{bb}Participants (except those in the sequential PK subgroup of Arm A) will undergo abbreviated PK sampling after the first dose of efanesoctocog alfa (Baseline).
^{cc}Participants in the sequential PK subgroup (n = 16) undergo more extensive PK sampling at Baseline and again at Week 26.
^{dd}Peak and trough sample only (collected within 30 min prior to rFVIIIFc-VWF-XTEN dosing and 15 min ± 3 min post injection. This trough sample should be collected at the same time point as the trough inhibitor and anti-drug antibody (ADA) samples taken predose.
^{ee}PROMIS Assessments to include Pain Intensity, Pain Interference (PROMIS-SF Pain Interference ≥18 years old or PROMIS Pediatric-SF Pain Interference <18 years old), and Physical Function (PROMIS-SF Physical Function ≥18 years old or PROMIS Pediatric-SF Physical Activity <18 years old).
^{ff}Patient Global Impression of Severity (PGIS) assessment includes a single question on patient perception of activity and a single question on patient perception of joint health.
^{gg}Treatment preference survey consists of 2 questions on patient preference and a single question on Patient Global Impression of Change (PGIC).
^{hh}Where available, assessments of physical activity (PA) are done by using a triaxial medical grade accelerometer (ActiGraph Activity Monitor) worn on the nondominant wrist. Device is worn daily for 8 consecutive days after the scheduled visits at Screening, Baseline, Weeks 4, 13, 26, 39, and for 8 consecutive days before the Week 52 visit.
ⁱⁱCoagulation parameters include activated partial thromboplastin time (aPTT)
^{jj}Please consider Italy specific requirements
^{kk}Interviews are conducted with a subset of participants and may be done up to 6 months after the Week 52/EOS Visit, but before the EOS is declared.
^{ll}ePD training only. ePD devices are given to the patients at the baseline visit [symbol]
indicates data missing or illegible when filed

### B. Study Population

### Inclusion Criteria

Participants are eligible to be included in the study only if all of the following criteria apply: 1-01. Participant must be equal to or greater than 12 years of age inclusive, at the time of signing the informed consent. 1-02. Patient has severe hemophilia A, defined as <1 IU/dL (<1%) endogenous FVIII activity as documented either by central laboratory testing at Screening or in historical medical records from a clinical laboratory demonstrating <1% FVIII coagulant activity (FVIII:C) or a documented genotype known to produce severe hemophilia A. 1-03. Previous treatment for hemophilia A (prophylaxis or on demand) with any recombinant and/or plasma-derived FVIII, or cryoprecipitate for at least 150 EDs. 1-04. Current regimen includes one of the following: (1) Prophylactic treatment regimen with a FVIII product or prophylactic emicizumab therapy for at least 6 months during the previous 12 months. (2) On-demand regimen with a FVIII product with a history of at least 12 bleeding episodes in the previous 12 months or at least 6 bleeding episodes in the previous 6 months prior to study enrollment (on-demand participant is accepting to move to a prophylaxis treatment regimen after 26-week on-demand period). 1-05. Platelet count >100,000 cells/μL at Screening. 1-06. A participant known to be human immunodeficiency virus (HIV) antibody positive, either previously documented or identified from screening assessments, must have the following results prior to enrollment: a) CD4 lymphocyte count >200 cells/mm³, and b) Viral load of <400 copies/mL. Documented results of CD4 lymphocyte count and viral load are accepted if samples were collected within 26 weeks prior to Screening or if samples were collected during Screening and evaluated by the central laboratory. Participants who have previously tested negative for HIV must have a repeat test by the central laboratory during Screening 1-07. Willingness and ability of the participant or surrogate (a caregiver or a family member >18 years of age) to complete training in the use of the study electronic Patient Diary (ePD) and to use the ePD throughout the study. 1-08. Male or female; with contraceptive use consistent with local regulations regarding the methods of contraception for those participating in clinical studies. a) Male participants—No contraceptive measures required for this study. b) Female participants—A female participant is eligible to participate if she is not pregnant or breastfeeding, and is either not a woman of childbearing potential (WOCBP) or is a WOCBP and using an acceptable contraceptive method during the intervention period (at a minimum until Safety Follow-up Call or Visit). A WOCBP must have a negative highly sensitive pregnancy test before the first dose of study intervention. 1-09. Participant must be capable of giving signed informed consent which includes compliance with the requirements and restrictions listed in the informed consent form (ICF) and in this protocol. 1-10. Ability of the participant or his or her legally authorized representative (e.g., parent or legal guardian) to understand the purpose and risks of the study and provide signed and dated informed consent or assent (as applicable) and authorization to use protected health information in accordance with national and local participant privacy regulations.

### Exclusion Criteria

Participants were excluded from the study if any of the following apply (medical conditions, prior/concomitant therapy, prior/concurrent clinical study experience, and other exclusions):

Medical conditions: E-01. Any concurrent clinically significant liver disease that, in the opinion of the Investigator, would make the participant unsuitable for enrollment. This may include, but is not limited to cirrhosis, portal hypertension, and acute hepatitis. E-02. Serious active bacterial or viral infection (other than chronic hepatitis or HIV) present within 30 days of Screening. E-03. Other known coagulation disorder(s) in addition to hemophilia A. E-04. History of hypersensitivity or anaphylaxis associated with any FVIII product. E-05. History of a positive inhibitor test defined as >0.6 BU/mL, or any value greater than or equal to the lower sensitivity cut-off for laboratories with cut-offs for inhibitor detection between 0.7 and 1.0 BU/mL, or clinical signs or symptoms of decreased response to FVIII administrations. Family history of inhibitors will not exclude the participant. E-06. Positive inhibitor result, defined as >0.6 BU/mL at Screening. E-07. Abnormal renal function, defined as serum creatinine >2.0 mg/dL taken at Screening. E-08. Serum alanine aminotransferase (ALT) or aspartate aminotransferase (AST) >5×upper limit of normal (ULN) taken at Screening. E-09. Serum total bilirubin >3×ULN, taken at Screening.

Prior/concomitant therapy: E-10. Vaccination within 30 days of Screening. E-11. Treatment with acetylsalicylic acid (ASA) or non-NSAID anti-platelet therapies within 2 weeks prior to screening. E-12. Treatment with non-steroidal anti-inflammatory drugs (NSAIDs) above the maximum dose specified in the regional prescribing information within 2 weeks prior to Screening. E-13. Systemic treatment within 12 weeks prior to Screening with chemotherapy and/or other immunosuppressive drugs (except for the treatment of hepatitis C virus [HCV] or HIV). Use of corticosteroids is allowed, except for systemic corticosteroid treatment given daily or on alternate days for >14 days. Local, topical, and/or inhaled steroid use is permitted.

Prior/concurrent clinical study experience: E-14. Emicizumab use within the 20 weeks prior to Screening. E-15. Previous enrollment in this study; participants who fail Screening may re-screen. E-16. Treatment with an investigational product within 30 days or 5.5 half-lives prior to Screening, whichever is longer. For investigational products with a pharmacodynamic effect that persists longer than the half-life, the maximal pharmacodynamic effect must return to baseline prior to Screening.

Other exclusions: E-17. Major surgery within 8 weeks prior to Screening. Major surgery is defined as any surgical procedure (elective or emergent) that usually, but not always, involves general anesthesia and/or respiratory assistance, in which a major body cavity is penetrated and exposed, or a substantial impairment of physical or physiological functions is produced (e.g., laparotomy, thoracotomy, craniotomy, joint replacement, or limb amputation). E-18. Individuals accommodated in an institution because of regulatory or legal order; prisoners or participants who are legally institutionalized. E-19. Any country-related specific regulation that would prevent the participant from entering the study. E-20. Participant not suitable for participation, whatever the reason, as judged by the Investigator, including medical or clinical conditions, or participants potentially at risk of noncompliance to study procedures. E-21. Participants are dependent on the Sponsor or Investigator. E-22. Participants are employees of the clinical study site or other individuals directly involved in the conduct of the study, or immediate family members of such individuals. E-23. Sensitivity to any of the study interventions, or components thereof, or drug or other allergy that, in the opinion of the Investigator, contraindicates participation in the study.

Any medication or vaccine (including over-the-counter or prescription medicines, vitamins, and/or herbal supplements) that the participant is receiving at the time of enrollment or receives during the study must be recorded along with: reason for use, dates of administration including start and end dates, and dosage information including dose and frequency. Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the Follow-up Visit, unless, the medication will not interfere with the study.

Participants who routinely administer an additional dose of FVIII prior to a sports activity or increased physical activity are not allowed to do so in this study. However, the day of dosing Efa in Arms A and B (prophylaxis period) could be chosen prior to a weekly recurring physical activity. Study Intervention & Dosing

For this study, study intervention is defined as any investigational intervention intended to be administered to a study participant according to the study protocol. An overview of study interventions administered is shown in Table 5.

TABLE 5
Overview of study interventions administered

| | | |
|---|---|---|
| ARM name | Prophylaxis | On-demand |
| Intervention name | Efanesoctocog alfa (rFVIIIFc- | Efanesoctocog alfa (rFVIIIFc- |
| | VWF-XTEN; BIVV001) | VWF-XTEN; BIVV001) |
| Type | Drug | Drug |
| Dose formulation | Lyophilized powder in a sterile | Lyophilized powder in a sterile |
| | vial that requires reconstitution | vial that requires reconstitution |
| | with Sterile Water for Injection | with Sterile Water for Injection |
| | (diluent) | (diluent) |
| Unit dose strength(s) | 250, 500, 1000, 2000, 3000 | 250, 500, 1000, 2000, 3000 |
| | and 4000 IU per vial | and 4000 IU per vial |
| Dosage level(s) | 50 IU/Kg every week | 50 IU/Kg |
| Route of administration | Intravenous | Intravenous |
| IMP and NIMP | IMP | IMP |
| Packaging and labeling | Study intervention will be | Study intervention will be |
| | provided in vials. Each vial | provided in vials. Each vial |
| | will be labeled as required | will be labeled as required |
| | per country requirement | per country requirement. |

### Prophylactic Treatment

The prophylactic treatment regimen is once weekly (every 7 days) dosing with 50 IU/kg efanesoctocog alfa. The aim of the 50 IU/kg IV QW treatment regimen is to provide sustained FVIII activity levels in the normal to mild hemophilia range (maintaining FVIII trough level between about 10% and 20% at Day 7) and to decrease treatment burden with once weekly dosing. During the scheduled study visit, efanesoctocog alfa is delivered via a slow push IV injection of 8±2 minutes, at a rate of administration determined by the participant's comfort level. For all efanesoctocog alfa injections performed at home efanesoctocog alfa are delivered via a slow push IV injection at a rate of administration determined by the participant's comfort level according to the vial injection rate recommendations.

Any missed doses should be taken as soon as possible or according to the instructions of the Investigator, taking into account that 2 consecutive prophylactic doses of 50 IU/kg should be separated by a period of at least 72 hours.

During the clinic visits and monthly telephone calls, the study site staff verify whether or not a bleeding episode has occurred, and if it was “spontaneous” or “traumatic”. During the monthly telephone call, the participant or participant's parent/caregiver is reminded about the requirement that ePD data be entered as soon as possible, (within a maximum of 7 days) following an injection, as well as verify treatment regimen compliance.

Bleeding episodes requiring treatment during prophylaxis with efanesoctocog alfa are treated with a single dose of 50 IU/kg. If a bleed does not improve, additional doses of 30 or 50 IU/kg every 2 to 3 days may be considered. For mild/moderate bleeds within 2-3 days of a recent prophylactic dose, an initial 30 IU/kg dose is also used. Treatment of any bleeding reported within 72 hours of a previous episode at the same location is considered a follow-up injection and the event should not be recorded as a new bleeding episode. If a bleeding episode occurs at the time when a regular prophylaxis dose of efanesoctocog alfa is due, the participant injects with a dose of 50 IU/kg.

### On-Demand Treatment

Bleeding episodes during the on-demand treatment regimen with efanesoctocog alfa (first 26 weeks of Arm B) are treated with a single dose of 50 IU/kg efanesoctocog alfa. Bleeding episodes are treated in the hospital/clinic or at home, as applicable. The participant is instructed to consult the investigational site prior to dosing a follow-up injection. Administration of a second dose of efanesoctocog alfa as follow-up treatment is determined by the Investigator based on the participant's clinical condition. If a bleeding episode does not improve, additional doses of 30 or 50 IU/kg every 2 to 3 days is considered. Treatment of any bleeding reported within 72 hours of a previous episode at the same location should be considered a follow-up injection and the event should not be recorded as a new bleeding episode.

### Surgical Dosing

Minor surgeries can be performed while participating in this trial by administering a dose of 50 IU/kg efanesoctocog alfa. Participants from any arm who undergo major surgery during the study will be included in the surgery subset. Major surgery is defined as any invasive operative procedure that requires any of the following: opening into a major body cavity (e.g., abdomen, thorax, skull), operation on a joint, removal of an organ, dental extraction of any molar teeth or >3 nonmolar teeth, operative alteration of normal anatomy, or crossing of a mesenchymal barrier (e.g., pleura, peritoneum, dura). It is recommended that any elective non-dental major surgery be performed at a study site, when possible. Minor surgery is defined as any invasive operative procedure in which only skin, mucous membranes, or superficial connective tissue is manipulated and does not meet the criteria for major surgery (e.g., dental extraction of <3 nonmolar teeth). Minor surgical procedures may be performed at a local health care provider institution.

For all participants in the surgery subgroup, the surgical period begin with their first dose of efanesoctocog alfa given for the surgery (i.e., the pre-operative loading dose) of 50 IU/kg IV. All patients receive a pre-operative loading dose prior to the surgical procedure. If needed, the dose level of efanesoctocog alfa should be adjusted to aim for a FVIII activity level of at least 100% and maintained during the surgery according to WFH Guidelines.

Post-operatively, FVIII activity levels are monitored by daily measurements of FVIII activity locally as long as the patient is hospitalized. FVIII levels are maintained at recommended levels and duration according to WFH Guideline. Depending on the targeted FVIII activity level this likely means a need to administer a second dose approximately 48-72 hours after the pre-operative loading dose. Additional doses of 30 or 50 IU/kg every 2 to 3 days may be administered depending on the desired FVIII activity levels and the severity of the procedure. Due to the long half-life of efanesoctocog alfa, the frequency of dosing in the post-surgical period may be extended after the first week post-surgery.

### C. Formulation—Drug Product

Efanesoctocog alfa drug product is supplied as a lyophilized form in a sterile vial that requires reconstitution with Sterile Water for Injection (diluent). For this study, each vial of drug product includes either of 250, 500, 1000, 2000, 3000 or 4000 IU of efanesoctocog alfa in a formulation buffer (10 mM L-histidine, 250 mM Arginine-HCl, 5 mM CaCl₂, 5% (w/v) sucrose, 0.05% (w/v) polysorbate 80, pH about 7.0).

### D. Efficacy: Objectives and Endpoints

The primary efficacy objective of the study is to evaluate the efficacy of efanesoctocog alfa as a prophylaxis treatment. The primary efficacy endpoint is annualized bleeding rate (ABR) for prophylaxis treatment (Arm A). ABR has traditionally been used to assess efficacy of FVIII products in the clinical trial setting and is considered an objective and measurable endpoint consistent with current EMA guidance and established regulatory precedents. Efficacy assessments in clinical studies of novel hemophilia therapies have typically compared prophylaxis to on-demand treatment regimens. However, it is now well recognized that prophylaxis is considered standard-of-care therapy for hemophilia A and reflected in current hemophilia treatment guidelines (see, e.g., World Federation of Hemophilia (WFH) Guidelines for the management of hemophilia, 2012, 2nd edition). Therefore, to more appropriately reflect the current practice patterns and treatment guidelines of hemophilia, the primary efficacy is assessed for prophylaxis treatment in Arm A. Given the robust information available from previously marketed FVIII products and the well-characterized efficacy of prophylaxis treatment, an estimation approach is used for the primary efficacy analysis.

The mean annualized bleeding rate and one-sided 97.5% confidence interval is estimated for prophylaxis treatment in Arm A using a Negative-Binomial regression model with a pre-defined success threshold based on published historical data from other marketed FVIII products. Notably, recently approved hemophilia therapies have also utilized the estimation approach for key efficacy endpoint analysis (see, e.g., Collins et al. Blood. 2014; 124(26): 3880-3886; Mahlangu et al. NEJM. 2018; 811-822.)

A key secondary efficacy endpoint compares ABR between efanesoctocog alfa weekly prophylaxis treatment and historical prophylaxis treatment using an intra-patient comparison for participants in Arm A who participated in observational study 242HA201/OBS16221. A non-inferiority test using a margin that preserves a substantial amount of the treatment effect provides meaningful data in the context of current standard of care therapy. The non-inferiority test margin was estimated using a meta-analysis that included the consideration of results from studies published in Manco-Johnson et al. J Thromb Haemost. 2013; 11:1119-27, Mahlangu et al. Blood. 2014; 123(3):317-25, Konkle et al. Blood. 2015; 126(9):1078-85, Kavakli et al. J Thromb Haemost. 2015; 13(3):360-9, Mahlangu et al. Blood. 2016. 2016; 128(5):630-7, Reding et al. J Thromb Haemost. 2017; 15(3):411-9, and Giangrande et al. Thromb Haemost. 2017; 117:252-61. The observational study allows prospective data collection on bleeding episodes and treatment administration for participants on marketed FVIII products. Utilization of an ePD for data collection in a similar manner to the data collection in this Phase 3 study facilitates a robust comparison to support the primary endpoint results.

Other secondary efficacy endpoints for this objective include ABR by type of bleed (spontaneous, traumatic, or unknown type) and location of bleed per study arm; ABR for all bleeding episodes (including untreated bleeding episodes) for prophylaxis treatment per study arm; intra-patient comparison of ABR during the QW prophylaxis treatment period versus the ABR during the on-demand treatment period in Arm B; and percentage of participants who maintain FVIII activity levels over 1%, 5%, 10%, 15%, and 20% in Arm A.

A secondary efficacy objective of the study is to evaluate the efficacy of efanesoctocog alfa in the treatment of bleeding episodes. Efficacy endpoints for this objective include the number of injections and dose of efanesoctocog alfa to treat a bleeding episode per study arm and treatment regimen; percentage of bleeding episodes treated with a single injection of efanesoctocog alfa per study arm and treatment regimen; assessment of response to efanesoctocog alfa treatment of individual bleeding episodes based on the International Society on Thrombosis and Haemostasis (ISTH) 4-point response scale per study arm and treatment regimen; and physician's global assessment (PGA) of participant's response to efanesoctocog alfa treatment based on a 4-point response scale per study arm and treatment regimen.

Another secondary efficacy objective of the study is to evaluate efanesoctocog alfa consumption for the prevention and treatment of bleeding episodes. Efficacy endpoints for this objective include total annualized efanesoctocog alfa consumption per participant.

Another secondary efficacy objective of the study is to evaluate the effect of efanesoctocog alfa prophylaxis on joint health outcomes. Efficacy endpoints for this objective include change from Baseline to Week 52 in total score and domain scores (e.g., swelling and strength) assessed by the Hemophilia Joint Health Score (HJHS) in Arm A; Annualized Joint Bleeding Rate (AJBR) per study arm and treatment regimen; and target joint resolution at Week 52 based on ISTH criteria in Arm A.

Another efficacy objective of the study is to evaluate the effect of efanesoctocog alfa prophylaxis on Quality of Life (QoL) outcomes. Efficacy endpoints for this objective include changes in Haem-A-QoL (17 years old) total score and physical health score measures from Baseline to Week 52 in Arm A; changes in PROMIS Pain Intensity 3a from Baseline to Week 52 in Arm A; and changes in PROMIS SF Physical Function (>18 years old) measures from Baseline to Week 52 in Arm A.

Another efficacy objective of the study is to evaluate the efficacy of efanesoctocog alfa for perioperative management. Efficacy endpoints for this objective include investigator or surgeon assessment of participant's hemostatic response to efanesoctocog alfa treatment on the ISTH 4-point response for surgical procedures scale; number of injections and dose to maintain hemostasis during perioperative period for major surgery; total efanesoctocog alfa consumption during perioperative period for major surgery; number and type of blood component transfusions used during perioperative period for major surgery; and estimated blood loss during perioperative period for major surgery.

### E. Efficacy Assessments

Planned time points for all efficacy assessments are provided in the SoA (Table 4).

In this study, a standardized definition of a bleeding episode based on International Society on Thrombosis and Haemostasis (ISTH) criteria is used (Blanchette et al. J Thromb Haemost. 2014; 12(11):1935-9).

As used in this Example 1, a “treated bleeding episode” is defined as a treated episode that starts from the first sign of bleeding and ends no more than 72 hours after the last injection to treat the bleeding episode. Any subsequent bleeding at the same location and injections administered 572 hours from the previous injection will be considered as the same bleeding episode. Multiple bleeding locations treated with a single injection will also be considered a single bleeding episode. Any injection to treat the bleeding episode that is administered >72 hours after the preceding one will be considered the first injection to treat a new bleeding episode in the same location. Any injection used to treat subsequent bleeding at a different location will be considered a separate bleeding episode, regardless of the time from the last injection to treat a bleeding episode.

If a bleeding episode occurs on the same day as a regularly scheduled prophylaxis dose, participants are instructed to record in their diary:
Treatment for bleeding episode (if the bleeding episode required efanesoctocog alfa treatment on the regular prophylaxis day)
Follow up injection (if an additional injection to is required to treat the bleed on the regular prophylaxis day). OR
Prophylaxis dose: if the bleeding episode did not require efanesoctocog alfa treatment on the regular prophylaxis day. In this case, the bleeding episode should be recorded and considered as an untreated bleeding episode.

Bleeding episodes or hemorrhages are classified as either spontaneous or traumatic. As used in Example 1, “spontaneous bleeding episodes” are bleeding episodes that occur when there is no known contributing factor, such as a definite trauma or antecedent “strenuous” activity, is at the discretion of the investigator, and the parent/caregiver/participant needs to be instructed by the investigator on the proper categorization. As used in this Example 1, “traumatic bleeding episodes” are bleeding episodes that occur when there is a known or believed reason for the bleed. For example, if a participant exercised strenuously and then had a bleeding episode in the absence of any obvious injury, the bleeding episode is still recorded as traumatic. Target joint bleeding episodes can be traumatic if a known action led to bleeding into the joint.

To assess clinical response to efanesoctocog alfa treatment for bleeding episodes, the ISTH 4-point assessment scale of treatment of acute bleeds will be used throughout the study (Blanchette 2014). The assessment should be performed approximately 72 hours after the initial treatment for the bleeding episode.

Target joint resolution: The Investigator will assess target joints at Baseline according to the International Society on Thrombosis and Haemostasis (ISTH) criteria. A target joint is defined as a major joint (e.g., hip, elbow, wrist, shoulder, knee or ankle) into which >3 spontaneous bleeding episodes occurred in a consecutive 6-month period. Target joint resolution is defined as ≤2 bleeding episodes in the target joint over 12 months of continuous exposure.

Surgery: The investigators/surgeons who complete the surgical procedures assess the participant's response to surgery with efanesoctocog alfa treatment using a 4-point scale as summarized in Table 6 (see Blanchette et al. 2014; 12(11):1935-9). This includes observations during surgery and the 24-hour post-operative time period. This assessment is performed 24 hours after the surgery.

TABLE 6
ISTH hemostatic response for surgical procedures scale

| | |
|---|---|
| Excellent | Intraoperative and postoperative blood loss similar (within 10%) to the non- |
| | hemophilic patient with no extra (unplanned) doses of FVIII/FIX/‘bypassing |
| | agents’ needed and bloodcomponent transfusions are similar to the non- |
| | hemophilic patient |
| Good | Intraoperative and/or postoperative blood loss slightly increased over |
| | expectation for the non-hemophilic patient (between 10 and 25% of |
| | expected), but the difference is judged by the involved |
| | surgeon/anesthetist/relevant healthcare professional to be clinically |
| | insignificant as evidenced by no extra (unplanned) doses of |
| | FVIII/FIX/‘bypassing agents’ needed and blood component transfusions |
| | are similar to the non-hemophilic patient |
| Fair | Intraoperative and/or postoperative blood loss increased over expectation |
| | (25-50%) for the non-hemophilic patient and additional treatment is needed |
| | such as extra (unplanned) doses of FVIII/FIX/‘bypassing agents’ or |
| | increased blood component use (within two-fold) of the anticipated |
| | transfusion requirement |
| Poor | Significant intraoperative and/or postoperative blood loss that is |
| | substantially increased over expectation (>50%) for the non-hemophilic |
| | patient, requires intervention, and is not explained by a surgical/medical |
| | issue other than hemophilia, unexpected hypotension or unexpected |
| | transfer to an Intensive Care Unit due to bleeding or substantially increased |
| | blood component use (>2 fold) of the anticipated transfusion requirement |

Patient reported outcomes: Table 7 shows the concepts of measurement and their related patient reported outcomes (PRO) questionnaires to be used in the trial. Where available, the PROs (excluding the HRU assessment) are distributed to adult and pediatric patients as specified in the SoA (Table 4). All PROs, excluding the HRU, are collected via an electronic tablet and transmitted to a third party vendor data server over public networks using encrypted data. The PROMIS instruments are administered at Baseline and Weeks 26 and 52.

TABLE 7
Patient reported outcome concepts and questionnaires

| | | Assessment |
|---|---|---|
| Concept | PRO questionnaire | schedule |
| Pain and Physical | 1. PROMIS Pain Intensity | Baseline, |
| Function (PROMIS | 2. PROMIS-SF Pain | Weeks 26, 52 |
| Instruments) | Interference (≥18 years old) | |
| | or PROMIS Pediatric-SF | |
| | Pain Interference (<18 | |
| | years old) | |
| | 3. PROMIS-SF Physical | |
| | Function (≥18 years old) or | |
| | PROMIS Pediatric-SF Pain | |
| | Physical Activity (<18 years | |
| | old) | |
| Hemophilia Specific | Haem-A-QoL (≥17 years | Baseline, |
| | old) or Haemo QoL (<17 | Weeks 26, 52 |
| | years old) | |
| Activities List | HAL (≥18 years old) or | Baseline, |
| | pedHAL (<18 years old) | Weeks 26, 52 |
| Treatment | TSQM-9 | Baseline, |
| satisfaction | | Weeks 26, 52 |
| Health Status | EQ-5D-5L | Baseline, |
| | | Weeks 26, 52 |
| Global impression | PGIC | Weeks 26, 52 |
| of change | | |
| Global impression | 1. PGIS - activity | Baseline, |
| of severity | component | Weeks 26, 52 |
| | 2. PGIS - joint component | |
| Patient preference | Preference questionnaire | Week 52 |
| survey | | |
| Healthcare resource | HRU questionnaire | Baseline, |
| utilization | | Weeks 26, 52 |

### F. Safety: Objectives and Endpoints

Another objective of the study is to evaluate the safety and tolerability of efanesoctocog alfa treatment. Safety endpoints for this objective include the occurrence of adverse events (AEs) and serious adverse events (SAEs); the occurrence of clinically significant changes from baseline in physical examination, vital signs, and laboratory tests; development of inhibitors (neutralizing antibodies directed against factor VIII [FVIII]) as determined via the Nijmegen modified Bethesda assay; and the occurrence of embolic and thrombotic events.

### G. Safety Assessments

Planned time points for all safety assessments and drug product tolerability investigations are provided in the Schedule of Activities (SoA) (Table 4). Non-limiting examples of these investigations include the following:

1. Physical examination: assessment of the cardiovascular, respiratory, gastrointestinal, neurological, dermatological, and musculoskeletal systems.

2. Vital signs: blood pressure, pulse rate, respiratory rate, and body temperature (° C.).

3. Laboratory tests, including:

Hematology: Platelet count; Red blood cell (RBC) count; White blood cell (WBC) count and differential; Hemoglobin (Hgb); Hematocrit (HCT).

Clinical chemistry: Alanine amino transferase (ALT); Aspartate amino transferase (AST); Alkaline phosphatase (ALP); Gamma glutamyl transferase (GGT); Bilirubin; Blood Urea Nitrogen (BUN); Creatinine; Glucose [non-fasting]; Total protein; Potassium; Sodium; Chloride.

Von Willebrand comprehensive panel: VWF ristocetin cofactor activity; VWF antigen

Immunogenicity: Nijmegen modified Bethesda inhibitor assay; Anti-drug antibodies (ADAs); ADA subtype.

Coagulation parameters: Activated partial thromboplastin time (aPTT).

Other tests and assessments: ultrasound assessment using the JADE and/or HEAD US protocol (select sites only); highly sensitive [Serum or urine] human chorionic gonadotropin (hCG) pregnancy test (as needed for women of childbearing potential); HIV (HIV-1 antibodies, HIV-2 antibodies, and HIV-1 p24 antigen), HBV (HBV surface antigen [HBsAg], anti-HBV surface antibody and anti-HBV core antibody) and HCV (anti-HCV antibodies) for patients who have been historically negative; follicle stimulating hormone and estradiol (as needed in women of non-child bearing potential only); CD4 count and viral load (for patients known to be HIV positive).

### Adverse Events

Adverse events (AE) are reported by the participant (or, when appropriate, by a caregiver, surrogate, or the participant's legally authorized representative).

In this study, an adverse event (AE) is defined as any untoward medical occurrence in a patient or clinical study participant, temporally associated with the use of study intervention, whether or not considered related to the study intervention. An AE can therefore be any unfavorable and unintended sign (including an abnormal laboratory finding), symptom, or disease (new or exacerbated) temporally associated with the use of study intervention.

Determination of whether an abnormal laboratory value and/or vital sign result meets the definition of an AE will be made by the investigator. Abnormal laboratory results are not considered AEs unless one or more of the following criteria are met: (1) symptomatic, (2) requiring either corrective treatment or consultation, (3) leading to IMP discontinuation or modification of dosing, (4) fulfilling a seriousness criterion, and/or (5) defined as an adverse event of special interest (AESI).

Bleeding episodes in this patient population were not considered AEs; however, the concomitant events associated with a bleeding episode were reported as AEs as appropriate (e.g., an elbow fracture). Bleeding episodes that meet the criteria for a serious adverse event (SAE) were be reported as a SAE as appropriate.

In this study, a serious adverse event (SAE) is defined as any untoward medical occurrence that at any dose results in death, or is life-threatening, or that requires inpatient hospitalization or prolongation of existing hospitalization, or results in persistent or significant disability/incapacity, or is a congenital anomaly/birth defect, or is a medically important event.

In this study, an adverse event of special interest (AESI) is defined as an adverse event (serious or nonserious) of scientific and medical concern, specific to the IMP or program, for which ongoing monitoring and rapid communication by the Investigator to the Sponsor may be appropriate. AESI may require further investigation in order to characterize and understand them. Examples of AESI include development of an inhibitor (e.g. an inhibitor result of >0.6 BU/mL that is confirmed by a second test result of >0.6 BU/mL drawn 2 to 4 weeks following the date when the original sample was drawn), or a Grade 3 or higher allergic reaction.

### H. Pharmacokinetics: Objectives and Endpoints

A pharmacokinetic (PK) objective of the study is to assess the PK of efanesoctocog alfa based on the one-stage activated partial thromboplastin time (aPTT) and two-stage chromogenic FVIII activity assays. PK endpoints or include maximum activity (Cₘₐₓ), elimination half-life (t_{1/2}), total clearance (CL), total clearance at steady state (CLss), accumulation index (AI), area under the activity time curve (AUC), volume of distribution at steady state (Vss), mean residence time (MRT), incremental recovery (IR), trough activity (C_{trough}), and time above predefined FVIII activity levels.

### 1. Pharmacokinetic Assessments

Pharmacokinetic (PK) assessments are conducted on participant samples collected over the course of the study. Abbreviated pharmacokinetic sampling or sequential pharmacokinetic sampling is used to estimate PK parameters, including but not limited to the following: Maximum activity (Cmax), Elimination half-life (t½), total clearance (CL), total clearance at steady state (CLss), Accumulation index (AI), Area under the activity-time curve (AUC), Volume of distribution at steady state (Vss), Mean residence time (MRT), Incremental recovery (IR), Trough activity (Ctrough), and time above 1% FVIII activity.

Arm A: For all participants in Arm A (prophylaxis treatment regimen), all participants receive a Day 1 dose, and the sampling times for blood collection are pre-injection, followed by 15 (±3) minutes, 3 hours (±15 minutes), 24 (±2) hours, 72 (±5) hours [Day 4], 168 (±5) hours [Day 8], from the start of the injection. A number of participants from Arm A enroll in the Sequential PK Subgroup to complete the PK sampling as required for the estimation of efanesoctocog alfa terminal half-life. For participants in the sequential PK subgroup, sampling times were extended to 240 (±5) hours [Day 11], and 336 (±5) hours [Day 15] from the start of the injection. Repeat PK profiling will be conducted at 26 weeks.

Arm B: For all participants in Arm B (on-demand switch to prophylaxis treatment regimen), all participants receive a Day 1 dose, and the sampling times for blood collection are pre-injection, followed by 15 (±3) minutes, 3 hours (±15 minutes), 24 (±2) hours, 72 (±5) hours [Day 4], and 168 (±5) hours [Day 8] from the start of the injection.

Trough measurement is made pre-injection. Peak measurement is made 15 (±3) minutes from the start of the injection, using two different assays: the 1-stage aPTT-based FVIII activity assay and the Efa capture chromogenic Coatest FVIII activity assay. The first assay to measure FVIII activity of Efa uses a modification of the aPTT using Dade Actin FSL activated PTT reagent (1-stage aPTT) on the BCS XP analyzer (Siemens Healthcare Diagnostics). The assay was validated using plasma standard that has been calibrated against the WHO (World Health Organization) 6th International Standard for FVIII. The 1-stage aPTT assay is able to measure both native and Efa FVIII activity. The LLOQ of the assay is at 1% of normal FVIII activity (0.010 IU/mL). The second assay for determining Efa activity in plasma is a modified chromogenic Coatest FVIII activity assay. In this assay, the analyte (Efa) is first captured using an anti-ELNN antibody. The activity of captured Efa is then determined with Coatest FVIII chromogenic assay, which is provided in the form of a chromogenic substrate assay that quantifies the activated FVIII (FVIlla)-mediated conversion of factor X (FX) to activated FX (FXa) in the presence of trace amounts of thrombin (for FVIII activation) and excess activated factor IX (FIXa), FX, Ca++, and phospholipids. Formation of FXa is measured by turnover of a chromogenic FXa peptide substrate. The LLOQ of the assay is at 0.4% of normal FVIII activity (0.004 IU/mL, or 4 mIU/mL). The assay was validated using Efa self-standard.

### J. Inhibitor Development

Blood samples are collected for the detection of inhibitors. Inhibitor development is assessed by the Nijmegen-modified Bethesda assay and is defined as an initial test result of >0.6 BU/mL confirmed by a second test result from an independent blood sample collected within 2 to 4 weeks of the first positive sample. Low titer inhibitor development is defined as inhibitor test results of >0.6 and <5.00 BU/mL. High titer inhibitor development is defined as inhibitor test results of >5.00 BU/mL. Participants with discrepant inhibitor test results (initial low titer result followed by high titer result or initial high titer result followed by low titer result) have repeat inhibitor testing performed from a separate blood sample collected 2 to 4 weeks following the previous sample. If 2 of 3 test results are <5.00 BU/mL, the inhibitor is considered low titer. If 2 of 3 test results are >5.00 BU/mL, the inhibitor is considered high titer.

### K. Exploratory Objectives and Endpoints

An exploratory objective of this study is to evaluate joint-health structural outcomes via ultrasound using the Joint Activity and Damage Exam (JADE) protocol and/or Hemophilia Early Arthropathy Detection with Ultrasound (HEAD-US). Exploratory endpoints for this objective include Changes in anatomical structural joint health outcomes via the JADE and/or HEAD-US protocol for ultrasound imaging from Baseline to Week 52 in a subpopulation at selected sites in Arm A.

Another exploratory objective of this study is to assess the impact of efanesoctocog alfa treatment on patient reported outcome (PRO) measurements and physical activity (measures per study arm and treatment regimen). Exploratory endpoints for this objective include changes in PRO measures and physical activity per study arm and treatment regimen, including PROMIS-SF Physical Function (>18 years old) or PROMIS Pediatric-SF Physical Activity (<18 years old); Haem-A-QoL (>17 years old) or Haemo-QoL (<17 years old) total score and subscale scores; PROMIS-SF Pain Interference (218 years old) or PROMIS Pediatric-SF Pain Interference (<18 years old); EQ-5D-5L; Treatment Satisfaction Questionnaire for Medications (TSQM-9); Patient Global Impression of Severity (PGIS) (activity component); PGIS (joint component); Change in physical activity measures (ActiGraph Activity Monitor); Hemophilia Activities List (HAL) (>18 years old) or pedHAL (<18 years old). Other exploratory endpoints for this objective include change in HJHS total score and domain score (e.g., swelling and strength) in Arm B by treatment regimen; Patient Global Impression of Change (PGIC) at Week 26 and Week 52 per study arm and treatment regimen; Patient Preference at Week 52 per study arm and treatment regimen; and exit interview at Week 52 (or at subsequent follow-up visit).

### L. Results

All ABRs referred to in the data in this Example 1 and the associated tables and figures are for treated bleeding episodes, unless specified otherwise.

### Demographics and Study Participation

The key Phase 3 study demographic and disease characteristics are shown in Table 8. One female participant was enrolled in the study.

TABLE 8
Key Demographic and Disease Characteristics in Phase 3 Study

| | Overall (n = 159) | |
|---|---|---|
| Age - mean (SD) | 35.4 | (15.1) |
| 12-17 years, n (%) | 25 | (15.7) |
| 18-64 years, n (%) | 129 | (81.1) |
| ≥65 years, n (%) | 5 | (3.1) |
| Race, n (%) | | |
| Asian | 29 | (18.2) |
| Black or African American | 3 | (1.9) |
| White | 97 | (61.0) |
| Not reported or Other | 30 | (18.9) |
| Family history of inhibitor, n (%) | | |
| Yes | 5 | (3.1) |
| No | 125 | (78.6) |
| Unknown | 29 | (18.2) |

Phase 3 study Participant Disposition is shown in Table 9. Reasons for discontinuation: 3 (1.9%) participants withdrew consent & 3 (1.9%) used prohibited concomitant medication for medical needs. The remaining reasons are Adverse Event, protocol violation, death (which was assessed as not related to Efa treatment) and other for 1 (0.6%) participant each.

TABLE 9
Phase 3 Study Participant Disposition

| | Overall (n = 159) | |
|---|---|---|
| Completed, n (%) | 149 | (93.7) |
| Discontinued, n (%) | 10 | (6.3) |
| Exposed to at least one dose of efanesoctocog alfa | 159 | (100) |
| At least 50 exposure days, n (%) | 115 | (72.3) |
| With at least one major surgery, n (%) | 13 | (8.2) |
| Number of major surgeries, n | 14 | |
| Sequential PK subgroup*, n (%) | 17 | (10.7) |

*Participants who have evaluable pharmacokinetic (PK) profiles for both baseline and Week 26 PK profiles

All subjects were previously treated. The types of FVIII products previously administered in the pre-study (242HA201/OBS16221) were plasma derived, recombinant, and cryoprecipitate (in 138 subjects on prophylaxis and 19 subjects treated on-demand; See Table 10). Most subjects on prophylaxis in the pre-study (242HA201/OBS16221) had been previously treated with a recombinant FVIII product (101/138 subjects [73.2%]), whereas most subjects treated on-demand had previously received a plasma derived FVIII product (14/19 subjects [73.7%]). Overall, most subjects (86/157 subjects [54.8%]) in the pre-study (242HA201/OBS16221) were on their respective regimen for >12 months prior to the screening date.

### Table 10: Types of FVIII Products Previously Administered in Pre-Study (242HA201/OBS16221)

TABLE 10
Types of FVIII Products Previously Administered
in Pre-Study (242HA201/OBS16221)

| | On-demand | | Prophylaxis | | Overall | |
|---|---|---|---|---|---|---|
| # of Subjects | 19 | | 138 | | 156 | |
| Cryoprecipitate | 8 | (42.1) | 20 | (14.5) | 28 | (17.9) |
| Plasma Derived | 14 | (73.7) | 65 | (47.1) | 78 | (50.0) |
| Recombinant | 6 | (31.6) | 101 | (73.2) | 107 | (68.6) |

For the pre-study (242HA201/OBS16221) prophylaxis regimen, most subjects had their dose administered either 3 times per week (44/139 subjects [31.7%]) or 2 times per week (40/139 subjects [28.8%]). Additional information regarding pre-study (242HA201/OBS16221) prophylaxis regimen participants can be found in Table 11. In Table 11, patients are included in each treatment regimen they participated in and may appear in more than one treatment regimen. Each patient is counted only once in the overall column. Additional demographic data, and treatment and bleeding history for participants in the Phase 3 Study can be found in Table 12 and Table 14, respectively. Patient FVIII genotype data can be found in Table 13.

TABLE 11
Summary of most recent pre-study
(242HA201/OBS16221) FVIII regimen

| | Treatment regimen | | | | | |
|---|---|---|---|---|---|---|
| | On-demand | | Prophylaxis | | Overall | |
| | (N = 19) | | (N = 139) | | (N = 157) | |
| Pre-study FVIII regimen | | | | | | |
| (most recent regimen | | | | | | |
| prior to enrollment) | | | | | | |
| Number | 19 | | 139 | | 157 | |
| Prophylaxis | 0 | | 138 | (99.3) | 138 | (87.9) |
| On-demand | 19 | (100) | 1 | (0.7) | 19 | (12.1) |
| Pre-study treatment at | | | | | | |
| time of study enrolment | | | | | | |
| n | 19 | | 138 | | | |
| EHL | 54 | (39.1) | 1 | (5.3) | | |
| SHL | 66 | (47.8) | 6 | (31.6) | | |
| pdFVIII | 16 | (11.6) | 12 | (63.2) | | |
| SHL/pdFVIII | 82 | (59.4) | 18 | (94.7) | | |
| Time on most recent pre- | | | | | | |
| study FVIII regimen | | | | | | |
| Number | 16 | | 118 | | 133 | |
| <6 months | 3 | (15.8) | 20 | (14.4) | 23 | (14.6) |
| 6-12 months | 3 | (15.8) | 21 | (15.1) | 24 | (15.3) |
| >12 months | 10 | (52.6) | 77 | (55.4) | 86 | (54.8) |

| Pre-study regimen: | | | | | |
|---|---|---|---|---|---|
| Prophylaxis | | | | | |
| Regular prophylaxis | | | | | |
| schedule | | | | | |
| Number | NA | 138 | | 138 | |
| Every day | NA | 3 | (2.2) | 3 | (1.9) |
| Every other day | NA | 21 | (15.1) | 21 | (13.4) |
| Every 3 days | NA | 7 | (5.0) | 7 | (4.5) |
| Every 4 days | NA | 6 | (4.3) | 6 | (3.8) |
| Every 5 days | NA | 2 | (1.4) | 2 | (1.3) |
| Every 7 days | NA | 13 | (9.4) | 13 | (8.3) |
| Day 1 Day 4 | NA | 2 | (1.4) | 2 | (1.3) |
| Two times per week | NA | 40 | (28.8) | 40 | (25.5) |
| Three times per week | NA | 44 | (31.7) | 44 | (28.0) |

TABLE 12
Summary of demographic and baseline characteristics by Arm and Subgroup.

| | | | Arm B | | | | | | | |
|---|---|---|---|---|---|---|---|---|---|---|
| | Arm A | | On- | | | | Surgery | | | |
| | Prophylaxis | | demand | | Prophylaxis | | Subgroup | | Overall | |
| | (N = 133) | | (N = 26) | | (N = 26) | | (N = 13) | | (N = 159) | |
| Age (years)^{a} | | | | | | | | | | |
| Number | 133 | | 26 | | 26 | | 13 | | 159 | |
| Mean (SD) | 33.9 | (15.3) | 42.8 | (11.7) | 42.8 | (11.7) | 44.3 | (12.8) | 35.4 | (15.1) |
| Median | 34.0 | | 39.0 | | 39.0 | | 46.0 | | 35.0 | |
| Min; Max | 12; 72 | | 23; 68 | | 23; 68 | | 12; 64 | | 12; 72 | |
| 12-17 | 25 | (18.8) | 0 | | 0 | | 1 | (7.7) | 25 | (15.7) |
| 18-64 | 104 | (78.2) | 25 | (96.2) | 25 | (96.2) | 12 | (92.3) | 129 | (81.1) |
| >=65 | 4 | (3.0) | 1 | (3.8) | 1 | (3.8) | 0 | | 5 | (3.1) |
| Sex, n (%) | | | | | | | | | | |
| Number | 133 | | 26 | | 26 | | 13 | | 159 | |
| Male | 132 | (99.2) | 26 | (100) | 26 | (100) | 13 | (100) | 158 | (99.4) |
| Female | 1 | (0.8) | 0 | | 0 | | 0 | | 1 | (0.6) |
| Ethnicity, n (%) | | | | | | | | | | |
| Number | 132 | | 26 | | 26 | | 13 | | 158 | |
| Hispanic or Latino | 12 | (9.1) | 13 | (50.0) | 13 | (50.0) | 1 | (7.7) | 25 | (15.8) |
| Not Hispanic or Latino | 112 | (84.8) | 13 | (50.0) | 13 | (50.0) | 9 | (69.2) | 125 | (79.1) |
| Not reported due to confidentiality | 8 | (6.1) | 0 | | 0 | | 3 | (23.1) | 8 | (5.1) |
| regulations | | | | | | | | | | |
| Race, n (%) | | | | | | | | | | |
| Number | 133 | | 26 | | 26 | | 13 | | 159 | |
| Asian | 29 | (21.8) | 0 | | 0 | | 3 | (23.1) | 29 | (18.2) |
| Black or African American | 3 | (2.3) | 0 | | 0 | | 0 | | 3 | (1.9) |
| White | 71 | (53.4) | 26 | (100) | 26 | (100) | 7 | (53.8) | 97 | (61.0) |
| Not reported due to confidentiality | 26 | (19.5) | 0 | | 0 | | 3 | (23.1) | 26 | (16.4) |
| regulations | | | | | | | | | | |
| Other | 4 | (3.0) | 0 | | 0 | | 0 | | 4 | (2.5) |
| Region^{b}, n (%) | | | | | | | | | | |
| Number | 133 | | 26 | | 26 | | 13 | | 159 | |
| Asia/Pacific | 33 | (24.8) | 0 | | 0 | | 4 | (30.8) | 33 | (20.8) |
| Europe | 67 | (50.4) | 14 | (53.8) | 14 | (53.8) | 5 | (38.5) | 81 | (50.9) |
| North America | 26 | (19.5) | 0 | | 0 | | 3 | (23.1) | 26 | (16.4) |
| South America | 7 | (5.3) | 12 | (46.2) | 12 | (46.2) | 1 | (7.7) | 19 | (11.9) |
| Weight (kg) | | | | | | | | | | |
| Number | 133 | | 26 | | 26 | | 13 | | 159 | |
| Mean (SD) | 78.00 | (19.29) | 80.80 | (18.04) | 80.80 | (18.04) | 77.31 | (9.66) | 78.46 | (19.06) |
| Median | 78.00 | | 77.90 | | 77.90 | | 78.00 | | 78.00 | |
| Min; Max | 33.9; 132.8 | | 50.0; 119.5 | | 50.0; 119.5 | | 63.0; 100.0 | | 33.9; 132.8 | |
| Body Mass Index(BMI) (kg/m2) | | | | | | | | | | |
| Number | 133 | | 25 | | 25 | | 13 | | 158 | |
| Mean (SD) | 25.59 | (5.00) | 26.91 | (5.56) | 26.91 | (5.56) | 25.68 | (1.50) | 25.80 | (5.10) |
| Median | 25.51 | | 26.35 | | 26.35 | | 25.47 | | 25.74 | |
| Min; Max | 15.0; 39.8 | | 16.7; 40.8 | | 16.7; 40.8 | | 22.6; 28.4 | | 15.0; 40.8 | |
| <25 | 57 | (42.9) | 9 | (36.0) | 9 | (36.0) | 3 | (23.1) | 66 | (41.8) |
| >=25-<30 | 52 | (39.1) | 9 | (36.0) | 9 | (36.0) | 10 | (76.9) | 61 | (38.6) |

| | | | | | | | | | |
|---|---|---|---|---|---|---|---|---|---|
| >=30 | 24 | (18.0) | 7 | (28.0) | 7 | (28.0) | 0 | 31 | (19.6) |

Note:
1: Percentages are based on the number of participants with non-missing data in the Full Analysis Set.
2: Participants are included in each study arm and treatment regimen (including surgery subgroup) they participated in for the duration of time on that regimen and, as such, may appear in more than one treatment regimen. Each participant is counted only once in the overall column.
^{a}Age = year of informed consent − year of birth.
^{b}Asia/Pacific includes Australia, Japan, Korea and Taiwan. Europe includes Belgium, Bulgaria, France, Germany, Greece, Hungary, Italy, Netherlands, Spain and the United Kingdom. North America includes Canada, Mexico, and the United States. South America includes Argentina and Brazil.

TABLE 13
Patient FVIII genotype

| | | | Arm B | | | |
|---|---|---|---|---|---|---|
| | Arm A | | On-demand and | | | |
| | Prophylaxis | | prophylaxis | | Overall | |
| FVIII genotype, n (%) | (n = 132) | | (n = 26) | | (N = 158) | |
| Intron 22 inversion | 51 | (38.6) | 6 | (23.1) | 57 | (36.1) |
| Frameshift | 25 | (18.9) | 8 | (30.8) | 33 | (20.9) |
| Missense | 20 | (15.2) | 2 | (7.7) | 22 | (13.9) |
| Nonsense | 11 | (8.3) | 4 | (15.4) | 15 | (9.5) |
| Large structural change | 9 | (6.8) | 1 | (3.8) | 10 | (6.3) |
| (>50 bp) | | | | | | |
| Small structural change | 1 | (0.8) | 0 | | 1 | (0.6) |
| (<50 bp) | | | | | | |
| Splice site change | 2 | (1.5) | 3 | (11.5) | 5 | (3.2) |

| | | | | | |
|---|---|---|---|---|---|
| Synonymous | 0 | 2 | (7.7) | 2 | (1.3) |

| | | | | | |
|---|---|---|---|---|---|
| N/A | 13 | (9.8) | 0 | 13 | (8.2) |

TABLE 14
Summary of hemophilia treatment and bleeding history - Full Analysis Set

| | | | Arm B | | | | | | | |
|---|---|---|---|---|---|---|---|---|---|---|
| | Arm A | | On- | | | | Surgery | | | |
| | Prophylaxis | | demand | | Prophylaxis | | Subgroup | | Overall | |
| | (N = 133) | | (N = 26) | | (N = 26) | | (N = 13) | | (N = 159) | |
| Type of Hemophilia treatment | | | | | | | | | | |
| products administered throughout | | | | | | | | | | |
| life^{a}, n (%) | | | | | | | | | | |
| Number | 127 | | 26 | | 26 | | 13 | | 153 | |
| FVIII Plasma derived | 83 | (65.4) | 24 | (92.3) | 24 | (92.3) | 7 | (53.8) | 107 | (69.9) |
| FVIII Recombinant | 106 | (83.5) | 8 | (30.8) | 8 | (30.8) | 11 | (84.6) | 114 | (74.5) |
| FVIII Cryoprecipitate | 32 | (25.2) | 9 | (34.6) | 9 | (34.6) | 8 | (61.5) | 41 | (26.8) |
| Non FVIII Product | 25 | (19.7) | 2 | (7.7) | 2 | (7.7) | 2 | (15.4) | 27 | (17.6) |
| Antifibrinolytic agents | 15 | (11.8) | 2 | (7.7) | 2 | (7.7) | 0 | | 17 | (11.1) |
| Desmopressin/DDAVP | 1 | (0.8) | 0 | | 0 | | 0 | | 1 | (0.7) |
| Emicizumab | 6 | (4.7) | 0 | | 0 | | 2 | (15.4) | 6 | (3.9) |
| Fitusiran | 0 | | 0 | | 0 | | 0 | | 0 | |
| FEIBA | 0 | | 0 | | 0 | | 0 | | 0 | |
| rFVIIa (Novoseven) | 0 | | 0 | | 0 | | 0 | | 0 | |
| Other | 4 | (3.1) | 0 | | 0 | | 0 | | 4 | (2.6) |
| Age at start of first prophylaxis | | | | | | | | | | |
| regimen (years) | | | | | | | | | | |
| Number | 126 | | 25 | | 25 | | 11 | | 151 | |
| Mean (SD) | 3.6 | (6.0) | 10.8 | (15.1) | 10.8 | (15.1) | 3.3 | (4.2) | 4.8 | (8.6) |
| Median | 1.0 | | 3.0 | | 3.0 | | 1.0 | | 1.0 | |
| Min; Max | 0; 35 | | 0; 62 | | 0; 62 | | 0; 12 | | 0; 62 | |
| <6 | 105 | (83.3) | 16 | (64.0) | 16 | (64.0) | 9 | (81.8) | 121 | (80.1) |
| 6 to <10 | 5 | (4.0) | 1 | (4.0) | 1 | (4.0) | 0 | | 6 | (4.0) |
| 10 to <18 | 10 | (7.9) | 1 | (4.0) | 1 | (4.0) | 2 | (18.2) | 11 | (7.3) |
| >=18 | 6 | (4.8) | 7 | (28.0) | 7 | (28.0) | 0 | | 13 | (8.6) |
| Number of prior exposure days to | | | | | | | | | | |
| FVIII, n (%) | | | | | | | | | | |
| Number | 133 | | 26 | | 26 | | 13 | | 159 | |
| <50 | 0 | | 0 | | 0 | | 0 | | 0 | |
| 50-<100 | 0 | | 0 | | 0 | | 0 | | 0 | |
| 100-<150 | 0 | | 0 | | 0 | | 0 | | 0 | |
| >=150 | 133 | (100) | 26 | (100) | 26 | (100) | 13 | (100) | 159 | (100) |
| <150 | 0 | | 0 | | 0 | | 0 | | 0 | |
| >=150 | 133 | (100) | 26 | (100) | 26 | (100) | 13 | (100) | 159 | (100) |
| Number of bleeds in the past | | | | | | | | | | |
| 12 months | | | | | | | | | | |
| Number | 122 | | 23 | | 23 | | 12 | | 145 | |
| Mean (SD) | 3.2 | (5.4) | 35.7 | (22.2) | 35.7 | (22.2) | 9.1 | (21.8) | 8.3 | (15.5) |
| Median | 1.0 | | 30.0 | | 30.0 | | 2.5 | | 2.0 | |
| Min; Max | 0; 32 | | 4; 78 | | 4; 78 | | 0; 78 | | 0; 78 | |
| 0 | 44 | (36.1) | 0 | | 0 | | 2 | (16.7) | 44 | (30.3) |
| >0-5 | 57 | (46.7) | 1 | (4.3) | 1 | (4.3) | 7 | (58.3) | 58 | (40.0) |
| >5-10 | 14 | (11.5) | 2 | (8.7) | 2 | (8.7) | 2 | (16.7) | 16 | (11.0) |
| >10-20 | 3 | (2.5) | 4 | (17.4) | 4 | (17.4) | 0 | | 7 | (4.8) |
| >20 | 4 | (3.3) | 16 | (69.6) | 16 | (69.6) | 1 | (8.3) | 20 | (13.8) |
| 0 | 44 | (36.1) | 0 | | 0 | | 2 | (16.7) | 44 | (30.3) |
| 1 | 22 | (18.0) | 0 | | 0 | | 2 | (16.7) | 22 | (15.2) |
| 2 | 9 | (7.4) | 0 | | 0 | | 2 | (16.7) | 9 | (6.2) |
| 3 | 14 | (11.5) | 0 | | 0 | | 2 | (16.7) | 14 | (9.7) |
| 4 | 6 | (4.9) | 1 | (4.3) | 1 | (4.3) | 0 | | 7 | (4.8) |
| 5 | 6 | (4.9) | 0 | | 0 | | 1 | (8.3) | 6 | (4.1) |
| >5 | 21 | (17.2) | 22 | (95.7) | 22 | (95.7) | 3 | (25.0) | 43 | (29.7) |
| Number of joint bleeds in the | | | | | | | | | | |
| past 12 months | | | | | | | | | | |
| Number | 121 | | 21 | | 21 | | 12 | | 142 | |
| Mean (SD) | 2.3 | (4.5) | 27.4 | (18.6) | 27.4 | (18.6) | 7.9 | (19.7) | 6.0 | (12.1) |
| Median | 1.0 | | 24.0 | | 24.0 | | 2.0 | | 1.0 | |
| Min; Max | 0; 30 | | 4; 70 | | 4; 70 | | 0; 70 | | 0; 70 | |
| Number of spontaneous joint bleeds | | | | | | | | | | |
| in the past 12 months | | | | | | | | | | |
| Number | 118 | | 20 | | 20 | | 12 | | 138 | |
| Mean (SD) | 1.3 | (2.5) | 20.9 | (14.2) | 20.9 | (14.2) | 5.6 | (14.2) | 4.1 | (9.0) |
| Median | 0.0 | | 17.0 | | 17.0 | | 0.5 | | 0.0 | |
| Min; Max | 0; 15 | | 4; 50 | | 4; 50 | | 0; 50 | | 0; 50 | |
| Number of traumatic joint bleeds | | | | | | | | | | |
| in the past 12 months | | | | | | | | | | |
| Number | 118 | | 20 | | 20 | | 12 | | 138 | |
| Mean (SD) | 1.1 | (2.5) | 7.2 | (8.1) | 7.2 | (8.1) | 2.3 | (5.6) | 2.0 | (4.4) |
| Median | 0.0 | | 5.5 | | 5.5 | | 1.0 | | 0.0 | |
| Min; Max | 0; 18 | | 0; 30 | | 0; 30 | | 0; 20 | | 0; 30 | |
| Target joint at baseline | | | | | | | | | | |
| Number | 133 | | 26 | | 26 | | 13 | | 159 | |
| 0 | 107 | (80.5) | 3 | (11.5) | 3 | (11.5) | 9 | (69.2) | 110 | (69.2) |
| 1 | 7 | (5.3) | 1 | (3.8) | 1 | (3.8) | 0 | | 8 | (5.0) |
| 2 | 5 | (3.8) | 8 | (30.8) | 8 | (30.8) | 1 | (7.7) | 13 | (8.2) |
| 3 | 5 | (3.8) | 7 | (26.9) | 7 | (26.9) | 0 | | 12 | (7.5) |
| 4 | 1 | (0.8) | 3 | (11.5) | 3 | (11.5) | 0 | | 4 | (2.5) |
| 5 | 4 | (3.0) | 1 | (3.8) | 1 | (3.8) | 2 | (15.4) | 5 | (3.1) |
| 6 | 4 | (3.0) | 2 | (7.7) | 2 | (7.7) | 1 | (7.7) | 6 | (3.8) |

| | | | | | | | | |
|---|---|---|---|---|---|---|---|---|
| >6 | 0 | 1 | (3.8) | 1 | (3.8) | 0 | 1 | (0.6) |

Note:
1: Percentages are based on the number of participants with non-missing data in the Full Analysis Set.
2: Participants are included in each study arm and treatment regimen (including surgery subgroup) they participated in and, as such, may appear in more than one treatment regimen. Each participant is counted only once in the overall column.
^{a}Participants may be counted in more than one category.

### Results of Observational Study

Additional data related to the endpoints of the Phase 3 study was collected for participants in the observational study. The mean (standard deviation [SD]) duration of the observational period was 42.3 (14.1) and 46.7 (8.4) weeks for patients on prophylaxis and on-demand, respectively. Mean (SD) number of routine prophylaxis injections per week was 2.2 (1.07). Median (quarter [Q]1, Q3) ABRs for patients on prophylaxis and on-demand were, respectively: overall, 2.0 (0.0, 5.7) and 29.1 (8.4, 40.0); spontaneous, 0.0 (0.0, 3.0) and 14.7 (7.7, 30.1); traumatic, 1.0 (0.0, 2.1) and 4.8 (0.0, 8.7). Additional results from the observational study are summarized in in Table 15.

TABLE 15
Summary of results of observational study

| | Treatment regimen | | | |
|---|---|---|---|---|
| | Prophylaxis | | On-demand | |
| | (n = 139) | | (n = 19) | |
| Median ABR (Q1, Q3) | | | | |
| Overall | 2.0 | (0.0, 5.7)^{a} | 29.1 | (8.4, 40.0) |
| Spontaneous | 0.0 | (0.0, 3.0)^{a} | 14.7 | (7.7, 30.1) |
| Traumatic | 1.0 | (0.0, 2.1)^{a} | 4.8 | (0.0, 8.7) |
| Joint | 1.1 | (0.0, 3.7)^{a} | 20.4 | (7.2, 34.5) |
| Patients with zero bleeds, n (%) | 48 | (35) | 0 | |
| Median annualized consumption, | 4106.4 | (3151.6, 5291.1) | 875.0 | (564.1, 1049.1) |
| IU/kg (Q1, Q3) | | | | |
| Mean injections per week, n (Q1, Q3) | 111.2 | (62.2) | 37.2 | (36.4) |
| Mean number of routine prophylaxis | 2.2 | (1.07; 0.25-6.98) | N/A | |
| injections per week, n (SD; range) | | | | |

| | Prophylaxis | | On-demand | | Overall | |
|---|---|---|---|---|---|---|
| | (n = 139) | | (n = 19) | | (n = 157) | |
| Median no. of injections required for bleed | 1.0 | (1.0, 1.0) | 1.0 | (1.0, 1.0) | 1.0 | (1.0, 1.0) |
| resolution, n (Q1, Q3)^{b} | | | | | | |
| Median total dose required for bleed | 37.1 | (26.4, 50.8) | 26.3 | (17.0, 30.3) | 30.2 | (23.3, 40.7) |
| resolution, IU/kg (Q1, Q3)^{b} | | | | | | |
| Median dose per injection required for bleed | 33.1 | (25.6, 37.3) | 24.9 | (14.7, 29.4) | 27.0 | (19.2, 35.6) |
| resolution, IU/kg (Q1, Q3)^{b} | | | | | | |
| Mean HJHS score (SD; n) | | | | | | |
| Baseline | 15.5 | (18.7; n = 77) | 26.2 | (12.9; n = 11) | 16.8 | (18.4; n = 88) |
| 12 months | 13.9 | (18.7; n = 52) | 16.0 | (8.4; n = 4) | 14.1 | (18.1; n = 56) |
| Mean change from baseline at 12 months | −1.7 | (6.7; n = 52) | −3.0 | (3.4; n = 4) | −1.8 | (6.5; n = 56) |
| Mean Haem-A-Qol Total Score (SD; n) | | | | | | |
| Baseline | 30.6 | (17.3; n = 111) | 40.8 | (19.5; n = 17) | 31.9 | (17.3; n = 128) |
| 12 months | 29.5 | (17.4; n = 98) | 41.7 | (16.2; n = 15) | 31.1 | (17.7; n = 113) |
| Mean change from baseline at 12 months | −0.9 | (8.6; n = 98) | −0.1 | (16.6; n = 15) | −0.8 | (9.9; n = 113) |
| Mean Haem-A-Qol Physical Health (SD; n) | | | | | | |
| Baseline | 36.9 | (24.9; n = 111) | 51.18 | (23.88; n = 17) | 38.8 | (25.2; n = 128) |
| 12 months | 33.1 | (23.7; n = 98) | 55.7 | (22.7; n = 15) | 36.1 | (24.7; n = 113) |
| Mean change from baseline at 12 months | −3.3 | (16.9; n = 98) | 3.0 | (21.9; n = 15) | −2.4 | (17.7; n = 113) |
| Mean PROMIS Pain Intensity 3a T-score (SD; n) | | | | | | |
| Baseline | 41.6 | (8.2; n = 72) | 48.4 | (7.3; n = 15) | 42.8 | (8.4; n = 87) |
| 12 months | 40.9 | (9.1; n = 113) | 43.3 | (10.9; n = 16) | 41.2 | (9.3; n = 129) |
| Mean change from baseline at 12 months | 0.1 | (6.3; n = 65) | −4.8 | (10.4; n = 14) | −0.75 | (7.3; n = 79) |

### Primary Endpoint of Phase 3 Study

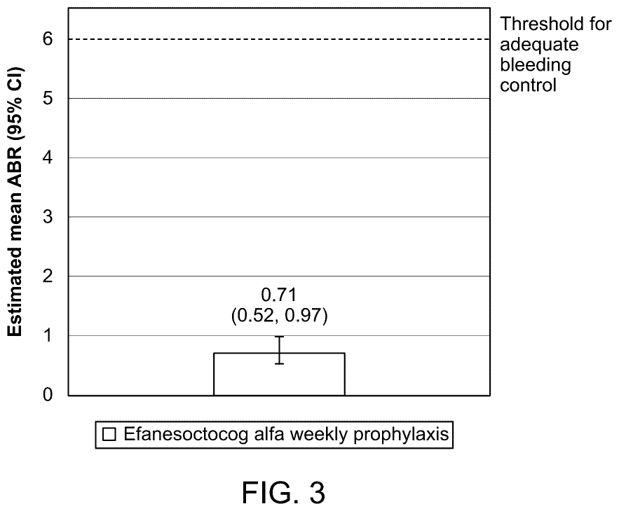

As shown in FIG. 3, once weekly prophylactic treatment with efanesoctocog alfa at 50 IU/kg provided effective protection against bleeding. In a statistical model analysis of ABR, the estimated mean ABR was 0.71 (95% CI 0.52; 0.97) (Adequate bleeding control if the upper limit of the CI 56). In Arm A, the mean (SD) overall ABR was 0.71 (1.43, n=133) over the full 12-month study duration and 0.59 (1.32, n=128) during the last 6 months of the study. Descriptive analysis of ABR is seen in Table 16 below.

TABLE 16
Summary of Primary Endpoint of Phase 3 Study (Arm A)

| Descriptive analysis of ABR | Value (n = 133) | |
|---|---|---|
| Observed Mean (SD) | 0.71 | (1.43) |
| Observed Median (IQR) | 0.00 | (0.00, 1.04) |
| Patients with zero bleeds, n (%) | 86 | (65) |

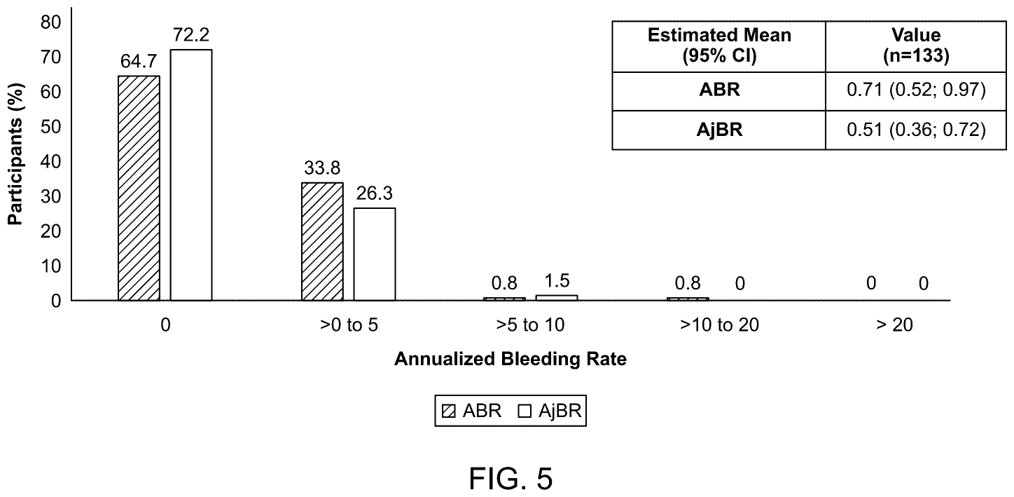

The distribution of Annualized Bleeding Rate (Table 18) and Annualized Joint Bleeding Rate (AjBR) (Table 17) among subjects in Arm A was analyzed (also see FIG. 5). Of the subjects enrolled and analyzed for ABR in Arm A (n=133), 86 patients (64.7%) had zero bleeds during the study. 45 subjects (33.8%) had 1 to 5 bleeding episodes during the study period. 1 subject (0.8%) had 6 to 10 bleeds. 1 subject (0.8%) had 11 to 20 bleeds. No subjects had 21 or more bleeding episodes during the study period. With respect to AjBR, 96 subjects (72.2%) had zero joint bleeds in the study period. 35 subjects (26.3%) had 1 to 5 joint bleeds. 2 subjects (1.5%) had 6 to 10 joint bleeds. No subjects had 11 to 20 or more than 21 joint bleeds during the study period. The historical treatment joint bleeding rates were derived from subjects enrolled in observational study 242HA201/OBS16221. Also see Table 17.

The mean (SD) efficacy period for the 6-month duration of prophylaxis in Arm B was 22.89 (6.00) weeks and 77% of participants experienced zero bleeds (see Table 18).

TABLE 17
Annualized Joint Bleeding Rate from Subjects in Observational Study 242HA201/OBS16221 and Phase 3 Study

| | | | | | | | Efanesoctocog Alfa | | | | | |
|---|---|---|---|---|---|---|---|---|---|---|---|---|
| | Historical Treatment | | | | | | Treatment in Phase 3 Study | | | | | |
| | 242HA201/OBS16221 | | | | | | Arm A | | Arm B | | | |
| | Prophylaxis | | On-demand | | Overall | | Prophylaxis | | On-demand | | Prophylaxis | |
| Subject | | | | | | | | | | | | |
| Level | | | | | | | | | | | | |
| Number | 139 | | 19 | | 157 | | 133 | | 26 | | 26 | |
| Mean (SD) | 4.07 | (11.00) | 21.06 | (15.94) | 6.12 | (12.86) | 0.52 | (1.09) | 17.45 | (7.31) | 0.61 | (1.33) |
| Median | 1.07 | | 20.37 | | 1.76 | | 0.00 | | 18.42 | | 0.00 | |
| Q1; Q3 | 0.00; 3.74 | | 7.16; 34.52 | | 0.00; 5.81 | | 0.00; 1.02 | | 10.80; 23.90 | | 0.00; 0.00 | |
| Min; Max | 0.0; 88.7 | | 0.0; 51.1 | | 0.0; 88.7 | | 0.0; 6.2 | | 4.1; 30.4 | | 0.0; 4.1 | |
| Population | | | | | | | | | | | | |
| level, | | | | | | | | | | | | |
| model based | | | | | | | | | | | | |
| Mean | 3.67 | (2.70; 4.99) | 21.12 | (13.63; 32.72) | 5.89 | (4.42; 7.87) | 0.51 | (0.36; 0.72) | 17.48 | (14.88; 20.54) | 0.62 | (0.25; 1.52) |
| (95% CI) | | | | | | | | | | | | |

Total Annualized Bleeding Rates for the observational study and the phase 3 study are shown in Table 18.

TABLE 18
Total Annualized Bleeding Rate from Subjects in Observational Study 242HA201/OBS16221 and Phase 3 Study

| | | | | | | | Efanesoctocog Alfa | | | | | |
|---|---|---|---|---|---|---|---|---|---|---|---|---|
| | Historical Treatment | | | | | | Treatment in Phase 3 Study | | | | | |
| | 242HA201/OBS16221 | | | | | | Arm A | | Arm B | | | |
| | Prophylaxis | | On-demand | | Overall | | Prophylaxis | | On-demand | | Prophylaxis | |
| Number of | 139 | | 19 | | 157 | | 133 | | 26 | | 26 | |
| Patients with | | | | | | | | | | | | |
| observational/ | | | | | | | | | | | | |
| efficacy period | | | | | | | | | | | | |
| Total # of | 536 | | 442 | | 978 | | 86 | | 268 | | 8 | |
| treated bleed- | | | | | | | | | | | | |
| ing episodes | | | | | | | | | | | | |
| Total Patient | 112.7 | | 17.0 | | 129.7 | | 121.2 | | 12.5 | | 11.4 | |
| years followed | | | | | | | | | | | | |
| Duration of | | | | | | | | | | | | |
| observational | | | | | | | | | | | | |
| period (weeks) | | | | | | | | | | | | |
| Number | 139 | | 19 | | 157 | | 133 | | 26 | | 26 | |
| Mean (SD) | 42.30 | (14.06) | 46.71 | (8.39) | 43.10 | (13.34) | 47.55 | (8.77) | 25.12 | (1.07) | 22.89 | (6.00) |
| Median | 48.38 | | 46.86 | | 48.44 | | 50.09 | | 25.00 | | 25.08 | |
| Q1; Q3 | 38.07; 52.07 | | 43.29; 54.14 | | 39.03; 52.12 | | 48.06; 51.09 | | 24.72; 25.02 | | 22.07; 27.06 | |
| Min; Max | 0.1; 54.0 | | 20.3; 54.1 | | 0.1; 54.7 | | 0.1; 54.1 | | 23.9; 29.3 | | 1.1; 27.1 | |
| ABR | | | | | | | | | | | | |
| Number | 139 | | 19 | | 157 | | 133 | | 26 | | 26 | |
| Mean (SD) | 5.11 | (11.58) | 25.23 | (17.51) | 7.55 | (13.98) | 0.71 | (1.43) | 21.42 | (7.41) | 0.69 | (1.35) |
| Median | 1.97 | | 29.10 | | 2.37 | | 0.00 | | 21.13 | | 0.00 | |
| Q1; Q3 | 0.00; 5.71 | | 8.36; 40.00 | | 0.00; 6.95 | | 0.00; 1.04 | | 15.12; 27.13 | | 0.00; 0.00 | |
| Min; Max | 0.0; 88.7 | | 1.1; 57.8 | | 0.0; 88.7 | | 0.0; 11.0 | | 8.3; 33.4 | | 0.0; 4.1 | |
| Population | | | | | | | | | | | | |
| level, model | | | | | | | | | | | | |
| based^{a} | | | | | | | | | | | | |
| Mean (95% CI) | 4.76 | (3.67; 6.18) | 25.29 | (17.10; 37.41) | 7.36 | (5.73; 9.46) | 0.71 | (0.52; 0.97) | 21.41 | (18.79; 24.40) | 0.70 | (0.32; 1.53) |

^{a}Estimated using a negative binomial model with the total number of treated bleeding episodes during the observational period as the response variable and log-transformed observational period duration (in years) as an offset variable.

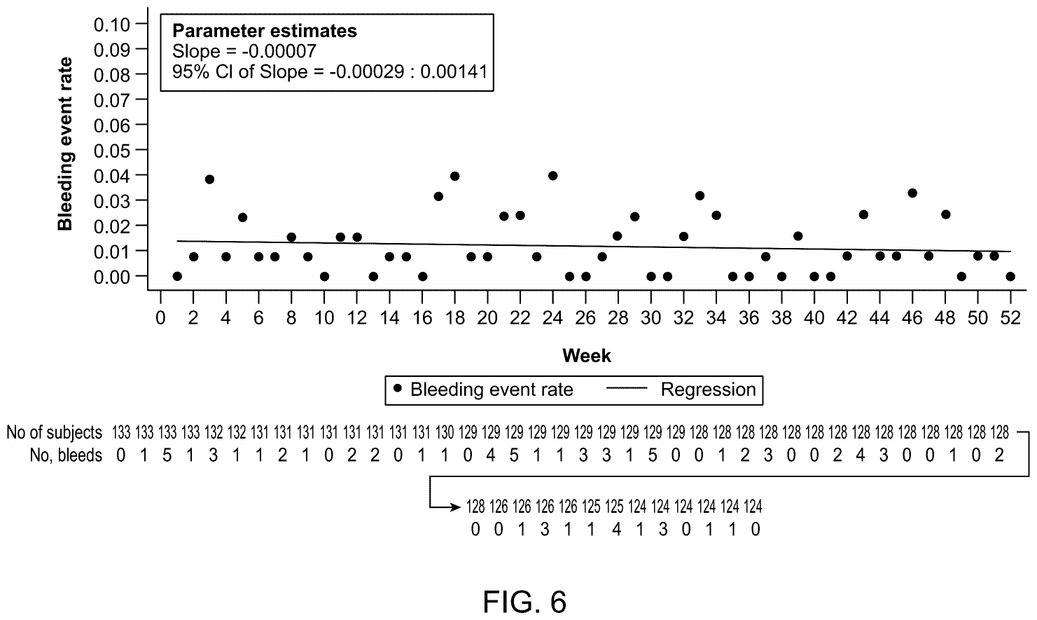

The bleeding event rate in Arm A was consistent throughout the duration of the study, indicating once-weekly efanesoctocog alfa prophylaxis provides immediate and constant protection against bleeds (FIG. 6).

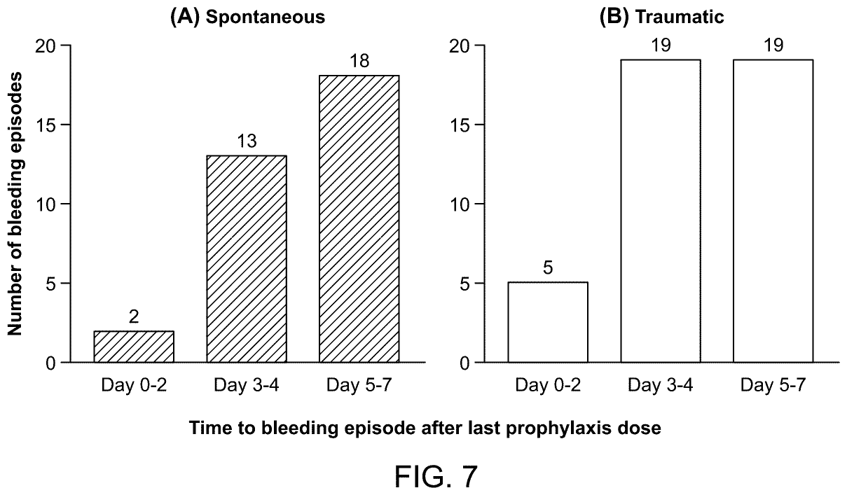

In Arm A, 18 spontaneous bleeding episodes occurred 5-7 days after the last prophylaxis dose, 13 occurred 3-4 days after the last dose, and 2 occurred 0-2 days after the last dose (FIG. 7, (A)) Overall, 25 patients in Arm A had a total of 33 spontaneous bleeding episodes, with 19 patients (76%) experiencing a single spontaneous bleeding episode. For traumatic bleeding episodes, 19 each occurred in the 3-4 days and 5-7 days following the last prophylaxis dose, and 5 occurred 0-2 days after the last prophylaxis dose (FIG. 7, (B)). A summary of ABRs and bleeding episodes in Arm A and Arm B is shown in Table 19.

TABLE 19
Summary of ABRs and bleeding episodes in Arm A and Arm B.

| | Arm A | | Arm B | | Arm B | |
|---|---|---|---|---|---|---|
| | prophylaxis | | on demand | | prophylaxis | |
| | (n = 133) | | (n = 26) | | (n = 26) | |
| Overall ABR^{a} | | | | | | |
| Mean (SD) | 0.71 | (1.43) | 21.42 | (7.41) | 0.69 | (1.35) |
| Median (IQR) | 0.00 | (0.00-1.04) | 21.13 | (15.12-27.13) | 0.00 | (0.00-0.00) |
| Spontaneous ABR | | | | | | |
| Mean (SD) | 0.29 | (0.73) | 15.87 | (9.28) | 0.45 | (1.13) |
| Median (IQR) | 0.00 | (0.00-0.00) | 16.69 | (8.64-23.76) | 0.00 | (0.00-0.00) |
| Traumatic ABR | | | | | | |
| Mean (SD) | 0.36 | (0.83) | 4.82 | (6.31) | 0.15 | (0.78) |
| Median (IQR) | 0.00 | (0.00-0.00) | 3.95 | (0.00-6.48) | 0.00 | (0.00-0.00) |
| Joint ABR | | | | | | |
| Mean (SD) | 0.52 | (1.09) | 17.45 | (7.31) | 0.61 | (1.33) |
| Median (IQR) | 0.00 | (0.00-1.02) | 18.42 | (10.80-23.90) | 0.00 | (0.00-0.00) |
| All Bleeds ABR | | | | | | |
| (treated and untreated) | | | | | | |
| Mean (SD) | 1.10 | (1.92) | 22.22 | (7.94) | 0.84 | (1.61) |
| Median (IQR) | 0.00 | (0.00-1.15) | 21.13 | (16.80; 27.13) | 0.00 | (0.00-1.93) |
| Participants with zero | | | | | | |
| bleeds, n (%)^{b} | | | | | | |
| Total | 86 | (64.7) | 0 | | 20 | (76.9) |
| Spontaneous | 107 | (80.5) | 1 | (3.8) | 22 | (84.6) |
| Traumatic | 103 | (77.4) | 8 | (30.8) | 25 | (96.2) |
| Joint | 96 | (72.2) | 0 | | 21 | (80.8) |
| Spontaneous joint | 112 | (84.2) | 1 | (3.8) | 23 | (88.5) |
| Total number of treated bleeding | | | | | | |
| episodes, n | 86 | | 268 | | 8 | |
| Type of bleeding episodes, n (%) | | | | | | |
| Spontaneous | 33 | (38.4) | 197 | (73.5) | 5 | (62.5) |
| Traumatic | 45 | (52.3) | 62 | (23.1) | 2 | (40) |
| Unknown | 8 | (9.3) | 9 | (3.4) | 1 | (12.5) |
| Location of bleeding episodes, | | | | | | |
| N (%)^{c} | | | | | | |
| Joint | 61 | (56.5) | 219 | (81.7) | 7 | (87.5) |
| Muscle | 25 | (23.1) | 27 | (10.0) | 0 | |
| Internal | 7 | (6.5) | 5 | (1.9) | 0 | |
| Skin/mucosa | 15 | (13.9) | 18 | (6.7) | 1 | (12.5) |

| | | | | |
|---|---|---|---|---|
| Unknown location | 0 | 1 | (0.4) | 0 |

ABR, annualized bleed rate; IQR, interquartile range; PK, pharmacokinetic; SD, standard deviation.
^{a}Modeled ABR estimated using a negative binomial model with the total number of treated bleeding episodes during the efficacy period as the response variable and long-transformed efficacy period duration (in years) as an offset variable.
^{b}Percentages are based on the number of participants in each study arm and treatment regimen with an evaluable efficacy period. Efficacy period was defined as the sum of all intervals of time during which patients are treated with efanesoctocog alfa according to the study arm and treatment regimen, excluding periods of PK evaluation, surgery/rehabilitation, and large injection intervals.
^{c}A single bleeding episode could occur at more than 1 location and so the total number of bleeding episodes by location differs from the overall total number of bleeding episodes. Percentages are based on the total number of bleeding episodes by location.

A summary of the treatment of bleeding episodes is shown in Table 20.

TABLE 20
Summary of bleed treatment in Arm A and Arm B.

| | | | |
|---|---|---|---|
| Arm A | Arm B | Arm B | |
| prophylaxis | on demand | prophylaxis | Overall |
| (n = 133) | (n = 26) | (n = 26) | (N = 159) |

Injections required
to resolve
a bleed, n (%)a

| 1 | 81 | (94.2) | 261 | (97.4) | 8 | (100) | 350 | (96.7) |
|---|---|---|---|---|---|---|---|---|
| 2 | 4 | (4.7) | 7 | (2.6) | 0 | | 11 | (3.0) |
| 3 | 1 | (1.2) | 0 | | 0 | | 1 | (0.3) |
| Dose per injection (IU/kg) | | | | | | | | |
| Mean (SD) | 46.72 | (9.22) | 50.58 | (2.47) | 45.74 | (9.52) | 49.56 | (5.42) |
| Median (IQR) | 50.85 (47.17- | | 50.90 (50.00- | | 50.30 (40.00- | | 50.85 (50.00- | |
| | 52.20) | | 51.43) | | 51.42) | | 51.51) | |
| Total dose per bleeding | | | | | | | | |
| episode (IU/kg) | | | | | | | | |
| Mean (SD) | 49.95 | (16.63) | 51.74 | (6.56) | 45.74 | (9.52) | 51.18 | (10.00) |
| Median (IQR) | 51.00 (50.00- | | 50.93 (50.03- | | 50.30 (40.00- | | 50.93 (50.00- | |
| | 52.42) | | 51.47) | | 51.42) | | 51.85) | |
| Number of evaluable | 73 | | 255 | | 6 | | 334 | |
| bleeds | | | | | | | | |
| Participants response | | | | | | | | |
| to treatment with | | | | | | | | |
| efanesoctocog alfa | | | | | | | | |
| for bleeding episode, n (%) | | | | | | | | |
| Excellent | 39 | (53.4) | 195 | (76.5) | 5 | (83.3) | 239 | (71.6) |
| Good | 21 | (28.8) | 56 | (22.0) | 1 | (16.7) | 78 | (23.4) |

| | | | | | | | |
|---|---|---|---|---|---|---|---|
| Moderate | 10 | (13.7) | 4 | (1.6) | 00 | 14 | (4.2) |

| | | | | | | | |
|---|---|---|---|---|---|---|---|
| None | 3 | (4.1) | 0 | 5 | (83.3) | 3 | (0 9) |

### Secondary Endpoints of Phase 3 Study

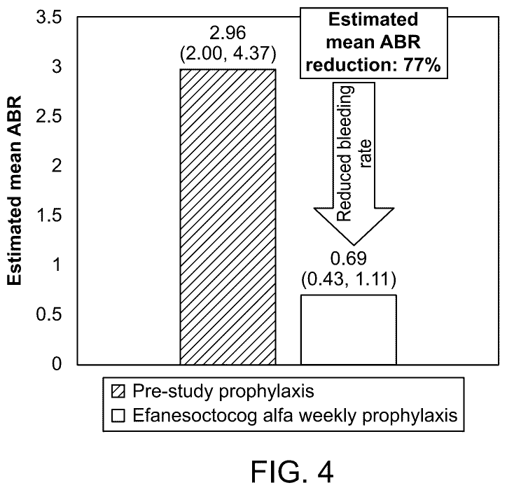

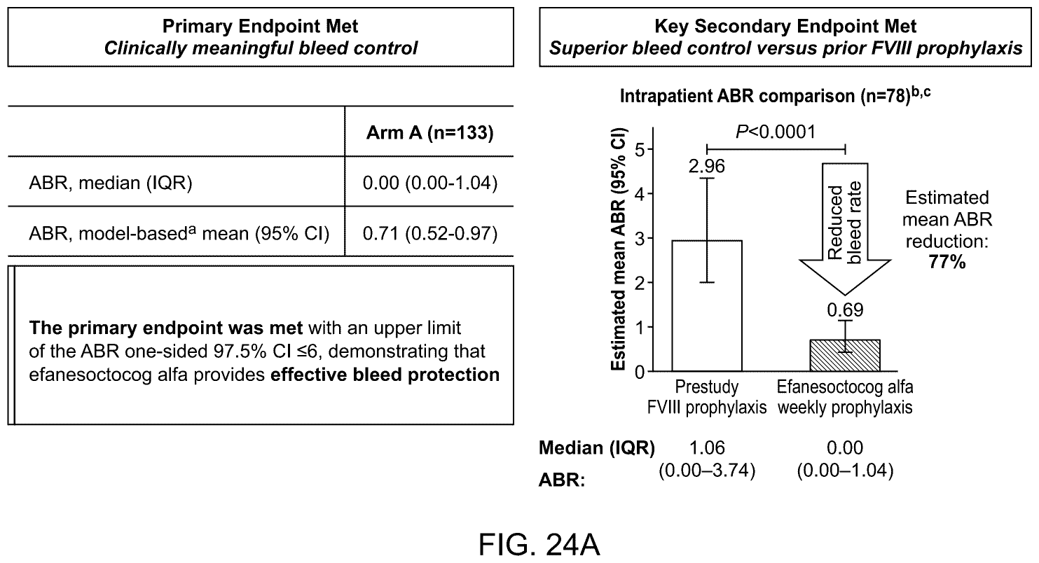

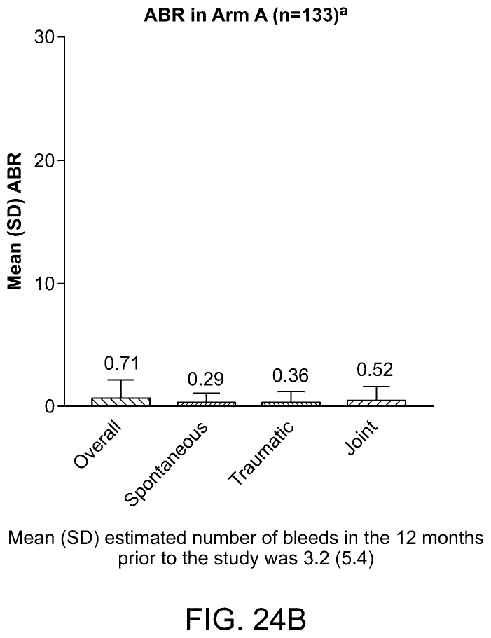

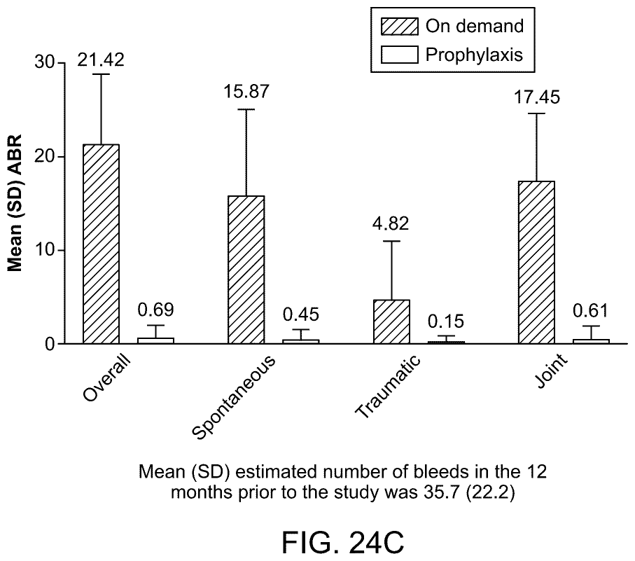

A key secondary efficacy endpoint of the study was the comparison between efanesoctocog alfa weekly prophylaxis treatment and historical prophylaxis treatment using an intra-patient (also referred to as intra-participant) comparison for participants in Arm A who participated in the observational study 242HA201/OBS16221. As shown in FIG. 4 (and FIG. 24A), the pre-study (242HA201/OBS16221) prophylaxis estimated mean ABR (i.e., prophylaxis with a medicinal product other than efanesoctocog alfa) for those in the observational study was 2.96 (2.00; 4.37) (n=78). Also see Table 22. The on-study estimated mean ABR for these same patients when on a once weekly prophylactic dose of efanesoctocog alfa was 0.69 (0.43; 1.11). This reduced bleeding rate from the historical prophylactic treatment of these patients was an estimated mean ABR reduction of 77% (see FIG. 24A).

The mean difference in overall ABR between historical prophylaxis and efanesoctocog alfa for the intra-participant comparison was −2.30 (95% CI −3.49; −1.11). The overall ABR results are shown in Table 21 (Per Protocol Set, n=77) and Table 22 (Full Analysis Set, n=78).

This shows that the conditions for non-inferiority of efanesoctocog alfa in comparison to the historical prophylactic treatment was met (upper bound of the 95% CI for ABR mean difference <4). The rate ratio comparing the efanesoctocog alfa weekly prophylactic treatment to historical prophylaxis was 0.23 (95% CI 0.13; 0.42; p<0.0001). This shows that the conditions to demonstrate superiority of efanesoctocog alfa compared to historical prophylactic treatment were met (Upper bound of the 95% CI for rate ratio <1; p value:≤0.05.

TABLE 21
Intra-participant comparison of annualized bleeding rates:
Efa (BIVV001) vs historical prophylaxis (Per Protocol Set)

| | Arm A | | | |
|---|---|---|---|---|
| | Historical Prophylaxis | | Efanesoctocog Alfa (BIVV001) | |
| | (OBS16221) | | Treatment in Phase 3 Study | |
| | (n = 77) | | (n = 77) | |
| # of Participants with an observation | 77 | | 77 | |
| or efficacy period | | | | |
| Total # of treated bleeding episodes | 212 | | 51 | |
| Total Participant-years followed | 69.7 | | 73.5 | |
| Duration of observation or efficacy | | | | |
| period (weeks) | | | | |
| Number | 77 | | 77 | |
| Mean (SD) | 47.26 | (6.72) | 49.80 | (2.47) |
| Median | 50.15 | | 50.09 | |
| Q1; Q3 | 43.86; 52.10 | | 49.07; 51.18 | |
| Min; Max | 27.4; 54.0 | | 39.1; 54.1 | |
| Annualized Bleeding Rate (ABR) | | | | |
| Number | 77 | | 77 | |
| Mean (SD) | 2.98 | (5.21) | 0.69 | (1.51) |
| Median | 1.07 | | 0.00 | |
| Q1; Q3 | 0.00; 3.74 | | 0.00; 1.04 | |
| Min; Max | 0.0; 35.6 | | 0.0; 11.0 | |
| 0 | 32 | (41.6) | 49 | (63.6) |
| >0-5 | 28 | (36.4) | 27 | (35.1) |
| >5-10 | 11 | (14.3) | 0 | |
| >10-20 | 5 | (6.5) | 1 | (1.3) |
| >20 | 1 | (1.3) | 0 | |
| Negative Binomial Regression Model | | | | |
| Mean ABR (95% CI) | 2.99 | (2.03; 4.42) | 0.69 | (0.43; 1.12) |
| mean difference (95% CI) | | | −2.30 | (−3.49; −1.11) |
| Rate Ratio (95% CI) | | | 0.23 | (0.13; 0.42) |
| p-value (superiority) | | | <.0001 | |
| Wilcoxon Signed Rank Test | | | | |
| Median ABR (Q1, Q3) | 1.07 | (0.00; 3.74) | 0.00 | (0.00; 1.04) |

| | | |
|---|---|---|
| mean difference (95% CI) | −1.36 | (−2.41; −0.57) |

| | |
|---|---|
| p-value (non-inferiority) | <.0001 |

TABLE 22
Intra-participant comparison of annualized bleeding rates:
Efa (BIVV001) vs historical prophylaxis (Full Analysis Set)

| | Arm A | | | |
|---|---|---|---|---|
| | Historical Prophylaxis | | Efanesoctocog Alfa (BIVV001) | |
| | (OBS16221) | | Treatment in Phase 3 Study | |
| | (N = 78) | | (N = 78) | |
| Number of participants with an observation or | 78 | | 78 | |
| efficacy period | | | | |
| Total number of treated bleeding episodes | 212 | | 51 | |
| Total participant-years followed | 70.5 | | 74.1 | |
| Duration of observation or efficacy period (weeks) | | | | |
| Number | 78 | | 78 | |
| Mean (SD) | 47.18 | (6.70) | 49.58 | (3.10) |
| Median | 50.05 | | 50.09 | |
| Q1; Q3 | 43.66; 52.10 | | 49.07; 51.18 | |
| Min; Max | 27.4; 54.0 | | 33.1; 54.1 | |
| Annualized bleeding rate (ABR) | | | | |
| Number | 78 | | 78 | |
| Mean (SD) | 2.95 | (5.19) | 0.68 | (1.50) |
| Median | 1.06 | | 0.00 | |
| Q1; Q3 | 0.00; 3.74 | | 0.00; 1.04 | |
| Min; Max | 0.0; 35.6 | | 0.0; 11.0 | |
| 0 | 33 | (42.3) | 50 | (64.1) |
| >0-5 | 28 | (35.9) | 27 | (34.6) |
| >5-10 | 11 | (14.1) | 0 | |
| >10-20 | 5 | (6.4) | 1 | (1.3) |
| >20 | 1 | (1.3) | 0 | |
| Negative Binomial regression model a | | | | |
| Mean ABR (95% CI) | 2.96 | (2.00; 4.37) | 0.69 | (0.43; 1.11) |
| mean difference (95% CI) | | | −2.27 | (−3.44; − 1.10) |
| Rate ratio (95% CI) | | | 0.23 | (0.13; 0.42) |
| p-value (superiority) b | | | <.0001 | |
| Wilcoxon Signed Rank test | | | | |
| Median ABR (Q1, Q3) | 1.06 | (0.00; 3.74) | 0.00 | (0.00; 1.04) |
| mean difference (95% CI) c | | | −1.36 | (−2.35; − 0.57) |

| | |
|---|---|
| p-value (non-inferiority) d | <.0001 |

Note:
1: Summaries are based on treated bleeds.
2: Percentages are based on the number of participants in the prophylaxis treatment regimen of each study with an evaluable observation or efficacy period.
3: The analysis is based on the Full Analysis Set and including participants in Arm A who have at least 6 months of efficacy period in the EFC16293 study and at least 6 months of observation period on prophylaxis collected in Study OBS16221.
4: The efficacy period reflects the sum of all intervals of time during which participants are treated with BIVV001 according to the study arms and treatment regimens excluding periods of PK evaluations, surgery/rehabilitation (minor and major), and large injection intervals (>28 days).
a Estimated using a negative binomial regression model with treatment (BIVV001 prophylaxis vs historical prophylaxis) as covariate.
b P-value relates to the null hypothesis: rate ratio (BIVV001 prophylaxis/historical prophylaxis) = 1.
c Estimated using the Hodges-Lehmann method.
d P-value relates to the null hypothesis: median of paired difference (BIVV001 prophylaxis − historical prophylaxis) = 4 based on Wilcoxon Signed Rank test.

Intra-participant annualized bleeding rates with efanesoctocog alfa weekly prophylaxis treatment was compared for participants who were historically treated with Eloctate® (efmoroctocog alfa). The results are shown in Table 23. These patients appeared to experience significantly fewer bleeds with weekly prophylaxis using efanesoctocog alfa than with historical prophylaxis using Eloctate® (efmoroctocog alfa). As shown in Table 23, the mean ABR for participants on efanesoctocog alfa was 0.57 versus 2.74 while on Eloctate®. The mean difference (95% CI) was −2.19 (−3.42; −0.96) and the rate ratio (95% CI) was 0.21 (0.12; 0.37) with a p-value <0.0001 corresponding to a rate reduction (95% CI) of 79% (63% to 88%), after the switch from Eloctate® to efanesoctocog alfa.

TABLE 23
Intra-participant comparison of annualized bleeding rates: Efa (BIVV001) weekly
prophylaxis vs historical prophylaxis using Eloctate ® (efmoroctocog alfa)

| | Arm A | | | |
|---|---|---|---|---|
| | Historical | | Efanesoctocog alfa | |
| | Prophylaxis | | Prophylaxis | |
| | (OBS16221) | | (EFC16293) | |
| | (N = 32) | | (N = 32) | |
| Number of participants with an observation or | 32 | | 32 | |
| efficacy period | | | | |
| Total number of treated bleeding episodes | 80 | | 18 | |
| Total participant-years followed | 27.7 | | 30.0 | |
| Duration of observation or efficacy | | | | |
| period (weeks) | | | | |
| Number | 32 | | 32 | |
| Mean (SD) | 45.22 | (7.13) | 48.98 | (4.10) |
| Median | 46.36 | | 50.24 | |
| Q1; Q3 | 41.19; 51.18 | | 48.17; 51.08 | |
| Min; Max | 29.1; 53.9 | | 33.1; 54.1 | |
| Annualized bleeding rate (ABR) | | | | |
| Number | 32 | | 32 | |
| Mean (SD) | 2.74 | (3.74) | 0.57 | (0.86) |
| Median | 1.18 | | 0.00 | |
| Q1; Q3 | 0.00; 4.73 | | 0.00; 1.02 | |
| Min; Max | 0.0; 13.0 | | 0.0; 3.1 | |
| 0 | 12 | (37.5) | 20 | (62.5) |
| >0-5 | 13 | (40.6) | 12 | (37.5) |
| >5-10 | 4 | (12.5) | 0 | |
| >10-20 | 3 | (9.4) | 0 | |
| >20 | 0 | | 0 | |
| Negative Binomial regression model ^{a} | | | | |
| Mean ABR (95% CI) | 2.78 | (1.74; 4.43) | 0.59 | (0.36; 0.98) |
| mean difference (95% CI) | | | −2.19 | (−3.42; −0.96) |
| Rate ratio (95% CI) | | | 0.21 | (0.12; 0.37) |

| | |
|---|---|
| p-value (superiority) ^{b} | <.0001 |

Note:
1: Summaries are based on treated bleeds.
2: Percentages are based on the number of participants in the prophylaxis treatment regimen of each study with an evaluable observation or efficacy period.
3: The analysis includes participants in Arm A who have at least 6 months of efficacy period in the EFC16293 study and at least 6 months of observation period on prophylaxis collected in Study OBS16221.
4: The efficacy period reflects the sum of all intervals of time during which participants are treated with Efa according to the study arms and treatment regimens excluding periods of PK evaluations, surgery/rehabilitation (minor and major), and large injection intervals (>28 days).
^{a} Estimated using a negative binomial regression model with treatment (Efa prophylaxis vs historical prophylaxis) as covariate.
^{b} P-value relates to the null hypothesis: rate ratio (Efa prophylaxis/historical prophylaxis) = 1.

Further intra-participant ABR evaluations were made for participants treated with efanesoctocog alfa weekly prophylaxis treatment (on-study) and compared with historical treatment with standard half-life (SHL) or extended half-life (EHL) FVIII products (pre-study). Of note, pre-study SHL includes SHL rFVIII and plasma-derived FVIII. Further intra-participant ABR evaluations included joint ABR, spontaneous ABR, and spontaneous joint ABR. Mean (95% confidence interval [CI]) change in joint, spontaneous, and spontaneous joint ABRs were −1.55 (−2.46, −0.64), −1.20 (−1.84, −0.57), and −0.93 (−1.42, −0.43), respectively. A summary of ABR results comparing historical treatment (pre-study) and efanesoctocog alfa treatment (on-study) is shown in Table 24. Overall, 42.3% of patients were bleed-free pre-study (median observation period: 50.05 weeks) versus 64.1% on-study (median: 50.09 weeks).

TABLE 24
Intra-participant pre-study ABR and on-study ABR with FVIII prophylaxis

| | Pre-study | On-study | Pre-study | On-study | Pre-study SHL | On-study |
|---|---|---|---|---|---|---|
| | SHL FVIII | efanesoctocog | EHL FVIII | efanesoctocog | and EHL FVIII | efanesoctocog |
| | prophylaxis^{a} | alfa prophylaxis | prophylaxis | alfa prophylaxis | prophylaxis^{a} | alfa prophylaxis |
| | (n = 44) | (n = 44) | (n = 34) | (n = 34) | (n = 78) | (n = 78) |
| Overall ABR | | | | | | |
| Mean (95% CI)^{a} | 3.23 (1.85, 5.63) | 0.79 (0.40, 1.56) | 2.61 (1.63, 4.20) | 0.55 (0.33, 0.92) | 2.96 (2.00, 4.37) | 0.69 (0.43, 1.11) |
| Median (IQR) | 1.05 (0.00, 3.42) | 0.00 (0.00, 1.04) | 1.10 (0.00, 4.50) | 0.00 (0.00, 1.02) | 1.06 (0.00, 3.74) | 0.00 (0.00, 1.04) |
| On-study vs pre-study | | | | | | |
| Mean difference (95% CI) | −2.44 (−4.31, −0.57) | | −2.06 (−3.23, −0.89) | | −2.27 (−3.44, −1.10) | |
| Rate ratio (95% CI) | 0.24 (0.10, 0.58) | | 0.21 (0.12, 0.37) | | 0.23 (0.13, 0.42) | |
| p-value | 0.0015 | | <0.0001 | | <0.0001 | |
| Joint ABR | | | | | | |
| Mean (95% CI)^{a} | 2.30 (1.23, 4.29) | 0.46 (0.22, 0.93) | 1.59 (0.98, 2.59) | 0.43 (0.26, 0.73) | 1.99 (1.27, 3.10) | 0.44 (0.28, 0.71) |
| Median (IQR) | 0.00 (0.00, 3.05) | 0.00 (0.00, 0.00) | 0.00 (0.00, 2.25) | 0.00 (0.00, 1.02) | 0.00 (0.00, 2.83) | 0.00 (0.00, 1.02) |
| On-study vs pre-study | | | | | | |
| Mean difference (95% CI) | −1.84 (−3.33, −0.36) | | −0.16 (−1.93, −0.40) | | −1.55 (−2.46, −0.64) | |
| Rate ratio (95% CI) | 0.20 (0.08, 0.52) | | 0.27 (0.14, 0.51) | | 0.22 (0.12, 0.43) | |
| p-value | 0.0011 | | <0.0001 | | <0.0001 | |
| Spontaneous ABR | | | | | | |
| Mean (95% CI)^{a} | 1.77 (1.06, 2.95) | 0.41 (0.22, 0.74) | 1.22 (0.65, 2.27) | 0.22 (0.10, 0.46) | 1.53 (1.02, 2.28) | 0.32 (0.20, 0.52) |
| Median (IQR) | 0.00 (0.00, 2.50) | 0.00 (0.00, 1.01) | 0.00 (0.00, 1.76) | 0.00 (0.00, 0.00) | 0.00 (0.00, 1.96) | 0.00 (0.00, 0.00) |
| On-study vs pre-study | | | | | | |
| Mean difference (95% CI) | −1.36 (−2.32, −0.40) | | −1.00 (−1.75, −0.25) | | −1.20 (−1.84, −0.57) | |
| Rate ratio (95% CI) | 0.23 (0.10, 0.53) | | 0.18 (0.07, 0.44) | | 0.21 (0.11, 0.40) | |
| p-value | 0.0005 | | 0.0002 | | <0.0001 | |
| Spontaneous joint ABR | | | | | | |
| Mean (95% CI)^{a} | 1.40 (0.83, 2.35) | 0.29 (0.15, 0.54) | 0.86 (0.46, 1.63) | 0.18 (0.08, 0.43) | 1.17 (0.77, 1.76) | 0.24 (0.15, 0.40) |
| Median (IQR) | 0.00 (0.00, 2.03) | 0.00 (0.00, 0.00) | 0.00 (0.00, 1.76) | 0.00 (0.00, 0.00) | 0.00 (0.00, 1.79) | 0.00 (0.00, 0.00) |
| On-study vs pre-study | | | | | | |

| | | | |
|---|---|---|---|
| Mean difference (95% CI) | −1.11 (−1.89, −0.34) | −0.68 (−1.21, −0.15) | −0.93 (−1.42, −0.43) |
| Rate ratio (95% CI) | 0.20 (0.09, 0.49) | 0.21 (0.09, 0.53) | 0.21 (0.11, 0.40) |
| p-value | 0.0004 | 0.0009 | <0.0001 |

ABR, annualized bleed rate; CI, confidence interval; EHL, extended half-life; FVIII, factor VIII; IQR, interquartile range; rFVIII, recombinant factor VIII; SHL standard half-life.

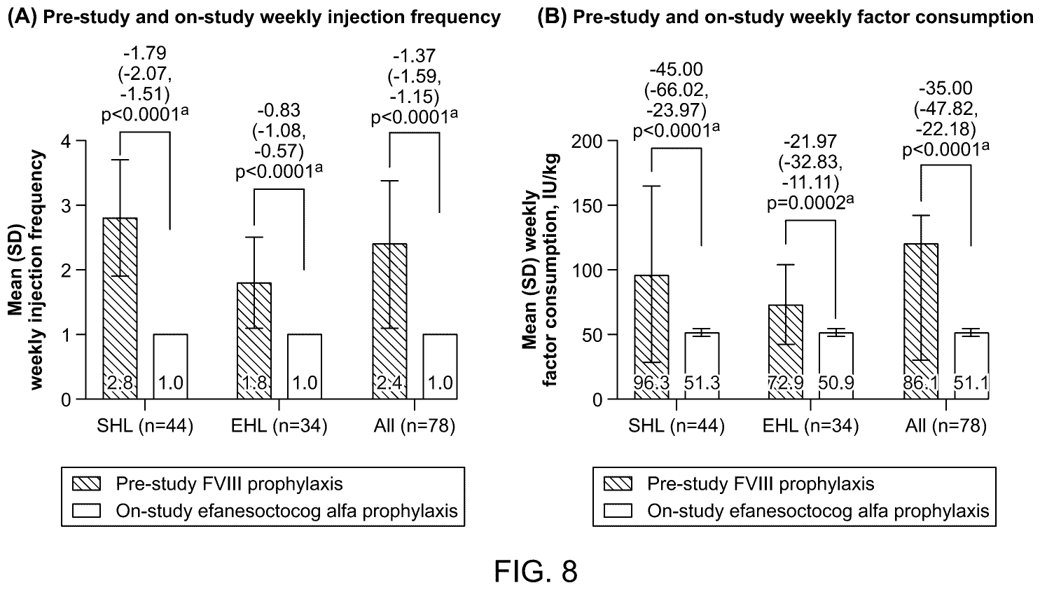

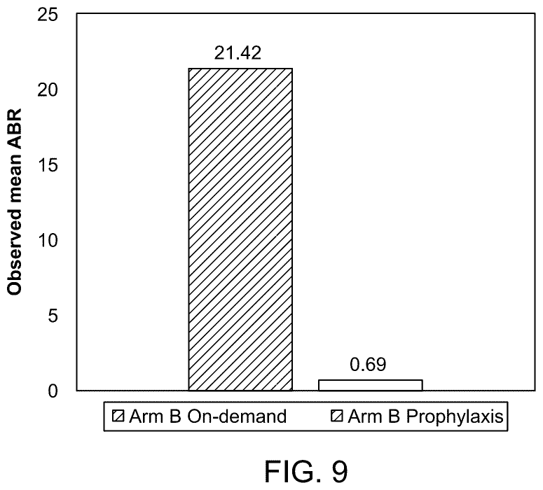

Intra-participant FVIII consumption and injection frequency were also analyzed with historical treatment using either SHL or EHL (pre-study) and compared with efanesoctocog alfa treatment (on-study). Mean (standard deviation) number of injections for bleed treatment was 1.8 (5.10) pre-study versus 1.1 (0.30) on-study. Mean (95% CI) change in weekly injection frequency was −1.37 (−1.59, −1.15) doses (see FIG. 8, (A)). Mean (95% CI) change in weekly in annualized consumption was −1828.22 (−2491.88, −1164.57) IU/kg (see FIG. 8, (B)). Pre-study SHL includes SHL rFVIII and plasma-derived FVIII. FVIII consumption and injection frequency results are shown in Table 25.

TABLE 25
Intra-participant pre-study and on-study comparison of weekly
injection frequency and FVIII consumption (Median IQR)

| | Weekly injection frequency | | Weekly factor (FVIII) consumption, IU/kg | | |
|---|---|---|---|---|---|
| | | On-study | | | On-study |
| | Pre-study FVIII | efanesoctocog alfa | Pre-study FVIII | | efanesoctocog alfa |
| | prophylaxis | prophylaxis | prophylaxis | | prophylaxis |
| SHL (n = 44) | 2.8 (2.2, 3.3) | 1.0 (1.0, 1.0) | 84.2 | (57.6, 122.3) | 51.5 (49.6, 52.9) |
| EHL (n = 34) | 1.8 (1.3, 2.3) | 1.0 (1.0, 1.0) | 69.8 | (53.7, 88.3) | 51.1 (49.4, 52.4) |
| All (n = 78) | 2.2 (1.8, 3.1) | 1.0 (1.0, 1.0) | 75.8 | (56.8, 103.6) | 51.3 (49.4, 52.8) |

### On-Demand Treatment Results

During the on-demand treatment portion of Arm B of the phase 3 study, the mean observed ABR of these 26 subjects was 21.42 (SD=7.41). The median observed ABR was 21.13 (Q1-15.12; Q3-27.13). When switched to a prophylactic dose of efanesoctocog alfa once weekly at 50 IU/kg, these subjects exhibited a mean observed ABR of 0.69 (SD=1.35). The median ABR in these subjects following the switch to a prophylactic treatment was 0.00. The Observed Total ABR data from Arm B (on-demand) and Arm B (prophylaxis) are shown in Table 26, while annualized joint bleeding rate data from Arm B is shown in Table 27.

TABLE 26
Summary of annualized bleeding rates - Arm B

| | | |
|---|---|---|
| | Arm B | Arm B |
| ABR | On-Demand (n = 26) | Prophylaxis (n = 26) |

| | | | | |
|---|---|---|---|---|
| Mean (SD) | 21.42 | (7.41) | 0.69 | (1.35) |

| Median | 21.13 | 0.00 | |
|---|---|---|---|
| Q1; Q3 | 15.12; 27.13 | 0.00; 0.00 | |
| 0, n (%) | 0 | 20 | (76.9) |
| >0-5, n (%) | 0 | 6 | (23.1) |

| | | | |
|---|---|---|---|
| >5-10, n (%) | 1 | (3.8) | 0 |
| >10-20, n (%) | 10 | (38.5) | 0 |
| >20, n (%) | 15 | (57.7) | 0 |

TABLE 27
Summary of annualized joint bleeding rates - Arm B

| | | |
|---|---|---|
| | Arm B | Arm B |
| AjBR | On-Demand (n = 26) | Prophylaxis (n = 26) |

| Mean (SD) | 17.45 | (7.31) | 0.61 | (1.33) |
|---|---|---|---|---|
| Median | 18.42 | | 0.00 | |
| Q1; Q3 | 10.80; 23.90 | | 0.00; 0.00 | |
| 0, n (%) | 0 | | 21 | (80.8) |
| >0-5, n (%) | 1 | (3.8) | 5 | (19.2) |

| | | | |
|---|---|---|---|
| >5-10, n (%) | 3 | (11.5) | 0 |
| >10-20, n (%) | 13 | (50.0) | 0 |
| >20, n (%) | 9 | (34.6) | 0 |

The distribution of the number of bleeding episodes among subjects was also examined in Arm B of the Phase 3 Study. During the on-demand period, 0 subjects had no bleeding episodes, and 0 subjects had between 1 and 5 bleeding episodes. 1 subject (3.8%) had between 6 and 10 bleeding episodes. 10 subjects (38.5%) had between 11 and 20 bleeding episodes. 15 subjects (57.7%) had 21 or more bleeding episodes. In comparison, when these 26 subjects switched to prophylactic treatment, 20 subjects had no bleeding episodes (76.9%). 6 subjects (23.1%) had between 1 and 5 bleeding episodes during the 26 week prophylactic period. No subjects had 6 to 10, 11 to 20, or more than 20 bleeding episodes.

All 45 target joints identified at baseline resolved in the 14 patients with target joints at baseline and at least 12 months of on-study prophylaxis. Additionally, a reduction in target joint bleeds was observed in Arm B following the switch from on-demand to prophylaxis.

A summary of annualized bleeding rates by location of bleed (Full Analysis Set) can be seen in Table 28.

Low bleed rates were observed after a switch to efanesoctocog alfa prophylaxis from historical factor treatment (Arm A). The overall annualized bleed rate on prophylaxis was 0.71 (See Table 20), while the annualized bleed rate due to spontaneous bleeds was 0.29 (Table 48). The annualized bleed rate for traumatic bleeds was 0.36 (0.83), and the annualized bleed rate for joint bleeds was 0.52 (FIG. 24B).

Lowered bleed rates were also observed while on prophylaxis following the on-demand portion of Arm B. During the on-demand treatment portion, the mean (SD) overall annualized bleed rate was 21.42 (7.41), the spontaneous bleed rate was 15.87, the mean (SD) traumatic bleed rate was 4.82 (6.31), and the mean (SD) joint bleed rate was 17.45 (7.31). Once patients were placed onto prophylaxis, these rates dropped to 0.69 (1.35), 0.45 (1.13), 0.15 (0.78), and 0.61 (1.33), respectively (FIG. 24C).

TABLE 28
Summary of annualized bleeding rates by location of bleed - Full Analysis Set

| | Arm A | | Arm B | | | |
|---|---|---|---|---|---|---|
| | Prophylaxis | | On-demand | | Prophylaxis | |
| | (N = 133) | | (N = 26) | | (N = 26) | |
| Location of bleed | | | | | | |
| Number of participants with an efficacy period | 133 | | 26 | | 26 | |
| Total number of treated bleeding episodes at joint | 61 | | 219 | | 7 | |
| Total number of treated bleeding episodes at muscle | 25 | | 27 | | 0 | |
| Total number of treated bleeding episodes at internal | 7 | | 5 | | 0 | |
| Total number of treated bleeding episodes at skin/mucosa | 15 | | 18 | | 1 | |
| Total number of treated bleeding episodes at unknown | 0 | | 1 | | 0 | |
| location | | | | | | |
| Joint | | | | | | |
| Participant-level | | | | | | |
| Number | 133 | | 26 | | 26 | |
| Mean (SD) | 0.52 | (1.09) | 17.45 | (7.31) | 0.61 | (1.33) |
| Median | 0.00 | | 18.42 | | 0.00 | |
| Q1; Q3 | 0.00; 1.02 | | 10.80; 23.90 | | 0.00; 0.00 | |
| Min; Max | 0.0; 6.2 | | 4.1; 30.4 | | 0.0; 4.1 | |
| 0 | 96 | (72.2) | 0 | | 21 | (80.8) |
| >0-5 | 35 | (26.3) | 1 | (3.8) | 5 | (19.2) |
| >5-10 | 2 | (1.5) | 3 | (11.5) | 0 | |
| >10-20 | 0 | | 13 | (50.0) | 0 | |
| >20 | 0 | | 9 | (34.6) | 0 | |
| Population-level, model baseda | | | | | | |
| Mean (95% CI) | 0.51 (0.36; | | 17.48 (14.88; | | 0.62 (0.25; | |
| | 0.72) | | 20.54) | | 1.52) | |
| Muscle | | | | | | |
| Participant-level | | | | | | |
| Number | 133 | | 26 | | 26 | |
| Mean (SD) | 0.20 | (0.93) | 2.20 | (3.28) | 0.00 | (0.00) |
| Median | 0.00 | | 0.00 | | 0.00 | |
| Q1; Q3 | 0.00; 0.00 | | 0.00; 4.15 | | 0.00; 0.00 | |
| Min; Max | 0.0; 7.7 | | 0.0; 13.0 | | 0.0; 0.0 | |
| 0 | 121 | (91.0) | 14 | (53.8) | 26 | (100) |
| >0-5 | 10 | (7.5) | 8 | (30.8) | 0 | |
| >5-10 | 2 | (1.5) | 3 | (11.5) | 0 | |
| >10-20 | 0 | | 1 | (3.8) | 0 | |
| >20 | 0 | | 0 | | 0 | |
| Population-level, model baseda | | | | | | |
| Mean (95% CI) | 0.21 (0.10; | | 2.18 (1.20; | | NC# | |
| | 0.44) | | 3.98) | | | |
| Internal | | | | | | |
| Participant-level | | | | | | |
| Number | 133 | | 26 | | 26 | |
| Mean (SD) | 0.05 | (0.26) | 0.40 | (1.67) | 0.00 | (0.00) |
| Median | 0.00 | | 0.00 | | 0.00 | |
| Q1; Q3 | 0.00; 0.00 | | 0.00; 0.00 | | 0.00; 0.00 | |
| Min; Max | 0.0; 2.0 | | 0.0; 8.3 | | 0.0; 0.0 | |
| 0 | 127 | (95.5) | 24 | (92.3) | 26 | (100) |
| >0-5 | 6 | (4.5) | 1 | (3.8) | 0 | |
| >5-10 | 0 | | 1 | (3.8) | 0 | |
| >10-20 | 0 | | 0 | | 0 | |
| >20 | 0 | | 0 | | 0 | |
| Population-level, model baseda | | | | | | |
| Mean (95% CI) | 0.06 (0.02; | | 0.40 (0.06; | | NC# | |
| | 0.13) | | 2.66) | | | |
| Skin/mucosa | | | | | | |
| Participant-level | | | | | | |
| Number | 133 | | 26 | | 26 | |
| Mean (SD) | 0.12 | (0.62) | 1.45 | (2.05) | 0.08 | (0.41) |
| Median | 0.00 | | 0.00 | | 0.00 | |
| Q1; Q3 | 0.00; 0.00 | | 0.00; 2.09 | | 0.00; 0.00 | |
| Min; Max | 0.0; 6.2 | | 0.0; 6.5 | | 0.0; 2.1 | |
| 0 | 125 | (94.0) | 15 | (57.7) | 25 | (96.2) |
| >0-5 | 7 | (5.3) | 9 | (34.6) | 1 | (3.8) |
| >5-10 | 1 | (0.8) | 2 | (7.7) | 0 | |
| >10-20 | 0 | | 0 | | 0 | |
| >20 | 0 | | 0 | | 0 | |
| Population-level, model baseda | | | | | | |
| Mean (95% CI) | 0.12 (0.05; | | 1.44 (0.83; | | NC# | |
| | 0.29) | | 2.52) | | | |
| Unknown location | | | | | | |
| Participant-level | | | | | | |
| Number | 133 | | 26 | | 26 | |
| Mean (SD) | 0.00 | (0.00) | 0.08 | (0.41) | 0.00 | (0.00) |
| Median | 0.00 | | 0.00 | | 0.00 | |
| Q1; Q3 | 0.00; 0.00 | | 0.00; 0.00 | | 0.00; 0.00 | |
| Min; Max | 0.0; 0.0 | | 0.0; 2.1 | | 0.0; 0.0 | |
| 0 | 133 | (100) | 25 | (96.2) | 26 | (100) |

| | | | | |
|---|---|---|---|---|
| >0-5 | 0 | 1 | (3.8) | 0 |

| >5-10 | 0 | 0 | 0 |
|---|---|---|---|
| >10-20 | 0 | 0 | 0 |
| >20 | 0 | 0 | 0 |
| Population-level, model baseda | | | |
| Mean (95% CI) | NC# | 0.08 (0.01; | NC# |
| | | 0.57) | |

Note:
1: Summaries are based on treated bleeds.
2: Percentages are based on the number of participants in each study arm and treatment regimen with an evaluable efficacy period.
3: The efficacy period reflects the sum of all intervals of time during which participants are treated with BIVV001 according to the study arms and treatment regimens excluding periods of PK evaluations, surgery/rehabilitation (minor and major), and large injection intervals (>28 days).
4: Participants are included in each study arm and treatment regimen they participated in for the duration of time on that regimen and, as such, may appear in more than one treatment regimen.
aEstimated using a negative binomial model with the total number of treated bleeding episodes during the efficacy period as the response variable and log-transformed efficacy period duration (in years) as an offset variable.
#The parameter cannot be estimated due to too few bleedings occurring in the category and regimen.

### Quality of Life (QoL) Outcomes

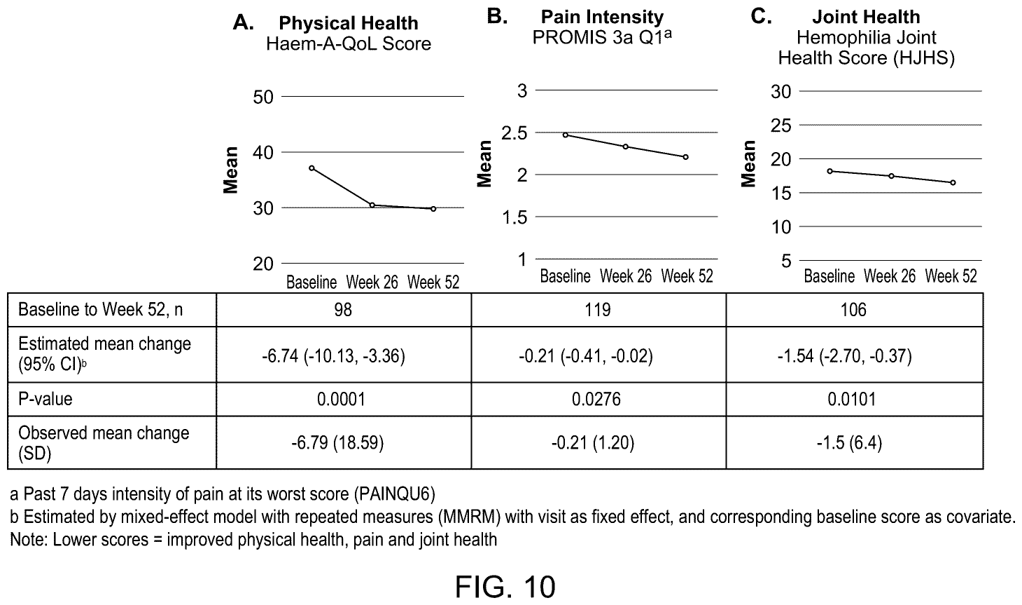

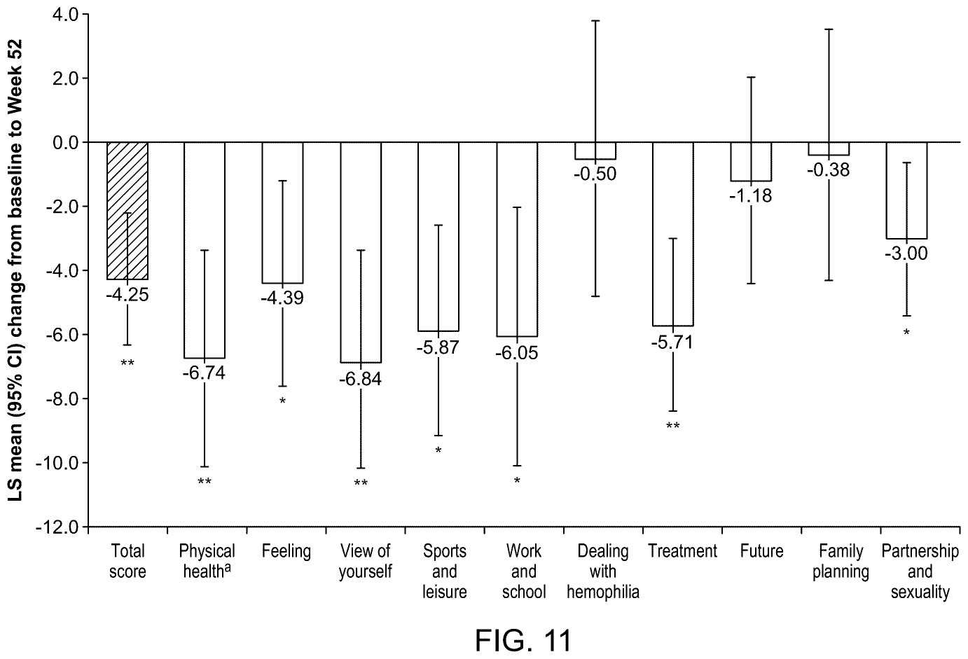

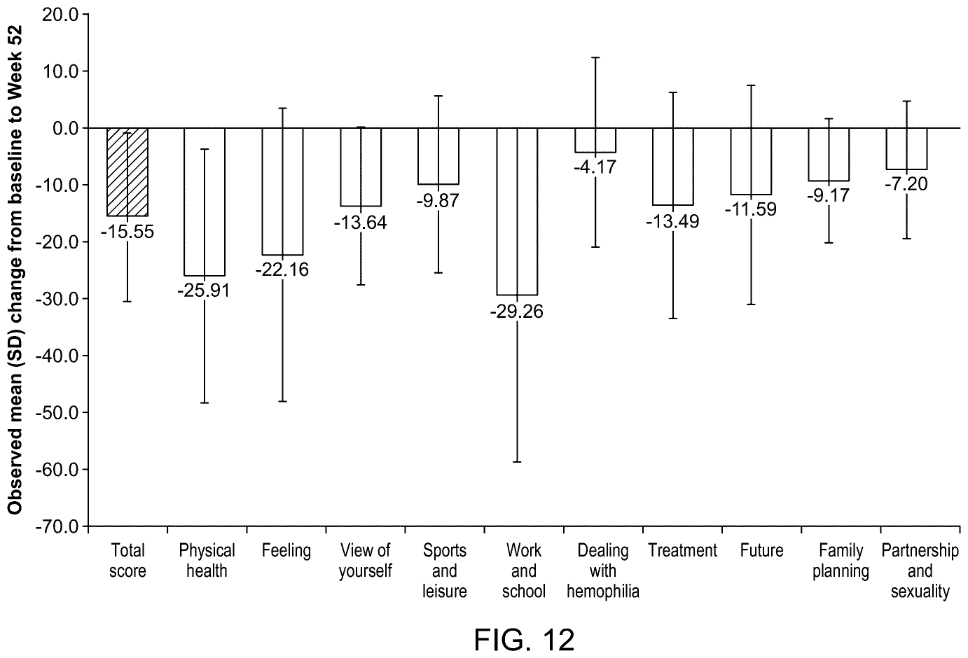

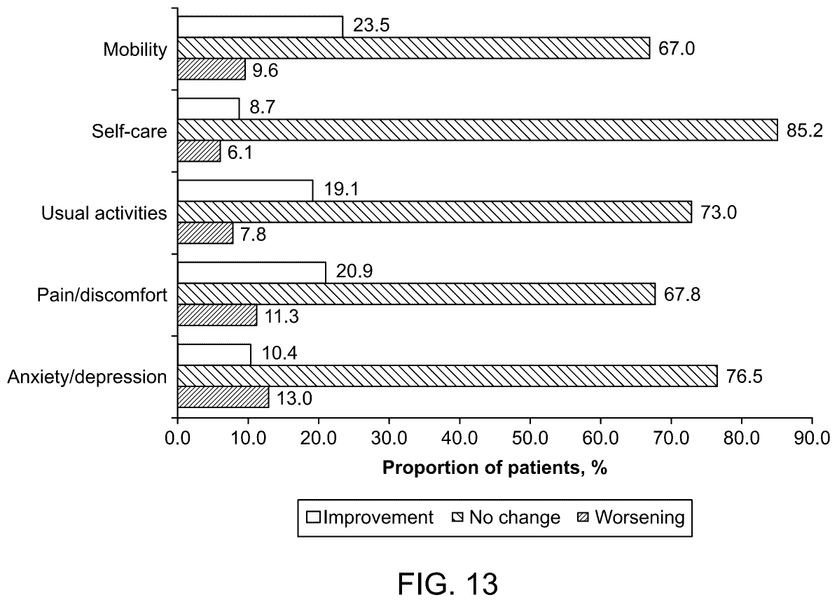

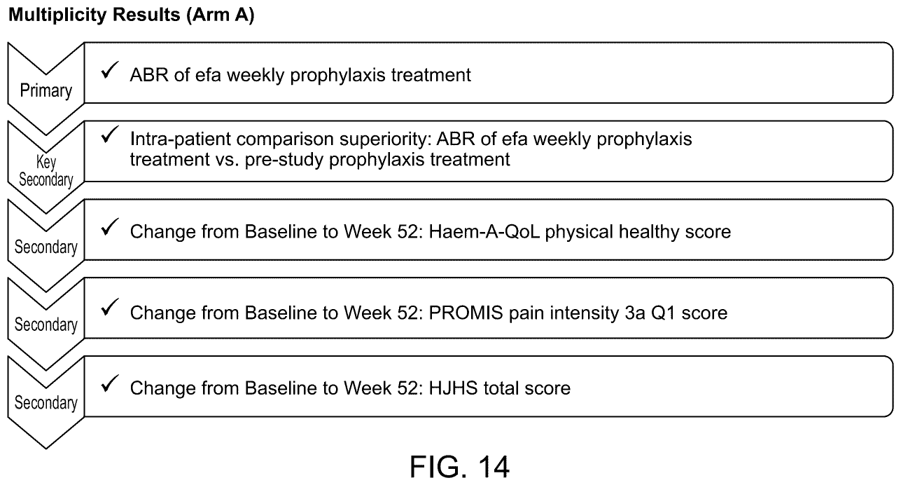

A secondary efficacy objective of the study was to evaluate the effect of efanesoctocog alfa prophylaxis on joint health outcomes and Quality of Life (QoL) outcomes. Efanesoctocog alfa treatment demonstrated statistically significant improvements in physical health, pain intensity, and joint health measures assessed as secondary endpoints as compared to baseline in patients previously treated with other FVIII products for prophylaxis (see, e.g., FIGS. 10A-C).

### Physical Health and Function

### Haem-A-QoL Score

Physical health was measured by Haem-A-QoL Score (see Von Mackensen S, et al. Haemophilia. 2017 23(3):383-91) in adult subjects (>17 years) baseline to week 52 (n=98) (FIG. 10A). The change from baseline to Week 52 in Haem-A-QoL physical health score in Arm A was analyzed as part of the hierarchical testing procedure. The mean change in Haem-A-QoL physical health subscale scores from baseline to Week 52 using Mixed Effect Model with Repeated Measures (MMRM) (>=17 years old) is shown in Table 29. For Arm A, mean (SD) Haem-A-QoL physical health subscale scores were 37.02 (23.83), 30.33 (23.17) and 29.66 (23.40) at baseline, week 26, and week 52, respectively.

The improvement in Haem-A-QoL Total Score were for all subjects receiving efanesoctocog alfa prophylaxis from baseline to Week 52 was −4.25 (LS mean 95% CI: −6.31 to −2.20, p<0.0001). The psychometric analysis based on Phase 3 data reported meaningful within-group improvement for Haem-A-QoL Total score of −5.8 to −3.5. Statistically significant improvements were observed in 7/10 domains of the Haem-A-QoL: View of yourself (−6.84 [−10.17; −3.50]; p<0.0001); Physical Health (−6.74, LS mean 95% CI: −10.13 to −3.36; p=0.0001; n=98); Work and School (−6.05, LS mean 95% CI: −10.09 to −2.02; p=0.0038); Sports and Leisure (−5.87, LS mean 95% CI: −9.16 to −2.58; p=0.0006); Treatment (−5.71, LS mean 95% CI: −8.41 to −3.01), p<0.0001); Feeling (−4.39, LS mean 95% CI: −7.59 to −1.18; p=0.0078); Partnership and Sexuality (−3.00, LS mean 95% CI: −5.40 to −0.60; p=0.0148). A summary of Haem-A-QoL total scores, individual domain scores, and change from baseline to Week 52 is provided in Table 30 for participants >17 years of age, Table 31 for participants 13-16 years of age, and Table 32 for participants of 12 years of age. Total and subscale scores are presented as the Transformed Scale Score (TSS) for Tables 30-32. The T score for Haem-A-QoL PH was calculated using the transformed raw score summed across all items.

As shown in Table 29, the adjusted mean change from baseline to Week 52 in Haem-A-QoL physical health score (n=98) was −6.74 (95% CI: −10.13 to −3.36, p-value=0.0001) demonstrating a statistically significant improvement in physical health. The adjusted change in Haem-A-QoL physical health score from Baseline to Week 52 of −6.74 corresponds to a 18.2% mean improvement. An extensive psychometric analysis based on Phase 3 data reported meaningful within-group improvement for Haem-A-QoL Physical Health score of −6.8 to −4.8.

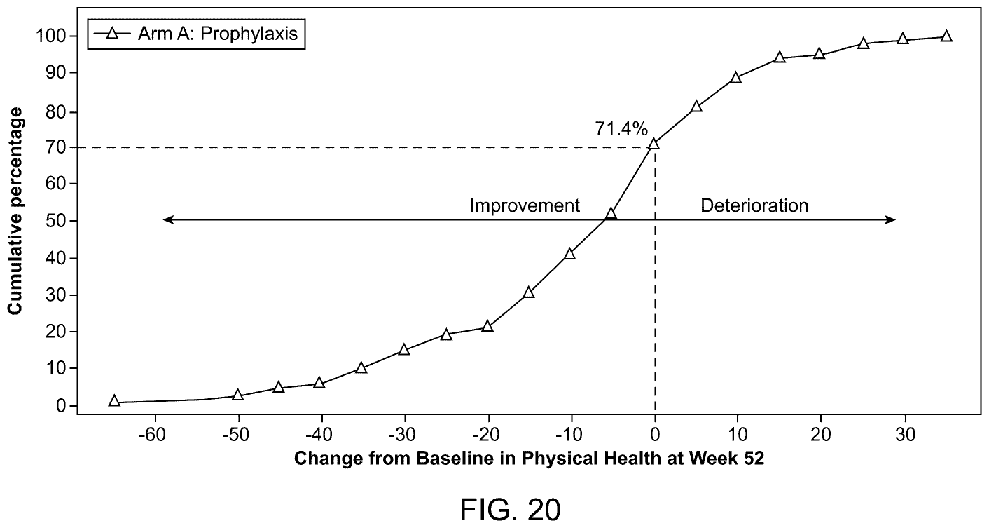

Most patients (71.4%; n=98) numerically maintained or improved their PH score from baseline to Week 52 (FIG. 20). Psychometric analyses supported a meaningful within-patient improvement threshold of −10 (range: −15.0 to −8.7). Almost 42% of patients reported a clinically meaningful improvement in PH score from baseline to Week 52.

TABLE 29
Mean change in Haem-A-QoL physical health subscale scores from baseline
to Week 52 using MMRM (>=17 years old) - Full Analysis Set

| | Arm A | | | | Arm B | | | | | | | |
|---|---|---|---|---|---|---|---|---|---|---|---|---|
| | Prophylaxis | | | | On-demand −> Prophylaxis | | | | Overall | | | |
| | (N = 110) | | | | (N = 26) | | | | (N = 136) | | | |
| | Actual | | Change from | | Actual | | Change from | | Actual | | Change from | |
| Visit | result | | baseline | | result | | baseline | | result | | baseline | |
| Physical | | | | | | | | | | | | |
| Health | | | | | | | | | | | | |
| Baseline | | | | | | | | | | | | |
| Number | 104 | | z | | 23 | | z | | 127 | | z | |
| Mean (SD) | 37.02 | (23.83) | | | 46.09 | (21.79) | | | 38.66 | (23.65) | | |
| Median | 35.00 | | | | 45.00 | | | | 40.00 | | | |
| Q1; Q3 | 20.00; 55.00 | | | | 35.00; 65.00 | | | | 20.00; 55.00 | | | |
| Min; Max | 0.0; 100.0 | | | | 0.0; 90.0 | | | | 0.0; 100.0 | | | |
| Week 26 | | | | | | | | | | | | |
| Number | 105 | | 99 | | 26 | | 23 | | 131 | | 122 | |
| Mean (SD) | 30.33 | (23.17) | −6.62 | (18.15) | 30.58 | (18.94) | −14.57 | (19.54) | 30.38 | (22.32) | −8.11 | (18.60) |
| Median | 30.00 | | −5.00 | | 30.00 | | −15.00 | | 30.00 | | −5.00 | |
| Q1; Q3 | 15.00; 45.00 | | −20.00; 5.00 | | 15.00; 45.00 | | −35.00; −5.00 | | 15.00; 45.00 | | −20.00; 5.00 | |
| Min; Max | 0.0; 85.0 | | −100.0; 30.0 | | 0.0; 75.0 | | −45.0; 35.0 | | 0.0; 85.0 | | −100.0; 35.0 | |
| Week 52 | | | | | | | | | | | | |
| Number | 104 | | 98 | | 25 | | 22 | | 129 | | 120 | |
| Mean (SD) | 29.66 | (23.40) | −6.79 | (18.59) | 18.40 | (17.24) | −25.91 | (22.29) | 27.48 | (22.73) | −10.29 | (20.60) |

| Median | 27.50 | −5.00 | | 15.00 | −27.50 | 25.00 | −5.00 |
|---|---|---|---|---|---|---|---|
| Q1; Q3 | 7.50; 45.00 | −15.00; 5.00 | | 5.00; 25.00 | −40.00; −10.00 | 5.00; 45.00 | −25.00; 2.50 |
| Min; Max | 0.0; 90.0 | −65.0; 35.0 | | 0.0; 60.0 | −70.0; 10.0 | 0.0; 90.0 | −70.0; 35.0 |
| LS Mean | | −6.74 | (1.71) | | | | |
| (SE)a | | | | | | | |

| | |
|---|---|
| 95% C.la | (−10.13, −3.36) |
| p-value | 0.0001 |

TABLE 30
Summary of Haem-A-QoL total scores, domain scores, and change
from baseline to Week 52 (≥17 years old) - Full Analysis Set

| | Arm A | | Arm B | | | |
|---|---|---|---|---|---|---|
| | Prophylaxis | | On-demand −> Prophylaxis | | Overall | |
| | (N = 110) | | (N = 26) | | (N = 136) | |
| Domain | Actual | Change from | Actual | Change from | Actual | Change from |
| Visit | result | baseline | result | baseline | result | baseline |
| Total Score | | | | | | |
| Baseline | | | | | | |
| Number | 104 | | 23 | | 127 | |
| Mean (SD) | 31.23 (17.65) | | 37.64 (15.22) | | 32.39 (17.35) | |
| Median | 30.95 | | 35.00 | | 32.07 | |
| Q1; Q3 | 17.39; 41.35 | | 27.78; 47.09 | | 18.18; 42.86 | |
| Min; Max | 0.0; 72.8 | | 10.3; 74.3 | | 0.0; 74.3 | |
| Week 52 | | | | | | |
| Number | 104 | 98 | 25 | 22 | 129 | 120 |
| Mean (SD) | 26.31 (16.97) | −4.56 (11.15) | 21.41 (10.95) | −15.55 (14.78) | 25.36 (16.06) | −6.57 (12.57) |
| Median | 23.04 | −3.80 | 19.59 | −11.63 | 21.67 | −4.42 |
| Q1; Q3 | 11.45; 37.93 | −8.70; 2.27 | 15.22; 23.75 | −23.89; −4.35 | 13.33; 35.90 | −12.40; 0.94 |
| Min; Max | 0.0; 72.2 | −41.3; 20.8 | 5.8; 59.0 | −48.3; 4.5 | 0.0; 72.2 | −48.3; 20.8 |
| Physical | | | | | | |
| Health | | | | | | |
| Baseline | | | | | | |
| Number | 104 | | 23 | | 127 | |
| Mean (SD) | 37.02 (23.83) | | 46.09 (21.79) | | 38.66 (23.65) | |
| Median | 35.00 | | 45.00 | | 40.00 | |
| Q1; Q3 | 20.00; 55.00 | | 35.00; 65.00 | | 20.00; 55.00 | |
| Min; Max | 0.0; 100.0 | | 0.0; 90.0 | | 0.0; 100.0 | |
| Week 52 | | | | | | |
| Number | 104 | 98 | 25 | 22 | 129 | 120 |
| Mean (SD) | 29.66 (23.40) | −6.79 (18.59) | 18.40 (17.24) | −25.91 (22.29) | 27.48 (22.73) | −10.29 (20.60) |
| Median | 27.50 | −5.00 | 15.00 | −27.50 | 25.00 | −5.00 |
| Q1; Q3 | 7.50; 45.00 | −15.00; 5.00 | 5.00; 25.00 | −40.00; −10.00 | 5.00; 45.00 | −25.00; 2.50 |
| Min; Max | 0.0; 90.0 | −65.0; 35.0 | 0.0; 60.0 | −70.0; 10.0 | 0.0; 90.0 | −70.0; 35.0 |
| Feeling | | | | | | |
| Baseline | | | | | | |
| Number | 104 | | 23 | | 127 | |
| Mean (SD) | 25.36 (23.32) | | 34.51 (25.55) | | 27.02 (23.90) | |
| Median | 25.00 | | 31.25 | | 25.00 | |
| Q1; Q3 | 6.25; 37.50 | | 18.75; 56.25 | | 6.25; 37.50 | |
| Min; Max | 0.0; 100.0 | | 0.0; 93.8 | | 0.0; 100.0 | |
| Week 52 | | | | | | |
| Number | 104 | 98 | 25 | 22 | 129 | 120 |
| Mean (SD) | 19.89 (22.57) | −4.34 (17.46) | 11.25 (15.93) | −22.16 (25.79) | 18.22 (21.67) | −7.60 (20.34) |
| Median | 12.50 | 0.00 | 6.25 | −18.75 | 12.50 | 0.00 |
| Q1; Q3 | 0.00; 31.25 | −12.50; 6.25 | 0.00; 12.50 | −31.25; −6.25 | 0.00; 25.00 | −18.75; 6.25 |
| Min; Max | 0.0; 93.8 | −68.8; 37.5 | 0.0; 68.8 | −93.8; 12.5 | 0.0; 93.8 | −93.8; 37.5 |
| View of | | | | | | |
| Yourself | | | | | | |
| Baseline | | | | | | |
| Number | 104 | | 23 | | 127 | |
| Mean (SD) | 38.56 (24.34) | | 42.17 (18.14) | | 39.21 (23.32) | |
| Median | 35.00 | | 40.00 | | 40.00 | |
| Q1; Q3 | 20.00; 52.50 | | 30.00; 50.00 | | 20.00; 50.00 | |
| Min; Max | 0.0; 95.0 | | 15.0; 90.0 | | 0.0; 95.0 | |
| Week 52 | | | | | | |
| Number | 104 | 98 | 25 | 22 | 129 | 120 |
| Mean (SD) | 30.91 (23.45) | −7.40 (18.25) | 27.80 (18.15) | −13.64 (13.99) | 30.31 (22.49) | −8.54 (17.66) |
| Median | 27.50 | −5.00 | 25.00 | −12.50 | 25.00 | −5.00 |
| Q1; Q3 | 10.00; 50.00 | −15.00; 5.00 | 20.00; 40.00 | −20.00; −5.00 | 10.00; 50.00 | −15.00; 2.50 |
| Min; Max | 0.0; 90.0 | −90.0; 55.0 | 0.0; 75.0 | −35.0; 15.0 | 0.0; 90.0 | −90.0; 55.0 |
| Work and School | | | | | | |
| Baseline | | | | | | |
| Number | 84 | | 21 | | 105 | |
| Mean (SD) | 48.61 (27.21) | | 66.55 (21.57) | | 52.20 (27.06) | |
| Median | 50.00 | | 75.00 | | 55.00 | |
| Q1; Q3 | 27.50; 70.00 | | 55.00; 85.00 | | 30.00; 75.00 | |
| Min; Max | 0.0; 100.0 | | 20.0; 100.0 | | 0.0; 100.0 | |
| Week 52 | | | | | | |
| Number | 82 | 71 | 20 | 17 | 102 | 88 |
| Mean (SD) | 40.98 (26.16) | −5.65 (18.66) | 38.13 (22.29) | −29.26 (29.45) | 40.42 (25.37) | −10.21 (22.97) |
| Median | 37.50 | −5.00 | 41.88 | −25.00 | 40.00 | −9.38 |
| Q1; Q3 | 20.00; 65.00 | −15.00; 5.00 | 21.88; 50.00 | −50.00; −5.00 | 20.00; 62.50 | −25.00; 5.00 |
| Min; Max | 0.0; 90.0 | −65.0; 35.0 | 0.0; 75.0 | −80.0; 15.0 | 0.0; 90.0 | −80.0; 35.0 |
| Sports and Leisure | | | | | | |
| Baseline | | | | | | |
| Number | 93 | | 21 | | 114 | |
| Mean (SD) | 18.35 (19.77) | | 23.21 (19.07) | | 19.24 (19.65) | |
| Median | 12.50 | | 18.75 | | 12.50 | |
| Q1; Q3 | 0.00; 25.00 | | 6.25; 31.25 | | 0.00; 31.25 | |
| Min; Max | 0.0; 87.5 | | 0.0; 62.5 | | 0.0; 87.5 | |
| Week 52 | | | | | | |
| Number | 93 | 84 | 23 | 19 | 116 | 103 |
| Mean (SD) | 11.42 (17.11) | −5.95 (18.52) | 11.41 (12.73) | −9.87 (15.49) | 11.42 (16.28) | −6.67 (17.99) |
| Median | 6.25 | 0.00 | 6.25 | −12.50 | 6.25 | −6.25 |
| Q1; Q3 | 0.00; 12.50 | −18.75; 0.00 | 0.00; 18.75 | −18.75; 0.00 | 0.00; 15.63 | −18.75; 0.00 |
| Min; Max | 0.0; 75.0 | −50.0; 75.0 | 0.0; 43.8 | −37.5; 25.0 | 0.0; 75.0 | −50.0; 75.0 |
| Dealing with | | | | | | |
| Hemophilia | | | | | | |
| Baseline | | | | | | |
| Number | 104 | | 23 | | 127 | |
| Mean (SD) | 15.79 (17.22) | | 17.75 (15.35) | | 16.14 (16.86) | |
| Median | 8.33 | | 16.67 | | 8.33 | |
| Q1; Q3 | 0.00; 25.00 | | 8.33; 33.33 | | 0.00; 25.00 | |
| Min; Max | 0.0; 83.3 | | 0.0; 50.0 | | 0.0; 83.3 | |
| Week 52 | | | | | | |
| Number | 104 | 98 | 25 | 22 | 129 | 120 |
| Mean (SD) | 16.03 (24.90) | −0.43 (23.09) | 16.67 (21.11) | −4.17 (16.62) | 16.15 (24.13) | −1.11 (22.03) |
| Median | 0.00 | 0.00 | 8.33 | 0.00 | 0.00 | 0.00 |
| Q1; Q3 | 0.00; 25.00 | −8.33; 0.00 | 0.00; 25.00 | −8.33; 0.00 | 0.00; 25.00 | −8.33; 0.00 |
| Min; Max | 0.0; 100.0 | −41.7; 100.0 | 0.0; 66.7 | −41.7; 41.7 | 0.0; 100.0 | −41.7; 100.0 |
| Treatment | | | | | | |
| Baseline | | | | | | |
| Number | 104 | | 23 | | 127 | |
| Mean (SD) | 32.72 (16.90) | | 40.35 (17.11) | | 34.10 (17.13) | |
| Median | 31.25 | | 37.50 | | 31.25 | |
| Q1; Q3 | 18.75; 46.88 | | 31.25; 59.38 | | 21.88; 46.88 | |
| Min; Max | 0.0; 68.8 | | 9.4; 68.8 | | 0.0; 68.8 | |
| Week 52 | | | | | | |
| Number | 104 | 98 | 25 | 22 | 129 | 120 |
| Mean (SD) | 26.26 (16.71) | −5.99 (15.10) | 26.25 (13.32) | −13.49 (19.87) | 26.26 (16.06) | −7.37 (16.25) |
| Median | 25.00 | −3.13 | 25.00 | −10.94 | 25.00 | −3.13 |
| Q1; Q3 | 14.06; 34.38 | −15.63; 6.25 | 18.75; 34.38 | −25.00; 0.00 | 15.63; 34.38 | −15.63; 3.13 |
| Min; Max | 0.0; 75.0 | −50.0; 25.0 | 0.0; 59.4 | −50.0; 28.1 | 0.0; 75.0 | −50.0; 28.1 |
| Future | | | | | | |
| Baseline | | | | | | |
| Number | 104 | | 23 | | 127 | |
| Mean (SD) | 38.70 (25.82) | | 41.09 (20.56) | | 39.13 (24.89) | |
| Median | 40.00 | | 40.00 | | 40.00 | |
| Q1; Q3 | 15.00; 55.00 | | 25.00; 50.00 | | 20.00; 55.00 | |
| Min; Max | 0.0; 100.0 | | 5.0; 85.0 | | 0.0; 100.0 | |
| Week 52 | | | | | | |
| Number | 104 | 98 | 25 | 22 | 129 | 120 |
| Mean (SD) | 36.59 (26.54) | −1.43 (16.99) | 27.00 (17.91) | −11.59 (19.23) | 34.73 (25.33) | −3.29 (17.78) |
| Median | 35.00 | 0.00 | 25.00 | −10.00 | 30.00 | −5.00 |
| Q1; Q3 | 10.00; 55.00 | −10.00; 5.00 | 15.00; 35.00 | −25.00; 5.00 | 10.00; 55.00 | −15.00; 5.00 |
| Min; Max | 0.0; 100.0 | −50.0; 65.0 | 0.0; 75.0 | −65.0; 15.0 | 0.0; 100.0 | −65.0; 65.0 |
| Family | | | | | | |
| Planning | | | | | | |
| Baseline | | | | | | |
| Number | 73 | | 16 | | 89 | |
| Mean (SD) | 22.57 (30.13) | | 12.63 (12.25) | | 20.79 (27.98) | |
| Median | 8.33 | | 12.50 | | 12.50 | |
| Q1; Q3 | 0.00; 37.50 | | 0.00; 21.88 | | 0.00; 31.25 | |
| Min; Max | 0.0; 100.0 | | 0.0; 33.3 | | 0.0; 100.0 | |
| Week 52 | | | | | | |
| Number | 71 | 59 | 18 | 15 | 89 | 74 |
| Mean (SD) | 24.35 (32.12) | −0.71 (15.97) | 4.98 (7.63) | −9.17 (10.88) | 20.44 (29.89) | −2.42 (15.40) |
| Median | 12.50 | 0.00 | 0.00 | −6.25 | 6.25 | 0.00 |
| Q1; Q3 | 0.00; 41.67 | 0.00; 6.25 | 0.00; 8.33 | −18.75; 0.00 | 0.00; 25.00 | −8.33; 2.08 |
| Min; Max | 0.0; 100.0 | −62.5; 50.0 | 0.0; 25.0 | −33.3; 0.0 | 0.0; 100.0 | −62.5; 50.0 |
| Partnership | | | | | | |
| and Sexuality | | | | | | |
| Baseline | | | | | | |
| Number | 104 | | 23 | | 127 | |
| Mean (SD) | 16.43 (27.73) | | 18.12 (24.32) | | 16.73 (27.06) | |
| Median | 0.00 | | 8.33 | | 0.00 | |
| Q1; Q3 | 0.00; 25.00 | | 0.00; 25.00 | | 0.00; 25.00 | |
| Min; Max | 0.0; 100.0 | | 0.0; 83.3 | | 0.0; 100.0 | |
| Week 52 | | | | | | |
| Number | 104 | 98 | 25 | 22 | 129 | 120 |
| Mean (SD) | 13.06 (24.10) | −3.23 (13.73) | 10.67 (25.63) | −7.20 (12.14) | 12.60 (24.32) | −3.96 (13.49) |
| Median | 0.00 | 0.00 | 0.00 | 0.00 | 0.00 | 0.00 |
| Q1; Q3 | 0.00; 16.67 | −8.33; 0.00 | 0.00; 0.00 | −16.67; 0.00 | 0.00; 16.67 | −8.33; 0.00 |
| Min; Max | 0.0; 100.0 | −50.0; 33.3 | 0.0; 100.0 | −33.3; 16.7 | 0.0; 100.0 | −50.0; 33.3 |

TABLE 31
Summary of Haemo-QoL total scores, domain scores, and change from
baseline to Week 52 (13-16 years old) - Full Analysis Set .

| | Arm A | | | |
|---|---|---|---|---|
| | Prophylaxis | | | |
| Domain | (N = 18) | | | |
| Scores | Actual result | | Change from baseline | |
| Total Score | | | | |
| Baseline | | | | |
| Number | 18 | | | |
| Mean (SD) | 17.77 | (11.43) | | |
| Median | 14.45 | | | |
| Q1; Q3 | 11.36; 16.88 | | | |
| Min; Max | 4.9; 51.0 | | | |
| Week 52 | | | | |
| Number | 18 | | 18 | |
| Mean (SD) | 14.32 | (8.86) | −3.45 | (8.83) |
| Median | 12.18 | | −3.25 | |
| Q1; Q3 | 8.77; 16.88 | | −9.74; 2.27 | |
| Min; Max | 1.6; 36.4 | | −19.5; 11.4 | |
| Physical Health | | | | |
| Baseline | | | | |
| Number | 18 | | | |
| Mean (SD) | 10.71 | (18.09) | | |
| Median | 3.57 | | | |
| Q1; Q3 | 0.00; 10.71 | | | |
| Min; Max | 0.0; 67.9 | | | |
| Week 52 | | | | |
| Number | 18 | | 18 | |
| Mean (SD) | 8.53 | (10.83) | −2.18 | (22.05) |
| Median | 3.57 | | 0.00 | |
| Q1; Q3 | 0.00; 17.86 | | −7.14; 10.71 | |
| Min; Max | 0.0; 35.7 | | −64.3; 25.0 | |
| Feeling | | | | |
| Baseline | | | | |
| Number | 18 | | | |
| Mean (SD) | 7.81 | (18.39) | | |
| Median | 0.00 | | | |
| Q1; Q3 | 0.00; 6.25 | | | |
| Min; Max | 0.0; 75.0 | | | |
| Week 52 | | | | |
| Number | 18 | | 18 | |
| Mean (SD) | 4.69 | (12.09) | −3.13 | (9.77) |
| Median | 0.00 | | 0.00 | |
| Q1; Q3 | 0.00; 3.13 | | −6.25; 0.00 | |
| Min; Max | 0.0; 50.0 | | −25.0; 15.6 | |
| View of Yourself | | | | |
| Baseline | | | | |
| Number | 18 | | | |
| Mean (SD) | 14.17 | (17.19) | | |
| Median | 7.50 | | | |
| Q1; Q3 | 2.50; 20.00 | | | |
| Min; Max | 0.0; 62.5 | | | |
| Week 52 | | | | |
| Number | 18 | | 18 | |
| Mean (SD) | 12.22 | (16.29) | −1.94 | (11.33) |
| Median | 3.75 | | 0.00 | |
| Q1; Q3 | 0.00; 20.00 | | −10.00; 2.50 | |
| Min; Max | 0.0; 57.5 | | −20.0; 22.5 | |
| Family | | | | |
| Baseline | | | | |
| Number | 18 | | | |
| Mean (SD) | 13.72 | (12.60) | | |
| Median | 12.50 | | | |
| Q1; Q3 | 0.00; 21.88 | | | |
| Min; Max | 0.0; 40.6 | | | |
| Week 52 | | | | |
| Number | 18 | | 18 | |
| Mean (SD) | 11.46 | (11.34) | −2.26 | (12.12) |
| Median | 9.38 | | −1.56 | |
| Q1; Q3 | 3.13; 18.75 | | −9.38; 3.13 | |
| Min; Max | 0.0; 37.5 | | −28.1; 25.0 | |
| Friends | | | | |
| Baseline | | | | |
| Number | 18 | | | |
| Mean (SD) | 49.65 | (33.31) | | |
| Median | 46.88 | | | |
| Q1; Q3 | 25.00; 75.00 | | | |
| Min; Max | 6.3; 100.0 | | | |
| Week 52 | | | | |
| Number | 18 | | 18 | |
| Mean (SD) | 40.63 | (33.57) | −9.03 | (38.12) |
| Median | 37.50 | | −3.13 | |
| Q1; Q3 | 25.00; 50.00 | | −25.00; 18.75 | |
| Min; Max | 0.0; 100.0 | | 100.0; 50.0 | |
| Support You Felt | | | | |
| You Were Receiving | | | | |
| Baseline | | | | |
| Number | 18 | | | |
| Mean (SD) | 50.69 | (28.19) | | |
| Median | 46.88 | | | |
| Q1; Q3 | 25.00; 75.00 | | | |
| Min; Max | 0.0; 100.0 | | | |
| Week 52 | | | | |
| Number | 18 | | 18 | |
| Mean (SD) | 38.89 | (27.67) | −11.81 | (24.99) |
| Median | 28.13 | | −3.13 | |
| Q1; Q3 | 25.00; 50.00 | | −25.00; 6.25 | |
| Min; Max | 0.0; 100.0 | | −81.3; 25.0 | |
| Other People | | | | |
| Baseline | | | | |
| Number | 18 | | | |
| Mean (SD) | 5.79 | (14.16) | | |
| Median | 0.00 | | | |
| Q1; Q3 | 0.00; 4.17 | | | |
| Min; Max | 0.0; 58.3 | | | |
| Week 52 | | | | |
| Number | 18 | | 18 | |
| Mean (SD) | 4.40 | (9.64) | −1.39 | (8.93) |
| Median | 0.00 | | 0.00 | |
| Q1; Q3 | 0.00; 4.17 | | 0.00; 0.00 | |
| Min; Max | 0.0; 37.5 | | −20.8; 12.5 | |
| Sports and School | | | | |
| Baseline | | | | |
| Number | 18 | | | |
| Mean (SD) | 15.90 | (14.22) | | |
| Median | 13.89 | | | |
| Q1; Q3 | 2.78; 25.00 | | | |
| Min; Max | 0.0; 50.0 | | | |
| Week 52 | | | | |
| Number | 18 | | 18 | |
| Mean (SD) | 6.79 | (9.68) | −9.10 | (13.30) |
| Median | 2.78 | | −4.17 | |
| Q1; Q3 | 0.00; 11.11 | | −11.11; 0.00 | |
| Min; Max | 0.0; 33.3 | | −50.0; 2.8 | |
| Dealing with | | | | |
| Hemophilia | | | | |
| Baseline | | | | |
| Number | 18 | | | |
| Mean (SD) | 22.62 | (23.11) | | |
| Median | 16.07 | | | |
| Q1; Q3 | 10.71; 28.57 | | | |
| Min; Max | 0.0; 100.0 | | | |
| Week 52 | | | | |
| Number | 18 | | 18 | |
| Mean (SD) | 24.01 | (25.75) | 1.39 | (15.84) |
| Median | 19.64 | | 1.79 | |
| Q1; Q3 | 0.00; 32.14 | | −10.71; 7.14 | |
| Min; Max | 0.0; 100.0 | | −28.6; 32.1 | |
| Treatment | | | | |
| Baseline | | | | |
| Number | 18 | | | |
| Mean (SD) | 16.32 | (11.06) | | |
| Median | 15.63 | | | |
| Q1; Q3 | 12.50; 25.00 | | | |
| Min; Max | 0.0; 34.4 | | | |
| Week 52 | | | | |
| Number | 18 | | 18 | |
| Mean (SD) | 12.67 | (13.45) | −3.65 | (11.80) |
| Median | 7.81 | | −1.56 | |
| Q1; Q3 | 3.13; 18.75 | | −9.38; 3.13 | |
| Min; Max | 0.0; 50.0 | | −28.1; 15.6 | |
| Future | | | | |
| Baseline | | | | |
| Number | 18 | | | |
| Mean (SD) | 26.04 | (15.04) | | |
| Median | 25.00 | | | |
| Q1; Q3 | 18.75; 37.50 | | | |
| Min; Max | 0.0; 50.0 | | | |
| Week 52 | | | | |
| Number | 18 | | 18 | |
| Mean (SD) | 29.17 | (20.89) | 3.13 | (20.37) |
| Median | 28.13 | | 0.00 | |
| Q1; Q3 | 12.50; 50.00 | | −6.25; 6.25 | |
| Min; Max | 0.0; 62.5 | | −25.0; 56.3 | |
| Romantic Relationships | | | | |
| Baseline | | | | |
| Number | 18 | | | |
| Mean (SD) | 3.47 | (10.33) | | |
| Median | 0.00 | | | |
| Q1; Q3 | 0.00; 0.00 | | | |
| Min; Max | 0.0; 37.5 | | | |
| Week 52 | | | | |
| Number | 18 | | 18 | |
| Mean (SD) | 0.00 | (0.00) | −3.47 | (10.33) |

| | | |
|---|---|---|
| Median | 0.00 | 0.00 |
| Q1; Q3 | 0.00; 0.00 | 0.00; 0.00 |
| Min; Max | 0.0; 0.0 | −37.5; 0.0 |

TABLE 32
Summary of Haemo-QoL total scores, domain scores, and change
from baseline to Week 52 (12 years old) - Full Analysis Set

| | Arm A | | |
|---|---|---|---|
| | Prophylaxis | | |
| Domain | (N = 5) | | |
| Scores | | Actual result | Change from baseline |

| Total Score | | | | |
|---|---|---|---|---|
| Baseline | | | | |
| Number | 4 | | | |
| Mean (SD) | 17.39 | (10.38) | | |
| Median | 18.77 | | | |
| Q1; Q3 | 8.61; 26.17 | | | |
| Min; Max | 5.9; 26.2 | | | |
| Week 52 | | | | |
| Number | 5 | | 4 | |
| Mean (SD) | 13.13 | (5.77) | −3.62 | (10.03) |
| Median | 10.55 | | −3.32 | |
| Q1; Q3 | 10.55; 19.14 | | −11.33; 4.09 | |
| Min; Max | 6.3; 19.2 | | −15.6; 7.8 | |
| Physical Health | | | | |
| Baseline | | | | |
| Number | 4 | | | |
| Mean (SD) | 8.04 | (9.39) | | |
| Median | 5.36 | | | |
| Q1; Q3 | 1.79; 14.29 | | | |
| Min; Max | 0.0; 21.4 | | | |
| Week 52 | | | | |
| Number | 5 | | 4 | |
| Mean (SD) | 3.57 | (7.99) | −8.04 | (9.39) |
| Median | 0.00 | | −5.36 | |
| Q1; Q3 | 0.00; 0.00 | | −14.29; −1.79 | |
| Min; Max | 0.0; 17.9 | | −21.4; 0.0 | |
| Feeling | | | | |
| Baseline | | | | |
| Number | 4 | | | |
| Mean (SD) | 7.14 | (8.25) | | |
| Median | 7.14 | | | |
| Q1; Q3 | 0.00; 14.29 | | | |
| Min; Max | 0.0; 14.3 | | | |
| Week 52 | | | | |
| Number | 5 | | 4 | |
| Mean (SD) | 2.86 | (3.91) | −5.36 | (6.84) |
| Median | 0.00 | | −3.57 | |
| Q1; Q3 | 0.00; 7.14 | | −10.71; 0.00 | |
| Min; Max | 0.0; 7.1 | | −14.3; 0.0 | |
| View of Yourself | | | | |
| Baseline | | | | |
| Number | 4 | | | |
| Mean (SD) | 15.21 | (17.12) | | |
| Median | 12.36 | | | |
| Q1; Q3 | 1.25; 29.17 | | | |
| Min; Max | 0.0; 36.1 | | | |
| Week 52 | | | | |
| Number | 5 | | 4 | |
| Mean (SD) | 6.67 | (9.13) | −8.26 | (24.55) |
| Median | 5.56 | | −9.58 | |
| Q1; Q3 | 0.00; 5.56 | | −26.39; 9.86 | |
| Min; Max | 0.0; 22.2 | | −36.1; 22.2 | |
| Family | | | | |
| Baseline | | | | |
| Number | 4 | | | |
| Mean (SD) | 18.28 | (22.90) | | |
| Median | 11.56 | | | |
| Q1; Q3 | 1.56; 35.00 | | | |
| Min; Max | 0.0; 50.0 | | | |
| Week 52 | | | | |
| Number | 5 | | 4 | |
| Mean (SD) | 11.00 | (13.42) | −9.53 | (9.51) |
| Median | 5.00 | | −9.06 | |
| Q1; Q3 | 0.00; 20.00 | | −17.50; −1.56 | |
| Min; Max | 0.0; 30.0 | | −20.0; 0.0 | |
| Friends | | | | |
| Baseline | | | | |
| Number | 4 | | | |
| Mean (SD) | 50.00 | (19.76) | | |
| Median | 46.88 | | | |
| Q1; Q3 | 34.38; 65.63 | | | |
| Min; Max | 31.3; 75.0 | | | |
| Week 52 | | | | |
| Number | 5 | | 4 | |
| Mean (SD) | 31.25 | (25.39) | −12.50 | (30.62) |
| Median | 25.00 | | −6.25 | |
| Q1; Q3 | 12.50; 43.75 | | 37.50; 12.50 | |
| Min; Max | 6.3; 68.8 | | −50.0; 12.5 | |
| Support You Felt | | | | |
| You Were Receiving | | | | |
| Baseline | | | | |
| Number | 4 | | | |
| Mean (SD) | 48.44 | (15.63) | | |
| Median | 46.88 | | | |
| Q1; Q3 | 37.50; 59.38 | | | |
| Min; Max | 31.3; 68.8 | | | |
| Week 52 | | | | |
| Number | 5 | | 4 | |
| Mean (SD) | 47.50 | (25.23) | 6.25 | (13.50) |
| Median | 50.00 | | 3.13 | |
| Q1; Q3 | 25.00; 68.75 | | −3.13; 15.63 | |
| Min; Max | 18.8; 75.0 | | −6.3; 25.0 | |
| Other People | | | | |
| Baseline | | | | |
| Number | 4 | | | |
| Mean (SD) | 5.21 | (7.89) | | |
| Median | 2.08 | | | |
| Q1; Q3 | 0.00; 10.42 | | | |
| Min; Max | 0.0; 16.7 | | | |
| Week 52 | | | | |
| Number | 5 | | 4 | |
| Mean (SD) | 0.83 | (1.86) | −5.21 | (7.89) |
| Median | 0.00 | | −2.08 | |
| Q1; Q3 | 0.00; 0.00 | | −10.42; 0.00 | |
| Min; Max | 0.0; 4.2 | | −16.7; 0.0 | |
| Sports and School | | | | |
| Baseline | | | | |
| Number | 4 | | | |
| Mean (SD) | 4.51 | (3.03) | | |
| Median | 5.90 | | | |
| Q1; Q3 | 2.78; 6.25 | | | |
| Min; Max | 0.0; 6.3 | | | |
| Week 52 | | | | |
| Number | 5 | | 4 | |
| Mean (SD) | 6.67 | (14.91) | 3.82 | (16.24) |
| Median | 0.00 | | −3.13 | |
| Q1; Q3 | 0.00; 0.00 | | −6.25; 13.89 | |
| Min; Max | 0.0; 33.3 | | −6.3; 27.8 | |
| Dealing with | | | | |
| Hemophilia | | | | |
| Baseline | | | | |
| Number | 4 | | | |
| Mean (SD) | 26.79 | (23.60) | | |
| Median | 28.57 | | | |
| Q1; Q3 | 7.14; 46.43 | | | |
| Min; Max | 0.0; 50.0 | | | |
| Week 52 | | | | |
| Number | 5 | | 4 | |
| Mean (SD) | 40.00 | (35.12) | 15.18 | (56.72) |
| Median | 32.14 | | −10.71 | |
| Q1; Q3 | 28.57; 32.14 | | −16.07; 46.43 | |
| Min; Max | 7.1; 100.0 | | −17.9; 100.0 | |
| Treatment | | | | |
| Baseline | | | | |
| Number | 4 | | | |
| Mean (SD) | 19.20 | (16.06) | | |
| Median | 20.54 | | | |
| Q1; Q3 | 6.25; 32.14 | | | |
| Min; Max | 0.0; 35.7 | | | |
| Week 52 | | | | |
| Number | 5 | | 4 | |
| Mean (SD) | 9.73 | (13.21) | −7.03 | (9.73) |

| | | |
|---|---|---|
| Median | 7.14 | −3.35 |
| Q1; Q3 | 0.00; 9.38 | −12.50; −1.56 |
| Min; Max | 0.0; 32.1 | −21.4; 0.0 |

Prophylactic treatment with efanesoctocog alfa resulted in an improvement in physical function. Physical function was measured according to the PROMIS-SF v2.0 physical function (PF) 6b at baseline and week 52. For PROMIS-SF PF 6b in Arm A the least square (LS) mean (standard error; SE), and 95% confidence interval (CI) were estimated by mixed-effect model with repeated measures (MMRM) with visit as fixed effect, and baseline score as covariate. Numerical improvement was observed in PROMIS PF 6b score (LS mean [Standard error] change from baseline: 0.56 [0.47]; N=108). The proportion of patients that were unable to go up and down the stairs at normal pace decreased from 12% (12/103) at baseline to 6% (6/102) at Week 52.

### Hemophilia Activities List (HAL) Score

Physical function was also measured using Hemophilia Activities List (HAL) in Arm A. For HAL overall score in Arm A the least square (LS) mean (standard error; SE), and 95% confidence interval (CI) were estimated by mixed-effect model with repeated measures (MMRM) with visit as fixed effect, and baseline score as covariate. At baseline, the overall HAL score mean [SD] was 76.11 [20.73]. Numerical improvement was observed in in total HAL score at Week 52 (LS mean change: 0.67 [95% CI: −2.03 to 3.38]; P=0.6229, N=96)). A summary of the overall HAL score, domain score, component score, and change from baseline to Week 52 is provided in Table 33 for participants 218 years old. A summary of the overall pediatric HAL (pedHAL) score, domain scores, and change from baseline to Week 52 is shown in Table 34 for participants <18 years old. No participants from Arm B met the age requirements for the pedHAL.

TABLE 33
Summary of Hemophilia Activities List (HAL) overall scores, domain scores, component
scores, and change from baseline to Week 52 (≥18 years old) - Full Analysis Set

| | Arm A | | Arm B | | | |
|---|---|---|---|---|---|---|
| | Prophylaxis | | On-demand −> Prophylaxis | | Overall | |
| | (N = 108) | | (N = 26) | | (N = 134) | |
| Score | Actual | Change from | Actual | Change from | Actual | Change from |
| Visit | result | baseline | result | baseline | result | baseline |
| Overall Score | | | | | | |
| Baseline | | | | | | |
| Number | 103 | | 23 | | 126 | |
| Mean (SD) | 76.11 (20.73) | | 72.50 (18.39) | | 75.45 (20.30) | |
| Median | 80.95 | | 73.68 | | 79.01 | |
| Q1; Q3 | 60.00; 95.24 | | 59.52; 91.28 | | 60.00; 94.29 | |
| Min; Max | 19.0; 99.5 | | 32.0; 97.1 | | 19.0; 99.5 | |
| Week 52 | | | | | | |
| Number | 101 | 96 | 25 | 22 | 126 | 118 |
| Mean (SD) | 76.22 (22.05) | 0.46 (14.04) | 82.26 (13.89) | 9.13 (9.83) | 77.42 (20.78) | 2.08 (13.75) |
| Median | 80.63 | 0.51 | 83.53 | 7.33 | 82.18 | 1.05 |
| Q1; Q3 | 61.05; 95.38 | −2.60; 6.49 | 77.14; 93.33 | 0.94; 15.84 | 64.39; 94.59 | −1.66; 8.31 |
| Min; Max | 0.0; 99.5 | −99.5; 27.0 | 45.5; 99.5 | −3.6; 32.0 | 0.0; 99.5 | −99.5; 32.0 |
| Lying down/ | | | | | | |
| Sitting/Kneeling/ | | | | | | |
| Standing | | | | | | |
| Baseline | | | | | | |
| Number | 103 | | 23 | | 126 | |
| Mean (SD) | 69.85 (25.41) | | 66.30 (22.66) | | 69.21 (24.89) | |
| Median | 72.50 | | 67.50 | | 67.50 | |
| Q1; Q3 | 50.00; 95.00 | | 47.50; 90.00 | | 50.00; 95.00 | |
| Min; Max | 15.0; 100.0 | | 20.0; 100.0 | | 15.0; 100.0 | |
| Week 52 | | | | | | |
| Number | 101 | 96 | 25 | 22 | 126 | 118 |
| Mean (SD) | 70.02 (25.06) | 1.04 (14.86) | 71.40 (17.90) | 4.55 (12.41) | 70.30 (23.76) | 1.69 (14.45) |
| Median | 70.00 | 0.00 | 75.00 | 5.00 | 72.50 | 0.00 |
| Q1; Q3 | 50.00; 95.00 | −2.50; 5.00 | 55.00; 85.00 | −5.00; 12.50 | 50.00; 92.50 | −2.50; 7.50 |
| Min; Max | 0.0; 100.0 | −100.0; 32.5 | 40.0; 100.0 | −17.5; 32.5 | 0.0; 100.0 | −100.0; 32.5 |
| Function | | | | | | |
| of the Legs | | | | | | |
| Baseline | | | | | | |
| Number | 103 | | 23 | | 126 | |
| Mean (SD) | 66.84 (27.50) | | 63.48 (24.94) | | 66.23 (26.99) | |
| Median | 66.67 | | 64.44 | | 65.56 | |
| Q1; Q3 | 48.89; 93.33 | | 42.22; 86.67 | | 46.67; 93.33 | |
| Min; Max | 8.9; 100.0 | | 20.0; 100.0 | | 8.9; 100.0 | |
| Week 52 | | | | | | |
| Number | 101 | 96 | 25 | 22 | 126 | 118 |
| Mean (SD) | 67.11 (28.75) | 1.06 (16.16) | 76.98 (20.27) | 13.54 (14.82) | 69.07 (27.49) | 3.39 (16.59) |
| Median | 68.89 | 0.00 | 82.22 | 11.11 | 71.11 | 1.11 |
| Q1; Q3 | 42.22; 97.78 | −2.22; 8.89 | 68.89; 93.33 | 0.00; 24.44 | 48.89; 97.78 | −2.22; 11.11 |
| Min; Max | 0.0; 100.0 | −100.0; 37.8 | 15.6; 100.0 | −8.9; 48.9 | 0.0; 100.0 | −100.0; 48.9 |
| Functions | | | | | | |
| of the Arms | | | | | | |
| Baseline | | | | | | |
| Number | 103 | | 23 | | 126 | |
| Mean (SD) | 75.24 (20.30) | | 71.96 (20.71) | | 74.64 (20.33) | |
| Median | 80.00 | | 80.00 | | 80.00 | |
| Q1; Q3 | 60.00; 95.00 | | 65.00; 85.00 | | 65.00; 95.00 | |
| Min; Max | 15.0; 95.0 | | 25.0; 95.0 | | 15.0; 95.0 | |
| Week 52 | | | | | | |
| Number | 101 | 96 | 25 | 22 | 126 | 118 |
| Mean (SD) | 75.54 (22.27) | −0.21 (16.02) | 78.60 (20.59) | 3.86 (18.70) | 76.15 (21.90) | 0.55 (16.55) |
| Median | 85.00 | 0.00 | 85.00 | 2.50 | 85.00 | 0.00 |
| Q1; Q3 | 65.00; 95.00 | −5.00; 5.00 | 75.00; 95.00 | 0.00; 15.00 | 65.00; 95.00 | −5.00; 10.00 |
| Min; Max | 0.0; 95.0 | −95.0; 35.0 | 30.0; 95.0 | −55.0; 35.0 | 0.0; 95.0 | −95.0; 35.0 |
| Use of | | | | | | |
| Transportation | | | | | | |
| Baseline | | | | | | |
| Number | 90 | | 20 | | 110 | |
| Mean (SD) | 84.44 (22.96) | | 70.00 (27.57) | | 81.82 (24.38) | |
| Median | 96.67 | | 73.33 | | 93.33 | |
| Q1; Q3 | 80.00; 100.00 | | 46.67; 96.67 | | 73.33; 100.00 | |
| Min; Max | 10.0; 100.0 | | 20.0; 100.0 | | 10.0; 100.0 | |
| Week 52 | | | | | | |
| Number | 87 | 76 | 22 | 17 | 109 | 93 |
| Mean (SD) | 84.10 (23.29) | 1.18 (18.33) | 79.24 (26.67) | 8.04 (11.96) | 83.12 (23.96) | 2.44 (17.49) |
| Median | 100.00 | 0.00 | 93.33 | 6.67 | 93.33 | 0.00 |
| Q1; Q3 | 80.00; 00.00 | 0.00; 5.00 | 60.00; 100.00 | 0.00; 20.00 | 73.33; 100.00 | 0.00; 6.67 |
| Min; Max | 0.0; 100.0 | −100.0; 66.7 | 10.0; 100.0 | −10.0; 33.3 | 0.0; 100.0 | −100.0; 66.7 |
| Self Care | | | | | | |
| Baseline | | | | | | |
| Number | 103 | | 23 | | 126 | |
| Mean (SD) | 89.13 (19.25) | | 93.04 (11.52) | | 89.84 (18.11) | |
| Median | 100.00 | | 100.00 | | 100.00 | |
| Q1; Q3 | 84.00; 100.00 | | 92.00; 100.00 | | 84.00; 100.00 | |
| Min; Max | 20.0; 100.0 | | 60.0; 100.0 | | 20.0; 100.0 | |
| Week 52 | | | | | | |
| Number | 101 | 96 | 25 | 22 | 126 | 118 |
| Mean (SD) | 88.67 (19.67) | −0.33 (17.47) | 95.04 (10.15) | 2.00 (9.86) | 89.94 (18.32) | 0.10 (16.31) |
| Median | 100.00 | 0.00 | 100.00 | 0.00 | 100.00 | 0.00 |
| Q1; Q3 | 84.00; 100.00 | 0.00; 0.00 | 96.00; 100.00 | 0.00; 4.00 | 88.00; 100.00 | 0.00; 4.00 |
| Min; Max | 0.0; 100.0 | −100.0; 80.0 | 64.0; 100.0 | −20.0; 32.0 | 0.0; 100.0 | −100.0; 80.0 |
| Household | | | | | | |
| Tasks | | | | | | |
| Baseline | | | | | | |
| Number | 103 | | 23 | | 126 | |
| Mean (SD) | 85.71 (19.64) | | 79.19 (23.28) | | 84.52 (20.41) | |
| Median | 93.33 | | 90.00 | | 93.33 | |
| Q1; Q3 | 76.67; 100.00 | | 50.00; 100.00 | | 76.67; 100.00 | |
| Min; Max | 20.0; 100.0 | | 33.3; 100.0 | | 20.0; 100.0 | |
| Week 52 | | | | | | |
| Number | 100 | 95 | 25 | 22 | 125 | 117 |
| Mean (SD) | 84.43 (22.30) | −1.17 (17.38) | 92.85 (13.78) | 11.36 (18.26) | 86.12 (21.10) | 1.19 (18.15) |
| Median | 96.67 | 0.00 | 100.00 | 3.33 | 100.00 | 0.00 |
| Q1; Q3 | 75.00; 100.00 | 0.00; 3.33 | 93.33; 100.00 | 0.00; 20.00 | 80.00; 100.00 | 0.00; 3.33 |
| Min; Max | 0.0; 100.0 | −100.0; 50.0 | 53.3; 100.0 | −15.0; 53.3 | 0.0; 100.0 | −100.0; 53.3 |
| Leisure | | | | | | |
| Activities | | | | | | |
| and Sports | | | | | | |
| Baseline | | | | | | |
| Number | 80 | | 19 | | 99 | |
| Mean (SD) | 74.43 (24.23) | | 70.77 (23.42) | | 73.73 (24.00) | |
| Median | 80.00 | | 68.57 | | 80.00 | |
| Q1; Q3 | 57.14; 96.57 | | 62.86; 94.29 | | 57.14; 95.00 | |
| Min; Max | 17.1; 100.0 | | 16.0; 100.0 | | 16.0; 100.0 | |
| Week 52 | | | | | | |
| Number | 85 | 69 | 21 | 16 | 106 | 85 |
| Mean (SD) | 78.98 (24.68) | 0.97 (20.15) | 89.27 (11.88) | 16.71 (16.95) | 81.02 (23.04) | 3.94 (20.45) |
| Median | 88.57 | 0.00 | 94.29 | 9.76 | 91.43 | 2.86 |
| Q1; Q3 | 65.00; 100.00 | −5.00; 8.57 | 82.86; 97.14 | 3.71; 27.14 | 73.33; 100.00 | 0.00; 10.95 |
| Min; Max | 0.0; 100.0 | −100.0; 56.0 | 62.9; 100.0 | 0.0; 61.1 | 0.0; 100.0 | −100.0; 61.1 |
| Upper Extremity | | | | | | |
| Activities | | | | | | |
| Baseline | | | | | | |
| Number | 103 | | 23 | | 126 | |
| Mean (SD) | 82.96 (18.12) | | 83.67 (14.36) | | 83.09 (17.44) | |
| Median | 88.89 | | 88.89 | | 88.89 | |
| Q1; Q3 | 75.56; 97.78 | | 75.56; 93.33 | | 75.56; 97.78 | |
| Min; Max | 17.8; 97.8 | | 44.4; 97.8 | | 17.8; 97.8 | |
| Week 52 | | | | | | |
| Number | 101 | 96 | 25 | 22 | 126 | 118 |
| Mean (SD) | 82.84 (19.77) | −0.28 (14.75) | 87.73 (12.26) | 2.83 (12.12) | 83.81 (18.58) | 0.30 (14.30) |
| Median | 93.33 | 0.00 | 93.33 | 2.22 | 93.33 | 0.00 |
| Q1; Q3 | 75.56; 97.78 | −2.22; 4.44 | 77.78; 97.78 | 0.00; 8.89 | 75.56; 97.78 | −2.22; 4.44 |
| Min; Max | 0.0; 97.8 | −97.8; 48.9 | 57.8; 97.8 | −24.4; 33.3 | 0.0; 97.8 | −97.8; 48.9 |
| Basic Lower | | | | | | |
| Extremity | | | | | | |
| Activities | | | | | | |
| Baseline | | | | | | |
| Number | 103 | | 23 | | 126 | |
| Mean (SD) | 71.68 (26.36) | | 70.43 (22.05) | | 71.46 (25.55) | |
| Median | 80.00 | | 73.33 | | 75.00 | |
| Q1; Q3 | 50.00; 96.67 | | 53.33; 90.00 | | 53.33; 93.33 | |
| Min; Max | 10.0; 100.0 | | 33.3; 100.0 | | 10.0; 100.0 | |
| Week 52 | | | | | | |
| Number | 101 | 96 | 25 | 22 | 126 | 118 |
| Mean (SD) | 71.68 (26.77) | 0.56 (16.34) | 82.40 (20.83) | 11.52 (16.64) | 73.81 (25.98) | 2.60 (16.88) |
| Median | 80.00 | 0.00 | 86.67 | 5.00 | 83.33 | 0.00 |
| Q1; Q3 | 53.33; 96.67 | −5.00; 8.33 | 76.67; 96.67 | 3.33; 20.00 | 56.67; 96.67 | −3.33; 10.00 |
| Min; Max | 0.0; 100.0 | −100.0; 43.3 | 16.7; 100.0 | −16.7; 53.3 | 0.0; 100.0 | −100.0; 53.3 |
| Complex Lower | | | | | | |
| Extremity | | | | | | |
| Activities | | | | | | |
| Baseline | | | | | | |
| Number | 103 | | 23 | | 126 | |
| Mean (SD) | 60.78 (30.66) | | 52.90 (28.70) | | 59.34 (30.35) | |
| Median | 60.00 | | 44.44 | | 54.17 | |
| Q1; Q3 | 37.50; 92.50 | | 33.33; 77.50 | | 35.56; 88.89 | |
| Min; Max | 2.2; 100.0 | | 4.4; 100.0 | | 2.2; 100.0 | |
| Week 52 | | | | | | |
| Number | 101 | 96 | 25 | 22 | 126 | 118 |
| Mean (SD) | 60.57 (31.85) | 1.13 (17.63) | 62.20 (24.92) | 10.03 (11.86) | 60.90 (30.51) | 2.79 (17.02) |
| Median | 62.22 | 0.00 | 66.67 | 12.64 | 62.36 | 1.11 |
| Q1; Q3 | 31.11; 91.11 | −4.44; 8.89 | 42.22; 86.67 | 0.00; 20.00 | 32.50; 88.89 | −4.44; 11.11 |
| Min; Max | 0.0; 100.0 | −100.0; 46.7 | 20.0; 100.0 | −15.0; 32.5 | 0.0; 100.0 | −100.0; 46.7 |

TABLE 34
Summary of Pediatric Hemophilia Activities List (pedHAL) overall
scores, domain scores, component scores, and change from baseline
to Week 52 (<18 years old) - Full Analysis Set

| | Arm A | | |
|---|---|---|---|
| | Prophylaxis | | |
| Score | (N = 25) | | |
| Visit | | Actual result | Change from baseline |

| Overall Score | | |
|---|---|---|
| Baseline | | |
| Number | 20 | |
| Mean (SD) | 97.93 | (2.78) |

| | | |
|---|---|---|
| Median | 99.24 | |
| Q1; Q3 | 96.18; 100.00 | |
| Min; Max | 89.8; 100.0 | |
| Week 52 | | |
| Number | 23 | 19 |

| | | | |
|---|---|---|---|
| Mean (SD) | 99.57 (1.35) | 1.66 | (3.05) |

| Median | 100.00 | | 0.38 | |
|---|---|---|---|---|
| Q1; Q3 | 100.00; 100.00 | | −0.01; 3.91 | |
| Min; Max | 93.9; 100.0 | | −2.4; 10.2 | |
| Sitting/Kneeling/ | | | | |
| Standing | | | | |
| Baseline | | | | |
| Number | 20 | | | |
| Mean (SD) | 96.26 | (5.12) | | |
| Median | 99.00 | | | |
| Q1; Q3 | 93.56; 100.00 | | | |
| Min; Max | 84.0; 100.0 | | | |
| Week 52 | | | | |
| Number | 23 | | 19 | |
| Mean (SD) | 99.30 | (1.87) | 3.10 | (5.98) |
| Median | 100.00 | | 0.00 | |
| Q1; Q3 | 100.00; 100.00 | | 0.00; 8.89 | |
| Min; Max | 92.0; 100.0 | | −8.0; 16.0 | |
| Functions of the | | | | |
| Legs | | | | |
| Baseline | | | | |
| Number | 19 | | | |
| Mean (SD) | 97.80 | (5.86) | | |
| Median | 100.00 | | | |
| Q1; Q3 | 98.18; 100.00 | | | |
| Min; Max | 74.5; 100.0 | | | |
| Week 52 | | | | |
| Number | 23 | | 18 | |
| Mean (SD) | 99.76 | (1.14) | 2.02 | (6.26) |
| Median | 100.00 | | 0.00 | |
| Q1; Q3 | 100.00; 100.00 | | 0.00; 1.82 | |
| Min; Max | 94.5; 100.0 | | 5.5; 25.5 | |
| Functions of the | | | | |
| Arms | | | | |
| Baseline | | | | |
| Number | 20 | | | |
| Mean (SD) | 97.33 | (6.89) | | |
| Median | 100.00 | | | |
| Q1; Q3 | 100.00; 100.00 | | | |
| Min; Max | 76.7; 100.0 | | | |
| Week 52 | | | | |
| Number | 23 | | 19 | |
| Mean (SD) | 99.42 | (2.78) | 2.11 | (5.80) |
| Median | 100.00 | | 0.00 | |
| Q1; Q3 | 100.00; 100.00 | | 0.00; 0.00 | |
| Min; Max | 86.7; 100.0 | | 0.0; 23.3 | |
| Use of | | | | |
| Transportation | | | | |
| Baseline | | | | |
| Number | 19 | | | |
| Mean (SD) | 100.00 | (0.00) | | |
| Median | 100.00 | | | |
| Q1; Q3 | 100.00; 100.00 | | | |
| Min; Max | 100.0; 100.0 | | | |
| Week 52 | | | | |
| Number | 23 | | 18 | |
| Mean (SD) | 99.71 | (1.39) | −0.37 | (1.57) |
| Median | 100.00 | | 0.00 | |
| Q1; Q3 | 100.00; 100.00 | | 0.00; 0.00 | |
| Min; Max | 93.3; 100.0 | | −6.7; 0.0 | |
| Self Care | | | | |
| Baseline | | | | |
| Number | 20 | | | |
| Mean (SD) | 100.00 | (0.00) | | |
| Median | 100.00 | | | |
| Q1; Q3 | 100.00; 100.00 | | | |
| Min; Max | 100.0; 100.0 | | | |
| Week 52 | | | | |
| Number | 23 | | 19 | |
| Mean (SD) | 100.00 | (0.00) | 0.00 | (0.00) |
| Median | 100.00 | | 0.00 | |
| Q1; Q3 | 100.00; 100.00 | | 0.00; 0.00 | |
| Min; Max | 100.0; 100.0 | | 0.0; 0.0 | |
| Household Tasks | | | | |
| Baseline | | | | |
| Number | 20 | | | |
| Mean (SD) | 100.00 | (0.00) | | |
| Median | 100.00 | | | |
| Q1; Q3 | 100.00; 100.00 | | | |
| Min; Max | 100.0; 100.0 | | | |
| Week 52 | | | | |
| Number | 23 | | 19 | |
| Mean (SD) | 100.00 | (0.00) | 0.00 | (0.00) |
| Median | 100.00 | | 0.00 | |
| Q1; Q3 | 100.00; 100.00 | | 0.00; 0.00 | |
| Min; Max | 100.0; 100.0 | | 0.0; 0.0 | |
| Leisure Activities | | | | |
| and Sports | | | | |
| Baseline | | | | |
| Number | 17 | | | |
| Mean (SD) | 96.58 | (5.05) | | |
| Median | 100.00 | | | |
| Q1; Q3 | 94.29; 100.00 | | | |
| Min; Max | 85.5; 100.0 | | | |
| Week 52 | | | | |
| Number | 20 | | 15 | |
| Mean (SD) | 99.43 | (2.07) | 2.23 | (5.18) |

| | | |
|---|---|---|
| Median | 100.00 | 0.00 |
| Q1; Q3 | 100.00; 100.00 | 0.00; 5.71 |
| Min; Max | 90.9; 100.0 | −5.5; 14.5 |

### EuroQol 5-Dimension 5-Level (EQ-5D-5L) Score

General health-related quality of life was assessed as an exploratory endpoint in arms A and B using the EuroQol 5-dimension 5-level (EQ-5D-5L) assessment, consisting of a descriptive system of five dimensions (mobility, usual activities, self-care, pain/discomfort, and anxiety/depression) and a visual analog scale (EQ-VAS; score range 0-100, higher score indicates better health-related quality of life). At baseline, a total of 18% (22/122) and 9% (11/122) patients reported moderate to severe problem with mobility and usual activities, respectively, as per EQ-5D-5L. A total of 23.5% (27/115) patients reported an improvement in at least 1 category in mobility at Week 52, while 67.0% (77/115) reported no change, and 9.6% (11/115) reported a worsening in a mobility category. A total of 19.1% (22/115) patients reported an improvement in at least 1 category in usual activities at Week 52, while 73.0% (84/115) reported no change, and 7.8% (9/115) reported a worsening in a usual activities category. A summary of the EQ-5D-5L descriptive system at baseline and Week 52 is provided in Table 35. A summary of the EQ visual analog scale (VAS) and change from baseline to Week 52 is provided in Table 36. A summary of the EQ-5D Index Score and change from baseline to Week 52 is provided in Table 37. For Tables 35-37, baseline characteristics for Arm B are reported at the beginning of the initial on-demand treatment period.

TABLE 35
Summary of EQ-5D-5L descriptive system at baseline and Week 52 - Full Analysis Set

| | Arm A | | Arm B | | | |
|---|---|---|---|---|---|---|
| Domain | Prophylaxis | | On-demand->Prophylaxis | | Overall | |
| Visit, n (%) | (N = 133) | | (N = 26) | | (N = 159) | |
| Mobility | | | | | | |
| Baseline | | | | | | |
| Number | 122 | | 22 | | 144 | |
| I have no problems walking | 63 | (51.6) | 7 | (31.8) | 70 | (48.6) |
| I have slight problems walking | 37 | (30.3) | 8 | (36.4) | 45 | (31.3) |
| I have moderate problems walking | 15 | (12.3) | 5 | (22.7) | 20 | (13.9) |
| I have severe problems walking | 7 | (5.7) | 2 | (9.1) | 9 | (6.3) |
| I am unable to walk | 0 | | 0 | | 0 | |
| Week 52 | | | | | | |
| Number | 126 | | 25 | | 151 | |
| I have no problems walking | 73 | (57.9) | 11 | (44.0) | 84 | (55.6) |
| I have slight problems walking | 31 | (24.6) | 10 | (40.0) | 41 | (27.2) |
| I have moderate problems walking | 16 | (12.7) | 3 | (12.0) | 19 | (12.6) |
| I have severe problems walking | 6 | (4.8) | 1 | (4.0) | 7 | (4.6) |
| I am unable to walk | 0 | | 0 | | 0 | |
| Self-care | | | | | | |
| Baseline | | | | | | |
| Number | 122 | | 22 | | 144 | |
| I have no problems washing or dressing myself | 107 | (87.7) | 16 | (72.7) | 123 | (85.4) |
| I have slight problems washing or dressing myself | 8 | (6.6) | 5 | (22.7) | 13 | (9.0) |
| I have moderate problems washing or dressing | 7 | (5.7) | 1 | (4.5) | 8 | (5.6) |
| myself | | | | | | |
| I have severe problems washing or dressing myself | 0 | | 0 | | 0 | |
| I am unable to wash or dress myself | 0 | | 0 | | 0 | |
| Week 52 | | | | | | |
| Number | 126 | | 25 | | 151 | |
| I have no problems washing or dressing myself | 108 | (85.7) | 21 | (84.0) | 129 | (85.4) |
| I have slight problems washing or dressing myself | 13 | (10.3) | 3 | (12.0) | 16 | (10.6) |
| I have moderate problems washing or dressing | 4 | (3.2) | 0 | | 4 | (2.6) |
| myself | | | | | | |
| I have severe problems washing or dressing myself | 1 | (0.8) | 1 | (4.0) | 2 | (1.3) |
| I am unable to wash or dress myself | 0 | | 0 | | 0 | |
| Usual activities | | | | | | |
| Baseline | | | | | | |
| Number | 122 | | 22 | | 144 | |
| I have no problems doing my usual activities | 80 | (65.6) | 9 | (40.9) | 89 | (61.8) |
| I have slight problems doing my usual activities | 31 | (25.4) | 8 | (36.4) | 39 | (27.1) |
| I have moderate problems doing my usual | 9 | (7.4) | 4 | (18.2) | 13 | (9.0) |
| activities | | | | | | |
| I have severe problems doing my usual activities | 2 | (1.6) | 1 | (4.5) | 3 | (2.1) |
| I am unable to do my usual activities | 0 | | 0 | | 0 | |
| Week 52 | | | | | | |
| Number | 126 | | 25 | | 151 | |
| I have no problems doing my usual activities | 90 | (71.4) | 20 | (80.0) | 110 | (72.8) |
| I have slight problems doing my usual activities | 25 | (19.8) | 4 | (16.0) | 29 | (19.2) |
| I have moderate problems doing my usual | 10 | (7.9) | 0 | | 10 | (6.6) |
| activities | | | | | | |
| I have severe problems doing my usual activities | 1 | (0.8) | 1 | (4.0) | 2 | (1.3) |
| I am unable to do my usual activities | 0 | | 0 | | 0 | |
| Pain/discomfort | | | | | | |
| Baseline | | | | | | |
| Number | 122 | | 22 | | 144 | |
| I have no pain or discomfort | 51 | (41.8) | 12 | (54.5) | 63 | (43.8) |
| I have slight pain or discomfort | 47 | (38.5) | 6 | (27.3) | 53 | (36.8) |
| I have moderate pain or discomfort | 19 | (15.6) | 3 | (13.6) | 22 | (15.3) |
| I have severe pain or discomfort | 4 | (3.3) | 1 | (4.5) | 5 | (3.5) |
| I have extreme pain or discomfort | 1 | (0.8) | 0 | | 1 | (0.7) |
| Week 52 | | | | | | |
| Number | 126 | | 25 | | 151 | |
| I have no pain or discomfort | 56 | (44.4) | 14 | (56.0) | 70 | (46.4) |
| I have slight pain or discomfort | 49 | (38.9) | 10 | (40.0) | 59 | (39.1) |
| I have moderate pain or discomfort | 17 | (13.5) | 0 | | 17 | (11.3) |
| I have severe pain or discomfort | 4 | (3.2) | 1 | (4.0) | 5 | (3.3) |
| I have extreme pain or discomfort | 0 | | 0 | | 0 | |
| Anxiety/depression | | | | | | |
| Baseline | | | | | | |
| Number | 122 | | 22 | | 144 | |
| I am not anxious or depressed | 81 | (66.4) | 15 | (68.2) | 96 | (66.7) |
| I am slightly anxious or depressed | 32 | (26.2) | 5 | (22.7) | 37 | (25.7) |
| I am moderately anxious or depressed | 9 | (7.4) | 2 | (9.1) | 11 | (7.6) |
| I am severely anxious or depressed | 0 | | 0 | | 0 | |
| I am extremely anxious or depressed | 0 | | 0 | | 0 | |
| Week 52 | | | | | | |
| Number | 126 | | 25 | | 151 | |
| I am not anxious or depressed | 81 | (64.3) | 20 | (80.0) | 101 | (66.9) |
| I am slightly anxious or depressed | 29 | (23.0) | 3 | (12.0) | 32 | (21.2) |
| I am moderately anxious or depressed | 13 | (10.3) | 2 | (8.0) | 15 | (9.9) |

| | | | | | |
|---|---|---|---|---|---|
| I am severely anxious or depressed | 2 | (1.6) | 0 | 2 | (1.3) |
| I am extremely anxious or depressed | 1 | (0.8) | 0 | 1 | (0.7) |

TABLE 36
Summary of EQ Visual Analog Scale (VAS) and change from baseline to Week 52 - Full Analysis Set

| | Arm A | | Arm B | | | |
|---|---|---|---|---|---|---|
| | Prophylaxis | | On-demand −> Prophylaxis | | Overall | |
| | (N = 133) | | (N = 26) | | (N = 159) | |
| | Actual | Change from | Actual | Change from | Actual | Change from |
| Visit | result | baseline | result | baseline | result | baseline |
| Baseline | | | | | | |
| Number | 122 | | 22 | | 144 | |
| Mean (SD) | 81.66 (15.53) | | 72.91 (21.36) | | 80.32 (16.76) | |
| Median | 85.00 | | 75.50 | | 83.50 | |
| Q1; Q3 | 75.00; 94.00 | | 66.00; 90.00 | | 72.50; 91.00 | |
| Min; Max | 26.0; 100.0 | | 9.0; 99.0 | | 9.0; 100.0 | |
| Week 52 | | | | | | |
| Number | 126 | 115 | 25 | 21 | 151 | 136 |
| Mean (SD) | 81.84 (16.34) | 0.83 (13.18) | 83.12 (11.59) | 10.19 (22.35) | 82.05 (15.63) | 2.28 (15.24) |
| Median | 89.00 | 0.00 | 87.00 | 5.00 | 89.00 | 1.00 |
| Q1; Q3 | 75.00; 92.00 | −5.00; 9.00 | 75.00; 90.00 | 0.00; 10.00 | 75.00; 91.00 | −4.00; 9.00 |
| Min; Max | 30.0; 100.0 | −40.0; 40.0 | 49.0; 95.0 | −16.0; 78.0 | 30.0; 100.0 | −40.0; 78.0 |

TABLE 37
Summary of EQ-5D Index Score and change from baseline to Week 52 - Full Analysis Set

| | Arm A | | Arm B | | | |
|---|---|---|---|---|---|---|
| | Prophylaxis | | On-demand −> Prophylaxis | | Overall | |
| | (N = 133) | | (N = 26) | | (N = 159) | |
| | Actual | Change from | Actual | Change from | Actual | Change from |
| | result | baseline | result | baseline | result | baseline |
| Visit | | | | | | |
| Baseline | | | | | | |
| Number | 122 | | 22 | | 144 | |
| Mean (SD) | 0.80 (0.18) | | 0.77 (0.18) | | 0.79 (0.18) | |
| Median | 0.77 | | 0.74 | | 0.77 | |
| Q1; Q3 | 0.71; 1.00 | | 0.68; 0.88 | | 0.70; 1.00 | |
| Min; Max | 0.1; 1.0 | | 0.4; 1.0 | | 0.1; 1.0 | |
| Week 52 | | | | | | |
| Number | 126 | 115 | 25 | 21 | 151 | 136 |
| Mean (SD) | 0.80 (0.18) | 0.02 (0.13) | 0.83 (0.19) | 0.05 (0.17) | 0.81 (0.18) | 0.02 (0.13) |
| Median | 0.79 | 0.00 | 0.84 | 0.04 | 0.80 | 0.00 |
| Q1; Q3 | 0.71; 1.00 | −0.02; 0.06 | 0.75; 1.00 | 0.00; 0.15 | 0.71; 1.00 | −0.01; 0.08 |
| Min; Max | −0.0; 1.0 | −0.5; 0.4 | 0.2; 1.0 | −0.5; 0.3 | −0.0; 1.0 | −0.5; 0.4 |

These improvements in physical function were aligned with the results from the exit interviews (N=29, including 17 patients in Arm A) reporting improvement in physical functioning in most patients (26/29). In addition, trends of improved physical activity were also observed in the Actigraphy data (mean [SD]: 5376 [3796] steps/day at baseline/versus 5758 [4472] steps/day at Week 52; overall patients, N=158, including 132 patients in Arm A).

These results show that once-weekly prophylaxis with efanesoctocog alfa resulted in improvement in physical functioning in patients with hemophilia A, positively impacting daily living activities, in patients who were on prior standard of care FVIII prophylaxis. Results from the Phase 3 trial demonstrated that even in patients receiving conventional standard-of-care FVIII prophylaxis, significant and clinically meaningful improvements in health-related quality of life (associated with the prevention of bleeding) were achieved when switching to weekly efanesoctocog alfa prophylaxis. The improvement in health-related quality of life may enable patients with hemophilia A to advance closer to the goal of optimal outcomes and achieve a “hemophilia-free mind”. See Krumb E and Hermans C. Res Pract Thromb Haemost. 2021;5(5):e12567.

### Pain Intensity

Pain intensity was measured as a secondary endpoint according to PROMIS-Short Form (SF) v.1.0 3a first question (intensity of pain at its worst in the past 7 days (PAINQU6 score)). Change in scores from baseline to week 52 were analyzed (n=119) (FIG. 10B). Mixed-effect model with repeated measures (MMRM) was used to estimate the change from baseline to Week 26 and Week 52 in PROMIS Pain intensity 3a scores. Results are shown in Table 38. Pain was also assessed as exploratory endpoints using the PROMIS-SF v1.0 pain intensity 3a total T-score, PROMIS-SF v1.0 pain interference 6a T-score (in patients ≥18 years of age), and EuroQol-5 Dimension 5-level (EQ-5D-5L) pain/discomfort domain. Results of exit interviews and pain medication usage were evaluated in the overall patients included in the trial.

At baseline, 53.6% (67/125) of patients in Arm A reported moderate, severe, or very severe pain (at its worst in the past 7 days) on PROMIS-SF Pain intensity 3a scale, while moderate, severe, or extreme pain/discomfort was experienced by 19.7% (24/122) patients on EQ-5D-5L. Mean (SD) PROMIS pain intensity 3a scores for Arm A were 2.47 (1.15), 2.34 (1.15), and 2.21 (1.21) at baseline, week 26, and week 52, respectively (see Table 38). Approximately 75% of adults (75/103) indicated that pain had interfered with their day-to-day activities.

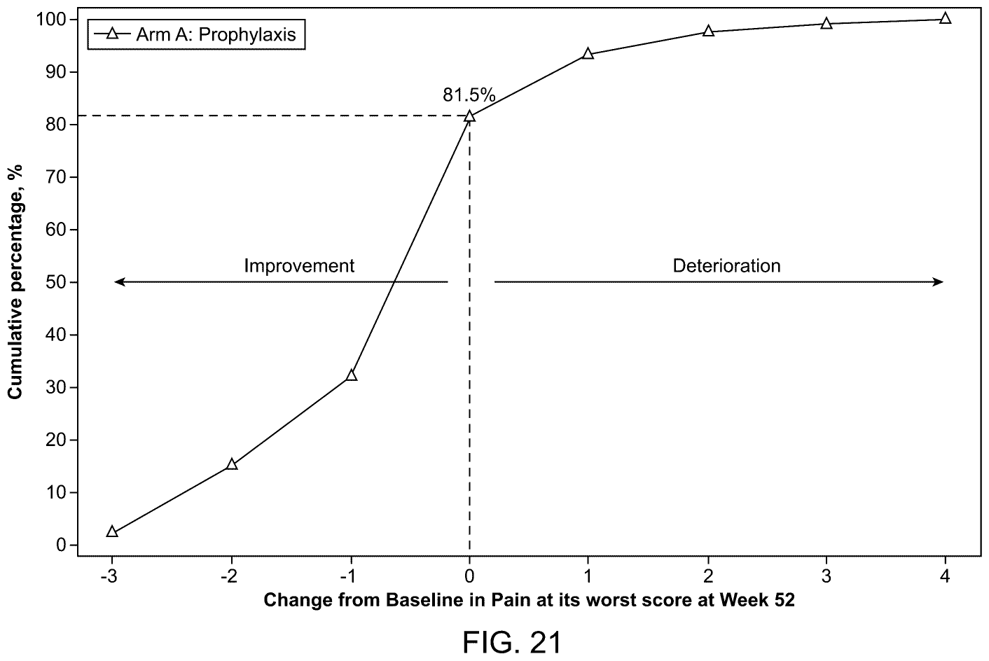

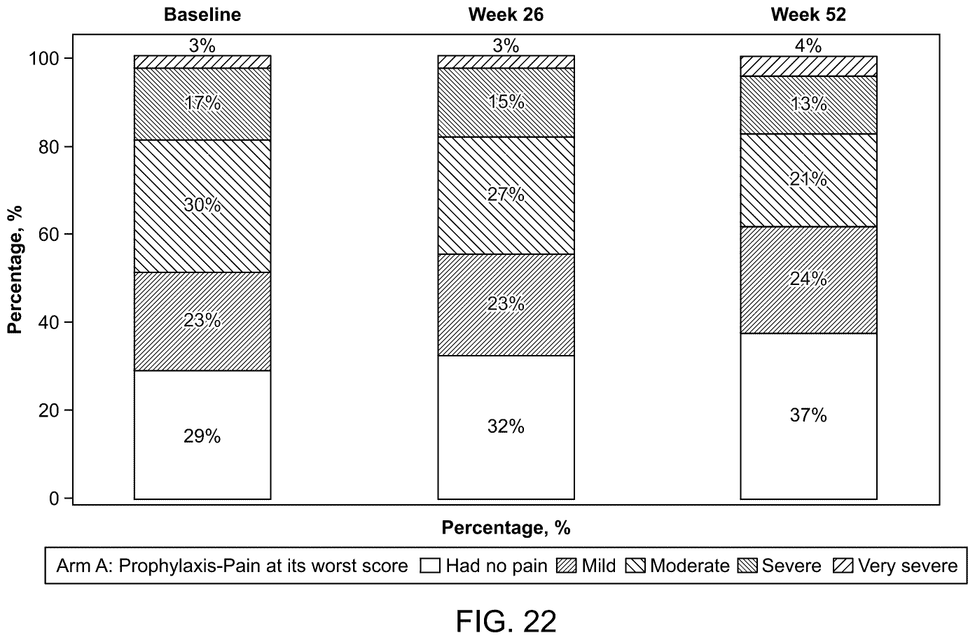

Pain intensity (at its worst) was improved or maintained (change from baseline ≤0) for most patients (81.5% [97/119]) at Week 52 (FIG. 21). A greater proportion of patients did not experience any pain (37% [44/119]) at Week 52 compared with the baseline (29% [35/119]) in PROMIS pain intensity 3a first item (pain at its worst) scale (FIG. 22). Improvement in the “average pain in the past 7 days” score was −0.20 (LS mean 95% CI: −0.33 to −0.63; p-value=0.0042. Improvement in the “pain intensity right now” score was −0.14 (LS mean 95% CI: −0.25 to −0.03; p-value=0.0128).

Prophylactic treatment with efanesoctocog alfa demonstrated statistically significant improvement in PROMIS Pain intensity 3a first item score. For Arm A, the least square (LS) mean change from baseline to Week 52 in PROMIS Pain Intensity score (n=119) was −0.21 (95% CI: −0.41 to −0.02, p-value=0.0276). This improvement was also clinically significant since it was within the range of the meaningful within-group change (−0.5 to −0.2) determined using psychometric analyses in the context of the study.

TABLE 38
Mean change in PROMIS Pain Intensity 3a past 7 days intensity of pain at its worst
score from baseline to Week 52 using MMRM (>=12 years old) - Full Analysis Set

| | Arm A | | Arm B | | | |
|---|---|---|---|---|---|---|
| | Prophylaxis | | On-demand −> Prophylaxis | | Overall | |
| | (N = 133) | | (N = 26) | | (N = 159) | |
| | Actual | Change from | Actual | Change from | Actual | Change from |
| Visit | result | baseline | result | baseline | result | baseline |
| Baseline | | | | | | |
| Number | 125 | | 23 | | 148 | |
| Mean (SD) | 2.47 (1.15) | | 2.74 (1.05) | | 2.51 (1.13) | |
| Median | 3.00 | | 3.00 | | 3.00 | |
| Q1; Q3 | 1.00; 3.00 | | 2.00; 4.00 | | 1.00; 3.00 | |
| Min; Max | 1.0; 5.0 | | 1.0; 4.0 | | 1.0; 5.0 | |
| Week 26 | | | | | | |
| Number | 128 | 120 | 26 | 23 | 154 | 143 |
| Mean (SD) | 2.34 (1.15) | −0.09 (1.16) | 2.08 (1.16) | −0.57 (1.59) | 2.29 (1.15) | −0.17 (1.24) |
| Median | 2.00 | 0.00 | 2.00 | −1.00 | 2.00 | 0.00 |
| Q1; Q3 | 1.00; 3.00 | −1.00; 0.00 | 1.00; 3.00 | −2.00; 0.00 | 1.00; 3.00 | −1.00; 0.00 |
| Min; Max | 1.0; 5.0 | −4.0; 3.0 | 1.0; 5.0 | −3.0; 3.0 | 1.0; 5.0 | −4.0; 3.0 |
| Week 52 | | | | | | |
| Number | 127 | 119 | 25 | 22 | 152 | 141 |
| Mean (SD) | 2.21 (1.21) | −0.21 (1.20) | 1.88 (0.88) | −0.77 (0.81) | 2.16 (1.16) | −0.30 (1.16) |
| Median | 2.00 | 0.00 | 2.00 | −1.00 | 2.00 | 0.00 |
| Q1; Q3 | 1.00; 3.00 | −1.00; 0.00 | 1.00; 2.00 | −1.00; 0.00 | 1.00; 3.00 | −1.00; 0.00 |
| Min; Max | 1.0; 5.0 | −3.0; 4.0 | 1.0; 4.0 | −2.0; 1.0 | 1.0; 5.0 | −3.0; 4.0 |
| LS Mean (SE)a | | −0.21 (0.10) | | | | |
| 95% C.la | | (−0.41, −0.02) | | | | |
| p-value | | 0.0276 | | | | |

These results were supported by statistically significant improvement in overall pain intensity in the PROMIS-SF Pain intensity 3a T-score (LS mean change from baseline: −1.94 [95% CI: −3.26 to −0.63]; P=0.0042). Numerical improvement was observed in PROMIS-SF Pain Interference 6a T-score (LS mean change from baseline to Week 52: −1.16 [95% CI: −2.45 to 0.14]; P=0.0793). (see Table 39).

TABLE 39
PROMIS-SF Pain intensity 3a T score
and PROMIS-SF Interference 6a T-score

| | | | Change from |
|---|---|---|---|
| | Baseline | Week 52 | baseline, LS mean |
| PROMIS-SF Pain | 42.93 | 40.46 | −1.94 [95% CI: −3.26 |
| intensity 3a T | (8.64) | (9.12) | to −0.63]; |
| score, mean (SD) | | | P = 0.0042 |
| PROMIS-SF | 52.86 | 51.45 | −1.16 [95% CI: −2.45 |
| Interference 6a | (7.88) | (8.16) | to −0.14] |
| T score, mean (SD) | | | P = 0.0793 |

These results were also supported by patient reporting from EQ-5D-5L surveys and exit interviews. For the EQ-5D-5L, at Week 52, 20.9% (24/115) patients reported improvement in at least 1 category versus baseline in the pain/discomfort domain. During exit interviews (N=29, including 17 patients in Arm A), most patients reported a meaningful improvement in pain (25/29). Exit interview results (n=29; 17 in Arm A) showed nearly all patients (96.6%; 28/29) experienced hemophilia-related pain pre-study. Among these 28 patients, 25 expressed an improvement in joint pain and one additional patient reported improvement in the ability to move without pain.

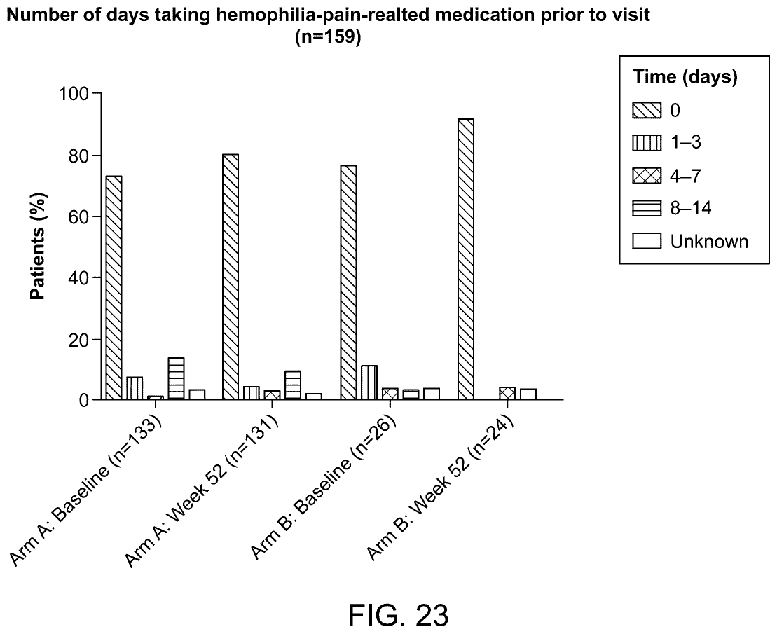

The percentage of patients from Arms A and B who did not take any hemophilia-related pain medication in the 14 days prior to visit increased from baseline (73.6%; [n=117]) to Week 52 (81.9% [n=127]), reflecting a trend towards a relative reduction in hemophilia-related pain medication usage at Week 52. (FIG. 23). The most frequently used concomitant medication during the study was paracetamol (40.9%; 65 patients) and celecoxib (15.7%; 25 patients).

### Joint Health

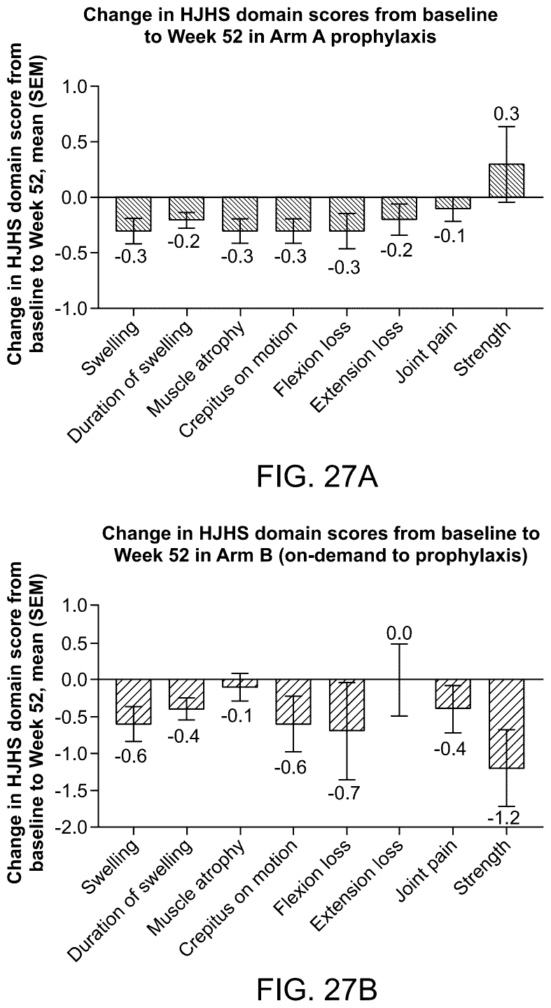

Joint health was measured by the Hemophilia Joint Health Score (HJHS) v2.1 baseline to week 52 (n=106) (FIG. 10C). Changes in Hemophilia Joint Health Score (HJHS) total score from Baseline to Week 52 for both study arms are shown in Table 40. Mean HJHS total score decreased from baseline to Week 52 (−1.54 [95% CI −2.70 to −0.37]; P=0.01) in Arm A (Table 40). Changes from baseline in HJHS domain scores at week 52 for the prophylaxis and on-demand arms are shown in Table 41. In Arm A, the HJHS domains with greatest mean improvement from baseline to Week 52 were swelling, muscle atrophy, crepitus on motion, and flexion loss (Table 41, FIG. 27A). Mean (SD) change from baseline to Week 52 (which was calculated if all 48 individual item scores, 8 domains×6 joints, were present) in HJHS total joint score was −1.4 (6.2); n=108. Mean (SD) change from baseline to Week 52 in HJHS gait score was −0.1 (0.8); n=120 (assessed on a range from 0-4).

In Arm B, In Arm B, the HJHS domains with greatest mean improvement from baseline to Week 52 were swelling, duration of swelling, crepitus on motion, flexion loss, joint pain, and strength (Table 41, FIG. 27B). Mean (SD) change from baseline to Week 52 in HJHS total joint score (which was calculated if all 48 individual item scores, 8 domains×6 joints, were present) was −3.9 (8.6); n=22. Mean (SD) change from baseline to Week 52 in HJHS gait score was −0.2 (0.5); n=23 (assessed on a range from 0-4).

In Arm A, which included participants on stable standard-of-care FVIII replacement prophylaxis prior to enrollment, the mean (SD) HJHS Total score at baseline was 18.1 (18.4). The adjusted mean change in HJHS Total score from baseline to Week 52 was −1.54 (95% CI: −2.70 to −0.37) (p-value=0.0101) in Arm A (n=107) and −4.1 (95% CI: −7.94 to −0.25)(p-value=0.0382; n=22) in Arm B (n=22), thus showing a statistically significant improvement in functional measure of joint health. See Table 40.

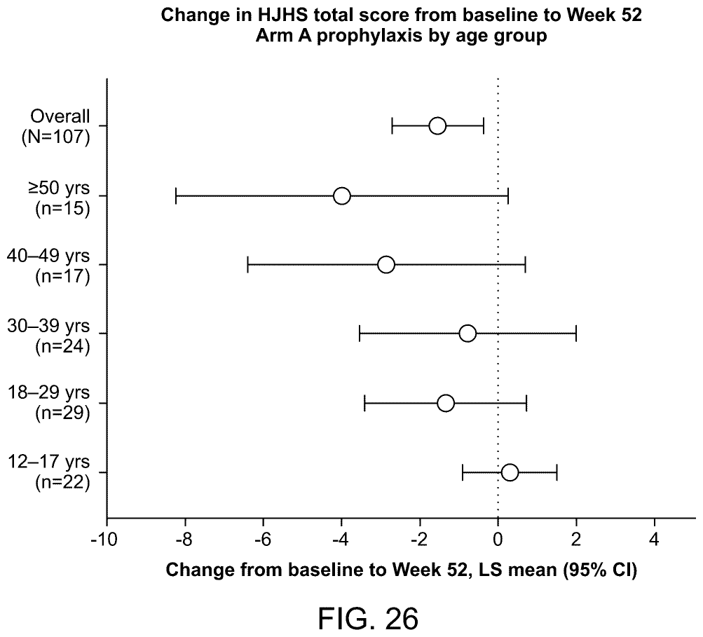

HJHS scores were further analyzed by demographic in both Arm A and Arm B (Table 42; FIG. 26), and the demographics of participants in Arm A were also analyzed by HJHS score (Table 43). Participants were grouped by total HJHS score reduction of >4 points, <4 points, >2 points, or <2 point (Table 44). Participants whose HJHS total score was reduced by >4 points tended to be older and have a higher BMI than those who had a HJHS total score reduction of less than 4 points.

The improvements reported for participants on efanesoctocog alfa prophylaxis after only 52 weeks represent a clinically meaningful improvement, especially given that most patients (80.5%) had no target joints at baseline and all were on standard of care prestudy prophylaxis.

Additionally, post hoc analyses indicated a numerical trend towards reduction (i.e., improvement) in Haem-A-QoL physical health score, PROMIS Pain Intensity 3a first item, and Hemophilia Joint Health Score (HJHS) total score after 52 weeks of efanesoctocog alfa prophylaxis treatment in patients who took Eloctate® prior to enrolling in the study. However, the 95% CIs of mean reductions did not exclude zero.

TABLE 40
Mean change in Hemophilia Joint Health Score (HJHS) total score
from Baseline to Week 52 using MMRM - Full Analysis Set

| | Arm A | | Arm B | |
|---|---|---|---|---|
| | Score | | | |
| | Prophylaxis | | On-demand−>Prophylaxis | |
| | (N = 133) | | (N = 26) | |
| Visit | Actual result | Change from baseline | Actual result | Change from baseline |
| Total Score | | | | |
| Baseline | | | | |
| Number | 116 | | 25 | |
| Mean (SD) | 18.1 (18.4) | | 26.3 (13.2) | |
| Median | 12.5 | | 29.0 | |
| Q1; Q3 | 2.0; 29.0 | | 21.0; 34.0 | |
| Min; Max | 0; 98 | | 2; 51 | |
| Week 26 | | | | |
| Number | 115 | 109 | 11 | 11 |
| Mean (SD) | 17.4 (18.4) | −0.9 (4.3) | 23.7 (14.3) | −1.6 (6.4) |
| Median | 12.0 | 0.0 | 22.0 | −1.0 |
| Q1; Q3 | 1.0; 31.0 | −2.0; 0.0 | 12.0; 35.0 | −3.0; 1.0 |
| Min; Max | 0; 98 | −18; 10 | 0; 42 | −16; 8 |
| Week 52 | | | | |
| Number | 110 | 107 | 22 | 22 |
| Mean (SD) | 16.5 (17.6) | −1.5 (6.4) | 21.1 (13.1) | −4.1 (8.7) |
| Median | 11.5 | 0.0 | 20.0 | −3.0 |
| Q1; Q3 | 1.0; 28.0 | −3.0; 0.0 | 8.0; 35.0 | −7.0; 2.0 |
| Min; Max | 0; 98 | −26; 24 | 2; 39 | −27; 8 |
| LS Mean (SE)a | | −1.54 (0.59) | | |
| 95% C.Ia | | (−2.70, −0.37) | | |
| p-value | | 0.0101 | | |

Note 1:
Higher HJHS scores denote worse joint health. The HJHS total score ranges from 0-124; total joint score ranges from 0-120; gait score ranges from 0-4.
2: The HJHS total score can be calculated if all 48 individual item scores (8 domains × 6 joints, total joint score) and the gait score were present. HJHS assessments within 2 weeks after a joint or muscle bleed are excluded. Joint scores post joint surgeries are replaced using the last observation carried forward (LOCF) method. Assessments during other major surgical periods are excluded.
aThe LS mean (SE) and 95% C.I. are estimated by mixed-effect model with repeated measures (MMRM) with visit as fixed effect, and baseline Hemophilia Joint Health Score (HJHS) total score as covariate.

TABLE 41
Change from Baseline in Hemophilia Joint Health Score
(HJHS) Domain Score at Week 52: Arms A and B

| | Arm A | Arm B |
|---|---|---|
| Number of | 133 | 26 |
| subjects analysed | | |
| HJHS Domain Score | | |
| arithmetic mean | | |
| (standard deviation) | | |
| Swelling | −0.3 ± 1.2 (n = 114) | −0.6 ± 1.1 (n = 22) |
| Duration Of Swelling | −0.2 ± 0.8 (n = 113) | −0.4 ± 0.7 (n = 22) |
| Muscle Atrophy | −0.3 ± 1.2 (n = 116) | −0.1 ± (0.9) (n = 22) |
| Crepitus On Motion | −0.3 ± 1.2 (n = 114) | −0.6 ± 1.8 (n = 22) |
| Flexion Loss | −0.3 ± 1.6 (n = 112) | −0.7 ± 3.1 (n = 22) |
| Extension Loss | −0.2 ± 1.5 (n = 113) | 0.0 ± (2.3) (n = 22) |
| Joint Pain | −0.1 ± 1.2 (n = 114) | −0.4 ± 1.5 (n = 22) |
| Strength | 0.3 ± 0.6 (n = 113) | −1.2 ± 2.4 (n = 22) |

TABLE 42
Demographics for Participants With HJHS
Total Score Assessed at Week 52

| | Arm A | | Arm B | |
|---|---|---|---|---|
| | (n = 110) | | (n = 22) | |
| Age, years | | | | |
| Mean (SD) | 32.4 | (14.6) | 42.7 | (12.0) |
| Median (range) | 31.5 | (12-67) | 39.0 | (23-68) |
| Age group, n (%)^{a} | | | | |
| 12-17 years | 22 | (20.0) | 0 | |
| 18-64 years | 87 | (79.1) | 21 | (95.5) |
| ≥64 years | 1 | (0.9) | 1 | (4.6) |
| Race, n (%) | | | | |
| Asian | 27 | (24.6) | 0 | |
| Black or African American | 3 | (2.7) | 0 | |
| White | 54 | (49.1) | 22 | (100) |
| Other | 24 | (21.8) | 0 | |
| Not reported | 2 | (1.8) | 0 | |
| BMI group, n (%) | | | | |
| <25 | 52 | (47.3) | 8 | (36.4) |
| ≥25-<30 | 40 | (36.4) | 7 | (31.8) |
| ≥30 | 18 | (16.4) | 6 | (27.3) |

| | | | |
|---|---|---|---|
| Not reported | 0 | 1 | (4.5) |

BMI, body mass index; HJHS, Hemophilia Joint Health Score; SD, standard deviation.
^{a}Percentages are based on the total number of participants in the column.
^{b}BMI not reported for 1 participant as height was not recorded.

TABLE 43
Greater Improvements in Joint Health Were Observed
in Participants in Arm A with Increased Age
and Higher HJHS Total Score at Baseline

| | | | | Change |
|---|---|---|---|---|
| | | | | from |
| | | | | baseline |
| | | HJHS at | HJHS at | to Week 52, |
| | | baseline, | Week 52, | LS mean |
| | | mean (SD) | mean (SD) | (95% CI)^{a,b} |
| Overall | | 18.1 (18.4) | 16.5 (17.6) | −1.54 (−2.70, −0.37) |
| | | n = 116 | n = 110 | n = 107; p = 0.0101 |
| ≥50 | yrs | 41.1 (19.6) | 37.1 (21.7) | −4.00 *−8.25, 0.25) |
| | | n = 17 | n = 15 | n = 15; p = 0.0634 |
| 40-49 | yrs | 27.7 (14.0) | 26.4 (12.3) | −2.85 (−6.39, 0.69) |
| | | N = 19 | N = 17 | N = 17; p = 0.1071 |
| 30-39 | yrs | 21.6 (15.6) | 20.7 (16.1) | −0.77 (−3.55, 2.01) |
| | | N = 27 | N = 26 | N = 24; p = 0.5733 |
| 18-29 | yrs | 7.7 (9.9) | 6.5 (8.2) | −1.34 (−3.42, 0.74) |
| | | N = 30 | N = 30 | N = 29; p = 0.1990 |
| 12-17 | yrs | 2.9 (5.5) | 3.3 (7.2) | 0.30 (−0.91, 1.51) |
| | | N = 23 | N = 22 | N = 22; p = 0.6104 |

CI, confidence interval; HJHS, Hemophilia Joint Health Score; LS, least squares.
^{a}LS mean (95% CI), and P value estimated by mixed-effect model with repeated measures with visit as a fixed effect, and baseline HJHS as covariate.
^{b}Includes only patients with HJHS measurements at both timepoints.

TABLE 44
Patient Demographics by Total HJHS Score Reduction

| | HJHS total | | HJHS total | | HJHS total | | HJHS total | | | |
|---|---|---|---|---|---|---|---|---|---|---|
| | score | | score | | score | | score | | Overall | |
| | reduced | | reduced | | reduced- | | reduced | | population | |
| | by ≥4 points | | by <4 points | | by ≥2 points | | by <2 points | | in Arm A | |
| | (n = 24) | | (n = 83) | | (n = 38) | | (n = 69) | | (N = 107) | |
| Age, years | | | | | | | | | | |
| Mean (SD) | 35.5 | (13.3) | 31.5 | (15.1) | 36.2 | (13.7) | 30.3 | (15.0) | 32.4 | (14.8) |
| Median (range) | 35.5 | (14-59) | 29.0 | (12-67) | 35.5 | (12-64) | 28.0 | (12-67) | 31.0 | (12-67) |
| Age group, n (%)^{a} | | | | | | | | | | |
| 12-17 years | 1 | (4.2) | 21 | (25.3) | 2 | (5.3) | 20 | (29.0) | 22 | (20.6) |
| 18-64 years | 23 | (95.8) | 61 | (73.5) | 36 | (94.7) | 48 | (69.6) | 84 | (78.5) |
| ≥64 years | 0 | | 1 | (1.2) | 0 | | 1 | (1.5) | 1 | (0.9) |
| BMI group, n (%)^{a} | | | | | | | | | | |
| <25 | 9 | (37.5) | 42 | (50.6) | 15 | (39.5) | 36 | (52.2) | 51 | (47.7) |
| ≥25-<30 | 9 | (37.5) | 31 | (37.4) | 15 | (39.5) | 25 | (36.2) | 40 | (37.4) |
| ≥30 | 6 | (25.0) | 10 | (12.1) | 8 | (21.1) | 8 | (11.6) | 16 | (15.0) |

BMI, body mass index; HJHS, Hemophilia Joint Health Score; SD, standard deviation.
^{a}Percentages are based on the total number of participants in the column.

Structural joint health was also assessed by Hemophilia Early Arthropathy Detection with Ultrasound (HEAD-US) in a subset of patients from this Phase 3 open-label, multicenter study. Power Doppler ultrasound (PDUS) in tissues of interest was evaluated semi-quantitatively to assess synovial hyperemia, an indicator of neo-angiogenesis and active synovitis (score 0-3).

The results of this sub-study are displayed in Table 45. Thirty-one patients participated in this sub-study, including 8 (26%) with ≥1 target joint (defined as a major joint with ≥3 spontaneous bleeds in a consecutive 6-month period) at baseline. Median (range) age was 39 (16-72) years. Mean (standard deviation [SD]) total HEAD-US score per patient decreased from 12.2 (11.8) at baseline to 10.7 (11.6) at Week 52 (mean [SD] change −1.3 [4.5]) (Table 45). A numerically greater reduction (improvement) in total HEAD-US score from baseline to Week 52 was observed in patients with no target joints at baseline compared to those with ≥1 target joint (mean [SD] change −1.5 [3.93] and −0.7 [6.15], respectively). Overall, 41% (9/22) of participants had improvements in their total HEAD-US score from baseline to Week 52, for 27% (6/22) of participants their score stayed the same, and for 32% (7/22) of participants their score worsened. At least 20% of patients had improvements in HEAD-US score from baseline to Week 52 in the left elbow (8/26; 31%), right ankle (7/26; 27%), right knee (6/27; 22%), and left ankle (5/25; 20%). A total of 46% (13/28) of participants had an improvement in their semiquantitative hypertrophic synovium score, and 33% (8/24) an improvement in their PDUS grading assessment. Improvements in semiquantitative bone and cartilage scores, were observed in 29% (8/28) and 21% (6/28) of participants, respectively.

TABLE 45
Summary of total HEAD-US scores and ultrasound parameter scores

| Summary of change in total HEAD-US score | | | |
|---|---|---|---|
| per patient from baseline to Week 52^{a,b} | | | |
| | | | Change from |
| | | | baseline to |
| | Baseline | Week 52 | Week 52 |

Overall study population

| | | | |
|---|---|---|---|
| n | 27 | 23 | 22 |

| | | | | | | |
|---|---|---|---|---|---|---|
| Mean (SD) | 12.2 | (11.8) | 10.7 | (11.6) | −1.3 | (4.5) |
| Median (range) | 8.0 | (0, 35) | 6.0 | (0, 32) | 0.0 | (−12, 6) |
| Patients with no target | | | | | | |
| joints at baseline | | | | | | |

| | | | |
|---|---|---|---|
| n | 20 | 16 | 16 |

| | | | | | | |
|---|---|---|---|---|---|---|
| Mean (SD) | 9.2 | (11.7) | 6.3 | (10.5) | −1.5 | (3.9) |
| Median (range) | 3.5 | (0, 35) | 0.0 | (0, 32) | 0.0 | (−9, 6) |
| Patients with ≥1 target | | | | | | |
| joint at baseline | | | | | | |

| | | | |
|---|---|---|---|
| n | 7 | 7 | 6 |

| | | | | | | |
|---|---|---|---|---|---|---|
| Mean (SD) | 20.7 | (7.2) | 20.7 | (6.6) | −0.7 | (6.2) |
| Median (range) | 23.0 | (9, 29) | 20.0 | (14, 26) | 1.5 | (−12, 5) |

Summary of improvement and worsening
in total HEAD-US score and ultrasound
parameters from baseline to Week 52^{a-c}

| | Worsened | | No change | | Improved | |
|---|---|---|---|---|---|---|
| Difference in | 7/22 | (31.8) | 6/22 | (27.3) | 9/22 | (40.9) |
| total HEAD-US score per | | | | | | |
| patient, n/N (%) | | | | | | |
| Difference in | | | | | | |
| total HEAD-US scores per | | | | | | |
| joint per patient, n/N (%) | | | | | | |
| Left elbow | 1/26 | (3.9) | 17/26 | (65.4) | 8/26 | (30.8) |
| Right elbow | 5/27 | (18.5) | 17/27 | (63.0) | 5/27 | (18.5) |
| Left knee | 5/25 | (20.0) | 18/25 | (72.0) | 2/25 | (8.0) |
| Right knee | 6/27 | (22.2) | 15/27 | (55.6) | 6/27 | (22.2) |
| Left ankle | 4/25 | (16.0) | 16/25 | (64.0) | 5/25 | (20.0) |
| Right ankle | 1/26 | (3.9) | 18/26 | (69.2) | 7/26 | (26.9) |
| Difference in ultrasound | | | | | | |
| parameter scores across all | | | | | | |
| the joints per patient, n/N (%) | | | | | | |
| Semiquantitative bone scoring | 6/28 | (21.4) | 14/28 | (50.0) | 8/28 | (28.6) |
| Semiquantitative cartilage scoring | 10/28 | (35.7) | 12/28 | (42.9) | 6/28 | (21.4) |
| Semiquantitative hypertrophic synovium | 6/28 | (21.4) | 9/28 | (32.1) | 13/28 | (46.4) |
| scoring | 7/24 | (29.2) | 9/24 | (37.5) | 8/24 | (33.3) |
| PDUS grading assessment | | | | | | |

Abbreviations: HEAD-US, Hemophilia Early Arthropathy Detection with Ultrasound; PDUS, power Doppler ultrasound; SD, standard deviation.
^{a}Total HEAD-US score calculated if all 18 items (3 HEAD-US parameters × 6 joints) are measured. Higher scores correspond to a worse outcome. The 6 joints included are left and right elbows, ankles, and knees, and the total ultrasound parameter score per joint ranges from 0-8.
The 3 HEAD-US parameters are semiquantitative bone scoring, semiquantitative cartilage, and semiquantitative hypertrophic synovium. Scoring ranges are as follows: semiquantitative bone scoring, 0-12; semiquantitative cartilage, 0-24; and semiquantitative hypertrophic synovium, 0-12. Score for PDUS assessment ranges from 0-18.
^{b}HEAD-US measurements within 14 days after a joint bleed were excluded, and measurements after joint surgeries were replaced using the last observation carried forward method. Summary is based on evaluable readings. Of a total of 2832 available readings across patients, visits, joints, and ultrasound parameters, 161 (5.6%) were not evaluable.
^{c}Where n is the number of participants satisfying the criteria and N is the number of participants with assessments at both baseline and Week 52. Percentages are based on N.

### Exit Interviews

Exit interviews were conducted to understand the symptoms and experiences of hemophilia and obtain feedback on the PROMIS Pain Intensity 3a, particularly the item addressing worst pain, and Haem-A-QoL Physical Health questionnaires that were included in the study. Overall, exit interviews supported the improvements observed in physical health according to these questionnaires.

Exit interviews were qualitative, semi-structured interviews with 29 of the 159 total participants (27 adults and 2 adolescents) from 6 countries (Argentina, France, Italy, South Korea, UK, and US) within 6 months of exiting the study but before the end of study was declared. Seventeen (58.6%) of the participants were enrolled in Arm A. Questions were asked relating to participants' pre-study experiences with hemophilia A, pain, physical functioning, and the impact of prior treatments; participants' on-study experiences with efanesoctocog alfa treatment on pain intensity and physical functioning; and meaningful changes, if any, that participants' reported with efanesoctocog alfa treatment.

Pre-study participant experiences: Among the 17 participants (58.6%; 17/29) enrolled from Arm A, 13 (76.5%) had reported “wearing off” feeling with prior prophylactic treatment, including more pain, stiffness, feeling unprotected, breakthrough bleeds, and limited physical activities.

Of the 29 interview participants, 28 (96.6%) reported that they experienced at least “moderate intensity” of hemophilia-related pain prior to the study. Most participants (26/28; 92.9%) experienced varying degrees of pain intensity in more than one joint (e.g., knees, joints, ankles, elbows). Participants also reported that hemophilia-related pain and other symptoms adversely affected their day-to-day activities, particularly their physical functioning. The majority of the concepts assessed in the exit interviews were experienced by at least 25/29 participants (86.2%) before the study. For pre-study experiences with hemophilia A, 41.4% of the participants experienced more time to get ready, 86.2% experienced inability to walk desired distance, 89.7% experienced pain with movement, 96.6% experienced pain in joints, and 86.2% experienced painful swelling.

On-study participant experiences: Among the 28 participants who had pre-study hemophilia-related pain, 25/28 (89.3%) reported meaningful improvements in joint pain after switching to efanesoctocog alfa. For on-study participant experiences with hemophilia A, 75.0% of the participants reported improvement in time to get ready, 80.0% reported improvement in ability to walk desired distance, 88.5% reported improvement in ability to move without pain, 89.3% reported improvement in pain in joints, and 84% reported improvement in painful swelling. The remaining three participants who reported no change in joint pain intensity with efanesoctocog alfa noted that the improvements were not likely as they had sustained extensive cumulative joint damage over the years from repeated joint bleeds.

Participants with mild impairments before the study indicated that the level of improvement after the study was substantial in terms of overall functioning and quality of life. Despite differences in the magnitude of changes, improvements in physical health were identified as meaningful by all the interview participants.

All 29 exit interview participants (100%) preferred weekly efanesoctocog alfa prophylaxis over their previous hemophilia treatment.

In addition to the decreased bleeding events and improvement in pain, participants also reported increased confidence in protection, less fatigue, and improved health related quality of life compared with previous treatments. These exit interviews demonstrated that participants with mild impairments pre-study reported substantial improvements in overall functioning and quality of life after switching to efanesoctocog alfa treatment. These qualitative findings are consistent with the quantitative improvements observed in the PROMIS Pain Intensity 3a and Haem-A-QoL PH subscale results in the study.

### Other Assessments

Physicians assessed subject's response to Efa treatment using the 4-point Physician's Global Assessment (PGA) response scale (see Table 2). Results are shown in Table 46. P_{GP}-6₅,T₁

TABLE 46
Physicians' Global Assessment (PGA) of Subject's Response to Efa Treatment

| | Arm A: Prophylaxis | Arm B: On-Demand | Arm B: Prophylaxis |
|---|---|---|---|
| Number of subjects analysed | 133 | 26 | 26 |
| Total responses | 622 | 77 | 44 |
| Percentage of responses | | | |
| with indicated response | | | |
| Excellent | 95.7 | 96.1 | 93.2 |
| Effective | 4.3 | 3.9 | 6.8 |
| Partially effective | 0 | 0 | 0 |
| Ineffective | 0 | 0 | 0 |

A participant assessment of response to efanesoctocog alfa treatment of bleeding episodes in the Phase 3 study was also analyzed. The results are seen in Table 47 below.

TABLE 47
Summary of participant's assessment of response to Efa treatment of bleeding episodes

| | Arm A | | Arm B | | | | | |
|---|---|---|---|---|---|---|---|---|
| | Prophylaxis | | On-demand | | Prophylaxis | | Overall | |
| n(%) | (N = 133) | | (N = 26) | | (N = 26) | | (N = 159) | |
| Number of participants with an | 133 | | 26 | | 26 | | 159 | |
| efficacy period | | | | | | | | |
| Each injection | | | | | | | | |
| Based on injections | | | | | | | | |
| with an evaluation | | | | | | | | |
| Number^{a} | 73 | | 255 | | 6 | | 334 | |
| Excellent or Good | 60 | (82.2) | 251 | (98.4) | 6 | (100) | 317 | (94.9) |
| Excellent | 39 | (53.4) | 195 | (76.5) | 5 | (83.3) | 239 | (71.6) |
| Good | 21 | (28.8) | 56 | (22.0) | 1 | (16.7) | 78 | (23.4) |
| Moderate | 10 | (13.7) | 4 | (1.6) | 0 | | 14 | (4.2) |
| None | 3 | (4.1) | 0 | | 0 | | 3 | (0.9) |
| Based on all injections | | | | | | | | |
| Number^{b} | 92 | | 275 | | 8 | | 375 | |
| Excellent or Good | 60 | (65.2) | 251 | (91.3) | 6 | (75.0) | 317 | (84.5) |
| Excellent | 39 | (42.4) | 195 | (70.9) | 5 | (62.5) | 239 | (63.7) |
| Good | 21 | (22.8) | 56 | (20.4) | 1 | (12.5) | 78 | (20.8) |
| Moderate | 10 | (10.9) | 4 | (1.5) | 0 | | 14 | (3.7) |
| None | 3 | (3.3) | 0 | | 0 | | 3 | (0.8) |
| Response not provided | 19 | (20.7) | 20 | (7.3) | 2 | (25.0) | 41 | (10.9) |
| First injection for | | | | | | | | |
| each bleeding episode | | | | | | | | |
| Based on injections | | | | | | | | |
| with an evaluation | | | | | | | | |
| Number^{a} | 72 | | 254 | | 6 | | 332 | |
| Excellent or Good | 59 | (81.9) | 250 | (98.4) | 6 | (100) | 315 | (94.9) |
| Excellent | 39 | (54.2) | 194 | (76.4) | 5 | (83.3) | 238 | (71.7) |
| Good | 20 | (27.8) | 56 | (22.0) | 1 | (16.7) | 77 | (23.2) |
| Moderate | 10 | (13.9) | 4 | (1.6) | 0 | | 14 | (4.2) |
| None | 3 | (4.2) | 0 | | 0 | | 3 | (0.9) |
| Based on all injections | | | | | | | | |
| Number^{b} | 86 | | 268 | | 8 | | 362 | |
| Excellent or Good | 59 | (68.6) | 250 | (93.3) | 6 | (75.0) | 315 | (87.0) |
| Excellent | 39 | (45.3) | 194 | (72.4) | 5 | (62.5) | 238 | (65.7) |
| Good | 20 | (23.3) | 56 | (20.9) | 1 | (12.5) | 77 | (21.3) |
| Moderate | 10 | (11.6) | 4 | (1.5) | 0 | | 14 | (3.9) |
| None | 3 | (3.5) | 0 | | 0 | | 3 | (0.8) |
| Response not provided | 14 | (16.3) | 14 | (5.2) | 2 | (25.0) | 30 | (8.3) |

Note:
1: ‘None’ means that there was no improvement, not that the participant did not provide a response.
2: The efficacy period reflects the sum of all intervals of time during which participants are treated with BIVV001 according to the study arms and treatment regimens excluding periods of PK evaluations, surgery/rehabilitation (minor and major), and large injection intervals (>28 days).
3: Participants are included in each study arm and treatment regimen they participated in for the duration of time on that regimen and, as such, may appear in more than one treatment regimen.
^{a}Number = number of injections (or bleeding episodes as appropriate) with a response. Percentages are based on the number during the efficacy period.
^{b}Number = number of injections (or bleeding episodes as appropriate) reported. Percentages are based on this number during the efficacy period.

Spontaneous bleeding rates were also analyzed as part of the Phase 3 study and compared to observational study 242HA201/OBS16221. The results are shown in Table 48.

TABLE 48
Spontaneous bleeding rates for subjects in Observational Study 242HA201/OBS16221 and Phase 3 Study

| | | | | | | | Efanesoctocog Alfa | | | | | |
|---|---|---|---|---|---|---|---|---|---|---|---|---|
| | Historical Treatment | | | | | | Treatment in Phase 3 Study | | | | | |
| | 242HA201/OBS16221 | | | | | | Arm A | | Arm B | | | |
| | On-Demand | | Prophylaxis | | Overall | | Prophylaxis | | On-demand | | Prophylaxis | |
| Number of | 19 | | 139 | | 157 | | 133 | | 26 | | 26 | |
| participants | | | | | | | | | | | | |
| with an | | | | | | | | | | | | |
| efficacy period | | | | | | | | | | | | |
| Total # of | | | | | | | 33 | | 197 | | 5 | |
| treated | | | | | | | | | | | | |
| spontaneous | | | | | | | | | | | | |
| bleeding | | | | | | | | | | | | |
| episodes | | | | | | | | | | | | |
| Spontaneous | | | | | | | | | | | | |
| Participant | | | | | | | | | | | | |
| level | | | | | | | | | | | | |
| Number | 19 | | 139 | | 157 | | 133 | | 26 | | 26 | |
| Mean (SD) | 18.84 | (14.29) | 2.60 | (7.52) | 4.55 | (10.00) | 0.29 | (0.73) | 15.87 | (9.28) | 0.45 | (1.13) |
| Median | 14.74 | | 0.00 | | 1.00 | | 0.00 | | 16.69 | | 0.00 | |
| Q1; Q3 | 7.72; 30.14 | | 0.00; 2.98 | | 0.00; 4.38 | | 0.00; 0.00 | | 8.64; 23.76 | | 0.00; 0.00 | |
| Min; Max | 0.0; 51.1 | | 0.0; 61.0 | | 0.0; 61.0 | | 0.00; 4.72 | | 0.00; 31.31 | | 0; 4.11406 | |
| Population- | | | | | | | | | | | | |
| level, model | | | | | | | | | | | | |
| based | | | | | | | | | | | | |
| Mean (95% CI) | 18.91 | (12.48; 28.64) | 2.70 | (1.92; 3.81) | 4.70 | (3.42; 6.46) | 0.2.7 | (0.18, 0.41) | 15.83 | (12.27, 20.43) | 0.44 | (0.16, 1.20) |

The number of spontaneous bleeds experienced by participants was also analyzed, and the results are shown in Table 49 for both arms. The ABR by type of bleed (Spontaneous, Traumatic, and Unknown Type) was also analyzed, and the results are shown in Table 49.

TABLE 49
Number of subjects grouped by the number of spontaneous bleeds each
had during the study period and ABR according to type of bleed.

| | Arm A | | Arm B | | | |
|---|---|---|---|---|---|---|
| | Prophylaxis | | On-demand | | Prophylaxis | |
| Number of participants with | 133 | | 26 | | 26 | |
| an efficacy period | | | | | | |
| Number of Spontaneous | | | | | | |
| Bleeds during Study Period | | | | | | |
| 0 | 107 | (80.5) | 1 | (3.8) | 22 | (84.6) |
| >0 to 5 | 26 | (19.5) | 4 | (15.4) | 4 | (15.4) |
| >5 to 10 | 0 | | 2 | (7.7) | 0 | |
| >10 to 20 | 0 | | 12 | (46.2) | 0 | |
| >20 | 0 | | 7 | (26.9) | 0 | |
| Annualized bleeding rate | | | | | | |
| according to type of bleed | | | | | | |
| (median (inter-quartile range | | | | | | |
| (Q1-Q3)) | | | | | | |
| Spontaneous | 0.00 | (0.00 to 0.00) | 16.69 | (8.64 to 23.76) | 0.00 | (0.00 to 0.00) |
| Traumatic | 0.00 | (0.00 to 0.00) | 3.95 | (0.00 to 6.48) | 0.00 | (0.00 to 0.00) |
| Unknown type | 0.00 | (0.00 to 0.00) | 0.00 | (0.00 to 0.00) | 0.00 | (0.00 to 0.00) |

The percentage of participants who maintained FVIII activity levels over 1%, 5%, 10%, 15%, and 20% was also analyzed for Arm A: prophylaxis (50 IU/kg once weekly). Results are shown in Table 50.

TABLE 50
Percentage of Participants Achieving Factor VIII (FVIII) Activity
Levels Above 1%, 5%, 10%, 15%, and 20% in Prophylaxis Arm.

| | Arm A: Prophylaxis |
|---|---|
| Number of subjects analyzed | 131 |
| Percentage of subjects who maintain | |
| indicated FVIII activity levels | |
| >1% | 100 |
| >5% | 99.0 |
| >10% | 83.5 |
| >15% | 40.8 |
| >20% | 17.5 |

### Pharmacokinetics in Phase 3 Study

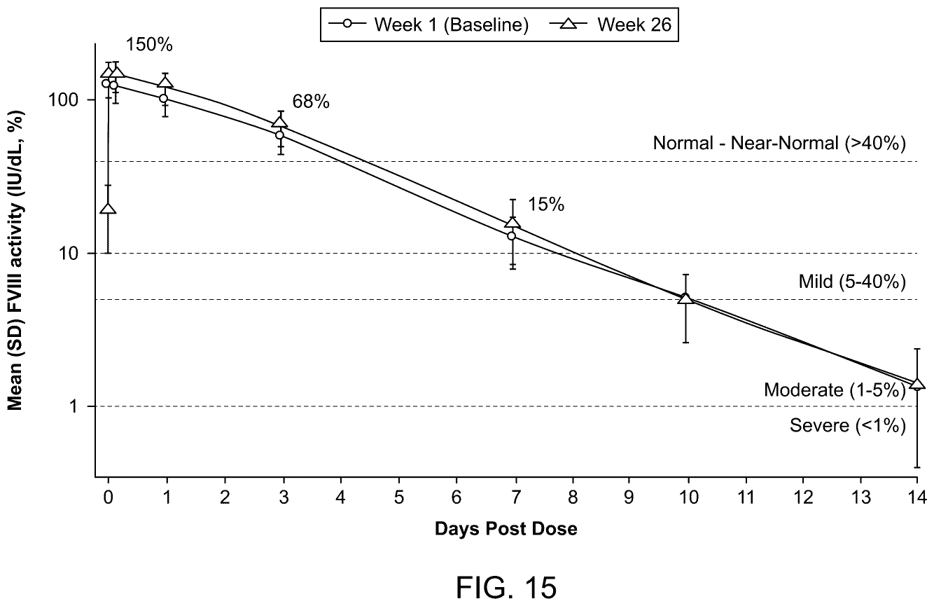

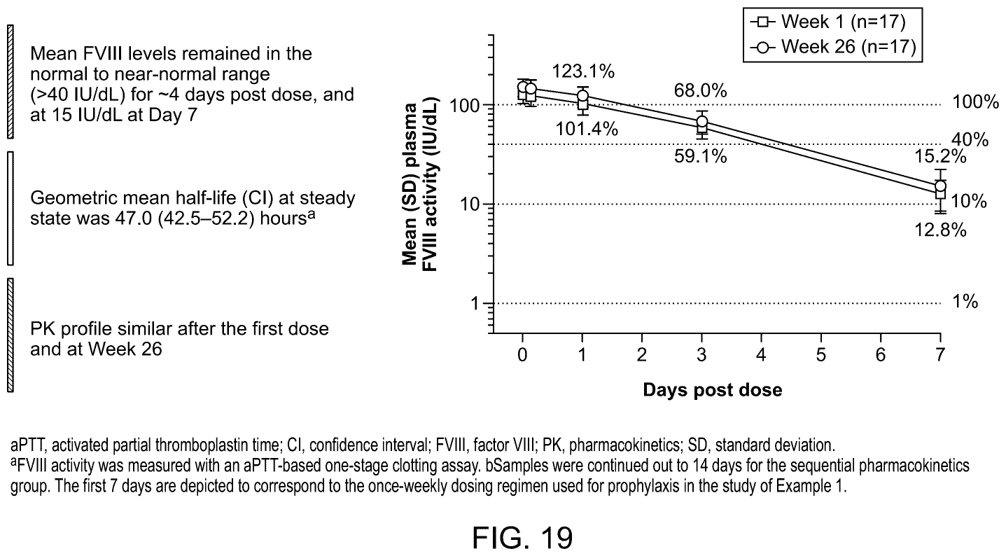

A pharmacokinetic (PK) objective of the study is to assess the PK of efanesoctocog alfa based on the one-stage activated partial thromboplastin time (aPTT) assay. Time above predefined FVIII activity levels was analyzed. Once weekly prophylactic treatment of 50 IU/kg efanesoctocog alfa resulted in mean FVIII activity levels at normal/near-normal (>40%) up to 4 days after dosing, and maintained mean FVIII activity levels >10% up to 7 days after dosing, as assessed by aPTT) at 15 IU/dL at day 7 (see FIG. 15, FIG. 19).

Several PK parameters at week 26 are shown in Table 51. At Week 26, patients had a geometric mean (95% CI) half-life of 47.0 (42.3-52.2) hours (FVIII activity was measured with an aPTT-based one-stage clotting assay). Indeed, PK profile was similar after the first dose and at Week 26. The geometric mean (95% CI) half-life, steady state clearance, maximum FVIII activity, and area under the activity-time curve from hour 0 to infinity were 47.0 (42.3 to 52.2) hours, 0.439 (0.390 to 0.493) mL/h/kg, 151 (137 to 167) IU/dL, and 11500 (10200 to 13000) h×IU/dL, respectively. This low variability in efanesoctocog alfa clearance suggests less need to adjust efanesoctocog alfa dose and/or dosing frequency.

Maximum FVIII Activity (Cmax) at baseline (day 1) for the Arm A: prophylaxis subjects (n=133) was 125.05±31.72 IU/dL (arithmetic mean (SD)). Cmax at week 52 for the Arm A: prophylaxis subjects (n=125) was 144.72±36.65 IU/dL (arithmetic mean (SD)).

Maximum FVIII Activity (Cmax) at baseline (day 1) for the Arm B: on-demand then prophylaxis subjects (n=26) was 138.36±48.66 IU/dL (arithmetic mean (SD)). Cmax at week 52 for the Arm B: on-demand then prophylaxis subjects (n=23) was 149.42±29.63 IU/dL (arithmetic mean (SD)).

Median elimination half-life (t_{1/2}) at baseline for 159 analyzed subjects was 46.5 hours (29.1 to 104) (median full range (min-max)). Median elimination half-life (t_{1/2}) at week 26 for 17 analyzed subjects was 46.7 hours (29.4 to 63.5) (median full range (min-max)).

Mean clearance (CL) at baseline (day 1) for 153 analyzed subjects was 0.508±0.124 mL/h/kg (arithmetic mean (SD)).

Total clearance at steady state (CLss) for 17 analyzed subjects was 0.449±0.101 (arithmetic mean (SD)). CLss is defined as the rate at which the drug is removed from the body at steady state. Analysis was performed on PK population. Here, number of subjects analyzed=subjects evaluable for this endpoint.

Mean accumulation index (AI) at week 26 for 15 analyzed subjects was 1.17±0.160 (arithmetic mean (SD)). AI is the ratio of accumulation of a drug under steady state conditions (i.e., after repeated administration) as compared to a single dose. AI was calculated as ratio of area under the curve (AUC) of Week 26 (Day 183) divided by AUC of Day 1, where AUC is the area under the plasma concentration versus time curve from time 0 to infinity. Analysis was performed on PK population. Here, number of subjects analyzed=subjects evaluable for this endpoint. This AI value suggests minimal accumulation of once-weekly dosing of efanesoctocog alfa.

Mean area under the plasma FVIII activity versus time curve (AUC₀₋ₜₐᵤ) was 9600±2010 hour*IU/dL (arithmetic mean (SD)) at baseline for 153 analyzed subjects and 11800±2720 hour*IU/dL (arithmetic mean (SD)) at week 26 for 17 analyzed subjects.

Mean residence time at baseline for 153 analyzed subjects was 61.9 hours (34.3 to 111) (median full range (min-max)). Mean residence time at week 26 for 17 analyzed subjects was 66.1 hours (46.7 to 92.3) (median full range (min-max)).

Mean incremental recovery (IR) was 2.60±0.648 kg*IU/dL/IU (arithmetic mean (SD)) at baseline for 153 analyzed subjects and 3.05±0.592 (arithmetic mean (SD)) at week 25 for 17 analyzed subjects.

Trough concentration for efanesoctocog alfa (Ctrough) pre-dose at baseline (day 1) was 0.00±0.00 IU/dL (arithmetic mean (SD)) for both Arm A and Arm B. Ctrough at week 52 for Arm A: prophylaxis for 133 analyzed subjects was 15.70±10.72 IU/dL (arithmetic mean (SD)). Ctrough at week 52 for Arm B: on-demand then prophylaxis for 26 analyzed subjects was 21.14±29.59 IU/dL (arithmetic mean (SD)).

Volume of Distribution at Steady State (Vss) was 31.7 (7.44) (arithmetic mean (SD)). at baseline for both Arm A and Arm B. Vss at week 26 was 29.6 (8.26) (arithmetic mean (SD)).

TABLE 51
Pharmacokinetic parameters of efanesoctocog alfa: week 26

| | Geometric mean PK parameters | |
|---|---|---|
| | for baseline-corrected | |
| Pharmacokinetic | FVIII activity at Week 26^{a,b} | |
| parameters | (n = 17) | |
| t_{1/2}, h | 47.0 | (42.3, 52.2) |
| Cₘₐₓ, IU/dL | 151 | (137, 167) |
| AUC₀₋ₜₐᵤ, h × IU/dL | 11500 | (10200, 13000) |
| CLₛₛ, mL/h/kg | 0.439 | (0.390, 0.493) |
| IR, IU/dL per IU/kg | 3.00 | (2.71, 3.31) |

All data are the geometric mean (95% confidence interval) and based on the aPTT-based OSC assay.
AUC₀₋ₜₐᵤ, area under the activity-time curve over the dosing interval; CLₛₛ, clearance at steady state; Cₘₐₓ, maximum activity; FVIII, factor VIII; IR, incremental recovery; OSC, one-stage clotting; SD, standard deviation; t_{1/2}, half-life.
^{a}Approximately Week 26 (included PK assessments starting at Days 183, 218, and 246).
^{b}Values in this table represent calculated pharmacokinetic parameters for the full 14-day sampling period.

Following efanesoctocog alfa injection, the median time above 10 IU/dL FVIII activity levels for 159 analyzed subjects was 185 hours (111 to 330) (median full range (min-max)). The median time above 40 IU/dL FVIII activity levels for 159 analyzed subjects was 85.1 hours (44.4 to 156) (median full range (min-max)).

To further evaluate the PK of efanesoctocog alfa in comparison to Eloctate®, sequential PK testing was performed in a subgroup of patients treated with efanesoctocog alfa. Efanesoctocog alfa demonstrated high sustained mean FVIII levels with post-injection values in the normal to near-normal range (>40 IU/dL) for 3 to 4 days, above 10 IU/dL for approximately 7 days (i.e., the end of the 50 IU/kg dosing interval), and reaching 3 IU/dL at 11.6 days and 1 IU/dL by day 15. In comparison, the sequential PK subgroup in the Eloctate® phase 3 study (n=28) showed mean FVIII levels above 1 IU/dL for 4.9 days (4.4-5.5) and above 3 IU/dL for 3.7 days (3.3-4.1) (95% CI) with a comparable dose.

### Safety in Phase 3 Study

Safety endpoints included the occurrence of adverse events (AEs) and serious adverse events (SAEs); the occurrence of clinically significant changes from baseline in physical examination, vital signs, and laboratory tests; development of inhibitors (neutralizing antibodies directed against factor VIII [FVIII]) as determined via the Nijmegen modified Bethesda assay; and the occurrence of embolic and thrombotic events.

All subjects were monitored for inhibitors to Factor VIII in the clinical program. Inhibitor development to FVIII was not detected in any study participants (0% [0, 0.02]) (overall incidence 0%, 95% CI 0.0 to 2.3) from the Phase 3 study with previously treated patients >12 years of age with severe hemophilia A.

During treatment (up to 49.64 weeks) in the combined Phase 3 study with previously treated patients >12 years of age with severe hemophilia A (Example 1) and study with previously treated children <12 years of age with severe hemophilia A (Example 3), 4/277 (2.2%) of patients receiving efanesoctocog alfa developed non-inhibitory anti-drug antibodies. No impact of ADAs on the FVIII activity-time profiles and PK exposure parameters was observed. No impact of ADAs with respect to bleeding episodes and in the pharmacodynamic response was noted.

An overview of Treatment-Emergent Adverse Events can be seen in Table 52. There were no reports of Grade 3 or higher (per Common Terminology Criteria for Adverse Events Version 5.0) allergic reaction or anaphylaxis, and no reports of embolic or thrombotic events. Eighteen TESAEs reported in 15 (9.4) participants. The most common Treatment-Emergent Serious Adverse Event (TESAE) was haemophilic arthropathy, reported in 2 (1.3) participants. All other TESAEs were reported in 1 (0.6) participant, each. Note that the single TEAE leading to death participant with Hepatitis C virus and metastatic pancreatic adenocarcinoma, which was assessed as not related to Efa treatment. The most common adverse events are shown in Table 53; the most common adverse event was headache (Table 53). Two subjects experienced adverse events that led to discontinuation of treatment (Table 54). Table 55 provides additional details on serious and non-serious adverse events according to treatment arm.

TABLE 52
Overview of Treatment-Emergent Adverse Events in Phase
3 Study and Safety Analysis Set of 242HA201/OBS16221

| | | | Treatment- | | |
|---|---|---|---|---|---|
| Treatment-Emergent | Phase 3 Study | | Emergent Adverse | 242HA201/OBS1622 | |
| Adverse Events | Overall | | Events (TEAEs), | Overall | |
| (TEAEs), n(%) | (n = 159) | | n(%) | (n = 51) | |
| Total number of TEAEs | 394 | | Total number of | 58 | |
| | | | TEAEs | | |
| Participants with | 123 | (77.4) | Participants with at | 29 | (56.9) |
| at least one TEAE | | | least one TEAE | | |
| Participants with | 8 | (5.0) | Participants with at | 0 | |
| at least one related TEAE | | | least one related | | |
| | | | TEAE | | |
| Participants with | 15 | (9.4) | Participants with at | 5 | (9.8) |
| at least one TESAE | | | least one TESAE | | |
| TEAEs leading to death* | 1 | (0.6) | TEAEs leading to | 1 | (2.0) |
| | | | death | | |
| TEAEs leading to treatment | 2 | (1.3) | TEAEs leading to | 1 | (2.0) |
| discontinuation | | | treatment | | |
| | | | discontinuation | | |

*Metastatic pancreactic carcinoma assessed as not related to Efa.

TABLE 53
Most Common (>3%) Treatment Emergent
Adverse Events in Phase 3 Study

| | Overall | |
|---|---|---|
| Preferred Term, n (%) | (n = 159) | |
| Headache | 32 | (20.1) |
| Arthralgia | 26 | (16.4) |
| Fall | 10 | (6.3) |
| Back Pain | 9 | (5.7) |
| COVID-19 | 7 | (4.4) |
| Fatigue | 7 | (4.4) |
| Contusion | 6 | (3.8) |
| Haemophilic arthropathy | 6 | (3.8) |
| Nasopharyngitis | 6 | (3.8) |
| Joint Injury | 5 | (3.1) |
| Pain in extremity | 5 | (3.1) |
| Toothache | 5 | (3.1) |

TABLE 54
Treatment-Emergent Adverse Events Leading to
Treatment Discontinuation in Phase 3 Study

| Treatment-Emergent Adverse | Overall |
|---|---|
| Events (TEAEs), n (%) | (n = 159) |
| Total number of TEAEs | 2 |
| Participants with at least one TEAE | 2 (1.3) |
| CD4 lymphocytes decreased | 1 (0.6) |
| Combined tibia-fibula fracture | 1 (0.6) |

TABLE 55
Serious and non-serious adverse events, sorted by treatment arm.

| | | | |
|---|---|---|---|
| Arm A: | Arm B: | Arm B: | |
| Prophylaxis | On-demand | Prophylaxis | Overall |

Serious adverse events

| | | | | |
|---|---|---|---|---|
| Total subjects affected by serious adverse events | | | | |
| subjects affected/exposed | 13/133 | 2/26 | 0/26 | 15/159 |
| | (9.77%) | (7.69%) | (0.00%) | (9.43%) |
| number of deaths (all causes) | 0 | 1 | 0 | 1 |
| number of deaths resulting from adverse | | | | |
| events | | | | |
| Injury, poisoning and procedural complications | | | | |
| Combined Tibia-Fibula Fracture | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Traumatic Haemorrhage | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Surgical and medical procedures | | | | |
| Central Venous Catheter Removal | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Investigations | | | | |
| Blood Glucose Increased | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Cd4 Lymphocytes Decreased | | | | |
| subjects affected/exposed | 1/133 | 0/26 | 0/26 | 1/159 |
| | (0.75%) | (0.00%) | (0.00%) | (0.63%) |
| occurrences causally related to treatment/all | 1/1 | 0/0 | 0/0 | 1/1 |
| deaths causally related to treatment/all | 0/0 | 0/0 | 0/0 | 0/0 |
| Neoplasms benign, malignant and | | | | |
| unspecified (incl cysts and polyps) | | | | |
| Basal Cell Carcinoma | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Pancreatic Carcinoma Metastatic | | | | |
| subjects affected/exposed | 0/133 | 1/26 | 0/26 | 1/159 |
| | (0.00%) | (3.85%) | (0.00%) | (0.63%) |
| occurrences causally related to treatment/all | 0/0 | 0/1 | 0/0 | 0/1 |
| deaths causally related to treatment/all | 0/0 | 0/1 | 0/0 | 0/1 |
| Cardiac disorders | | | | |
| Angina Pectoris | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Nervous system disorders | | | | |
| Status Epilepticus | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/2 | 0/0 | 0/0 | 0/2 |
| deaths causally related to treatment/all | 0/0 | 0/0 | 0/0 | 0/0 |
| Cubital Tunnel Syndrome | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Ulnar Tunnel Syndrome | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Product issues | | | | |
| Device Breakage | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Musculoskeletal and connective tissue disorders | | | | |
| Arthropathy | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Mobility Decreased | | | | |
| subjects affected/exposed | 1/133 | 0/26 | 0/26 | 1/159 |
| | (0.75%) | (0.00%) | (0.00%) | (0.63%) |
| occurrences causally related to treatment/all | 0/1 | 0/0 | 0/0 | 0/1 |
| deaths causally related to treatment/all | 0/0 | 0/0 | 0/0 | 0/0 |
| Haemophilic Arthropathy | | | | |
| subjects affected/exposed | 2/133 | 0/26 | 0/26 | 2/159 |
| | (1.50%) | (0.00%) | (0.00%) | (1.26%) |
| occurrences causally related to treatment/all | 0/2 | 0/0 | 0/0 | 0/2 |
| deaths causally related to treatment/all | 0/0 | 0/0 | 0/0 | 0/0 |
| Infections and infestations | | | | |
| Covid-19 Pneumonia | | | | |
| subjects affected/exposed | 0/133 | 1/26 | 0/26 | 1/159 |
| | (0.00%) | (3.85%) | (0.00%) | (0.63%) |
| occurrences causally related to treatment/all | 0/0 | 0/1 | 0/0 | 0/1 |
| deaths causally related to treatment/all | 0/0 | 0/0 | 0/0 | 0/0 |

Non-serious adverse events
(frequency threshold for reporting non-serious adverse events: 5%)

| | | | | |
|---|---|---|---|---|
| Total subjects affected by non serious | | | | |
| adverse events | | | | |
| subjects affected/exposed | 56/133 | 7/26 | 1/26 | 64/159 |
| | (42.11%) | (26.92%) | (3.85%) | (40.25%) |
| Injury, poisoning and procedural complications | | | | |
| Fall | | | | |
| subjects affected/exposed | 10/133 | 0/26 | 0/26 | 10/159 |
| | (7.52%) | (0.00%) | (0.00%) | (6.29%) |
| occurrences all number | 11 | 0 | 0 | 11 |
| Nervous system disorders | | | | |
| Headache | | | | |
| subjects affected/exposed | 26/133 | 5/26 | 1/26 | 32/159 |
| | (19.55%) | (19.23%) | (3.85%) | (20.13%) |
| occurrences all number | 40 | 5 | 1 | 46 |
| General disorders and administration | | | | |
| site conditions | | | | |
| Fatigue | | | | |
| subjects affected/exposed | 7/133 | 0/26 | 0/26 | 7/159 |
| | (5.26%) | (0.00%) | (0.00%) | (4.40%) |
| occurrences all number | 8 | 0 | 0 | 8 |
| Musculoskeletal and connective tissue disorders | | | | |
| Arthralgia | | | | |
| subjects affected/exposed | 25/133 | 1/26 | 0/26 | 26/159 |
| | (18.80%) | (3.85%) | (0.00%) | (16.35%) |
| occurrences all number | 31 | 1 | 0 | 32 |
| Back Pain | | | | |
| subjects affected/exposed | 8/133 | 1/26 | 0/26 | 9/159 |
| | (6.02%) | (3.85%) | (0.00%) | (5.66%) |
| occurrences all number | 9 | 1 | 0 | 10 |

### Total Annualized FVIII Consumption in Observational Study 242HA201/OBS16221

Total annualized FVIII consumption was measured and analyzed as part of the observational study 242HA201/OBS16221. Overall, the historical mean (SD) annualized FVIII consumption was 4172.60 IU/kg (3003.64), with a mean (SD) of 4605.19 IU/kg (2924.86) for subjects on prophylaxis and 904.43 IU/kg (545.13) for subjects treated on-demand during observational study 242HA201/OBS16221. The population-level mean for subjects on prophylaxis was 4439.2 IU/kg and for subjects treated on-demand was 925.9 IU/kg.

Total Annualized FVIII Consumption in Phase 3 study

Total annualized efanesoctocog alfa consumption (in IU/kg) was also measured and analyzed as part of the secondary objectives of the Phase 3 study. Total annualised efanesoctocog alfa consumption (in IU/kg) was calculated for each subject as: Total IU/kg of efanesoctocog alfa during EP divided by total number of days during EP* 365.25. Efficacy period reflects the sum of all intervals of time during which subjects were treated with efanesoctocog alfa according to the study arms and treatment regimens. For subjects in Arm A: Prophylaxis (n=133), the median (full range ((min-max)) was 2756.99 (2385.1 to 50171.7). For subjects in Arm B: On-Demand (n=26), the median (full range ((min-max)) was 1212.27 (435.9 to 2023.8). For subjects in Arm B: Prophylaxis (n=6), the median (full range ((min-max)) was 2737.53 (2486.4 to 2924.8).

### Total Injections to Resolve Bleeding Episodes in Observational Study 242HA201/OBS16221

The number of injections required to resolve a bleeding episode was measured and analyzed as part of the secondary objectives of the study. When analyzed per bleeding episode in the historical analysis of observational study 242HA201/OBS16221, the overall total number of bleeding episodes was 978, with 536 bleeding episodes in subjects on prophylaxis and 442 bleeding episodes in subjects treated on-demand (see Table 56). The mean (SD) number of injections required to resolve one bleeding episode were 1.9 (5.1) in subjects on prophylaxis (range: 1 to 71); and 1.3 (0.6) in subjects treated on-demand (range: 1 to 5). Majority of the bleeding episodes in both treatment regimens required 1 injection for resolution (414/536 bleeding episodes [77.2%] in subjects on prophylaxis and 360/442 bleeding episodes [81.4%] in subjects treated on-demand).

### Total Injections to Resolve Bleeding Episodes in Phase 3 Study

A single 50 IU/kg dose of efanesoctocog alfa effectively treated 97% of bleeding episodes regardless of bleed type and location in participants receiving prophylaxis or on-demand treatment.

The overall total number of bleeding episodes in the efanesoctocog alfa trial was 362, with 86 bleeding episodes in subjects on prophylaxis in Arm A (n=133), 268 bleeding episodes in subjects treated on-demand in Arm B (n=26), and 8 bleeding episodes in subjects on prophylaxis in Arm B (n=26). Of the bleeding episodes in Arm A, 33 (38%) were spontaneous, 45 (52%) traumatic, and 8 (9%) of unknown type. For the bleeding episodes in the on-demand group of Arm B, 197 (74%) were spontaneous, 62 (23%) traumatic, and 9 (3%) of unknown type. For the bleeding episodes in the prophylaxis group of Arm B, 5 (63%) were spontaneous, 2 (25%) traumatic, and 1 (13%) of unknown type. The mean (SD) number of injections required to resolve one bleeding episode were 1.1 (0.3) in subjects on prophylaxis in Arm A, 1.0 (0.2) in subjects treated on-demand in Arm B, 1.0 (0.0) in subjects on prophylaxis in Arm B (Table 56). A summary of efanesoctocog alfa injections and exposure days (EDs) (Safety Analysis Set) is seen in Table 57.

For subjects receiving efanesoctocog alfa, the total dose (IU/kg) used to resolve each bleeding episode was averaged across all bleeding episodes per subject. Results are shown in Tables 56-57.

TABLE 56
Summary of number of injections required for resolution of a bleeding
episode in Observational Study 242HA201/OBS16221 and Phase 3 Study

| | | | | | | | Efanesoctocog alfa Treatment | | | | | | | |
|---|---|---|---|---|---|---|---|---|---|---|---|---|---|---|
| | Historical Treatment 242HA201/OBS16221 | | | | | | Arm A | | Arm B | | | | | |
| | On-demand | | Prophylaxis | | Overall | | Prophylaxis | | On-demand | | Prophylaxis | | Overall | |
| Number of patients | 19 | | 139 | | 157 | | 133 | | 26 | | 26 | | 159 | |
| with an observational/ | | | | | | | | | | | | | | |
| efficacy period | | | | | | | | | | | | | | |
| Per bleeding Episode | | | | | | | | | | | | | | |
| Number | 442 | | 536 | | 978 | | 86 | | 268 | | 8 | | 362 | |
| Mean (SD) | 1.3 | (0.6) | 1.9 | (5.1) | 1.6 | (3.8) | 1.1 | (0.3) | 1.0 | (2) | 1.0 | (0.0) | 1.0 | (0.2) |
| Median | 1.0 | | 1.0 | | 1.0 | | 1.0 | | 1.0 | | 1.0 | | 1.0 | |
| Q1; Q3 | 1.0; 1.0 | | 1.0; 1.0 | | 1.0; 1.0 | | 1.0; 1.0 | | 1.0; 1.0 | | 1.0; 1.0 | | 1.0; 1.0 | |
| Min; Max | 1; 5 | | 1; 71 | | 1; 71 | | 1; 3 | | 1; 2 | | 1; 1 | | 1; 3 | |
| 1 (%) | 360 | (81.4) | 414 | (77.2) | 774 | (79.1) | 81 | (94.2) | 261 | (97.4) | 8 | (100) | 350 | (96.7) |

| | | | | | | | | | | | | | |
|---|---|---|---|---|---|---|---|---|---|---|---|---|---|
| 2 (%) | 61 | (13.8) | 69 | (12.9) | 130 | (13.3) | 4 | (4.7) | 7 | (2.6) | 0 | 11 | (3.0) |

| | | | | | | | | | | | | |
|---|---|---|---|---|---|---|---|---|---|---|---|---|
| 3 (%) | 16 | (3.6) | 26 | (4.9) | 42 | (4.3) | 1 | (1.2) | 0 | 0 | 1 | (0.3) |

| | | | | | | | | | | |
|---|---|---|---|---|---|---|---|---|---|---|
| 4 (%) | 2 | (0.5) | 10 | (1.9) | 12 | (1.2) | 0 | 0 | 0 | 0 |
| >4 (%) | 3 | (0.7) | 17 | (3.2) | 20 | (2.0) | 0 | 0 | 0 | 0 |

TABLE 57
Summary of efanesoctocog alfa injections and exposure days (EDs) - Safety Analysis Set

| | Arm A | | Arm B | | | | | |
|---|---|---|---|---|---|---|---|---|
| | Prophylaxis | | On-demand | | Prophylaxis | | Overall | |
| | (N = 133) | | (N = 26) | | (N = 26) | | (N = 159) | |
| Total exposure days (EDs)a, n (%) | | | | | | | | |
| <5 | 2 | (1.5) | 0 | | 1 | (3.8) | 2 | (1.3) |
| 5-<10 | 0 | | 7 | (26.9) | 0 | | 0 | |
| 10-<25 | 3 | (2.3) | 19 | (73.1) | 8 | (30.8) | 5 | (3.1) |
| 25-<50 | 13 | (9.8) | 0 | | 17 | (65.4) | 37 | (23.3) |
| >=50 | 115 | (86.5) | 0 | | 0 | | 115 | (72.3) |
| At least 5 exposure days | 131 | (98.5) | 26 | (100) | 25 | (96.2) | 157 | (98.7) |
| At least 10 exposure days | 131 | (98.5) | 19 | (73.1) | 25 | (96.2) | 157 | (98.7) |
| At least 25 exposure days | 128 | (96.2) | 0 | | 17 | (65.4) | 152 | (95.6) |
| At least 50 exposure days | 115 | (86.5) | 0 | | 0 | | 115 | (72.3) |
| Number | 133 | | 26 | | 26 | | 159 | |
| Mean (SD) | 50.9 | (9.1) | 11.9 | (4.3) | 23.8 | (6.1) | 48.4 | (10.5) |
| Median | 53.0 | | 12.0 | | 26.0 | | 53.0 | |
| Min; Max | 2; 63 | | 5; 21 | | 2; 28 | | 2; 63 | |
| Total number of injections per | | | | | | | | |
| participant | | | | | | | | |
| Overall | | | | | | | | |
| Number | 133 | | 26 | | 26 | | 159 | |
| Mean (SD) | 51.1 | (9.2) | 12.0 | (4.2) | 23.9 | (6.0) | 48.6 | (10.6) |
| Median | 53.0 | | 12.0 | | 26.0 | | 53.0 | |
| Min; Max | 2; 63 | | 5; 21 | | 2:28 | | 2; 63 | |
| For Prophylaxis b | | | | | | | | |
| Number | 133 | | 26 | | 26 | | 159 | |
| Mean (SD) | 50.0 | (9.0) | 1.0 | (0.0) | 22.2 | (7.9) | 45.6 | (13.3) |
| Median | 52.0 | | 1.0 | | 25.5 | | 52.0 | |
| Min; Max | 2; 62 | | 1; 1 | | 1; 28 | | 2; 62 | |
| For Spontaneous bleed | | | | | | | | |
| Number | 133 | | 26 | | 26 | | 159 | |
| Mean (SD) | 0.3 | (0.7) | 7.7 | (4.3) | 0.2 | (0.5) | 1.5 | (3.4) |
| Median | 0.0 | | 8.0 | | 0.0 | | 0.0 | |
| Min; Max | 0; 5 | | 1; 15 | | 0; 2 | | 0; 17 | |
| For Traumatic bleed | | | | | | | | |
| Number | 133 | | 26 | | 26 | | 159 | |
| Mean (SD) | 0.3 | (0.7) | 2.4 | (3.4) | 0.1 | (0.4) | 0.7 | (1.7) |
| Median | 0.0 | | 2.0 | | 0.0 | | 0.0 | |
| Min; Max | 0; 4 | | 0; 15 | | 0; 2 | | 0; 15 | |
| For Follow-up injection after | | | | | | | | |
| initial treatment of a bleed | | | | | | | | |
| Number | 133 | | 26 | | 26 | | 159 | |
| Mean (SD) | 0.1 | (0.7) | 0.6 | (1.1) | 0.0 | (0.2) | 0.2 | (0.8) |
| Median | 0.0 | | 0.0 | | 0.0 | | 0.0 | |
| Min; Max | 0; 8 | | 0; 4 | | 0; 1 | | 0; 8 | |
| For Surgical | | | | | | | | |
| Number | 133 | | 26 | | 26 | | 159 | |
| Mean (SD) | 0.3 | (1.1) | 0.2 | (0.6) | 0.5 | (2.4) | 0.4 | (1.5) |
| Median | 0.0 | | 0.0 | | 0.0 | | 0.0 | |
| Min; Max | 0; 9 | | 0; 3 | | 0; 12 | | 0; 15 | |
| For Other | | | | | | | | |
| Number | 133 | | 26 | | 26 | | 159 | |
| Mean (SD) | 0.1 | (0.3) | 0.2 | (0.5) | 0.8 | (2.8) | 0.2 | (1.2) |

| Median | 0.0 | 0.0 | 0.0 | 0.0 |
|---|---|---|---|---|
| Min; Max | 0; 2 | 0; 2 | 0; 13 | 0; 13 |

Note:
1: Percentages are based on the number of participants in the Safety Analysis Set.
2: Participants are included in each study arm and treatment regimen they participated in for the duration of time on that regimen and, as such, may appear in more than one treatment regimen. Each participant is counted only once in the overall column.
aAn exposure day is a 24-hour period in which one or more BIVV001 injections are given. All injections over the study course are counted. The count from the overall column is the summation of exposure days from Arm A and Arm B overall i.e., the total exposure days are based on the final treatment regimen that patients arrive at, thus, the overall count might not be equal to the summation of the count from columns of treatment arm and regimen.
b Baseline injection for PK is counted in prophylaxis block.

### Surgeries

Thirteen patients underwent 14 major surgeries during the study. A major surgery is defined as any invasive operative procedure that required opening into a major body cavity (e.g., abdomen, thorax, skull), operation on a joint, removal of an organ, dental extraction of any molar teeth or >3 nonmolar teeth, operative alteration of normal anatomy, or crossing of a mesenchymal barrier (e.g., pleura, peritoneum, dura).

Two major surgeries took place after the last dose of efanesoctocog alfa and were not assessed. For the 12 evaluable surgeries (6 orthopedic; 6 nonorthopedic), all hemostatic responses to efanesoctocog alfa were deemed excellent by the investigator or surgeon (Table 58). Investigator's/Surgeon's assessment of the patient's hemostatic response to efanesoctocog alfa treatment was collected 24 hours post-surgery based on the ISTH 4-point response scale of excellent, good, fair, and poor. Total perioperative management data can be seen in Table 59.

Of the 13 subjects in the surgery subgroup, 11 required a single preoperative injection (day −1 or day 0) of a median (range) dose of 49.9 (12.7-51.7) IU/kg efanesoctocog alfa to maintain hemostasis during the perioperative period for major surgery. Median number of injections post-surgery was 1.0 for Day 1-3 and 2.0 for Day 4-14. Median (range) consumption was 31.8 (24.3-103.0) IU/kg for Day 1-3 and 103.20 (95.5-206.1) IU/kg for Day 4-14. Median (range) number of injections was 1.0 (1-2) for Day 1-3 and 2.0 (2-4) for Day 4-14 (n=11). The mean (standard deviation [SD]) estimated blood loss was 143.3 (189.4) mL during major surgery. Median (range) estimated blood loss was 75 (0-500) mL during surgery (n=6). The mean estimated postoperative blood loss (ie, from the day following the end of surgery to the date of hospital discharge) was 65.6 (132.0) mL; median (range) estimated blood loss was 0 (0-400) mL (n=9). Three bleeding episodes occurred, all on Day-1 to Day 0 (the day of surgery); 1 occurred post-surgery and 2 occurred at the time of surgery. No patients required blood transfusions during the surgical period.

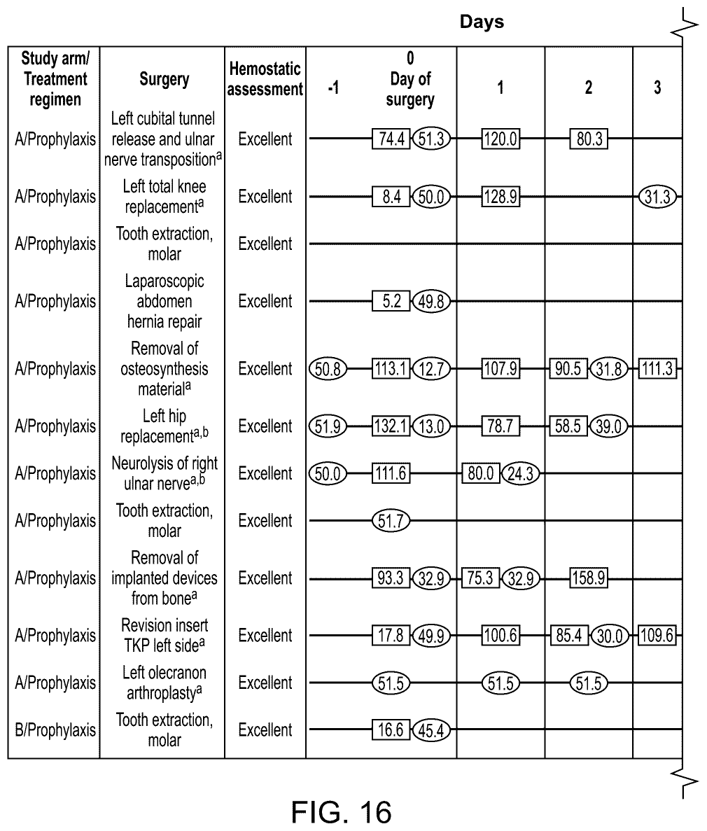

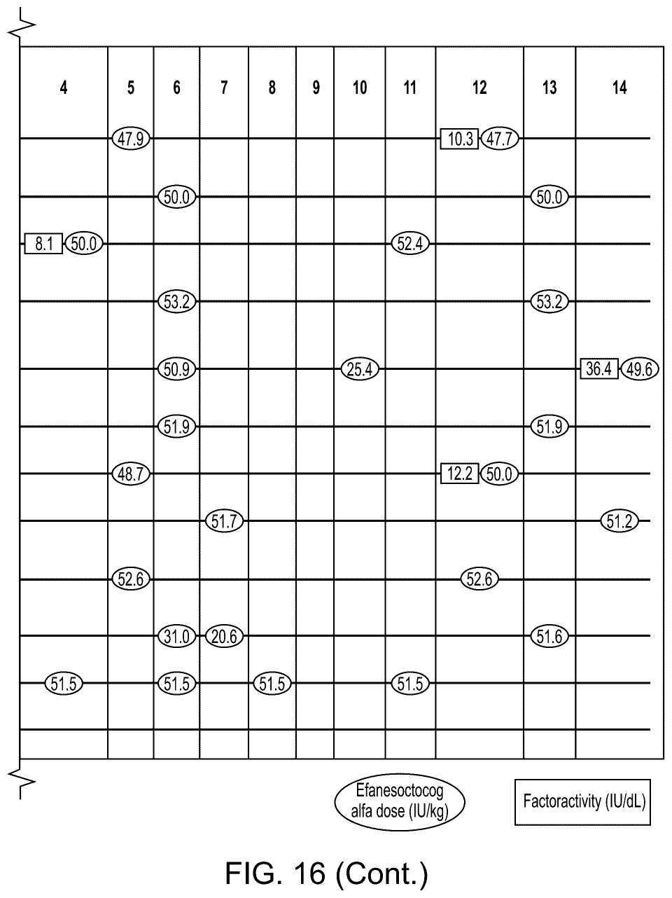

Details of the perioperative management of each surgery during the study are provided in FIG. 16.

Three treatment-emergent adverse events (TEAEs) were reported during a major surgical/rehabilitation period, which were all in 1 patient and included infusion site rash, tooth fracture, and synovial disorder. All TEAE were classified as non-serious, mild in severity, and not related to efanesoctocog alfa treatment. No thromboembolic complications or inhibitor development were reported.

For the 6 orthopedic surgeries, surgical procedures were total knee replacement; hip replacement; elbow arthroplasty; revision insert total knee prosthesis; removal of implanted device from bone; and removal of osteosynthesis material. The average dose they received on the day of surgery was 41 IU/kg. For these orthopedic surgeries, from Day 1 to Day 14, 3 patients required 3 further injections, 2 patients had 4 further injections, 1 patient required >4 further injections, and median total consumption was 140.5 IU/kg. Blood loss was 10-500 mL during surgery and 0-400 mL in the postoperative period, which is similar to what would be expected for a patient without hemophilia undergoing a similar operation. In this series of 6 major orthopedic surgeries in patients with severe hemophilia A, a single injection of efanesoctocog alfa was sufficient to maintain hemostasis during surgery. Hemostatic response was rated by physicians as excellent for all surgeries; consumption and number of injections of efanesoctocog alfa during the surgical/rehabilitation period were low.

For minor surgeries, fifteen participants underwent 18 minor surgeries while receiving efanesoctocog alfa. Thirteen minor surgeries in 12 participants required 1 dose of efanesoctocog alfa on the day of surgery, with a median (range) dose on the day of minor surgery of 51.2 (48-52) IU/kg. Five minor dental surgeries required no preoperative dose of efanesoctocog alfa. Hemostatic response was rated excellent for all minor surgeries where assessed (n=13). Four TEAEs were reported during a minor surgical/rehabilitation period, of which 1 was serious. No thromboembolic complications or inhibitor development were reported for minor surgeries.

TABLE 58
Summary of investigators'/surgeons' assessment of participants'
hemostatic response to Efa (BIVV001) treatment - Surgery Subgroup

| | Surgery subgroup | |
|---|---|---|
| | (N = 13) | |
| Number of major surgeries | 12 | |
| Assessment of response, n (%) | | |
| Excellent or Good | 12 | (100) |
| Excellent (=1) | 12 | (100) |
| Good (=2) | 0 | |
| Fair (=3) | 0 | |
| Poor/none (=4) | 0 | |
| Number | 12 | |
| Mean (SD) | 1.0 | (0.0) |

| Median | 1.0 |
|---|---|
| Q1; Q3 | 1.0; 1.0 |
| Min; Max | 1; 1 |

Note:
1: Percentages are based on the number of major surgeries with assessments.:
2: The analysis in the Table body is based on the major surgeries conducted during the treatment regimen, excluding the surgeries conducted after the last BIVV001 dosing. Those excluded major surgeries are counted in the capital N in the header.

TABLE 59
Perioperative management data.

| | Surgery subgroup | |
|---|---|---|
| Perioperative management | (N = 13)^{e} | |
| Number (%) of major surgeries with hemostatic | 12 | (100%) |
| response rated as excellent or good by the | | |
| Investigator/Surgeon | | |
| Mean (SD) number of BIVV001 injections to | 1.0 | (0.0) |
| maintain hemostasis per major surgery ^{g} | | |
| Mean dose of BIVV001 to maintain hemostasis | 41.65 | (15.21) |
| during major surgery (IU/kg) | | |
| Total BIVV001 consumption (IU/kg) ^{h}: | | |
| Day −1 to Day 0 (day of surgery) | 41.65 | (15.21) |
| Day 1 to Day 3 post surgery | 41.75 | (27.36) |
| Day 4 to Day 14 post surgery | 113.65 | (31.63) |
| Day −1 to Day 14 post surgery | 166.71 | (72.48) |
| Number of blood component transfusions | 0 | |
| per major surgery | | |
| Mean (SD) estimated blood loss during | 143.33 | (189.38) |
| major surgery (mL) | | |

AsBR: annualized spontaneous bleeding rate;
AJBR: annualized joint bleeding rate;
B: baseline
a Estimated using a negative binomial model with the total number of treated bleeding episodes during the efficacy period as the response variable and log-transformed efficacy period duration (in years) as an offset variable.
b Estimated using a repeated negative binomial model with treatment (BIVV001 prophylaxis vs historical prophylaxis) as covariate.
c Achieving trough FVIII activity levels above x % are based on average trough samples from each scheduled visit using the aPTT-based one-stage clotting assay. Participants with trough samples outside 168 +/− 5 hours from previous dose are excluded from this analysis.
d A target joint at baseline is defined as a major joint with ≥3 spontaneous bleeds in a consecutive 6-month period prior to study entry
^{e}Analysis is based on the major surgeries conducted during the treatment regimen, excluding the surgeries conducted after the last BIVV001 dosing. Those excluded major surgeries are counted in the capital N in the header.
f A target joint resolved is defined as ≤2 bleeds (regardless of type) into that joint during 12 months of continuous exposure. Percentage is calculated out of Participants with at least 12 months continuous exposure.
^{g} The number of injections to maintain hemostasis during surgery includes all injections, from loading dose (i.e., the preoperative injection, administered either on the day of surgery or one day prior to the surgery), to the end of surgery.
^{h} Day 0 is defined as the surgery day. The loading dose for a given surgery is the preoperative injection, administered either on the day of surgery or one day prior to the surgery (i.e., Day −1).

### Japanese Participants

Twelve Japanese males were enrolled in Arm A. For these participants, the mean±SD ABR was 0.51±0.54 (median [IQR]: 0.51 [0.00-1.02]). Switching from prestudy standard-of-care FVIII prophylaxis to efanesoctocog alfa prophylaxis decreased mean (SD) ABR from 1.36±2.43 to 0.56±0.54 (n=11). All bleeds (6) resolved with 1 efanesoctocog alfa injection (50 IU/kg). Once-weekly efanesoctocog alfa provided FVIII activity in the normal to near-normal range (>40%) for most of the week and mean activity of 13.4% on day 7 (n=3; Week 1). From baseline to Week 52, there were improvements in Haem-A-QoL PH score (mean±SD score change: −9.2±20.2), PROMIS-Short Form (SF) v1.0 pain intensity 3a first item score (−0.2±0.6), and HJHS joint health score (−4.1±7.6). There were no treatment-emergent serious adverse events and inhibitor development was not detected.

### M. Conclusions

This Phase 3 study met the primary endpoint, showing a clinically meaningful prevention of bleeds in people with severe hemophilia A receiving weekly prophylaxis with efanesoctocog alfa over a period of 52 weeks. The median and mean annualized bleeding rates (ABR) were 0.00 (IQR: 0.00-1.04) and 0.71 (SD: 1.43) respectively. The study also met the key secondary endpoint, demonstrating superior bleed protection (p<0.0001) over prior factor VIII prophylaxis with an estimated ABR reduction of 77% and a mean ABR of 0.69 compared to 2.96 on prior prophylaxis, based on an intra-patient comparison (n=78). In a subset of participants (n=17) studied at baseline and week 26, mean factor VIII levels remained in the normal to near-normal range (>40 IU/dL) for the majority of the week, and at 15 IU/dL at Day seven post-dose, providing increased factor activity level protection for patients with once-weekly prophylaxis. Data show adults and adolescents treated with once-weekly efanesoctocog alfa experienced statistically significant and clinically meaningful improvements in physical health (p=0.0001), pain intensity (p=0.0276), and joint health (p=0.0101) when comparing week 52 and baseline measurements (physical health was assessed with the Haem-A-QoL Physical Health score; pain intensity was assessed using the PROMIS Pain Intensity 3a past 7 days intensity of pain at its worst score; joint health was assessed using the Hemophilia Joint Health score). Moreover, efanesoctocog alfa was effective at treating bleeds, including in target joints; 96.7% of bleeds were resolved with a single 50 IU/kg dose. Efanesoctocog alfa was well-tolerated, and inhibitor development to factor VIII was not detected. The most common treatment emergent adverse events (>5% of participants overall) were headache, arthralgia, fall, and back pain.

Up to 46% of people with hemophilia report living with chronic pain (Paredes et al. (2021) J Pain; 22:1134-4), which can negatively impact physical and psychological health (Stromer et al. (2021) Wien Klin Wochenschr 2021; 133:1042-56). Prophylaxis with efanesoctocog alfa significantly improved physical health (Haem-A-QoL) and pain (PROMIS) in patients who were on prestudy standard-of-care FVIII prophylaxis. Also, the percentage of patients reporting no use of pain medication during the prior 14 days increased during the study. Collectively, these results suggest prophylaxis with efanesoctocog alfa has the potential to provide meaningful improvements in physical health and pain for people with severe hemophilia A. In addition, efanesoctocog alfa prophylaxis positively impacted joint health, as evidenced by the complete resolution of target joints, absence of joint bleeds in 72% (Arm A) and 81% (Arm B) of patients during prophylaxis, and a modest but significant improvement in HJHS score in patients on prior FVIII prophylaxis.

Efanesoctocog alfa (50 IU/kg) had a long mean half-life of approximately 47 hours and maintained mean FVIII levels in the normal to near-normal range (>40 IU/dL) for 4 days and at 15 IU/dL by the end of the 7-day dosing interval, based on the aPTT-based one-stage clotting (OSC) assay. In addition to maintaining high sustained factor levels, uncoupling rFVIII from VWF may also reduce interpatient PK variability of efanesoctocog alfa and increase the predictability of the FVIII profile overtime, as circulating VWF levels vary widely between people, as well as within the same subject (Turecek et al. (2020) Haemophilia 26:575-83). This is supported by a recent Phase 1 sequential pharmacokinetic study, showing a coefficient of variation for clearance of 45% and 33% for octocog alfa and rurioctocog alfa pegol, respectively, and 19% for efanesoctocog alfa (Lissitchkov T. (2022) WFH Abstract). The low variability in efanesoctocog alfa clearance allows the use of a standard weekly dose of 50 IU/kg for prophylaxis and reduces the need for monitoring of FVIII activity levels for individualized PK-based prophylaxis. Decoupling rFVIII from VWF does not adversely affect clot formation, as demonstrated by the efficacy results in the current study, and by previous in vitro and in vivo results (Demers et al. (2022) J Thromb Haemost (first published Apr. 25, 2022)).

Collectively, these results show that by maintaining high sustained factor activity, once-weekly efanesoctocog alfa provides substantial improvements in clinical outcomes and QoL for patients with severe hemophilia A. These positive data show a very low annualized bleeding rate, and support efanesoctocog alfa's potential to transform hemophilia A therapy. Efanesoctocog alfa provides higher protection for longer duration with reduced treatment burden of once-weekly dosing.

### SUMMARY

Efficacy: Prophylaxis treatment with efanesoctocog alfa demonstrated clinically meaningful prevention of bleeding events. The primary endpoint of the clinical study was therefore met. Prophylaxis treatment with efanesoctocog alfa demonstrated superior improvement in bleed prevention vs prior FVIII replacement therapy, thus meeting a key secondary endpoint of the clinical study. Results underscore the ability of efanesoctocog alfa to sustain normal to near-normal factor levels and the potential to transform prophylactic treatment, providing people with hemophilia A with higher protection for longer. Once-weekly prophylaxis with efanesoctocog alfa (50 IU/kg) provided highly effective prevention of bleeds and superior bleed protection compared to prestudy standard-of-care FVIII prophylaxis. 80% (Arm A) and 85% (Arm B) of patients experienced zero spontaneous bleeds during the prophylaxis treatment periods. When bleeding events did occur, a single injection of 50 IU/kg efanesoctocog alfa provided effective resolution for about 97% of bleeds.

Efanesoctocog alfa prophylaxis demonstrated significant improvement from baseline in physical health, pain intensity, and joint health outcomes. Data also showed efanesoctocog alfa prophylaxis resulted in statistically significant and clinically meaningful improvements in physical health, pain intensity and joint health in patients on prior factor VIII prophylaxis.

Pharmacokinetics: Once weekly efanesoctocog alfa treatment (50 IU/kg) provided high sustained FVIII activity levels in the normal to near-normal (>40%) range for up to 4 days and average activity levels >10% at Day 7 post-injection.

Safety: Efanesoctocog alfa was well-tolerated. Reported treatment-emergent adverse events were generally consistent with what is anticipated in an adult and adolescent population with severe hemophilia A. Inhibitor development to FVIII was not detected and there were no reports of serious allergic reaction, anaphylaxis or thromboembolic events. Inhibitor development to FVIII was not detected, and there were no reports of serious allergic reactions, anaphylaxis, or vascular thrombotic events.

### Example 2: A Phase 1, Single-Site, Open-Label Study to Assess Pharmacokinetics of Efanesoctocog Alfa, Standard Half-Life and Extended Half-Life FVIII after Each Single Intravenous Injection in a Fixed Sequence, in Previously Treated Adults with Severe Hemophilia a

Efanesoctocog alfa, also known as “BIW001”, “efanesoctocogum alfa”, “Efa”, and “rFVIIIFc-VWF-XTEN”, was designed to be a new class of blood clotting factor VIII (FVIII), engineered to have an extended half-life (t₂) that is independent of von Willebrand factor (VWF). This study was designed to further characterize the pharmacokinetics (PK) of efanesoctocog alfa and available standard half-life [Octocog Alfa; Advate® (SHL)] and extended half-life [Rurioctocog Alfa Pegol; Adynovi® (EHL)] recombinant FVIII (rFVIII) products at a dose of 50 IU/kg.

### A. Study Design

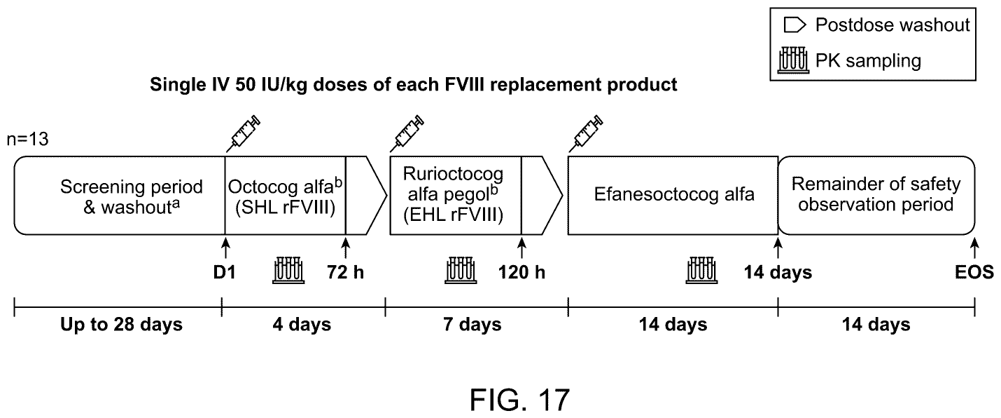

This study was a Phase 1, single-center, single-arm, non-randomized, open label, 3-period, fixed sequence, PK comparison study assessing the PK profiles of efanesoctocog alfa, Advate® (octocog alfa), and Adynovi® (rurioctocog alfa pegol) after a single IV injection in previously treated patients with severe hemophilia. A graphical representation of the design of this study is shown in FIG. 17.

The primary objective of the study was to assess the half-life of octocog alfa (SHL rFVIII), rurioctocog alfa pegol (EHL rFVIII) and efanesoctocog alfa after a single IV injection. The primary endpoint was the half-lives of the 3 FVIII products, as assessed by the 1-stage aPTT clotting assay.

Secondary objectives of the study included characterization of additional PK parameters of all FVIII 3 products after a single IV injection and evaluation of the safety and tolerability of efanesoctocog alfa after a single IV injection. Secondary endpoints were additional PK parameters (e.g. AUC_{0-inf}, Cₘₐₓ, CL, MRT, and IR), occurrence of AEs and SAEs, and assessment of clinically significant laboratory abnormalities, including FVIII inhibitor development.

### Number of Participants (Planned and Analyzed):

Approximately 12 participants were planned to be enrolled in order to have at least 10 to 12 hemophilia A patients who completed the study for PK analysis. In total, 14 participants were screened with 13 participants enrolled for treatment. Each of the 13 participants received all 3 three study interventions, were included in the safety population; and were included in the PK population.

### Statistical Analysis

Pharmacokinetic parameters (Cₘₐₓ, AUC, elimination half-life (t_{1/2}), CL, Vss, IR, and MRT) were computed and analyzed by a noncompartmental method using baseline corrected FVIII activity. FVIII activity was measured by the aPTT-based one-stage clotting (OSC) assay with Actin FSL as the activator. The assay was validated using plasma standard calibrated against World Health Organization (WHO) international FVIII standard. The lower limit of quantitation (LLOQ) was 0.5 IU/dL (Advate®), 0.6 IU/dL (Adynovi®) and 1 IU/dL (BIVV001).

Baseline corrected FVIII activity and pharmacokinetic parameters were summarized by study intervention group (treatment) using descriptive statistics. Log-transformed t_{1/2}, Cₘₐₓ, AUC, and CL were analyzed with a linear model with fixed terms for treatment and a random term for participant, using the Statistical Analysis Software (SAS) Proc Mixed® procedure. As an exploratory analysis, point estimates and 90% CIs for the geometric means ratio between treatments (efanesoctocog alfa versus Advate® and efanesoctocog alfa versus Adynovi®) were provided.

### B. Study Population

### Inclusion and Exclusion Criteria

Participants were male, 18 to 65 years of age (inclusive) at the time of informed consent, with documented severe hemophilia A (defined as <1 IU/dL [<1%] endogenous FVIII) with prior treatment, of at least 150 exposure days, with any recombinant and/or plasma-derived FVIII and/or cryoprecipitate products, and no history of a positive inhibitor test including at screening.

### Demographics

All participants were male and white and were 26 through 47 years of age (mean (SD) age: 36.8 (6.5) years). The mean (SD) body weight for participants at baseline was 87.77 (18.91) kg with a BMI of less than 25 kg/m² for 4 participants, between 25 and 30 kg/m² for 6 participants, and 30 kg/m² or more for 3 participants. Hemophilia history: participants had a mean (SD) age at diagnosis of severe hemophilia of 1.5 (1.3) years; median of 1.0 year; minimum of 0 years; and maximum of 5 years. The mean (SD) total reported number of bleeding events occurring in the 12 months prior to screening were 38.0 (25.8) events; median of 36.0 events; minimum of 2 events; and maximum 80 events. Of the 13 participants, 3 (23.1%) were on prophylactic therapy while most participants [10 (76.9%)] received “on-demand” treatments. The mean (SD) age since the start of prophylactic treatment (3 participants) was 23.7 (5.5) years; median of 21.0 years; minimum of 20 years; and maximum of 30 years. All 13 participants had a minimum of 150 prior exposure days (EDs) to FVIII products.

### C. Study Intervention, Dosing, and Intervention Information

Investigational medicinal product(s) used in this study were efanesoctocog alfa (BIVV001, rFVIIIFc-VWF-XTEN), SHL factor VIII (full length recombinant coagulation factor VIII, Advate®), and EHL factor VIII (PEGylated full length recombinant coagulation factor VIII, Adynovi®). Efanesoctocog alfa is formulated as lyophilized powder in a sterile vial that requires reconstitution with sterile water for injection (diluent). Advate® is formulated as lyophilized powder in single-use vials containing 1000 IU, and includes 2 mL of sterile water for injection. Adynovi® is formulated as lyophilized powder in single-use vials containing 1000 IU. The solvent vial contains 2 mL of sterile water for injections. All enrolled participants received single, sequential, intravenous dose of 50 IU/kg of Advate®, Adynovi®, and efanesoctocog alfa after appropriate washout periods.

### D. Safety

Safety analysis was based on the review of individual values and descriptive statistics. The safety analysis focused on the treatment emergent (TE) period, defined as the time from the first study intervention administration of a study period up to the next study period (excluded) or up to the end of study (EOS) visit (included) for the last study period. All AEs were coded according to the Medical Dictionary for Regulatory Activities version 24.1. The number (%) of participants experiencing TEAEs were summarized. Potentially clinically significant abnormalities (PCSAs) for laboratory and vital signs variables were flagged and summarized using frequency tables.

### E. Results

### Pharmacokinetic Results

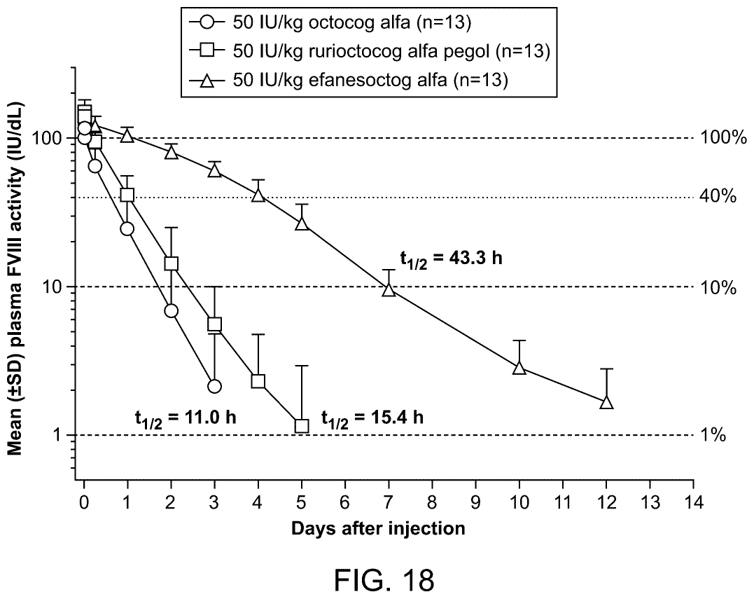

As shown in FIG. 18, efanesoctocog alfa (BIVV001) maintains mean FVIII activity level >40 IU/dL up to 96 hours (4 days,) whereas Advate® and Adynovi® maintain this up to 6 hours (<1 day) and up to 24 hours (1 day), respectively. Efanesoctocog alfa maintains mean FVIII activity level >10 IU/dL approximately up to 168 hours (7 days) whereas Advate® and Adynovi® maintain this up to 24 hours (1 day) and up to 48 hours (2 days), respectively. Efanesoctocog alfa maintains mean FVIII activity level >1 IU/dL up to 288 hours (12 days) whereas Advate® and Adynovi® maintain up to 72 hours (3 days) and up to 120 hours (5 days), respectively. Tables 60-61 show additional PK parameters for all 3 treatments tested.

Following a single 50 IU/kg IV dose of Advate®, Adynovi®, and efanesoctocog alfa, the median tₘₐₓ of the baseline-corrected FVIII activity generally occurred at the first collected sample (0.17 hours post-dose) across all participants and treatments (range 0.17 to 1.00 hours) (Table 60). The Cₘₐₓ and IR values indicated consistent recovery across the three FVIII products.

TABLE 60
Mean ± SD (geometric mean) [CV %] pharmacokinetic
parameters for baseline-corrected factor VIII activity

| | | | | | Efanesoctocog | |
|---|---|---|---|---|---|---|
| PK parameters | Advate | | Adynovi | | Alfa | |
| N | 13 | | 13 | | 13 | |
| IR | 2.37 ± 0.295 | | 3.02 ± 0.605 | | 2.80 ± 0.338 | |
| ((IU/dL)/(IU/kg)) | (2.36) | [12] | (2.96) | [20] | (2.78) | [12] |
| Cₘₐₓ | 119 ± 14.7 | | 151 ± 30.3 | | 140 ± 16.9 | |
| (IU/dL) | (118) | [12] | (148) | [20] | (139) | [12] |
| tₘₐₓ^{a} | 0.17 | | 0.17 | | 0.17 | |
| (h) | (0.17, 0.50) | | (0.17, 1.00) | | (0.17, 1.00) | |
| AUC | 1820 ± 748 | | 2950 ± 905 | | 10200 ± 1560 | |
| (IU h/dL) | (1670) | [41] | (2820) | [31] | (10100) | [15] |
| t_{1/2z} | 11.7 ± 4.55 | | 16.3 ± 5.63 | | 44.4 ± 10.4 | |
| (h) | (11.0) | [39] | (15.4) | [35] | (43.4) | [24] |
| CL | 3.26 ± 1.48 | | 1.86 ± 0.618 | | 0.503 ± 0.0974 | |
| (mL/(h*kg)) | (2.99) | [45] | (1.77) | [33] | (0.496) | [19] |
| Vss | 40.1 ± 6.82 | | 34.5 ± 6.60 | | 31.3 ± 3.44 | |
| (mL/kg) | (39.5) | [17] | (33.9) | [19] | (31.1) | [11] |
| MRT | 14.6 ± 6.76 | | 20.4 ± 7.73 | | 63.4 ± 9.41 | |
| (h) | (13.2) | [46] | (19.2) | [38] | (62.7) | [15] |

^{a}Median (Min, Max),
CV—coefficient of variation

A single 50 IU/kg dose of efanesoctocog alfa exhibited reduced CL (0.17- and 0.28-fold), which resulted in longer t_{1/2} (3.94- and 2.82-fold) and higher exposure (AUC, 6.03- and 3.57-fold) compared with Advate® and Adynovi®, respectively (Table 61).

TABLE 61
Comparison of Advate ®, Adynovi ®, and
Efanesoctocog alfa (BIVV001) PK parameters for baseline-
corrected FVIII activity following a single 50 IU/kg dose

| | | Point | |
|---|---|---|---|
| Parameter | Comparison/Treatment | Estimate | 90% CI |
| t_{1/2z} (h) | Efanesoctocog Alfa vs. | 3.94 | (3.47 to 4.48) |
| | Advate | | |
| | Efanesoctocog Alfa vs. | 2.82 | (2.48 to 3.20) |
| | Adynovi | | |
| Cₘₐₓ (IU/dL) | Efanesoctocog Alfa vs | 1.18 | (1.10 to 1.26) |
| | Advate | | |
| | Efanesoctocog Alfa vs. | 0.94 | (0.88 to 1.01) |
| | Adynovi | | |
| AUC (h*IU/dL) | Efanesoctocog Alfa vs | 6.03 | (5.32 to 6.83) |
| | Advate | | |
| | Efanesoctocog Alfa vs. | 3.57 | (3.15 to 4.05) |
| | Adynovi | | |
| CL (mL/h/kg) | Efanesoctocog Alfa vs | 0.17 | (0.15 to 0.19) |
| | Advate | | |
| | Efanesoctocog Alfa vs. | 0.28 | (0.25 to 0.32) |
| | Adynovi | | |

Abbreviations:
AUC = area under the activity-time curve;
CL = clearance;
Cmax = maximum concentration;
CI = confidence interval;
t_{1/2z} = terminal half-life
Point estimates and 90% CIs for the geometric means ratio of treatments (Efanesoctocog Alfa vs Advate, Efanesoctocog Alfa vs. Adynovi) were obtained by computing point estimates and 90% CIs for the differences between treatment means using a linear model with fixed term for treatment and a random term for participant using SAS Proc Mixed ®, and then converting to ratios by the antilog transformation.

### Safety Results

There were no TEAEs reported during the Advate® or Adynovi® treatment periods. Three TEAEs were reported in 1 (7.7%) participant during the efanesoctocog alfa treatment period and included headache, cough, and positive SARS-CoV-2 test. There were no serious or severe TEAEs reported and no TEAEs resulted in permanent study discontinuation. Inhibitor development to FVIII was not detected and there were no reports of serious hypersensitivity, anaphylaxis, or vascular thrombotic events.

Potentially clinically significant abnormalities (PCSAs) in laboratory or vital sign parameters were not identified during the Advate or Adynovi treatment periods. PCSAs were identified during the efanesoctocog alfa treatment period for white blood cell parameters of monocytes >0.7×10⁹/L and eosinophils >0.5×10⁹/L or >ULN (if ULN >0.5×10⁹/L) in a single participant. Both PCSAs were detected 14 days after administration of efanesoctocog alfa and resolved at the end of study visit, 28 days after administration of efanesoctocog alfa. There were no other PCSAs identified in any laboratory or vital sign parameter during the efanesoctocog alfa treatment period.

### F. Conclusions

Following a single 50 IU/kg IV dose, efanesoctocog alfa exhibited an approximately 4- to 3-fold longer mean t_{1/2} (44.4 hours; SD±10.4 hours) compared with Advate® (11.7 hours; SD±4.55 hours) and Adynovi® (16.3 hours; SD±5.63 hours), respectively, based on FVIII activity levels measured by a one-stage aPTT assay. Efanesoctocog alfa maintains mean FVIII activity level >40 IU/dL up to 4 days, whereas Advate® and Adynovi® maintain up to 1 day. Efanesoctocog alfa also maintains mean FVIII activity level >10 IU/dL approximately up to 7 days, whereas Advate® and Adynovi® maintain up to 2 days. In addition, single dose efanesoctocog alfa was well tolerated, and no safety concerns were identified, Efanesoctocog alfa maintained high sustained FVIII activity in the normal to near-normal range for a longer duration compared with two currently marketed FVIII therapies.

### Example 3: A Phase 3 Open-Label, Multicenter Study of the Safety, Efficacy, and Pharmacokinetics of Intravenous Efanesoctocog Alfa in Previously Treated Pediatric Patients <12 Years of Age with Severe Hemophilia a

The object of the present study was to demonstrate the safety and efficacy of efanesoctocog alfa, also sometimes referred to as “BIVV001”, in preventing bleeding when used in a weekly prophylactic regimen, in treating bleeding episodes, and in maintaining hemostasis when used in a perioperative setting and to confirm the prolonged half-life of efanesoctocog alfa in previously treated patients (PTPs)<12 years of age with severe hemophilia A.

### A. Study Design

This is a multinational, multicenter, open-label Phase 3 study of the safety, efficacy, and PK of intravenous efanesoctocog alfa in previously treated patients (PTPs)<12 years of age with severe hemophilia A (defined as <1 IU/dL [<1%] endogenous FVIII). The study comprised two age cohorts of children (<6 years and 6 to <12 years), and participants were to receive efanesoctocog alfa at a dose of 50 IU/kg IV once weekly for 52 weeks.

PK assessments at Baseline were performed after a washout period of at least 3 to 4 days depending on age. At least 12 participants in each age cohort were to be included in the PK subgroup, undergoing PK sampling after their first dose of efanesoctocog alfa at Baseline up to 7 days (168 hours). In addition, peak and trough FVIII sampling was performed in all participants throughout the study.

FVIII activities were measured using WHO plasma standard with the aPTT-based one-stage clotting (OSC) assay as the primary evaluation of PK endpoints. The lower limit of quantification (LLOQ) for one stage clotting assay was 1 IU/dL. Plasma FVIII activity was corrected for baseline using predose FVIII activity at Day 1. Summary statistics were computed, and baseline-corrected FVIII activity level-time profiles are presented.

The participants who underwent major surgery during the study were included in the surgery subgroup.

### B. Study Population

### Inclusion Criteria

Participants enrolled in this study were PTPs with severe hemophilia A (defined as <1 IU/dL [<1%] endogenous FVIII), younger than 12 years of age. Previous treatment of hemophilia A was defined as any treatment with any recombinant and/or plasma derived FVIII, or cryoprecipitate for at least 150 exposure days (EDs) for patients aged 6-11 years and for at least 50 EDs for patients aged <6 years. Participants with a history of a positive inhibitor test or with a positive inhibitor test result at Screening were excluded.

Efficacy analyses was based on Full Analysis Set (FAS) defined in this particular study as all enrolled and exposed to at least on dose of efanesoctocog alfa. An efficacy period was used for the evaluation of bleeding and consumption endpoints. For a participant to have an evaluable efficacy period over the duration of study, the patient had to have received at least 2 prophylactic doses of efanesoctocog alfa. The efficacy period is defined in this particular study as the treatment period reduced by the interruption of PK periods, surgical/rehabilitation periods (major and minor) or long injection intervals (>28 days).

Safety Analysis Set was the same as FAS and included all participants who received at least one dose of efanesoctocog alfa. PK Analysis Set (PKAS) is defined in this particular study as all participants who had completed adequate blood sample collection to assess key PK parameters, as determined by the PK scientist.

### C. Study Intervention

The investigational medicinal product used in this study was efanesoctocog alfa. Enrolled subjects received efanesoctocog alfa at a dose of 50 IU/kg IV once weekly for up to 52 weeks.

### D. Objectives and Endpoints

The primary objective of the study is to evaluate the safety of efanesoctocog alfa in previously treated participants <12 years of age with hemophilia A. The primary endpoint is the occurrence of inhibitor development (neutralizing antibodies directed against FVIII as determined via the Nijmegen-modified Bethesda assay).

One secondary objective of the study is to evaluate the efficacy of efanesoctocog alfa as a prophylaxis treatment in this patient population. One secondary endpoint for this efficacy objective is annualized bleeding rate (ABR) for treated bleeding episodes. Other secondary endpoints for this objective are ABR (for treated bleeding episodes) by type and location, ABR for all bleeding episodes (including untreated bleeding episodes), and percentage of participants who maintain FVIII activity levels over 1%, 3%, 5%, 10%, 15%, and 20% at Day 7 post-injection.

Another secondary objective of the study is to evaluate the efficacy of efanesoctocog alfa in the treatment of bleeding episodes in this patient population. Secondary endpoints for this efficacy objective include the number of injections and dose of efanesoctocog alfa to treat a bleeding episode, the percentage of bleeding episodes treated with a single injection of efanesoctocog alfa, assessment of response to efanesoctocog alfa treatment of individual bleeding episodes based on the International Society on Thrombosis and Haemostasis (ISTH) 4-point response scale, and physician's global assessment (PGA) of participant's response to efanesoctocog alfa treatment based on a 4-point response scale.

Another secondary objective of the study is to evaluate the efanesoctocog alfa consumption for prevention and treatment of bleeding episodes. One secondary endpoint for this objective is total annualized efanesoctocog alfa consumption per participant.

Another secondary objective of the study is to evaluate the effect of efanesoctocog alfa prophylaxis on joint health outcomes. Secondary endpoints for this objective include annualized joint bleeding rate (AJBR), target joint resolution at Week 52, based on ISTH criteria, and change from Baseline to Week 52 in total score and domain scores (e.g., swelling and strength) assessed by the Hemophilia Joint Health Score (HJHS).

Another secondary objective of the study is to evaluate the effect of efanesoctocog alfa prophylaxis on Quality of Life (QoL) outcomes. Secondary endpoints for this objective include changes in Haemophilia Quality of Life Questionnaire for Children (Haemo-QoL) total score and physical health domain score from baseline to Week 52 (>4 years old) and via parent proxy version (>4 years old).

Another secondary objective of the study is to evaluate the efficacy of efanesoctocog alfa for perioperative management. Secondary endpoints for this efficacy objective include Investigators or Surgeons' assessment of participant's hemostatic response to efanesoctocog alfa treatment on the ISTH 4 point response for surgical procedures scale, Number of injections and dose to maintain hemostasis during perioperative period for major surgery, total efanesoctocog alfa consumption during perioperative period for major surgery, number and type of blood component transfusions used during perioperative period for major surgery, and estimated blood loss during perioperative period for major surgery.

### Safety

One secondary safety objective of the study is to evaluate the safety and tolerability of efanesoctocog alfa treatment. Secondary endpoints for this safety objective include the occurrence of adverse events (AEs) and serious adverse events (SAEs), the occurrence of clinically significant changes from baseline in physical examination, vital signs, and laboratory tests, and the occurrence of embolic and thrombotic events.

### Pharmacokinetics

One secondary objective of the study is to assess the pharmacokinetics (PK) of efanesoctocog alfa based on the one-stage activated partial thromboplastin time (aPTT) and two-stage chromogenic FVIII activity assays. Secondary endpoints for this objective include PK parameters including, but not limited to, maximum activity (Cₘₐₓ), elimination half-life (t_{1/2}), total clearance (CL), total clearance at steady state (CLss), volume of distribution at steady state (Vss), area under the activity time curve (AUC), dose-normalized area under the activity-time curve (DNAUC), mean residence time (MRT), incremental recovery (IR), trough activity (C_{trough}), and time above predefined FVIII activity levels.

### E. Statistical Methods

For the primary endpoint, inhibitor development in this study was defined as an inhibitor result of ≥0.6 BU/mL that is confirmed by a second test result from a separate sample, drawn 2 to 4 weeks following the date when the original sample was drawn. Both tests must be performed by the central laboratory using the Nijmegen-modified Bethesda assay.

The efficacy endpoint of ABR was analyzed using the FAS including participants with an efficacy period of at least 26 weeks. The mean and 95% CI of ABR was estimated using a Negative-Binomial model. The model included the number of treated bleeding episodes during the efficacy period as response variable, log-transformed duration of efficacy period as offset variable to account for variable duration.

Analysis of safety endpoints: The incidence, seriousness, severity, and relatedness of AEs were assessed by age (under 6 years and 6 years to under 12 years) and overall.

Analysis of PK endpoints: Plasma FVIII activity was corrected for baseline using predose FVIII activity at Day 1. FVIII activity post start of injection was summarized by overall population and age cohort with descriptive statistics.

### F. Interim Results

### Patient Disposition: Interim

At the time of the interim analysis, the study included 67 subjects. 31 subjects were <6 years of age and 36 were 6 to <12 years of age. Participant disposition at the time of the interim analysis is shown in Table 62.

TABLE 62
Participant disposition at the time of interim analysis

| | Age cohort | | | | | |
|---|---|---|---|---|---|---|
| | | | 6 to <12 | | | |
| | <6 years | | years | | Overall | |
| n (%) | (N = 31) | | (N = 36) | | (N = 67) | |
| Number of participants | | | | | | |
| Full Analysis Set ^{a} | 31 | (100) | 36 | (100) | 67 | (100) |
| Participants with an | 27 | (87.1) | 36 | (100) | 63 | (94.0) |
| efficacy period ^{b} | | | | | | |
| Safety Analysis Set ^{c} | 31 | (100) | 36 | (100) | 67 | (100) |
| Surgery subgroup ^{d} | 1 | (3.2) | 0 | | 1 | (1.5) |
| Number of major surgeries | 1 | | 0 | | 1 | |
| Participants with at least one | 1 | (3.2) | 3 | (8.3) | 4 | (6.0) |
| minor surgery | | | | | | |
| Number of minor surgeries | 1 | | 3 | | 4 | |
| Completion status | | | | | | |
| Ongoing | 30 | (96.8) | 36 | (100) | 66 | (98.5) |
| Completed | 0 | | 0 | | 0 | |
| Discontinued | 1 | (3.2) | 0 | | 1 | (1.5) |
| Adverse event | 0 | | 0 | | 0 | |
| Lost to Follow-Up | 0 | | 0 | | 0 | |
| Protocol violation | 0 | | 0 | | 0 | |
| Death | 0 | | 0 | | 0 | |
| Consent withdrawn | 0 | | 0 | | 0 | |
| Inability/Unwillingness to | 0 | | 0 | | 0 | |
| comply with protocol | | | | | | |
| Prohibited concomitant | 0 | | 0 | | 0 | |
| medications due to medical need | | | | | | |
| as determined by investigator | | | | | | |

| Investigator decision | 0 | | 0 | 0 | |
|---|---|---|---|---|---|
| Withdrawal Criteria | 0 | | 0 | 0 | |
| Other | 1 | (3.2) | 0 | 1 | (1.5) |

1: Percentages are based on the number of participants in the All-Enrolled Analysis Set.
^{a} All participants who received at least one dose of study drug.
^{b} FAS participants with at least 2 prophylactic injections.
^{c} All participants who received at least one dose of study drug.
^{d} Participants who have undergone major surgery after the first dose of study drug.

### Exposure—Interim

At the time of the interim analysis, 23 (34.3%) study participants reached at least 25 exposure days (EDs): 11 (35.5%) participants in the <6 years age cohort and 12 (33.3%) participants in the 6 to <12 years age cohort. The mean (SD) total exposure to efanesoctocog alfa was 19.3 (14.9) EDs (range: 1 to 46). The mean (SD) total number of injections per participant was 19.4 (15.1) (range: 1 to 48). (Table 63)

The median dosing interval was 7.00 days during the efficacy period. No participants required shortening of the weekly dosing interval.

TABLE 63
Summary of efanesoctocog alfa injections and exposure
days (EDs) - Safety analysis set - Interim Results

| | Age cohort | | | | | |
|---|---|---|---|---|---|---|
| | | | 6 to <12 | | | |
| | <6 years | | years | | Overall | |
| | (N = 31) | | (N = 36) | | (N = 67) | |
| Total exposure days | | | | | | |
| (EDs) ^{a}, n (%) | | | | | | |
| <5 | 11 | (35.5) | 2 | (5.6) | 13 | (19.4) |
| 5-<10 | 4 | (12.9) | 3 | (8.3) | 7 | (10.4) |
| 10-<25 | 5 | (16.1) | 19 | (52.8) | 24 | (35.8) |
| 25-<50 | 11 | (35.5) | 12 | (33.3) | 23 | (34.3) |
| >=50 | 0 | | 0 | | 0 | |
| At least 5 exposure | 20 | (64.5) | 34 | (94.4) | 54 | (80.6) |
| days | | | | | | |
| At least 10 exposure | 16 | (51.6) | 31 | (86.1) | 47 | (70.1) |
| days | | | | | | |
| At least 25 exposure | 11 | (35.5) | 12 | (33.3) | 23 | (34.3) |
| days | | | | | | |
| At least 50 exposure | 0 | | 0 | | 0 | |
| days | | | | | | |
| Number | 31 | | 36 | | 67 | |
| Mean (SD) | 16.3 | (15.1) | 22.0 | (14.5) | 19.3 | (14.9) |
| Median | 10.0 | | 15.5 | | 14.0 | |
| Min; Max | 1; 41 | | 2; 46 | | 1; 46 | |
| Total number of | | | | | | |
| injections per | | | | | | |
| participant | | | | | | |
| Overall | | | | | | |
| Number | 31 | | 36 | | 67 | |
| Mean (SD) | 16.3 | (15.1) | 22.1 | (14.7) | 19.4 | (15.1) |
| Median | 10.0 | | 15.5 | | 14.0 | |
| Min; Max | 1; 41 | | 2; 48 | | 1; 48 | |
| For Prophylaxis | | | | | | |
| Number | 31 | | 36 | | 67 | |
| Mean (SD) | 16.1 | (15.1) | 21.3 | (14.3) | 18.9 | (14.8) |
| Median | 10.0 | | 14.5 | | 13.0 | |
| Min; Max | 1; 41 | | 2; 44 | | 1; 44 | |
| For Spontaneous bleed | | | | | | |
| Number | 31 | | 36 | | 67 | |
| Mean (SD) | 0.1 | (0.2) | 0.0 | (0.2) | 0.0 | (0.2) |
| Median | 0.0 | | 0.0 | | 0.0 | |
| Min; Max | 0; 1 | | 0; 1 | | 0; 1 | |
| For Traumatic bleed | | | | | | |
| Number | 31 | | 36 | | 67 | |
| Mean (SD) | 0.0 | (0.2) | 0.3 | (0.8) | 0.2 | (0.6) |

| | | | |
|---|---|---|---|
| Median | 0.0 | 0.0 | 0.0 |
| Min; Max | 0; 1 | 0; 4 | 0; 4 |

### Efficacy Results—Interim

### Prevention and Treatment of Bleedings

Efanesoctocog alfa was effective for routine prophylaxis in children under 12 years of age with severe hemophilia A. At the time of the interim analysis, once-weekly efanesoctocog alfa 50 IU/kg routine prophylaxis resulted in an estimated mean ABR of 0.54 (95% CI: 0.23 to 1.26) in the study participants with >26 weeks; the observed mean ABR was 0.52 (1.12). Low ABRs were observed in both age cohorts receiving once-weekly efanesoctocog alfa. The majority of the participants reported no bleeding episodes across both age cohorts.

Once-weekly efanesoctocog alfa 50 IU/kg routine prophylaxis provides high sustained FVIII activity levels in the normal to near normal range for 2 to 3 days and remained in the mild hemophilia range at the end of the dosing interval. At the study visits 7 days postdose, all participants maintained FVIII activity levels >3% and the majority (89.5%) >5%. Trough FVIII activity levels >10% were maintained in 40.9% of children aged 6 to <12 years and 12.5% of children under 6 years. FVIII activity levels >15% and >20% were maintained in one participant each, both were children under 6 years. All participants remained on a once-weekly prophylactic regimen.

Efanesoctocog alfa was effective for the treatment of bleeds in children under 12 years of age. Among 67 participants <12 years of age who received at least one dose of efanesoctocog alfa once weekly 50 IU/kg prophylaxis, 56 participants had no treated bleeding episodes. Six out of 36 participants in the 6 to <12 years of age cohort reported 1 treated bleeding episode each, while 2 participants in that cohort reported more than 1 treated bleeding episode (traumatic). Three out of 31 participants in the <6 years of age cohort reported 1 treated bleeding episode each. All bleeding episodes were resolved with one or two injections. Sixteen out of a total of 19 (84.2%) bleeding episodes in the efficacy period were treated with one injection only. The response to treatment of the bleeding episodes with efanesoctocog alfa was rated by the participants as excellent (good for one).

Physician's global assessment of participant's response to efanesoctocog alfa was rated as excellent for all participants assessed.

### Total Efanesoctocog Alfa Consumption

The median annualized efanesoctocog alfa consumption during the efficacy period was 2877.16 IU/kg (IQR: 2752.68-3136.77) overall and was similar in the 2 age cohorts.

### Perioperative Management

One participant in the <6 years of age cohort underwent a major surgery for extraction of a molar tooth. The FVIII activity level on the day of surgery was 32.7 IU/dL. After this blood sample was drawn, he received a preoperative (loading) dose of 61.9 IU/kg efanesoctocog alfa. Investigator'/Surgeon's assessment of the participant's hemostatic response to efanesoctocog alfa was rated as excellent. There was no need of blood component transfusion during the surgical period.

### Safety Results—Interim

At the time of the interim analysis, all 67 participants enrolled in the study received at least one dose of efanesoctocog alfa and were included in the Safety Analysis Set. There were 23 (34.3%) participants who achieved at least 25 EDs. The median total number of EDs per participant was 14 EDs (range: 1 to 46).

Regarding the primary endpoint, inhibitor development to FVIII was not detected. In all 67 treated participants, the incidence of inhibitor development to FVIII was 0% (95% CI 0.0, 5.4) (Table 64).

TABLE 64
Summary of inhibitor development by Nijmegen-modified
Bethesda assay - Safety analysis set - Interim results

| | Age cohort | | |
|---|---|---|---|
| | <6 | 6 to <12 | |
| | years | years | Overall |
| | (N = 31) | (N = 36) | (N = 67) |
| Participants with ≥50 exposure days | | | |
| to efanesoctocog alfa | | | |
| Participants with an inhibitor test or | 0 | 0 | 0 |
| inhibitor | | | |
| Participants with an inhibitor | 0 | 0 | 0 |
| Incidence of inhibitor formation (%) | NC | NC | NC |
| 95% CI (%) ^{a} | NC | NC | NC |
| Participants with ≥25 exposure days | | | |
| to efanesoctocog alfa | | | |
| Participants with an inhibitor test or | 9 | 12 | 21 |
| inhibitor | | | |
| Participants with an inhibitor | 0 | 0 | 0 |
| Incidence of inhibitor formation (%) | 0.00 | 0.00 | 0.00 |
| 95% CI (%) ^{a} | 0.00; 0.34 | 0.00; 0.26 | 0.00; 0.16 |
| All treated participants | | | |
| Participants with an inhibitor test or | 31 | 36 | 67 |
| inhibitor | | | |
| Participants with an inhibitor | 0 | 0 | 0 |
| Incidence of inhibitor formation (%) | 0.00 | 0.00 | 0.00 |
| 95% CI (%) ^{a} | 0.00; 0.11 | 0.00; 0.10 | 0.00; 0.05 |

1: All tests were performed using the Nijmegen-modified Bethesda assay and confirmatory tests had to be carried out at least 2 weeks after the date when the original sample was drawn. All inhibitor sample analyses only use central lab data.
2: The numerator for the analysis includes the number of participants who tested positive for an inhibitor, regardless of how many days they were exposed to efanesoctocog alfa.
3: Percentages are based on the number of participants with any inhibitor as well as any other participants who reached the exposure day milestone. For participants with >=X exposure days to efanesoctocog alfa, the test must have been performed subsequent to the subsequent to the Xth exposure day.
^{a} 95% CI is calculated using Clopper-Pearson exact method.

Efanesoctocog alfa was well tolerated and reported treatment emergent adverse events (TEAEs) were generally consistent with what is anticipated in a population <12 years of age with severe hemophilia A. A summary of TEAEs at the time of the interim analysis is provided in Table 65.

Of the 67 participants who received at least one dose of efanesoctocog alfa, 36 (53.7%) had at least one TEAE, with a total of 75 TEAEs reported. The most frequently reported TEAEs (>3% of participants overall) were upper respiratory tract infection (6 [9.0%] participants), cough (5 [7.5%]), nasopharyngitis and pyrexia (4 [6.0%] each), asymptomatic COVID-19, constipation, and SARS-CoV-2 test positive (3 [4.5%] participants, each), and COVID-19, ear infection, and eczema (2 [3.0%] participants, each). There were no TESAEs reported at the time of the interim analysis.

There were no reports of serious allergic reaction, anaphylaxis, or vascular thrombotic events. There were no clinically meaningful patterns or trends identified in laboratory or vital sign parameters.

TABLE 65
Overview summary of treatment-emergent adverse events - Safety analysis set - interim results

| | | | Surgical/rehab. | |
|---|---|---|---|---|
| | | | period - | |
| | Age cohort | | Surgery | |
| | <6 years | 6 to <12 years | subgroup^{a} | Overall |
| n (%) | (N = 31) | (N = 36) | (N = 1) | (N = 67) |
| Total number of TEAEs | 40 | 34 | 1 | 75 |
| Participants with at least one TEAE | 20 (64.5) | 16 (44.4) | 1 (100) | 36 (53.7) |
| Participants with at least one related TEAE | 0 | 0 | 0 | 0 |
| Total number of TESAEs | 0 | 0 | 0 | 0 |
| Participants with at least one TESAE | 0 | 0 | 0 | 0 |
| Participants with at least one related TESAE | 0 | 0 | 0 | 0 |
| Total number of TEAESIs | 0 | 0 | 0 | 0 |
| Participants with at least one TEAESI | 0 | 0 | 0 | 0 |
| Participants with at least one related TEAESI | 0 | 0 | 0 | 0 |
| TEAEs leading to death | 0 | 0 | 0 | 0 |
| TEAEs leading to treatment discontinuation | 0 | 0 | 0 | 0 |

1: Percentages are based on the number of participants in the Safety Analysis Set.
2: AEs with missing causality assessment are included in the related TEAE, related TESAE or related TEAESI.
^{a}Includes AEs emergent during the major surgical/rehabilitation period; these AEs are excluded from each age cohort column but are included in the overall column. Each participant is counted only once in the overall column.
Abbreviations: TEAESI = treatment-emergent adverse event with special interest; TESAE = treatment-emergent serious adverse event; TEAE = treatment-emergent adverse event; AE = adverse event.

### Pharmacokinetic Results:

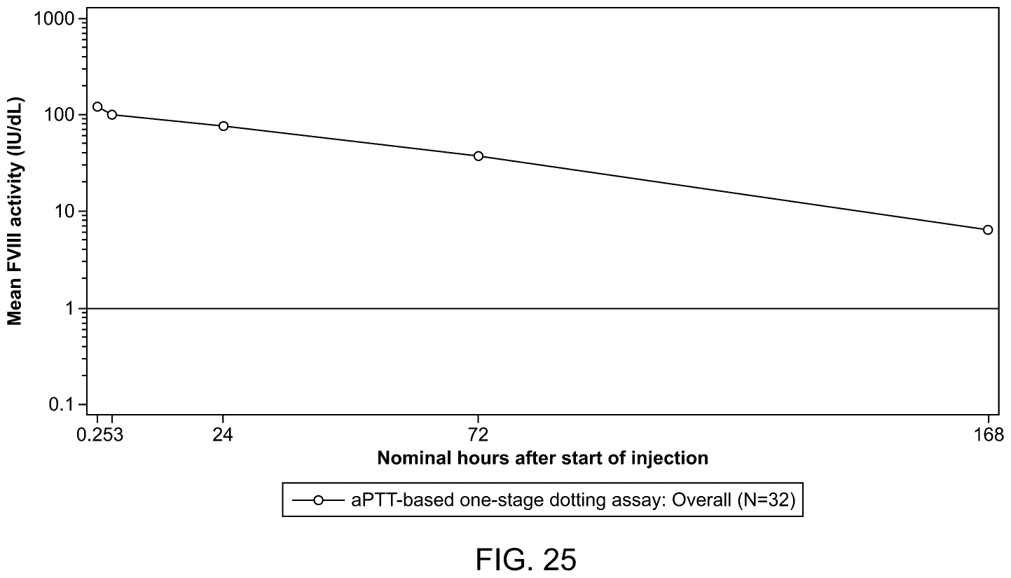

A single efanesoctocog alfa dose of 50 IU/kg resulted in mean FVIII activities in the normal to near-normal range (>40%) for 2 to 3 days (FIG. 25). The mean (SD) FVIII activity, based on OSC assay, was 6.93 (1.76) IU/dL and 5.84 (1.73) IU/dL in 6 to <12 years and <6 years of age cohorts, respectively at the end of the 7-day dosing interval.

The mean (SD) Cmax was 125 (47.4) IU/dL. The majority of participants maintained Cmax within upper physiological limit of 150 IU/dL, while indicating a consistent recovery across study duration.

The mean (SD) terminal half-life of efanesoctocog alfa was 42.4 (3.70) hours and 39.9 (5.71) hours in 6 to <12 years and <6 years of age cohorts, respectively.

There were no participants with a treatment-emergent anti-drug antibody response (i.e., treatment-boosted or treatment-induced anti-drug antibodies).

### G. Final Results

### Patient Disposition: Completed Study

The completed study included 74 subjects. 38 subjects were <6 years of age and 36 were 6 to <12 years of age. Participant disposition for the completed study (n=74) is shown in Table 66.

TABLE 66
Participant disposition for the completed study

| Participant | <6 years | | 6 to <12 years | | Overall | |
|---|---|---|---|---|---|---|
| Disposition | (n = 38) | | (n = 36) | | (n = 74) | |
| Completed, n (%) | 36 | (94.7) | 36 | (100) | 72 | (97.3) |
| Discontinued, n (%) | 2 | (5.3) | 0 | | 2 | (2.7) |
| Exposed to at least | 38 | (100) | 36 | (100) | 74 | (100) |
| one dose of BIVV001, | | | | | | |
| n (%) | | | | | | |
| With ≥50 EDs and an | 30 | (78.9) | 35 | (97.2) | 65 | (87.8) |
| inhibitor test, n (%) | | | | | | |

| | | | | | |
|---|---|---|---|---|---|
| With at least one | 2 | (5.3) | 0 | 2 | (2.7) |

| major surgery, n (%) | | | |
|---|---|---|---|
| Number of major | 2 | 0 | 2 |
| surgeries, n | | | |
| PK Subgroup*, n | 19 | 18 | 37 |

*Subset of participants with full pharmacokinetic (PK) profiles at baseline

### Efficacy Results—Final

### Prevention and Treatment of Bleedings

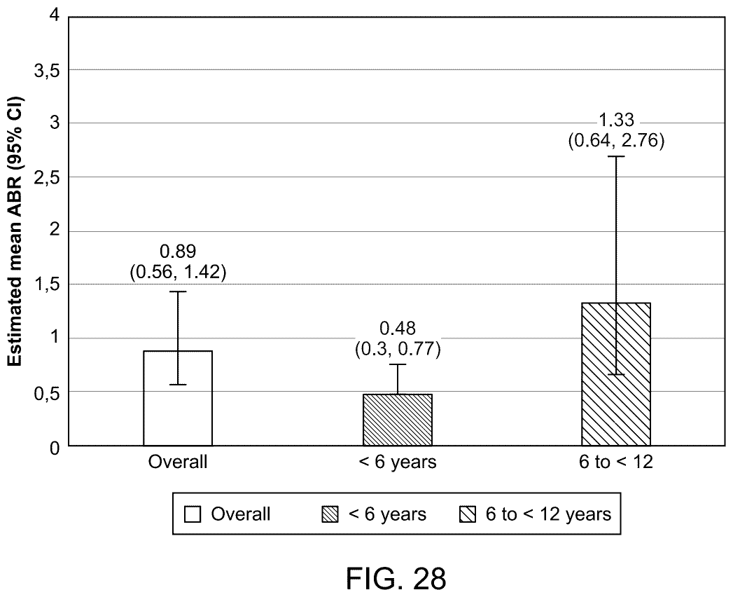

The final results of the study confirmed that efanesoctocog alfa was effective for routine prophylaxis in children under 12 years of age with severe hemophilia A. For all study participants (n=74), once-weekly efanesoctocog alfa 50 IU/kg routine prophylaxis resulted in an estimated mean ABR of 0.89 (95% CI: 0.56 to 1.42). The estimated mean was determined using a negative binomial model with the total number of treated bleeding episodes during the efficacy period as the response variable and log-transformed efficacy period duration (in years) as an offset variable. The observed mean (SD) ABR for all participants was 0.88 (2.62), and the observed median (IQR) ABR for all participants was 0.00 (0.00; 1.02). The final estimated mean ABR for all participants and among both age cohorts is shown in FIG. 28.

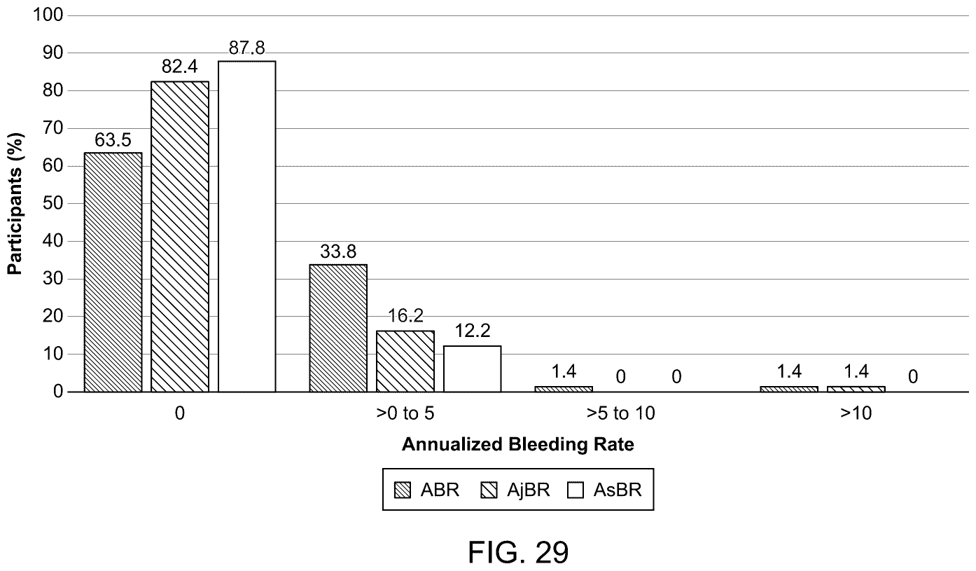

The final results of the study confirmed that efanesoctocog alfa weekly prophylaxis provided effective protection against bleeding episodes in children under 12 years of age with severe hemophilia A. Of the 74 participants, 47 (63.5%) participants had zero bleeding episodes, 61 (82.4%) had zero joint bleeds, and 65 (87.8%) had zero spontaneous bleeds. For all study participants, the estimated mean (95% CI) for annualized joint bleeding rate (AjBR) was 0.59 (0.27; 1.28). For all study participants, the estimated mean (95% CI) for annualized spontaneous bleeding rate (AsBR) was 0.16 (0.08; 0.30). A graph summarizing the participant's ABR, AjBR, and AsBR is shown in FIG. 29.

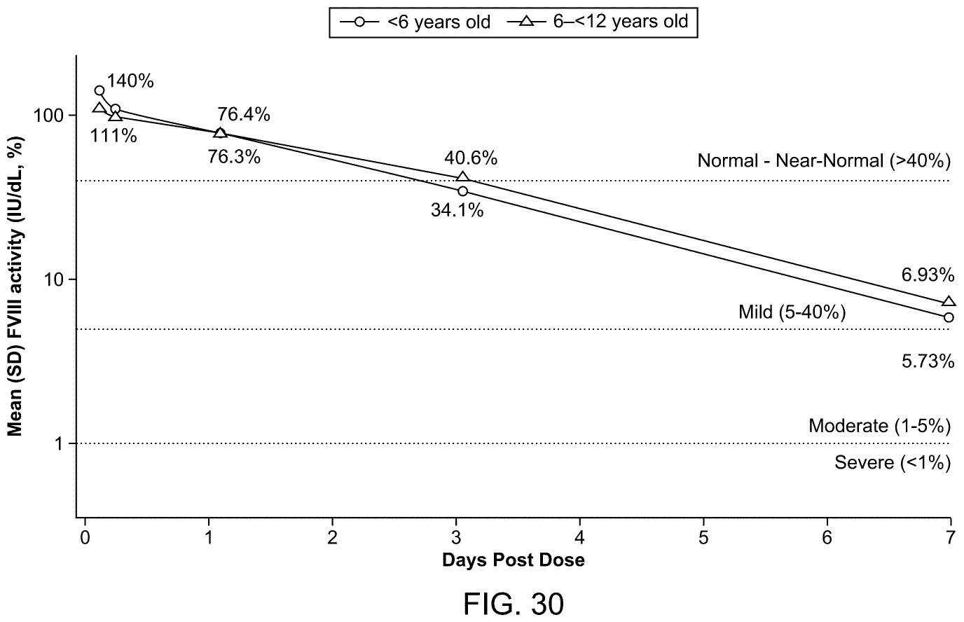

The final results of the study confirmed that once-weekly efanesoctocog alfa at 50 IU/kg for routine prophylaxis provides high sustained FVIII activity levels in the normal to near normal range for 2 to 3 days and remained in the mild hemophilia range at the end of the dosing interval. Mean (SD) FVII activity (IU/dL, %) for 7 days after the first 50 IU/kg dose is shown in FIG. 30 for the PK Analysis Set of participants (n=19 for <6 years old; n=18 for 6-12 years old).

The final results of the study confirmed that once-weekly efanesoctocog alfa was highly effective for the treatment of bleeding episodes. A summary of the treatment of bleeding episodes by age group in the completed study is shown in Table 67.

TABLE 67
Treatment of bleeding episodes by age cohort for the completed study

| | | <6 years | | 6 to <12 years | | Overall | |
|---|---|---|---|---|---|---|---|
| | | (n = 38) | | (n = 36) | | (n = 74) | |
| Number of bleeding | | 17 | | 47 | | 64 | |
| episodes, n | | | | | | | |
| Number of injections | 1 injection | 15 | (88.2) | 37 | (78.7) | 52 | (81.3) |
| to treat a bleed, n % | | | | | | | |
| | 2 injections | 2 | (11.8) | 8 | (17.0) | 10 | (15.6) |
| | >2 injections | 0 | | 2 | (4.3) | 2 | (3.1) |
| Median total dose to | | 57.14 (50.00; | | 52.58 (51.89; | | 52.63 (51.64; | |
| treat a bleeding episode | | 60.98) | | 55.56) | | 60.98) | |
| (IU/kg) (Q1; Q3) | | | | | | | |
| Response to first | Based on injections | 16 | | 24 | | 40 | |
| treatment, n (%) | with an evaluation (n) | | | | | | |
| | Excellent/Good | 15 | (93.8) | 24 | (100.0) | 39 | (97.5) |

| | | | | | |
|---|---|---|---|---|---|
| Moderate | 1 | (6.3) | 0 | 1 | (2.5) |

| | | | |
|---|---|---|---|
| No response | 0 | 0 | 0 |

### Perioperative Management

At study completion, perioperative hemostasis was assessed in 2 major and 9 minor surgeries. Surgeries were hemostatically assessed by the investigator/surgeon. A summary of the 11 surgeries performed during the completed study and their assessment is shown in Table 68.

TABLE 68
Summary of surgeries from the completed study.

| Age of | | Investigator/surgeon's |
|---|---|---|
| participant | Major surgeries | hemostatic assessment |
| 4 Years | Dental restorations and | Excellent |
| | one tooth extraction | |
| 5 Years | Circumcision operation | Excellent |

| Age of | | | Investigator/surgeon's |
|---|---|---|---|
| participant | | Minor surgeries | hemostatic assessment |
| 6 | Years | Tooth extraction | Excellent |
| 6 | Years | Revision port-a-cath | Excellent |
| 9 | Years | Gastroscopy without biopsy | Excellent |
| 9 | Years | Gastroscopy with biopsy | Excellent |
| 4 | Years | Vascuport removal | Excellent |
| 17 | months | Esophagogastroduodenoscopy | Excellent |
| | | with biopsies | |
| 7 | Years | Port replacement | Excellent |
| 11 | Years | Tooth extraction | — |
| 2 | Years | Port replacement | Excellent |

### Safety Results—Final

Final safety results confirmed efanesoctocog alfa was well tolerated and reported treatment emergent adverse events (TEAEs), Treatment Emergent Serious Adverse Event (TESAE), and Treatment Emergent Adverse Event of Special Interest (TEAESI) were generally consistent with what is anticipated in a population <12 years of age with severe hemophilia. An overview of TEAE for all participants (n=74) is presented in Table 69. The most frequently reported TEAEs (>5% of participants overall) in the completed study are presented in Table 70.

In the completed study (n=74), inhibitor development to FVIII was not detected in any of the study participants: 0% (95% CI [0, 4.9]).

TABLE 69
Overview of treatment-emergent adverse events - completed study

| | | | Age 6 | | | |
|---|---|---|---|---|---|---|
| Treatment Emergent Adverse | Age <6 | | to <12 years | | Overall | |
| Events (TEAEs), n(%) | (n = 38) | | (n = 36) | | (n = 74) | |
| Total number of TEAEs | 146 | | 108 | | 254 | |
| Participants with at least one | 33 | (86.8) | 29 | (80.6) | 62 | (83.8) |
| TEAE | | | | | | |
| Participants with at least one | 3 | (7.9) | 0 | | 3 | (4.1) |
| related TEAE | | | | | | |
| Participants with at least one | 5 | (13.2) | 4 | (11.1) | 9 | (12.2) |
| TESAE | | | | | | |

| | | | | | |
|---|---|---|---|---|---|
| Participants with at least one | 1 | (2.6) | 0 | 1 | (1.4) |
| TEAESI | | | | | |

| | | | |
|---|---|---|---|
| TEAEs leading to treatment | 0 | 0 | 0 |
| discontinuation | | | |

TABLE 70
Overview of most Common (>5%) Treatment Emergent
Adverse Events (TEAEs) - completed study

| | | | 6 to <12 | | | |
|---|---|---|---|---|---|---|
| | <6 years | | years | | Overall | |
| Preferred Term, n(%) | (n = 38) | | (n = 36) | | (n = 74) | |
| SARS-CoV-2 test positive | 7 | (18.4) | 4 | (11.1) | 11 | (14.9) |
| Upper respiratory tract | 6 | (15.8) | 5 | (13.9) | 11 | (14.9) |
| infection | | | | | | |
| Pyrexia | 8 | (21.1) | 1 | (2.8) | 9 | (12.2) |
| Asymptomatic COVID-19 | 2 | (5.3) | 5 | (13.9) | 7 | (9.5) |
| Gastroenteritis viral | 5 | (13.2) | 1 | (2.8) | 6 | (8.1) |
| Head injury | 1 | (2.6) | 5 | (13.9) | 6 | (8.1) |
| Nasopharyngitis | 3 | (7.9) | 3 | (8.3) | 6 | (8.1) |

### H. Conclusions

The interim and final results from this study show that once weekly efanesoctocog alfa 50 IU/kg IV was well tolerated and effective as routine prophylactic to protect against bleeding episodes in previously treated patients <12 years of age with severe hemophilia A. The majority of study participants reported no bleeding events across both age cohorts, and most bleeding episodes were resolved with one efanesoctocog alfa injection. In addition, efanesoctocog alfa was effective for the control of bleeding episodes and provided hemostatic efficacy during surgical procedures. Inhibitor development to FVIII was not detected. efanesoctocog alfa PK showed high sustained FVIII activity throughout the 7-day dosing interval. All available data supports the use of efanesoctocog alfa in previously treated patients <12 years of age with hemophilia A.

### V. Embodiments of the Disclosure

The present disclosure includes (and is not limited to) the following exemplary embodiments:

E1. A method of achieving an annualized bleeding rate (ABR) of 2 or less, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

E2. A method of reducing an annualized bleeding rate (ABR) compared to treatment with a Factor VIII (FVIII) replacement protein that is capable of being bound by endogenous von Willebrand Factor (VWF), comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a VWF fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E3. A method of reducing an amount of pain medication needed to decrease or manage pain associated with hemophilia A, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

E4. The method of embodiment 3, wherein the amount of pain medication is decreased compared to the amount needed when the subject is treated for hemophilia A with (i) a FVIII replacement protein that is capable of being bound by endogenous von Willebrand Factor (VWF); (ii) a complex, wherein the complex comprises a FVIII protein and a wild-type VWF protein or a portion thereof, wherein the FVIII protein and the VWF protein or portion thereof are not directly or indirectly covalently attached to each other; or (iii) emicizumab.

E5. The method of embodiment 3, wherein the amount of pain medication is decreased (i) compared to the amount needed when the subject is treated for hemophilia A with prophylactic treatment with emicizumab, (ii) compared to the amount needed when the subject is treated for hemophilia A with prophylactic treatment with a FVIII replacement protein other than the chimeric protein, wherein the FVIII replacement protein is capable of being bound by endogenous VWF, (iii) compared to the amount a corresponding subject who receives prophylactic treatment with emicizumab needs, or (iv) compared to the amount a corresponding subject who receives prophylactic treatment for hemophilia A that comprises administration of an FVIII replacement protein other than the chimeric protein needs, wherein the FVIII replacement protein is capable of being bound by endogenous VWF.

E6. The method of any one of embodiments 3 to 5, wherein the dose of the pain medication is reduced.

E7. The method of any one of embodiments 3 to 6, wherein the number of doses of the pain medication over the course of a day or a week is reduced.

E8. The method of any one of embodiments 3 to 7, wherein the pain medication is a nonsteroidal anti-inflammatory drug (NSAID), an opioid, or a nonopioid pain reliever.

E9. The method of embodiment 8 wherein the pain medication comprises fentanyl, hydromorphone, morphine, oxycodone, oxymorphone, tramadol, oxycodone, oxycodone, acetaminophen, ibuprofen, naproxen sodium, celecoxib, ketorolac, or aspirin.

E10. The method of embodiment 2, for reducing the ABR compared to prophylactic treatment with the FVIII replacement protein that is capable of being bound by endogenous VWF.

E11. The method of embodiment 10, wherein the prophylactic treatment with the FVIII replacement protein that is capable of being bound by endogenous VWF comprises intravenous administration of a dose of 20 IU/kg to 65 IU/kg of the FVIII replacement protein 2 to 4 times per week.

E12. The method of any one of embodiments 2 or 4 to 11, wherein the FVIII replacement protein that is capable of being bound by endogenous VWF is not a fusion protein.

E13. The method of any one of embodiments 2 or 4 to 12, wherein the FVIII replacement protein is plasma derived FVIII.

E14. The method of any one of embodiments 2 or 4 to 12, wherein the FVIII replacement protein is a recombinant FVIII.

E15. The method of embodiment 14, wherein the recombinant FVIII comprises a full or partial B domain deletion.

E16. The method of any one of embodiments 2 or 4 to 12, 14 or 15, wherein the FVIII replacement protein that is capable of being bound by endogenous VWF is pegylated.

E17. The method of any one of embodiments 2 or 4 to 12, 14 or 16, wherein the FVIII replacement protein that is capable of being bound by endogenous VWF is fused to an FcRn binding partner.

E18. The method of embodiment 17, wherein the FcRn binding partner is an Fc region.

E19. The method of any one of embodiments 2, 4 to 12, or 14 to 18, wherein the FVIII replacement protein that is capable of being bound by endogenous VWF is lonoctocog alfa, octocog alfa, rurioctocog alfa pegol, damoctocog alfa pegol, efmoroctocog alfa, simoctocog alfa, moroctocog alfa, beroctocog alfa, or turoctocog alfa.

E20. The method of embodiment 19, for reducing the ABR compared to prophylactic treatment with lonoctocog alfa, wherein the prophylactic treatment with lonoctocog alfa comprises intravenous administration of a dose of 20 IU/kg to 50 IU/kg of the lonoctocog alfa 2 to 3 times per week.

E21. The method of embodiment 19, for reducing the ABR compared to prophylactic treatment with octocog alfa, wherein the prophylactic treatment with octocog alfa comprises intravenous administration of a dose of 20 IU/kg to 40 IU/kg of the octocog alfa every other day or 3 to 4 times per week.

E22. The method of embodiment 19, for reducing the ABR compared to prophylactic treatment with rurioctocog alfa pegol, wherein the prophylactic treatment with rurioctocog alfa pegol comprises intravenous administration of a dose of 40 IU/kg to 50 IU/kg of the rurioctocog alfa pegol 2 times per week.

E23. The method of embodiment 19, for reducing the ABR compared to prophylactic treatment with damoctocog alfa pegol, wherein the prophylactic treatment with damoctocog alfa pegol comprises intravenous administration of a dose of 30 IU/kg to 40 IU/kg of the damoctocog alfa pegol 2 to per week, or 45 IU/kg to 60 IU/kg every 5 days.

E24. The method of embodiment 19, for reducing the ABR compared to prophylactic treatment with efmoroctocog alfa, wherein the prophylactic treatment with efmoroctocog alfa comprises intravenous administration of a dose of 25 IU/kg to 65 IU/kg of the efmoroctocog alfa every 3 to 5 days, or 50 IU/kg every 4 days.

E25. The method of embodiment 19, for reducing the ABR compared to prophylactic treatment with simoctocog alfa, wherein the prophylactic treatment with simoctocog alfa comprises intravenous administration of a dose of 30 IU/kg to 40 IU/kg of the simoctocog alfa every other day.

E26. The method of embodiment 19, for reducing the ABR compared to prophylactic treatment with moroctocog alfa, wherein the prophylactic treatment with moroctocog alfa comprises intravenous administration of a dose of 30 IU/kg of the moroctocog alfa at least 2 to 3 times per week.

E27. A method of reducing an annualized bleeding rate (ABR) compared to prophylactic treatment with a complex, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide sequence, (c) a thrombin-cleavable linker comprising at least a portion of the a2 region of FVIII, and (d) a second Fc region, wherein the first Fc region and the second Fc region are covalently attached to each other by at least one disulfide bond and wherein the complex comprises a FVIII protein and a wild-type VWF protein or a portion thereof, wherein the FVIII protein and the VWF protein or portion thereof are not directly or indirectly covalently attached to each other.

E28. The method of embodiment 27, wherein the prophylactic treatment with the complex comprises intravenous administration of a dose of 30 IU/kg of the complex 3 times per week.

E29. A method of reducing an annualized bleeding rate (ABR) compared to prophylactic treatment with emicizumab, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide sequence, (c) a thrombin-cleavable linker comprising at least a portion of the a2 region of FVIII, and (d) a second Fc region, wherein the first Fc region and the second Fc region are covalently attached to each other by at least one disulfide bond.

E30. The method of embodiment 29, wherein the prophylactic treatment with the emicizumab comprises subcutaneous administration of a dose of 1.5 mg/kg of the emicizumab once per week.

E31. The method of embodiment 29, wherein the prophylactic treatment with the emicizumab comprises subcutaneous administration of a dose of 3 mg/kg of the emicizumab once every two weeks.

E32. The method of embodiment 29, wherein the prophylactic treatment with the emicizumab comprises subcutaneous administration of a dose of 6 mg/kg of the emicizumab once every 4 weeks.

E33. The method of embodiment 29, wherein the prophylactic treatment with the emicizumab comprises subcutaneous administration of a loading dose of 3 mg/kg by subcutaneous injection once weekly for the first 4 weeks followed by a maintenance dose of
(a) a dose of 3 mg/kg of the emicizumab once every two weeks;
(b) a dose of 6 mg/kg of the emicizumab once every 4 weeks; or
(c) a dose of 3 mg/kg of the emicizumab once every two weeks.

E34. The method of any one of embodiments 29 to 33, wherein the emicizumab is emicizumab-kxwh.

E35. The method of any one of embodiments 1, 2, or 10 to 34, wherein the ABR is reduced by from 50% to 90%.

E36. The method of any one of embodiments 1, 2, or 10 to 35, wherein the ABR is reduced by at least 75%.

E37. The method of any one of embodiments 1, 2, or 10 to 36, wherein the ABR is less than about 1.

E38. The method of any one of embodiments 1, 2, or 10 to 37, wherein the ABR is from about 0.5 to about 1.

E39. The method of any one of embodiments 1, 2, or 10 to 38, wherein the ABR is from about 0.5 to about 0.75.

E40. The method of any one of embodiments 1, 2, or 10 to 37, wherein the ABR is from about 0 to about 0.5.

E41. The method of any one of embodiments 1, 2, or 10 to 37, wherein the ABR is from about 0.25 to about 0.75.

E42. The method of any one of embodiments 1, 2, or 10 to 37, wherein the ABR is from 0 to about 0.25.

E43. The method of any one of embodiments 1, 2, or 10 to 37, wherein the ABR is 0.

E44. The method of any one of embodiments 1, 2, or 10 to 43, wherein the ABR is the ABR of spontaneous bleeding episodes.

E45. The method of any one of embodiments 1, 2, or 10 to 43, wherein the ABR is the ABR of traumatic bleeding episodes.

E46. The method of any one of embodiments 1, 2, or 10 to 43, wherein the ABR is the ABR of spontaneous and traumatic bleeding episodes.

E47. The method of any one of embodiments 1, 2, or 10 to 43 or 46, wherein the ABR is the ABR of spontaneous and traumatic bleeding episodes other than normal bleeds.

E48. The method of any one of embodiments 1, 2, or 10 to 44, wherein the ABR is the ABR of spontaneous treated bleeding episodes.

E49. The method of any one of embodiments 1, 2, or 10 to 43 or 45, wherein the ABR is the ABR of traumatic treated bleeding episodes.

E50. The method of any one of embodiments 1, 2, or 10 to 43, wherein the ABR is the ABR of spontaneous treated bleeding episodes and traumatic treated bleeding episodes.

E51. The method of any one of embodiments 1 to 50, wherein a total of from about 2600 IU/kg to about 3000 IU/kg of the chimeric protein is administered to the subject per year.

E52. The method of any one of embodiments 1 to 50, wherein a total of from about 2600 IU/kg to about 2750 IU/kg of the chimeric protein is administered to the subject per year.

E53. The method of any one of embodiments 1 to 50, wherein a total of from about 2600 IU/kg to about 2700 IU/kg of the chimeric protein is administered to the subject per year.

E54. The method of any one of embodiments 1 to 50, wherein a total of from about 2600 IU/kg to about 2650 IU/kg of the chimeric protein is administered to the subject per year.

E55. The method of any one of embodiments 1 to 50, wherein a total of about 2600 IU/kg of the chimeric protein is administered to the subject per year.

E56. The method of any one of embodiments 1, 2, or 10 to 55, wherein the ABR is less than 2 and the method comprises administering a total of about 2600 IU/kg/year of the chimeric protein.

E57. The method of any one of embodiments 1, 2, or 10 to 56, wherein the ABR is less than 1 and the method comprises administering a total of about 2600 IU/kg/year of the chimeric protein.

E58. The method of any one of embodiments 1, 2, or 10 to 57, wherein the ABR is between 1 and 0.5 and the method comprises administering a total of about 2600 IU/kg/year of the chimeric protein.

E59. The method of any one of embodiments 1, 2, or 10 to 58, wherein the ABR is between 0.75 and 0.5 and the method comprises administering a total of about 2600 IU/kg/year of the chimeric protein.

E60. The method of any one of embodiments 1, 2, or 10 to 57, wherein the ABR is between 0.5 and 0.25 and the method comprises administering a total of about 2600 IU/kg/year of the chimeric protein.

E61. The method of any one of embodiments 1, 2, or 10 to 57, wherein the ABR is between 0.25 and 0 and the method comprises administering a total of about 2600 IU/kg/year of the chimeric protein.

E62. A method of maintaining a FVIII activity level of at least 2%, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide sequence, (c) a thrombin-cleavable linker comprising at least a portion of the a2 region of FVIII, and (d) a second Fc region, wherein the first Fc region and the second Fc region are covalently attached to each other by at least one disulfide bond.

E63. The method of embodiment 62, wherein a FVIII activity level of from about 10% to about 15% is maintained.

E64. The method of embodiment 62, wherein a FVIII activity level of from about 15% to about 20% is maintained.

E65 The method of any one of embodiments 1 to 64, wherein a FVIII activity level of from about 35% to about 45% is maintained for at least 4 days after each administration of the chimeric protein.

E66. The method of any one of embodiments 1 to 65, wherein a FVIII activity level of from about 10% to about 15% is maintained for at least 7 days after each administration of the chimeric protein.

E67. The method of any one of embodiments 1 to 66, wherein a FVIII activity level of from about 2% to about 5% is maintained for at least 14 days after each administration of the chimeric protein.

E68. The method of any one of embodiments 1 to 66, wherein a FVIII activity level of from about 3% to about 5% is maintained for at least 14 days after each administration of the chimeric protein.

E69. A method of reducing the risk of a bleeding episode that requires more than one supplemental on-demand administration of a FVIII replacement protein during the prophylactic treatment of hemophilia A, wherein the prophylactic treatment of hemophilia A comprises administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E70. The method of embodiment 69, wherein the supplemental on-demand administration of the FVIII replacement protein is supplemental on-demand administration of the chimeric protein.

E71. The method of embodiment 69 or 70, wherein the risk is decreased by at least 70% compared to the risk of a corresponding subject who receives prophylactic treatment for hemophilia A that comprises administration of an FVIII replacement protein other than the chimeric protein, wherein the FVIII replacement protein other than the chimeric protein is capable of being bound by endogenous VWF.

E72. The method of any one of embodiments 69 to 71, wherein the risk is decreased by at least 90% compared to the risk of a corresponding subject who receives prophylactic treatment for hemophilia A that comprises administration of an FVIII replacement protein other than the chimeric protein, wherein the FVIII replacement protein other than the chimeric protein is capable of being bound by endogenous VWF.

E73. The method of any one of embodiments 69 to 72, wherein the supplemental on-demand dose is 45 IU/kg to 70 IU/kg.

E74. The method of any one of embodiments 69 to 73, wherein the supplemental on-demand dose is about 50 IU/kg.

E75. A method of resolving a bleeding episode, comprising administering to a human subject in need thereof a single on-demand dose of a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E76. The method of embodiment 75, wherein the single on-demand dose is about 50 IU/kg.

E77. The method of embodiment 75 or 76, wherein the bleeding episode is a spontaneous bleeding episode or a traumatic bleeding episode.

E78. The method of any one of embodiments 75 to 77, wherein the bleeding episode is in a joint.

E79. A method of prophylactically treating hemophilia A, the method comprising administering to a subject in need thereof a therapeutically effective amount of a chimeric protein, wherein a total of less than 3500 IU/kg/year, less than 3400 IU/kg/year, less than 3300 IU/kg/year, less than 3200 IU/kg/year, less than 3100 IU/kg/year, less than 3000 IU/kg/year, less than 2900 IU/kg/year, less than 2800 IU/kg/year, less than 2700 IU/kg/year, or about 2600 IU/kg/year of the chimeric protein is administered to the subject, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E80. The method of embodiment 79, wherein the therapeutically effective amount is a dose of the chimeric protein from 45 IU/kg to 70 IU/kg.

E81. The method of embodiment 79 or 80, the chimeric protein is administered at a dosing interval of about 6 days to about 14 days.

E82. A method of prophylactically treating hemophilia A, comprising administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, wherein the subject is not administered any on-demand treatment for hemophilia A before engaging in strenuous activity.

E83. The method of embodiment 82, wherein the strenuous activity is an activity that raises the subject's heart rate to at least 115 beats per minute.

E84. The method of embodiment 82 or 83, wherein the strenuous activity comprises parkour, running, swimming, bicycling, or climbing.

E85. The method of embodiment 82 or 83, wherein the strenuous activity is hiking.

E86. The method of embodiment 84, wherein the climbing is rock climbing or mountain climbing.

E87. The method of any one of embodiments 82 to 84, wherein the strenuous activity is a sport.

E88. The method of any one of embodiments 82 to 87, wherein the strenuous activity is soccer, tennis, skiing, snowboarding, skateboarding, or basketball.

E89. The method of embodiment 87, wherein the sport is a contact sport.

E90. The method of embodiment 89, wherein the contact sport is football, hockey, rugby, boxing, wrestling, or a martial art.

E91. The method of any one of embodiments 82 to 90, wherein the subject needs less pain medication to decrease or manage pain associated with hemophilia A before, during, or within 1, 3, or 6 hours after the strenuous activity (i) compared to the amount needed when the subject is treated for hemophilia A with prophylactic treatment with emicizumab, (ii) compared to the amount needed when the subject is treated for hemophilia A with prophylactic treatment with a FVIII replacement protein other than the chimeric protein, wherein the FVIII replacement protein is capable of being bound by endogenous VWF, (iii) compared to the amount a corresponding subject who receives prophylactic treatment with emicizumab needs, or (iv) compared to the amount a corresponding subject who receives prophylactic treatment for hemophilia A that comprises administration of an FVIII replacement protein other than the chimeric protein needs, wherein the FVIII replacement protein is capable of being bound by endogenous VWF.

E92. The method of embodiment 91, wherein the subject does not need pain medication to decrease or manage pain associated with hemophilia A before, during, or within 1, 3, or 6 hours after the strenuous activity.

E93. A method of improving quality of life with the prophylactic treatment of hemophilia A, comprising administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E94. The method of embodiment 93, wherein the quality of life is improved compared to (i) prophylactic treatment with emicizumab, (ii) prophylactic treatment with a FVIII replacement protein other than the chimeric protein, wherein the FVIII replacement protein is capable of being bound by endogenous VWF, (iii) to the quality of life of a corresponding subject who receives prophylactic treatment with emicizumab, or (iv) the quality of life of a corresponding subject who receives prophylactic treatment for hemophilia A that comprises administration of an FVIII replacement protein other than the chimeric protein, wherein the FVIII replacement protein is capable of being bound by endogenous VWF.

E95. The method of embodiment 94, wherein the quality of life is improved compared to the quality of life of the subject during previous prophylactic treatment with emicizumab or the quality of life of a corresponding subject who receives prophylactic treatment with emicizumab.

E96. The method of embodiment 95, wherein the prophylactic treatment with the emicizumab comprises subcutaneous administration of a dose of 1.5 mg/kg of the emicizumab once per week.

E97. The method of embodiment 95, wherein the prophylactic treatment with the emicizumab comprises subcutaneous administration of a dose of 3 mg/kg of the emicizumab once every two weeks.

E98. The method of embodiment 95, wherein the prophylactic treatment with the emicizumab comprises subcutaneous administration of a dose of 6 mg/kg of the emicizumab once every 4 weeks.

E99. The method of embodiment 95, wherein the prophylactic treatment with the emicizumab comprises subcutaneous administration of a loading dose of 3 mg/kg by subcutaneous injection once weekly for the first 4 weeks followed by a maintenance dose of (a) a dose of 1.5 mg/kg of the emicizumab once every week; (b) a dose of 6 mg/kg of the emicizumab once every 4 weeks; or (c) a dose of 3 mg/kg of the emicizumab once every two weeks

E100. The method of embodiment 99, wherein the prophylactic treatment with the emicizumab comprises a loading dose of 3 mg/kg by subcutaneous injection once weekly for the first 4 weeks followed by a maintenance dose of 1.5 mg/kg once every week.

E101. The method of any one of embodiments 95 to 100, wherein the emicizumab is emicizumab-kxwh.

E102. The method of any one of embodiments 93 to 101, wherein improving the quality of life of the subject comprises reducing the Haem-A-QoL score of the subject.

E103. The method of embodiment 102, wherein the Haem-A-QoL score of the subject improves by at least 5 compared to the Haem-A-QoL score of the subject before the subject first received prophylactic treatment with the chimeric protein.

E104. The method of embodiment 102 or 103, wherein the Haem-A-QoL score of the subject improves by at least 6 compared to the Haem-A-QoL score of the subject before the subject first received prophylactic treatment with the chimeric protein.

E105. The method of any one of embodiments 102 to 104, wherein the Haem-A-QoL score of the subject improves by at least 7 compared to the Haem-A-QoL score of the subject before the subject first received prophylactic treatment with the chimeric protein.

E106. The method of any one of embodiments 102 to 105, wherein the Haem-A-QoL score of the subject improves by at least 10% compared to the Haem-A-QoL score of the subject before the subject first received prophylactic treatment with the chimeric protein.

E107. The method of any one of embodiments 93 to 106, wherein improving the quality of life of the subject comprises reducing the pain of the subject.

E108. The method of embodiment 107, wherein improving the quality of life of the subject comprises reducing the subject's self-reported pain intensity, wherein the subject's self-reported pain intensity changes from severe to moderate, mild, or no pain.

E109. The method of embodiment 108, wherein the subject's self-reported pain intensity is a rating of the most intense hemophilia A-related pain over the last 7 days.

E110. The method of any one of embodiments 1 to 109, wherein, prior to administration of the chimeric protein, the subject is administered a pain medication to reduce or manage pain associated with hemophilia A, and the pain is reduced upon treatment with the chimeric protein such that the subject needs less pain medication to reduce or manage the pain to the same degree as experienced prior to administration of the chimeric protein.

E111. The method of embodiment 110, wherein the dose of the pain medication is reduced upon treatment with the chimeric protein.

E112. The method of embodiment 110 or 111, wherein the number of doses of the pain medication over the course of a day or a week is reduced upon treatment with the chimeric protein.

E113. The method of any one of embodiments 110 to 112, wherein the pain medication is a nonsteroidal anti-inflammatory drug (NSAID), an opioid, or a nonopioid pain reliever.

E114. The method of embodiment 113, wherein the pain medication comprises fentanyl, hydromorphone, morphine, oxycodone, oxymorphone, tramadol, oxycodone, oxycodone, acetaminophen, ibuprofen, naproxen sodium, celecoxib, ketorolac, or aspirin.

E115. The method of any one of embodiments 107 to 114, wherein the subject's self-reported pain intensity improves by at least 10%.

E116. The method of embodiment 115, wherein the subject's self-reported pain intensity is a rating of the most intense hemophilia A-related pain over the last 7 days as related on a numeric scale.

E117. The method of embodiment 116, wherein the numeric scale is from 1 to 5 or from 1 to 10.

E118. The method of any one of embodiments 93 to 117, wherein improving the quality of life of the subject comprises reducing the Hemophilia Joint Health (HJHS) score of the subject.

E119. The method of embodiment 118, wherein the HJHS score of the subject improves by at least 1 compared to the HJHS score of the subject before the subject first received prophylactic treatment with the chimeric protein.

E120. The method of embodiment 118 or 119, wherein the HJHS score of the subject improves by at least 2 compared to the HJHS score of the subject before the subject first received prophylactic treatment with the chimeric protein.

E121. The method of any one of embodiments 118 to 120, wherein the HJHS score of the subject improves by at least 10% compared to the HJHS score of the subject before the subject first received prophylactic treatment with the chimeric protein.

E122. A method of improving one or more joint outcomes, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E123. The method of embodiment 122, wherein the one or more joint outcomes is improved compared to the outcomes of a corresponding subject who receives prophylactic treatment for hemophilia A that comprises administration of an FVIII replacement protein other than the chimeric protein, wherein the FVIII replacement protein is capable of being bound by endogenous VWF.

E124. The method of embodiment 123, wherein the one or more joint outcomes are improved compared to such outcomes of a corresponding subject who receives prophylactic treatment with emicizumab.

E125. The method of any one of embodiments 122 to 124, wherein improving one or more joint outcomes comprises reducing hemarthrosis.

E126. The method of any one of embodiments 122 to 125, wherein improving one or more joint outcomes comprises reducing the incidence of bleeds.

E127. The method of embodiment 126, wherein treatment with the chimeric protein results in an annualized joint bleeding rate (AjBR) of less than about 1.

E128. The method of embodiment 126, wherein treatment with the chimeric protein results in an AjBR of about 0.

E129. The method of embodiment 126, wherein treatment with the chimeric protein results in an AjBR of from about 0.25 to about 0.75.

E130. The method of any one of embodiments 122 to 129, wherein improving one or more joint outcomes comprises reversing hemophilic arthropathy of a target joint.

E131. The method of embodiment 130, wherein improving one or more joint outcomes comprises wherein the reversible hemophilic arthropathy comprises synovitis, a microbleed, or both.

E132. The method of any one of embodiments 122 to 133, wherein improving one or more joint outcomes comprises reducing vascular remodeling in one or more target joints.

E133. The method of any one of embodiments 122 to 132, wherein improving one or more joint outcomes comprises reducing pain or swelling in surrounding soft tissue of a joint.

E134. The method of any one of embodiments 122 to 133, wherein the joint is an elbow, a knee, an ankle, a shoulder joint, a hip, a wrist, a hand joint, or a foot joint.

E135. The method of any one of embodiments 122 to 134, wherein improving one or more joint outcomes comprises increased joint flexion ability, increased joint extension, increased range of movement, increased muscle strength, decreased swelling, decreased duration of swelling, decreased crepitus, decreased pain, or decreased muscle atrophy of one or more joints.

E136. The method of any one of embodiments 122 to 135, wherein improving one or more joint outcomes comprises improved walking, improved ability to use stairs, improved running ability, or improved ability to hop on one leg.

E137. The method of any one of embodiments 122 to 136, wherein improving one or more joint outcomes comprises achieving an excellent outcome for at least 90% of joint bleeds as assessed using the International Society on Thrombosis and Haemostasis (ISTH) 4-point assessment scale.

E138. The method of embodiment 137, wherein improving one or more joint outcomes comprises achieving an excellent outcome for at least 95% of joint bleeds as assessed using the International Society on Thrombosis and Haemostasis (ISTH) 4-point assessment scale.

E139. The method of any one of embodiments 122 to 138, wherein improving one or more joint outcomes comprises achieving an excellent outcome for at least 90% of joint bleeds as assessed using the Physician's Global Assessment scale.

E140. The method of embodiment 139, wherein improving one or more joint outcomes comprises achieving an excellent outcome for at least 95% of joint bleeds as assessed using the Physician's Global Assessment scale.

E141. The method of any one of embodiments 1 to 140, wherein the treatment for hemophilia A is routine prophylaxis.

E142. The method of any one of embodiments 1 to 72 or 79 to 141, wherein the therapeutically effective amount is a dose of about 45 IU/kg to about 70 IU/kg of a chimeric protein at a dosing interval of about 6 days to about 14 days.

E143. The method of any one of embodiments 1 to 142, wherein the therapeutically effective amount is a dose of about 50 IU/kg of the chimeric protein at a dosing interval of about 6 days to about 14 days.

E144. The method of any one of embodiments 1 to 143, wherein the therapeutically effective amount is a dose of about 50 IU/kg of the chimeric protein at a dosing interval of about 6 days to about 8 days.

E145. The method of any one of embodiments 1 to 143, wherein the therapeutically effective amount is a dose of about 50 IU/kg of the chimeric protein at a dosing interval of about 10 days to about 14 days.

E146. The method of any one of embodiments 1 to 144, wherein the therapeutically effective amount is a dose of about 50 IU/kg of the chimeric protein at a dosing interval of about 7 days.

E147. A method of on-demand treatment of a minor bleed, a moderate bleed, or a major bleed, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E148. The method of embodiment 147, for on-demand treatment of a minor bleed or a moderate bleed.

E149. The method of embodiment 148, wherein the minor bleed or moderate bleed is an uncomplicated joint bleed, a minor muscular bleed, a mucosal bleed, or a subcutaneous bleed.

E150. The method of embodiment 147, for on-demand treatment of a major bleed.

E151. The method of embodiment 150, wherein the major bleed is an intracranial bleed, a retroperitoneal bleed, an iliopsoas bleed, a neck bleed, a muscle bleed with compartment syndrome, and/or a bleed associated with a significant decrease in hemoglobin level.

E152. The method of any one of embodiments 147 to 151, wherein the therapeutically effective amount is a dose from 30 IU/kg to 50 IU/kg.

E153. The method of any one of embodiments 147 to 152, wherein the therapeutically effective amount is a dose of 30 IU/kg.

E154. The method of any one of embodiments 147 to 152, wherein the therapeutically effective amount is a dose of 50 IU/kg.

E155. The method of any one of embodiments 147 to 154, wherein the dose is administered once.

E156. The method of any one of embodiments 147 to 154, wherein the dose is administered every 2 or 3 days until the bleed has stopped.

E157. The method of any one of embodiments 147 to 156, wherein the subject is receiving prophylactic treatment for hemophilia A with the chimeric protein, and the dose is administered 2 to 3 days or less after a prophylactic dose.

E158. The method of any one of embodiments 147 to 157, wherein the subject is receiving prophylactic treatment for hemophilia A with the chimeric protein, and at least 3 days pass after the last on-demand dose to treat the bleed before another prophylactic dose is administered.

E159. A method for the perioperative management of bleeding, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

E160. The method of embodiment 159, wherein the subject is expected to receive surgery within 72 hours.

E161. The method of embodiment 159, wherein the subject is receiving surgery.

E162. The method of embodiment 159, wherein the subject has received surgery within the last week.

E163. The method of any one of embodiments 159 to 162, wherein the surgery is major surgery.

E164. The method of any one of embodiments 159 to 162, wherein the surgery is minor surgery.

E165. The method of any one of embodiments 159 to 164, wherein the therapeutically effective amount achieves an excellent hemostatic response as assessed by a surgeon.

E166. The method of any one of embodiments 159 to 165, wherein the therapeutically effective amount is from about 25 IU/kg to about 65 IU/kg.

E167. The method of any one of embodiments 159 to 166, wherein the therapeutically effective amount is about 30 IU/kg or about 50 IU/kg.

E168. The method of any one of embodiments 159 to 166, wherein the therapeutically effective amount comprises a preoperative loading dose of 50 IU/kg, followed by one or more doses of 30 or 50 IU/kg every 2 to 3 days.

E169. The method of any one of embodiments 1 to 168, wherein the FVIII polypeptide has a deletion of amino acids 746 to 1648 corresponding to mature FVIII (SEQ ID NO: 8), the first ELNN polypeptide is inserted within the FVIII polypeptide immediately downstream of amino acid 745 corresponding to mature FVIII (SEQ ID NO: 8), and the first ELNN polypeptide comprises an amino acid sequence that is least about 90% identical to the amino acid sequence of SEQ ID NO: 9.

E170. The method of any one of embodiments 1 to 169, wherein the VWF fragment comprises a D′ domain of VWF and a D3 domain of VWF, the VWF fragment is mutated to substitute cysteines involved in VWF dimerization to alanine, the second ELNN polypeptide comprises an amino acid sequence that is at least about 90% identical to the amino acid sequence of SEQ ID NO: 14, and the linker comprises an amino acid sequence that is at least about 90% identical to the amino acid sequence of SEQ ID NO: 15.

E171. The method of any one of embodiments 1 to 179, wherein the first ELNN polypeptide comprises the sequence of SEQ ID NO: 9 and the second ELNN polypeptide comprises the amino acid sequence of SEQ ID NO: 14.

E172. The method of any one of embodiments 1 to 171, wherein the first polypeptide comprises an amino acid sequence that is at least about 95% identical to the amino acid sequence of SEQ ID NO: 1 and the second polypeptide comprises an amino acid sequence that is at least about 95% identical to the amino acid sequence of SEQ ID NO: 2, wherein the first polypeptide and the second polypeptide are covalently linked by two disulfide bonds between the first Fc region and the second Fc region.

E173. The method of any one of embodiments 1 to 172, wherein the first polypeptide comprises the amino acid sequence set forth as SEQ ID NO: 1 and the second polypeptide comprises the amino acid sequence set forth as SEQ ID NO: 2, wherein the first polypeptide and the second polypeptide are covalently linked by two disulfide bonds between the first Fc region and the second Fc region.

E174. The method of any one of embodiments 1 to 173, wherein the chimeric protein is efanesoctocog alfa.

E175. The method of any one of embodiments 1 to 174, wherein every dose of the chimeric protein that is administered to the subject is about the same for at least 1 month.

E176. The method of any one of embodiments 1 to 175, wherein every dose of the chimeric protein that is administered to the subject is about the same for at least 3 months.

E177. The method of any one of embodiments 1 to 176, wherein every dose of the chimeric protein that is administered to the subject is about the same for at least 6 months.

E178. The method of any one of embodiments 1 to 177, wherein every dose of the chimeric protein that is administered to the subject is about the same for at least 9 months.

E179. The method of any one of embodiments 1 to 178, wherein every dose of the chimeric protein that is administered to the subject is about the same for at least 12 months.

E180. The method of any one of embodiments 1 to 179, wherein the FVIII activity level in the subject is not measured for at least 1 month.

E181. The method of any one of embodiments 1 to 180, wherein the FVIII activity level in the subject is not measured for at least 3 months.

E182. The method of any one of embodiments 1 to 181, wherein the FVIII activity level in the subject is not measured for at least 6 months.

E183. The method of any one of embodiments 1 to 182, wherein the FVIII activity level in the subject is not measured for at least 9 months.

E184. The method of any one of embodiments 1 to 183, wherein the FVIII activity level in the subject is not measured for at least 12 months.

E185. The method of any one of embodiments 1 to 184, wherein the subject has severe hemophilia A.

E186. The method of any one of embodiments 1 to 185, wherein the subject has previously been treated for hemophilia A with a FVIII replacement protein other than the chimeric protein.

E187. The method of any one of embodiments 1 to 185, wherein the subject has not previously been treated for hemophilia A with a FVIII replacement protein.

E188. The method of any one of embodiments 1 to 187, wherein the subject is at least 12 years old.

E189. The method of any one of embodiments 1 to 188, wherein the subject is at least 18 years old.

E190. The method of any one of embodiments 1 to 187, wherein the subject is less than 12 years old.

E191. The method of any one of embodiments 1 to 190, wherein the subject has at least 1 target joint.

E192. The method of embodiment 191, wherein the subject has at least 3 target joints.

E193. The method of embodiment 191, wherein the subject has at least 6 target joints.

E194. The method of any one of embodiments 191 to 193, wherein treatment with the chimeric protein resolves the subject's target joint(s).

E195. The method of any one of embodiments 1 to 194, wherein the subject has received on-demand treatment for at least 2 bleeding episodes within the last year.

E196. The method of embodiment 195, wherein the subject has received on-demand treatment for at least 3 bleeding episodes within the last year.

E197. The method of embodiment 195, wherein the subject has received on-demand treatment for at least 5 bleeding episodes within the last year.

E198. The method of anyone of embodiments 195 to 197, wherein the bleeding episodes were traumatic bleeding episodes.

E199. The method of anyone of embodiments 195 to 197, wherein the bleeding episodes were spontaneous bleeding episodes.

E200. The method of any one of embodiments 1 to 199, wherein the chimeric protein is administered intravenously.

E201. A chimeric protein for use in achieving an annualized bleeding rate (ABR) of 2 or less, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E202. A chimeric protein for use in reducing an annualized bleeding rate (ABR) compared to treatment with a Factor VIII (FVIII) replacement protein that is capable of being bound by endogenous VWF, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E203. A chimeric protein for use in reducing an amount of pain medication needed to decrease or manage pain associated with hemophilia A, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

E204. The chimeric protein for use according to embodiment 203, wherein the amount of pain medication is decreased compared to the amount needed when the subject is treated for hemophilia A with (i) a FVIII replacement protein that is capable of being bound by endogenous von Willebrand Factor (VWF); (ii) a complex, wherein the complex comprises a FVIII protein and a wild-type VWF protein or a portion thereof, wherein the FVIII protein and the VWF protein or portion thereof are not directly or indirectly covalently attached to each other; or (iii) emicizumab.

E205. The chimeric protein for use according to embodiment 203, wherein the amount of pain medication is decreased (i) compared to the amount needed when the subject is treated for hemophilia A with prophylactic treatment with emicizumab, (ii) compared to the amount needed when the subject is treated for hemophilia A with prophylactic treatment with a FVIII replacement protein other than the chimeric protein, wherein the FVIII replacement protein is capable of being bound by endogenous VWF, (iii) compared to the amount a corresponding subject who receives prophylactic treatment with emicizumab needs, or (iv) compared to the amount a corresponding subject who receives prophylactic treatment for hemophilia A that comprises administration of an FVIII replacement protein other than the chimeric protein needs, wherein the FVIII replacement protein is capable of being bound by endogenous VWF.

E206. The chimeric protein for use according to embodiments 203 to 205, wherein the dose of the pain medication is reduced.

E207. The chimeric protein for use according to embodiments 203 to 206, wherein the number of doses of the pain medication over the course of a day or a week is reduced.

E208. The chimeric protein for use according to any one of embodiments 203 to 207, wherein the pain medication is a nonsteroidal anti-inflammatory drug (NSAID), an opioid, or a nonopioid pain reliever.

E209. The chimeric protein for use according to embodiment 208 wherein the pain medication comprises fentanyl, hydromorphone, morphine, oxycodone, oxymorphone, tramadol, oxycodone, oxycodone, acetaminophen, ibuprofen, naproxen sodium, celecoxib, ketorolac, or aspirin.

E210. The chimeric protein for use according to embodiment 202, for reducing the ABR compared to prophylactic treatment with the FVIII replacement protein that is capable of being bound by endogenous VWF.

E211. The chimeric protein for use according to embodiment 210, wherein the prophylactic treatment with the FVIII replacement protein that is capable of being bound by endogenous VWF comprises intravenous administration of a dose of 20 IU/kg to 65 IU/kg of the FVIII replacement protein 2 to 4 times per week.

E212. The chimeric protein for use according to any one of embodiments 202 or 204 to 211, wherein the FVIII replacement protein that is capable of being bound by endogenous VWF is not a fusion protein.

E213. The chimeric protein for use according to any one of embodiments 202 or 204 to 212, wherein the FVIII replacement protein is plasma derived FVIII.

E214. The chimeric protein for use according to any one of embodiments 202 or 204 to 212, wherein the FVIII replacement protein is a recombinant FVIII.

E215. The chimeric protein for use according to embodiment 214, wherein the recombinant FVIII comprises a full or partial B domain deletion.

E216. The chimeric protein for use according to any one of embodiments 202 or 204 to 212, 124 or 215, wherein the FVIII replacement protein that is capable of being bound by endogenous VWF is pegylated.

E217. The chimeric protein for use according to any one of embodiments 202 or 204 to 212, 214 or 216, wherein the FVIII replacement protein that is capable of being bound by endogenous VWF is fused to an FcRn binding partner.

E218. The chimeric protein of embodiment 217, wherein the FcRn binding partner is an Fc region.

E219. The chimeric protein for use according to any one of embodiments 202 or 204 to 218, wherein the FVIII replacement protein that is capable of being bound by endogenous VWF is lonoctocog alfa, octocog alfa, rurioctocog alfa pegol, damoctocog alfa pegol, efmoroctocog alfa, simoctocog alfa, moroctocog alfa, beroctocog alfa, or turoctocog alfa.

E220. The chimeric protein for use according to embodiment 219, for reducing the ABR compared to prophylactic treatment with lonoctocog alfa, wherein the prophylactic treatment with lonoctocog alfa comprises intravenous administration of a dose of 20 IU/kg to 50 IU/kg of the lonoctocog alfa 2 to 3 times per week.

E221. The chimeric protein for use according to embodiment 219, for reducing the ABR compared to prophylactic treatment with octocog alfa, wherein the prophylactic treatment with octocog alfa comprises intravenous administration of a dose of 20 IU/kg to 40 IU/kg of the octocog alfa every other day or 3 to 4 times per week.

E222. The chimeric protein for use according to embodiment 219, for reducing the ABR compared to prophylactic treatment with rurioctocog alfa pegol, wherein the prophylactic treatment with rurioctocog alfa pegol comprises intravenous administration of a dose of 40 IU/kg to 50 IU/kg of the rurioctocog alfa pegol 2 times per week.

E223. The chimeric protein for use according to embodiment 219, for reducing the ABR compared to prophylactic treatment with damoctocog alfa pegol, wherein the prophylactic treatment with damoctocog alfa pegol comprises intravenous administration of a dose of 30 IU/kg to 40 IU/kg of the damoctocog alfa pegol 2 to per week, or 45 IU/kg to 60 IU/kg every 5 days.

E224. The chimeric protein for use according to embodiment 219, for reducing the ABR compared to prophylactic treatment with efmoroctocog alfa, wherein the prophylactic treatment with efmoroctocog alfa comprises intravenous administration of a dose of 25 IU/kg to 65 IU/kg of the efmoroctocog alfa every 3 to 5 days, or 50 IU/kg every 4 days.

E225. The chimeric protein for use according to embodiment 219, for reducing the ABR compared to prophylactic treatment with simoctocog alfa, wherein the prophylactic treatment with simoctocog alfa comprises intravenous administration of a dose of 30 IU/kg to 40 IU/kg of the simoctocog alfa every other day.

E226. The chimeric protein for use according to embodiment 219, for reducing the ABR compared to prophylactic treatment with moroctocog alfa, wherein the prophylactic treatment with moroctocog alfa comprises intravenous administration of a dose of 30 IU/kg of the moroctocog alfa at least 2 to 3 times per week.

E227. A chimeric protein for use in reducing an annualized bleeding rate (ABR) compared to prophylactic treatment with a complex, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and wherein the complex comprises a FVIII protein and a wild-type VWF protein or a portion thereof, wherein the FVIII protein and the VWF protein or portion thereof are not directly or indirectly covalently attached to each other.

E228. The chimeric protein for use according to embodiment 227, wherein the prophylactic treatment with the complex comprises intravenous administration of a dose of 30 IU/kg of the complex 3 times per week.

E229. A chimeric protein for use in reducing an annualized bleeding rate (ABR) compared to prophylactic treatment with emicizumab, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E230. The chimeric protein for use according to embodiment 229, wherein the prophylactic treatment with the emicizumab comprises subcutaneous administration of a dose of 1.5 mg/kg of the emicizumab once per week.

E231. The chimeric protein for use according to embodiment 229, wherein the prophylactic treatment with the emicizumab comprises subcutaneous administration of a dose of 3 mg/kg of the emicizumab once every two weeks.

E232. The chimeric protein for use according to embodiment 229, wherein the prophylactic treatment with the emicizumab comprises subcutaneous administration of a dose of 6 mg/kg of the emicizumab once every 4 weeks.

E233. The chimeric protein for use according to embodiment 229, wherein the prophylactic treatment with the emicizumab comprises subcutaneous administration of a loading dose of 3 mg/kg by subcutaneous injection once weekly for the first 4 weeks followed by a maintenance dose of (a) a dose of 3 mg/kg of the emicizumab once every two weeks; (b) a dose of 6 mg/kg of the emicizumab once every 4 weeks; or (c) a dose of 3 mg/kg of the emicizumab once every two weeks.

E234. The chimeric protein for use according to any one of embodiments 229 to 233, wherein the emicizumab is emicizumab-kxwh.

E235. The chimeric protein for use according to any one of embodiments 201, 202, or 210 to 234, wherein the ABR is reduced by from 50% to 90%.

E236. The chimeric protein for use according to any one of embodiments 201, 202, or 210 to 235, wherein the ABR is reduced by at least 75%.

E237. The chimeric protein for use according to any one of embodiments 201, 202, or 210 to 236, wherein the ABR is less than about 1.

E238. The chimeric protein for use according to any one of embodiments 201, 202, or 210 to 237, wherein the ABR is from about 0.5 to about 1.

E239. The chimeric protein for use according to any one of embodiments 201, 202, or 210 to 238, wherein the ABR is from about 0.5 to about 0.75.

E240. The chimeric protein for use according to any one of embodiments 201, 202, or 210 to 237, wherein the ABR is from about 0 to about 0.5.

E241. The chimeric protein for use according to any one of embodiments 201, 202, or 210 to 237, wherein the ABR is from about 0.25 to about 0.75.

E242. The method of any one of embodiments 201, 202, or 210 to 237, wherein the ABR is from 0 to about 0.25.

E243. The method of any one of embodiments 201, 202, or 210 to 237, wherein the ABR is 0.

E244. The chimeric protein for use according to any one of embodiments 201, 202, or 210 to 243, wherein the ABR is the ABR of spontaneous bleeding episodes.

E245. The chimeric protein for use according to any one of embodiments 201, 202, or 210 to 243, wherein the ABR is the ABR of traumatic bleeding episodes.

E246. The chimeric protein for use according to any one of embodiments 201, 202, or 210 to 243, wherein the ABR is the ABR of spontaneous and traumatic bleeding episodes.

E247. The chimeric protein for use according to any one of embodiments 201, 202, or 210 to 243, wherein the ABR is the ABR of spontaneous and traumatic bleeding episodes other than normal bleeds.

E248. The chimeric protein for use according to any one of embodiments 201, 202, or 210 to 244, wherein the ABR is the ABR of spontaneous treated bleeding episodes.

E249. The chimeric protein for use according to any one of embodiments 201, 202, or 210 to 243 or 245, wherein the ABR is the ABR of traumatic treated bleeding episodes.

E250. The chimeric protein for use according to any one of embodiments 201, 202, or 210 to 243, wherein the ABR is the ABR of spontaneous treated bleeding episodes and traumatic treated bleeding episodes.

E251. The chimeric protein for use according to any one of embodiments 201 to 250, wherein a total of from about 2600 IU/kg to about 3000 IU/kg of the chimeric protein is administered to the subject per year.

E252. The chimeric protein for use according to any one of embodiments 201 to 250, wherein a total of from about 2600 IU/kg to about 2750 IU/kg of the chimeric protein is administered to the subject per year.

E253. The chimeric protein for use according to any one of embodiments 201 to 250, wherein a total of from about 2600 IU/kg to about 2700 IU/kg of the chimeric protein is administered to the subject per year.

E254. The chimeric protein for use according to any one of embodiments 201 to 250, wherein a total of from about 2600 IU/kg to about 2650 IU/kg of the chimeric protein is administered to the subject per year.

E255. The chimeric protein for use according to any one of embodiments 201 to 250, wherein a total of about 2600 IU/kg of the chimeric protein is administered to the subject per year.

E256. The chimeric protein for use according to any one of embodiments 201 to 250, wherein the ABR is less than 2 and the method comprises administering a total of about 2600 IU/kg/year of the chimeric protein.

E257. The chimeric protein of any one of embodiments 201, 202, or 210 to 255, wherein the ABR is less than 1 and the method comprises administering a total of about 2600 IU/kg/year of the chimeric protein.

E258. The chimeric protein of any one of embodiments 201, 202, or 210 to 256, wherein the ABR is between 1 and 0.5 and the method comprises administering a total of about 2600 IU/kg/year of the chimeric protein.

E259. The chimeric protein of any one of embodiments 201, 202, or 210 to 257, wherein the ABR is between 0.75 and 0.5 and the method comprises administering a total of about 2600 IU/kg/year of the chimeric protein.

E260. The chimeric protein of any one of embodiments 201, 202, or 210 to 257, wherein the ABR is between 0.5 and 0.25 and the method comprises administering a total of about 2600 IU/kg/year of the chimeric protein.

E261. The chimeric protein of any one of embodiments 201, 202, or 210 to 257, wherein the ABR is between 0.25 and 0 and the method comprises administering a total of about 2600 IU/kg/year of the chimeric protein.

E262. A chimeric protein for use in maintaining a FVIII activity level of at least 2%, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E263. The chimeric protein for use according to embodiment 262, wherein a FVIII activity level of from about 10% to about 15% is maintained.

E264. The chimeric protein for use according to embodiment 262, wherein a FVIII activity level of from about 15% to about 20% is maintained.

E265. The chimeric protein for use according to any one of embodiments 201 to 264, wherein a FVIII activity level of from about 35% to about 45% is maintained for at least 4 days after each administration of the chimeric protein.

E266. The chimeric protein of any one of embodiments 201 to 265, wherein a FVIII activity level of from about 10% to about 15% is maintained for at least 7 days after each administration of the chimeric protein.

E267. The chimeric protein of any one of embodiments 201 to 266, wherein a FVIII activity level of from about 2% to about 5% is maintained for at least 14 days after each administration of the chimeric protein.

E268. The chimeric protein of any one of embodiments 201 to 267, wherein a FVIII activity level of from about 3% to about 5% is maintained for at least 14 days after each administration of the chimeric protein.

E269. A chimeric protein for use in reducing the risk of a bleeding episode that requires more than one supplemental on-demand administration of a FVIII replacement protein during the prophylactic treatment of hemophilia A, wherein the prophylactic treatment of hemophilia A comprises administering to a human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E270. The chimeric protein for use according to embodiment 269, wherein the supplemental on-demand administration of the FVIII replacement protein is supplemental on-demand administration of the chimeric protein.

E271. The chimeric protein for use according to embodiment 269 or 270, wherein the risk is decreased by at least 70% compared to the risk of a corresponding subject who receives prophylactic treatment for hemophilia A that comprises administration of an FVIII replacement protein other than the chimeric protein, wherein the FVIII replacement protein other than the chimeric protein is capable of being bound by endogenous VWF.

E272. The chimeric protein for use according to any one of embodiments 269 to 271, wherein the risk is decreased by at least 90% compared to the risk of a corresponding subject who receives prophylactic treatment for hemophilia A that comprises administration of an FVIII replacement protein other than the chimeric protein, wherein the FVIII replacement protein other than the chimeric protein is capable of being bound by endogenous VWF.

E273. The chimeric protein for use according to any one of embodiments 269 to 272, wherein the supplemental on-demand dose 45 IU/kg to 70 IU/kg.

E274. The chimeric protein for use according to any one of embodiments 269 to 273, wherein the supplemental on-demand dose is about 50 IU/kg.

E275. A chimeric protein for use in resolving a bleeding episode, comprising administering to a human subject in need thereof a single on-demand dose of a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E276. The chimeric protein for use according to embodiment 275, wherein the single on-demand dose is about 50 IU/kg.

E277. The chimeric protein for use according to embodiment 275 or 276, wherein the bleeding episode is a spontaneous bleeding episode or a traumatic bleeding episode.

E278. The chimeric protein for use according to any one of embodiments 275 to 277, wherein the bleeding episode is in a joint.

E279. A chimeric protein for use in prophylactically treating hemophilia A, the method comprising administering to a human subject in need thereof a therapeutically effective amount of the chimeric protein, wherein a total of less than 3500 IU/kg/year, less than 3400 IU/kg/year, less than 3300 IU/kg/year, less than 3200 IU/kg/year, less than 3100 IU/kg/year, less than 3000 IU/kg/year, less than 2900 IU/kg/year, less than 2800 IU/kg/year, less than 2700 IU/kg/year, or about 2600 IU/kg/year of a chimeric protein is administered to the subject, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and wherein the chimeric protein is administered to the at a dose of 45 IU/kg to 70 IU/kg of a chimeric protein at a dosing interval of about 6 days to about 14 days.

E280. The chimeric protein for use according to embodiment 279, wherein the therapeutically effective amount is a dose of the chimeric protein is from 45 IU/kg to 70 IU/kg.

E281. The chimeric protein for use according to embodiment 279 or 280, the chimeric protein is administered at a dosing interval of about 6 days to about 14 days.

E282. A chimeric protein for use in reducing the need to administer on-demand prior to strenuous activity in the prophylactic treatment of hemophilia A, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E283. The chimeric protein for use according to embodiment 282, wherein the strenuous activity is an activity that raises the subject's heart rate to at least 115 beats per minute.

E284. The chimeric protein for use according to embodiment 282 or 283, wherein the strenuous activity comprises parkour, running, swimming, bicycling, or climbing.

E285. The chimeric protein for use according to embodiment 282 or 283, wherein the strenuous activity is hiking.

E286. The chimeric protein for use according to embodiment 284, wherein the climbing is rock climbing or mountain climbing.

E287. The chimeric protein for use according to any one of embodiments 282 to 284, wherein the strenuous activity is a sport.

E288. The chimeric protein for use according to any one of embodiments 282 to 287, wherein the strenuous activity is soccer, tennis, skiing, snowboarding, skateboarding, or basketball.

E289. The chimeric protein for use according to embodiment 288, wherein the sport is a contact sport.

E290. The chimeric protein for use according to embodiment 289, wherein the contact sport is football, hockey, rugby, boxing, wrestling, or a martial art.

E291. The chimeric protein for use according to any one of embodiments 282 to 290, wherein the subject needs less pain medication to decrease or manage pain associated with hemophilia A before, during, or within 1, 3, or 6 hours after the strenuous activity (i) compared to the amount needed when the subject is treated for hemophilia A with prophylactic treatment with emicizumab, (ii) compared to the amount needed when the subject is treated for hemophilia A with prophylactic treatment with a FVIII replacement protein other than the chimeric protein, wherein the FVIII replacement protein is capable of being bound by endogenous VWF, (iii) compared to the amount a corresponding subject who receives prophylactic treatment with emicizumab needs, or (iv) compared to the amount a corresponding subject who receives prophylactic treatment for hemophilia A that comprises administration of an FVIII replacement protein other than the chimeric protein needs, wherein the FVIII replacement protein is capable of being bound by endogenous VWF.

E292. The chimeric protein for use according to embodiment 291, wherein the subject does not need pain medication to decrease or manage pain associated with hemophilia A before, during, or within 1, 3, or 6 hours after the strenuous activity.

E293. A chimeric protein for use in improving quality of life with the prophylactic treatment of hemophilia A, wherein a therapeutically effective amount of the chimeric protein is administered to a human subject, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E294. The chimeric protein for use according to embodiment 293, wherein the quality of life is improved compared to (i) prophylactic treatment with emicizumab, (ii) prophylactic treatment with a FVIII replacement protein other than the chimeric protein, wherein the FVIII replacement protein is capable of being bound by endogenous VWF, (iii) to the quality of life of a corresponding subject who receives prophylactic treatment with emicizumab, or (iv) the quality of life of a corresponding subject who receives prophylactic treatment for hemophilia A that comprises administration of an FVIII replacement protein other than the chimeric protein, wherein the FVIII replacement protein is capable of being bound by endogenous VWF.

E295. The chimeric protein for use according to embodiment 294, wherein the quality of life is improved compared to previous prophylactic treatment with emicizumab or the quality of life of a corresponding subject who receives prophylactic treatment with emicizumab.

E296. The chimeric protein for use according to embodiment 295, wherein the prophylactic treatment with the emicizumab comprises subcutaneous administration of a dose of 1.5 mg/kg of the emicizumab once per week.

E297. The chimeric protein for use according to embodiment 295, wherein the prophylactic treatment with the emicizumab comprises subcutaneous administration of a dose of 3 mg/kg of the emicizumab once every two weeks.

E298. The chimeric protein for use according to embodiment 295, wherein the prophylactic treatment with the emicizumab comprises subcutaneous administration of a dose of 6 mg/kg of the emicizumab once every 4 weeks.

E299. The chimeric protein for use according to embodiment 295, wherein the prophylactic treatment with the emicizumab comprises subcutaneous administration of a loading dose of 3 mg/kg by subcutaneous injection once weekly for the first 4 weeks followed by a maintenance dose of (a) a dose of 1.5 mg/kg of the emicizumab once every week; (b) a dose of 6 mg/kg of the emicizumab once every 4 weeks; or (c) a dose of 3 mg/kg of the emicizumab once every two weeks.

E300. The chimeric protein for use according to embodiment 299, wherein the prophylactic treatment with the emicizumab comprises subcutaneous administration of a loading dose of 3 mg/kg by subcutaneous injection once weekly for the first 4 weeks followed by a maintenance dose of a dose of 1.5 mg/kg of the emicizumab once every week.

E301. The chimeric protein for use according to any one of embodiments 295 to 300, wherein the emicizumab is emicizumab-kxwh.

E302. The chimeric protein for use according to any one of embodiments 293 to 301, wherein improving the quality of life of the subject comprises reducing the Haem-A-QoL score of the subject.

E303. The chimeric protein for use according to embodiment 302, wherein the Haem-A-QoL score of the subject improves by at least 5 compared to the Haem-A-QoL score of the subject before the subject first received prophylactic treatment with the chimeric protein.

E304. The chimeric protein for use according to embodiment 302, wherein the Haem-A-QoL score of the subject improves by at least 6 compared to the Haem-A-QoL score of the subject before the subject first received prophylactic treatment with the chimeric protein.

E305. The chimeric protein for use according to embodiment 302, wherein the Haem-A-QoL score of the subject improves by at least 7 compared to the Haem-A-QoL score of the subject before the subject first received prophylactic treatment with the chimeric protein.

E306. The chimeric protein for use according to any one of embodiments 302 to 305, wherein the Haem-A-QoL score of the subject improves by at least 10% compared to the Haem-A-QoL score of the subject before the subject first received prophylactic treatment with the chimeric protein.

E307. The chimeric protein for use according to any one of embodiments 293 to 306, wherein improving the quality of life of the subject comprises reducing the pain of the subject.

E308. The chimeric protein for use according to embodiment 307, wherein improving the quality of life of the subject comprises reducing the subject's self-reported pain intensity, wherein the subject's self-reported pain intensity changes from severe to moderate, mild, or no pain.

E309. The chimeric protein for use according to embodiment 308, wherein the subject's self-reported pain intensity is a rating of the most intense hemophilia A-related pain over the last 7 days.

E310. The chimeric protein for use according to any one of embodiments 201 to 309, wherein, prior to administration of the chimeric protein, the subject is administered a pain medication to reduce or manage pain associated with hemophilia A, and the pain is reduced upon treatment with the chimeric protein such that the subject needs less pain medication to reduce or manage the pain to the same degree as experienced prior to administration of the chimeric protein.

E311. The chimeric protein for use according to embodiment 310, wherein the dose of the pain medication is reduced upon treatment with the chimeric protein.

E312. The chimeric protein for use according to embodiment 310 or 311, wherein the number of doses of the pain medication over the course of a day or a week is reduced upon treatment with the chimeric protein.

E313. The chimeric protein for use according to any one of embodiments 310 to 312, wherein the pain medication is a nonsteroidal anti-inflammatory drug (NSAID), an opioid, or a nonopioid pain reliever.

E314. The chimeric protein for use according to embodiment 313, wherein the pain medication comprises fentanyl, hydromorphone, morphine, oxycodone, oxymorphone, tramadol, oxycodone, oxycodone, acetaminophen, ibuprofen, naproxen sodium, celecoxib, ketorolac, or aspirin.

E315. The chimeric protein for use according to any one of embodiments 307 to 314, wherein the subject's self-reported pain intensity improves by at least 10%.

E316. The chimeric protein for use according to embodiment 315, wherein the subject's self-reported pain intensity is a rating of the most intense hemophilia A-related pain over the last 7 days as related on a numeric scale.

E317. The chimeric protein for use according to embodiment 316, wherein the numeric scale is from 1 to 5 or from 1 to 10.

E318. The chimeric protein for use according to any one of embodiments 293 to 317, wherein improving the quality of life of the subject comprises reducing the Hemophilia Joint Health Score (HJHS) score of the subject.

E319. The chimeric protein for use according to embodiment 318, wherein the HJHS score of the subject improves by at least 1 compared to the HJHS score of the subject before the subject first received prophylactic treatment with the chimeric protein.

E320. The chimeric protein for use according to embodiment 318, wherein the HJHS score of the subject improves by at least 2 compared to the HJHS score of the subject before the subject first received prophylactic treatment with the chimeric protein.

E321. The chimeric protein for use according to any one of embodiments 318 to 320, wherein the HJHS score of the subject improves by at least 10% compared to the HJHS score of the subject before the subject first received prophylactic treatment with the chimeric protein.

E322. A chimeric protein for use in improving one or more joint outcomes, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E323. The chimeric protein for use according to embodiment 322, wherein the one or more joint outcomes is improved compared to the outcomes of a corresponding subject who receives prophylactic treatment for hemophilia A that comprises administration of an FVIII replacement protein other than the chimeric protein, wherein the FVIII replacement protein is capable of being bound by endogenous VWF.

E324. The chimeric protein for use according to embodiment 323, wherein the one or more joint outcomes are improved compared to the outcomes of a corresponding subject who receives prophylactic treatment with emicizumab.

E325. The chimeric protein for use according to any one of embodiments 322 to 324, wherein improving one or more joint outcomes comprises reducing hemarthrosis.

E326. The chimeric protein for use according to any one of embodiments 322 to 325, wherein improving one or more joint outcomes comprises reducing the incidence of bleeds.

E327. The chimeric protein for use according to embodiment 326, wherein treatment with the chimeric protein results in an annualized joint bleeding rate (AjBR) of less than about 1.

E328. The chimeric protein for use according to embodiment 326, wherein treatment with the chimeric protein results in an AjBR of about 0.

E329. The chimeric protein for use according to embodiment 326, wherein treatment with the chimeric protein results in an AjBR of from about 0.25 to about 0.75.

E330. The chimeric protein for use according to any one of embodiments 322 to 329, wherein improving one or more joint outcomes comprises reversing hemophilic arthropathy of a target joint.

E331. The chimeric protein for use according to embodiment 330, wherein improving one or more joint outcomes comprises wherein the reversible hemophilic arthropathy comprises synovitis, a microbleed, or both.

E332. The chimeric protein for use according to any one of embodiments 322 to 333, wherein improving one or more joint outcomes comprises reducing vascular remodeling in one or more target joints.

E333. The chimeric protein for use according to any one of embodiments 322 to 332, wherein improving one or more joint outcomes comprises reducing paint or swelling in surrounding soft tissue of a joint.

E334. The chimeric protein for use according to any one of embodiments 322 to 333, wherein the joint is an elbow, a knee, an ankle, a shoulder joint, a hip, a wrist, a hand joint, or a foot joint.

E335. The chimeric protein for use according to any one of embodiments 322 to 334, wherein improving one or more joint outcomes comprises increased joint flexion ability, increased joint extension, increased range of movement, increased muscle strength, decreased swelling, decreased duration of swelling, decreased crepitus, decreased pain, or decreased muscle atrophy of one or more joints.

E336. The chimeric protein for use according to any one of embodiments 322 to 335, wherein improving one or more joint outcomes comprises improved walking, improved ability to use stairs, improved running ability, or improved ability to hop on one leg.

E337. The chimeric protein for use according to any one of embodiments 322 to 336, wherein improving one or more joint outcomes comprises achieving an excellent outcome for at least 90% of joint bleeds as assessed using the International Society on Thrombosis and Haemostasis (ISTH) 4-point assessment scale.

E338. The chimeric protein for use according to embodiment 337, wherein improving one or more joint outcomes comprises achieving an excellent outcome for at least 95% of joint bleeds as assessed using the International Society on Thrombosis and Haemostasis (ISTH) 4-point assessment scale.

E339. The chimeric protein for use according to any one of embodiments 322 to 338, wherein improving one or more joint outcomes comprises achieving an excellent outcome for at least 90% of joint bleeds as assessed using the Physician's Global Assessment scale.

E340. The chimeric protein for use according to embodiment 339, wherein improving one or more joint outcomes comprises achieving an excellent outcome for at least 95% of joint bleeds as assessed using the Physician's Global Assessment scale.

E341. The chimeric protein for use according to any one of embodiments 201 to 340, wherein the treatment for hemophilia A is routine prophylaxis.

E342. The chimeric protein for use according to any one of embodiments 201 to 272 or 279 to 3415, wherein the therapeutically effective amount is a dose of about 45 IU/kg to about 70 IU/kg of a chimeric protein at a dosing interval of about 6 days to about 14 days.

E343. The chimeric protein for use according to any one of embodiments 201 to 342, wherein the therapeutically effective amount is a dose of about 50 IU/kg the chimeric protein at a dosing interval of about 6 days to about 14 days.

E344. The chimeric protein for use according to any one of embodiments 201 to 343, wherein the therapeutically effective amount is a dose of about 50 IU/kg the chimeric protein at a dosing interval of about 6 days to about 8 days.

E345. The chimeric protein for use according to any one of embodiments 201 to 343, wherein the therapeutically effective amount is a dose of about 50 IU/kg the chimeric protein at a dosing interval of about 10 days to about 14 days.

E346. The chimeric protein for use according to any one of embodiments 201 to 344, wherein the therapeutically effective amount is a dose of about 50 IU/kg the chimeric protein at a dosing interval of about 7 days.

E347. A chimeric protein for use in the on-demand treatment of a minor bleed, a moderate bleed, or a major bleed, wherein a therapeutically effective amount of the chimeric protein is administered to a human subject who has hemophilia A, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E348. The chimeric protein for use according to embodiment 347, for on-demand treatment of a minor bleed or a moderate bleed.

E349. The chimeric protein for use according to embodiment 348, wherein the minor bleed or moderate bleed is an uncomplicated joint bleed, a minor muscular bleed, a mucosal bleed, or a subcutaneous bleed.

E350. The chimeric protein for use according to embodiment 347, for on-demand treatment of a major bleed.

E351. The chimeric protein for use according to embodiment 350, wherein the major bleed is an intracranial bleed, a retroperitoneal bleed, an iliopsoas bleed, a neck bleed, a muscle bleed with compartment syndrome, and/or a bleed associated with a significant decrease in the hemoglobin level.

E352. The chimeric protein for use according to any one of embodiments 347 to 351, wherein the therapeutically effective amount is a dose of from 30 IU/kg to 50 IU/kg.

E353. The chimeric protein for use according to any one of embodiments 347 to 352, wherein the therapeutically effective amount is a dose of 30 IU/kg.

E354. The chimeric protein for use according to any one of embodiments 347 to 352, wherein the therapeutically effective amount is a dose of 50 IU/kg.

E355. The chimeric protein for use according to any one of embodiments 347 to 354, wherein the dose is administered once.

E356. The chimeric protein for use according to any one of embodiments 347 to 355, wherein the dose is administered every 2 or 3 days until the bleed has stopped.

E357. The chimeric protein for use according to any one of embodiments 347 to 356, wherein the subject is receiving prophylactic treatment for hemophilia A with the chimeric protein, and the dose is administered 2 to 3 days or less after a prophylactic dose.

E358. The chimeric protein for use according to any one of embodiments 347 to 357, wherein the subject is receiving prophylactic treatment for hemophilia A with the chimeric protein, and at least 3 days pass after the last on-demand dose to treat the bleed before another prophylactic dose is administered.

E359. A chimeric protein for use in the perioperative management of bleeding, comprising administering to a human subject who has hemophilia A in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

E360. The chimeric protein for use according to embodiment 359, wherein the subject is expected to receive surgery within 72 hours.

E361. The chimeric protein for use according to embodiment 359, wherein the subject is receiving surgery.

E362. The chimeric protein for use according to embodiment 359, wherein the subject has received surgery within the last week.

E363. The chimeric protein for use according to any one of embodiments 359 to 362, wherein the surgery is major surgery.

E364. The chimeric protein for use according to any one of embodiments 359 to 362, wherein the surgery is minor surgery.

E365. The chimeric protein for use according to any one of embodiments 359 to 364, wherein the therapeutically effective amount achieves an excellent hemostatic response as assessed by a surgeon.

E366. The chimeric protein for use according to any one of embodiments 359 to 365, wherein the therapeutically effective amount is from about 25 IU/kg to about 65 IU/kg.

E367. The chimeric protein for use according to any one of embodiments 159 to 366, wherein the therapeutically effective amount is about 30 IU/kg or about 50 IU/kg.

E368. The chimeric protein for use according to any one of embodiments 359 to 366, wherein the therapeutically effective amount comprises a preoperative loading dose of 50 IU/kg, followed by one or more doses of 30 or 50 IU/kg every 2 to 3 days.

E369. The chimeric protein for use according to any one of embodiments 201 to 368, wherein the FVIII polypeptide has a deletion of amino acids 746 to 1648 corresponding to mature FVIII (SEQ ID NO: 8), the first ELNN polypeptide is inserted within the FVIII polypeptide immediately downstream of amino acid 745 corresponding to mature FVIII (SEQ ID NO: 8), and the first ELNN polypeptide comprises an amino acid sequence that is least about 90% identical to the amino acid sequence of SEQ ID NO: 9.

E370. The chimeric protein for use according to any one of embodiments 201 to 369, wherein the VWF fragment comprises a D′ domain of VWF and a D3 domain of VWF, the VWF fragment is mutated to substitute cysteines involved in VWF dimerization to alanine, the second ELNN polypeptide comprises an amino acid sequence that is at least about 90% identical to the amino acid sequence of SEQ ID NO: 14, and the linker comprises an amino acid sequence that is at least about 90% identical to the amino acid sequence of SEQ ID NO: 15.

E371. The chimeric protein for use according to any one of embodiments 201 to 370, wherein the first ELNN polypeptide comprises the sequence of SEQ ID NO: 9 and the second ELNN polypeptide comprises the amino acid sequence of SEQ ID NO: 14.

E372. The chimeric protein for use according to any one of embodiments 201 to 371, wherein the first polypeptide comprises an amino acid sequence that is at least about 95% identical to the amino acid sequence of SEQ ID NO: 1 and the second polypeptide comprises an amino acid sequence that is at least about 95% identical to the amino acid sequence of SEQ ID NO: 2, wherein the first polypeptide and the second polypeptide are covalently linked by two disulfide bonds between the first Fc region and the second Fc region.

E373. The chimeric protein for use according to any one of embodiments 201 to 372, wherein the first polypeptide comprises the amino acid sequence set forth as SEQ ID NO: 1 and the second polypeptide comprises the amino acid sequence set forth as SEQ ID NO: 2, wherein the first polypeptide and the second polypeptide are covalently linked by two disulfide bonds between the first Fc region and the second Fc region.

E374. The chimeric protein for use according to any one of embodiments 201 to 373, wherein the chimeric protein is efanesoctocog alfa.

E375. The chimeric protein for use according to any one of embodiments 201 to 374, wherein every dose of the chimeric compound that is administered to the subject is about the same for at least 1 month.

E376. The chimeric protein for use according to any one of embodiments 201 to 375, wherein every dose of the chimeric compound that is administered to the subject is about the same for at least 3 months.

E377. The chimeric protein for use according to any one of embodiments 201 to 376, wherein every dose of the chimeric compound that is administered to the subject is about the same for at least 6 months.

E378. The chimeric protein for use according to any one of embodiments 201 to 377, wherein every dose of the chimeric compound that is administered to the subject is about the same for at least 9 months.

E379. The chimeric protein for use according to any one of embodiments 201 to 378, wherein every dose of the chimeric compound that is administered to the subject is about the same for at least 12 months.

E380. The chimeric protein for use according to any one of embodiments 201 to 379, wherein the FVIII activity level in the subject is not measured for at least 1 month.

E381. The chimeric protein for use according to any one of embodiments 201 to 380, wherein the FVIII activity level in the subject is not measured for at least 3 months.

E382. The chimeric protein for use according to any one of embodiments 201 to 381, wherein the FVIII activity level in the subject is not measured for at least 6 months.

E383. The chimeric protein for use according to any one of embodiments 201 to 182, wherein the FVIII activity level in the subject is not measured for at least 9 months.

E384. The chimeric protein for use according to any one of embodiments 201 to 183, wherein the FVIII activity level in the subject is not measured for at least 12 months.

E385. The chimeric protein for use according to any one of embodiments 201 to 184, wherein the subject has severe hemophilia A.

E386. The chimeric protein for use according to any one of embodiments 201 to 385, wherein the subject has previously been treated for hemophilia A with a FVIII replacement protein.

E387. The chimeric protein for use according to any one of embodiments 201 to 385, wherein the subject has not previously been treated for hemophilia A with a FVIII replacement protein.

E388. The chimeric protein for use according to any one of embodiments 201 to 387, wherein the subject is at least 12 years old.

E389. The chimeric protein for use according to any one of embodiments 201 to 387, wherein the subject is at least 18 years old.

E390. The chimeric protein for use according to any one of embodiments 201 to 387, wherein the subject is less than 12 years old.

E391. The chimeric protein for use according to any one of embodiments 201 to 390, wherein the subject has at least 1 target joint.

E392. The chimeric protein for use according to embodiment 391, wherein the subject has at least 3 target joints.

E393. The chimeric protein for use according to embodiment 392, wherein the subject has at least 6 target joints.

E394. The chimeric protein for use according to any one of embodiments 391 to 393, wherein treatment with the chimeric protein resolves the subject's target joint(s).

E395. The chimeric protein of any one of embodiments 201 to 394, wherein the subject has received on-demand treatment for at least 2 bleeding episode within the last year.

E396. The chimeric protein for use according to embodiment 395, wherein the subject has received on-demand treatment for at least 3 bleeding episodes within the last year.

E397. The chimeric protein for use according to embodiment 396, wherein the subject has received on-demand treatment for at least 5 bleeding episodes within the last year.

E398. The chimeric protein for use according to anyone of embodiments 395 to 3973, wherein the bleeding episodes were traumatic bleeding episodes.

E399. The chimeric protein for use according to anyone of embodiments 395 to 397, wherein the bleeding episodes were spontaneous bleeding episodes.

E400. The chimeric protein for use according to any one of embodiments 201 to 399, wherein the chimeric protein is administered intravenously.

E401. The method of any one of embodiments 1 to 200 or the chimeric protein for use according to any one of embodiments 201 to 399, wherein the chimeric protein is administered in a pharmaceutical composition that comprises (a) 5% (w/v) to 7.5% (w/v) sucrose; (b) 7.5 mM to 12.5 mM histidine; (c) 225 mM to about 300 mM arginine; (d) 5 mM to 6 mM calcium chloride; and (e) 0.01% (w/v) to about 0.075% (w/v) polysorbate 80.

E402. The method of any one of embodiments 1 to 200 or the chimeric protein for use according to any one of embodiments 201 to 399, wherein the chimeric protein is administered in a pharmaceutical composition that comprises about 10 mM L-histidine, about 250 mM Arginine-HCl, about 5 mM CaCl₂, about 5% (w/v) sucrose, and about 0.05% (w/v) polysorbate 80.

E403. A kit comprising a pharmaceutical label and a chimeric protein, wherein the pharmaceutical label comprises a recommendation or instruction for practicing a method of any one of embodiments 1 to 200, and wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E404. The kit of embodiment 403, wherein the pharmaceutical label comprises a recommendation for practicing a method of any one of embodiments 1 to 200.

E405. The kit of embodiment 403, wherein the pharmaceutical label comprises an instruction for practicing a method of any one of embodiments 1 to 200.

E406. A kit comprising a pharmaceutical label and a chimeric protein, wherein the pharmaceutical label comprises information that is sufficient to enable a human subject, a caregiver, or a medical practitioner to practice the method of any one of embodiments 1 to 200, and wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E407. The kit of any one of embodiments 403 to 406, wherein the chimeric protein is in a lyophilized pharmaceutical composition, the kit further comprises a diluent for reconstituting the lyophilized pharmaceutical composition.

E408. The kit of embodiment 407, wherein the diluent is sterile water.

E409. The kit of embodiment 403 or 408, wherein the diluent is in a pre-filled syringe.

E410. The method of any one of embodiments 93 to 117, wherein improving the quality of life of the subject comprises reducing or maintaining the Hemophilia Early Arthropathy Detection with Ultrasound (HEAD-US) score of the subject.

E411. The method of embodiment 410, wherein the subject had no target joints before the subject first received prophylactic treatment with the chimeric protein.

E412. The method of embodiment 410 or 411, wherein the HEAD-US score of the subject improves by at least 1 compared to the HEAD-US score of the subject before the subject first received prophylactic treatment with the chimeric protein.

E413. The method of any one of embodiments 410 to 412, wherein the HEAD-US score of the subject improves by at least 1.5 compared to the HEAD-US score of the subject before the subject first received prophylactic treatment with the chimeric protein.

E414. The method of embodiment 410, wherein the subject had one or more target joints before the subject first received prophylactic treatment with the chimeric protein.

E415. The method of embodiment 414, wherein the subject had at least 2, 3, 4, or 5 target joints before the subject first received prophylactic treatment with the chimeric protein.

E416. The method of embodiment 414 or 415, wherein the HEAD-US score of the subject improves by at least 0.5 compared to the HEAD-US score of the subject before the subject first received prophylactic treatment with the chimeric protein.

E417. The method of any one of embodiments 410 to 416, wherein the HEAD-US score of the subject improves in the subject's left elbow, right elbow, left knee, right knee, left ankle, and/or right ankle.

E418. The method of any one of embodiments 410 to 417, wherein the subject is at least 50 years old, from 40 to 49 years old, from 30 to 39 years old, from 18 to 29 years old, or from 12 to 17 years old.

E419. The method of embodiment 417, wherein the subject is at least 50 years old.

E420. The method of embodiment 417, wherein the subject is from 40 to 49 years old.

E421. The method of embodiment 417, wherein the subject is from 30 to 39 years old.

E422. The method of any one of embodiments 410 to 421, wherein the subject's body mass index is less than 25.

E423. The method of any one of embodiments 410 to 421, wherein the subject's body mass index is at least 25 but less than 30.

E424. The method of any one of embodiments 410 to 421, wherein the subject's body mass index is at least 30.

E425. The chimeric protein for use according to any one of embodiments 293 to 317, wherein improving the quality of life of the subject comprises reducing or maintaining the Hemophilia Early Arthropathy Detection with Ultrasound (HEAD-US) score of the subject.

E426. The chimeric protein for use according to embodiment 425, wherein the subject had no target joints before the subject first received prophylactic treatment with the chimeric protein.

E427. The chimeric protein for use according to embodiment 425 or 426, wherein the HEAD-US score of the subject improves by at least 1 compared to the HEAD-US score of the subject before the subject first received prophylactic treatment with the chimeric protein.

E428. The chimeric protein for use according to any one of embodiments 425-427, wherein the HEAD-US score of the subject improves by at least 1.5 compared to the HEAD-US score of the subject before the subject first received prophylactic treatment with the chimeric protein.

E429. The chimeric protein for use according to embodiment 425, wherein the subject had one or more target joints before the subject first received prophylactic treatment with the chimeric protein.

E430. The chimeric protein for use according to embodiment 429, wherein the subject had at least 2, 3, 4, or 5 target joints before the subject first received prophylactic treatment with the chimeric protein.

E431. The chimeric protein for use according to embodiment 429 or 430, wherein the HEAD-US score of the subject improves by at least 0.5 compared to the HEAD-US score of the subject before the subject first received prophylactic treatment with the chimeric protein.

E432. The chimeric protein for use according to any one of embodiments 425 to 431, wherein the HEAD-US score of the subject improves in the subject's left elbow, right elbow, left knee, right knee, left ankle, and/or right ankle.

E433. The chimeric protein for use according to any one of embodiments 425 to 432, wherein the subject is at least 50 years old, from 40 to 49 years old, from 30 to 39 years old, from 18 to 29 years old, or from 12 to 17 years old.

E434. The chimeric protein for use according to embodiment 433, wherein the subject is at least 50 years old.

E435. The chimeric protein for use according to embodiment 433, wherein the subject is from 40 to 49 years old.

E436. The chimeric protein for use according to embodiment 433, wherein the subject is from 30 to 39 years old.

E437. The chimeric protein for use according to any one of embodiments 425 to 436, wherein the subject's body mass index is less than 25.

E438. The chimeric protein for use according to any one of embodiments 425 to 436, wherein the subject's body mass index is at least 25 but less than 30.

E439. The chimeric protein for use according to any one of embodiments 423 to 436, wherein the subject's body mass index is at least 30.

E440. A method of improving joint health in a human subject during the prophylactic treatment of hemophilia A, wherein the prophylactic treatment of hemophilia A comprises administering to the human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E441. The method of embodiment 440, wherein joint health is measured by ultrasound.

E442. The method of embodiment 441, wherein the ultrasound is Power Doppler Ultrasound (PDUS).

E443. The method of any one of embodiments 440 to 442, wherein an improvement in joint health is measured by an improvement in PDUS grading assessment score.

E444. The method of embodiment 440, wherein joint health is measured by HEAD-US scoring system.

E445. The method of embodiment 444, wherein the subject has a hypertrophic synovium and improvement in joint health comprises an improvement or reduction of the hypertrophy in the synovium.

E446. The method of embodiment 444 or 445, wherein the improvement in joint health comprises an improvement in bone in the subject.

E447. The method of any one of embodiments 444 to 445, wherein the improvement in joint health comprises an improvement in cartilage in the subject.

E448. The method of embodiment 447, wherein the improvement in cartilage comprises increased cartilage thickness.

E449. The method of any one of embodiments 440 to 448, wherein the HEAD-US score of the subject improves in the subject's left elbow, right elbow, left knee, right knee, left ankle, and/or right ankle.

E450. The method of any one of embodiments 440 to 449, wherein the subject is at least 50 years old, from 40 to 49 years old, from 30 to 39 years old, from 18 to 29 years old, or from 12 to 17 years old.

E451. The method of embodiment 450, wherein the subject is at least 50 years old.

E452. The method of embodiment 450, wherein the subject is from 40 to 49 years old.

E453. The method of embodiment 450, wherein the subject is from 30 to 39 years old.

E454. The method of any one of embodiments 440 to 453, wherein the subject's body mass index is less than 25.

E455. The method of any one of embodiments 440 to 453, wherein the subject's body mass index is at least 25 but less than 30.

E456. The method of any one of embodiments 440 to 453, wherein the subject's body mass index is at least 30.

E457. The method of any one of embodiments 440 to 451, wherein the subject is at least 50 years old and has a body mass index of at least 25.

E458. A chimeric protein for use in improving joint health in a human subject during the prophylactic treatment of hemophilia A, wherein the prophylactic treatment of hemophilia A comprises administering to the human subject in need thereof a therapeutically effective amount of a chimeric protein, wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E459. The chimeric protein for use according to embodiment 458, wherein joint health is measured by ultrasound.

E460. The chimeric protein for use according to embodiment 459, wherein the ultrasound is Power Doppler Ultrasound (PDUS).

E461. The chimeric protein for use according to any one of embodiments 458 to 460, wherein an improvement in joint health is measured by an improvement in PDUS grading assessment score.

E462. The chimeric protein for use according to embodiment 458, wherein joint health is measured by HEAD-US scoring system.

E463. The chimeric protein for use according to embodiment 462, wherein the subject has a hypertrophic synovium and improvement in joint health comprises an improvement or reduction of the hypertrophy in the synovium.

E464. The chimeric protein for use according to embodiment 462 or 463, wherein the improvement in joint health comprises an improvement in bone in the subject.

E465. The chimeric protein for use according to any one of embodiments 462 to 463, wherein the improvement in joint health comprises an improvement in cartilage in the subject.

E466. The chimeric protein for use according to embodiment 465, wherein the improvement in cartilage comprises increased cartilage thickness.

E467. The chimeric protein for use according to any one of embodiments 458 to 466, wherein the HEAD-US score of the subject improves in the subject's left elbow, right elbow, left knee, right knee, left ankle, and/or right ankle.

E468. The chimeric protein for use according to any one of embodiments 458 to 467, wherein the subject is at least 50 years old, from 40 to 49 years old, from 30 to 39 years old, from 18 to 29 years old, or from 12 to 17 years old.

E469. The chimeric protein for use according to embodiment 468, wherein the subject is at least 50 years old.

E470. The chimeric protein for use according to embodiment 468, wherein the subject is from 40 to 49 years old.

E471. The chimeric protein for use according to embodiment 468, wherein the subject is from 30 to 39 years old.

E472. The chimeric protein for use according to any one of embodiments 458 to 471, wherein the subject's body mass index is less than 25.

E473. The chimeric protein for use according to any one of embodiments 458 to 471, wherein the subject's body mass index is at least 25 but less than 30.

E474. The chimeric protein for use according to any one of embodiments 458 to 471, wherein the subject's body mass index is at least 30.

E475. The chimeric protein for use according to any one of embodiments 458 to 469, wherein the subject is at least 50 years old and has a body mass index of at least 25.

E476. A kit comprising a pharmaceutical label and a chimeric protein, wherein the pharmaceutical label comprises a recommendation or instruction for practicing a method of any one of embodiments 410 to 424 or 440 to 457, and wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E477. The kit of embodiment 476, wherein the pharmaceutical label comprises a recommendation for practicing a method of any one of embodiments 410 to 424 or 440 to 457.

E478. The kit of embodiment 476, wherein the pharmaceutical label comprises an instruction for practicing a method of any one of embodiments 410 to 424 or 440 to 457.

E479. A kit comprising a pharmaceutical label and a chimeric protein, wherein the pharmaceutical label comprises information that is sufficient to enable a human subject, a caregiver, or a medical practitioner to practice the method of any one of embodiments 410 to 424 or 440 to 457, and wherein the chimeric protein comprises a first polypeptide and a second polypeptide, wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond.

E480. The kit of any one of embodiments 476 to 479, wherein the chimeric protein is in a lyophilized pharmaceutical composition, the kit further comprises a diluent for reconstituting the lyophilized pharmaceutical composition.

E481. The kit of embodiment 480, wherein the diluent is sterile water.

E482. The kit of embodiment 476 or 481, wherein the diluent is in a pre-filled syringe.

### VI. References

Xyntha [package insert]. Philadelphia, PA: Wyeth Pharmaceuticals Inc; 2015.
Kogenate [package insert]. Whippany, NJ: Bayer HealthCare LLC; 2016
Nuwiq [package insert]. SE-112 75, Sweden: Octapharma; 2017
Kavakli K, Yang R, Rusen L, Beckmann H, Tseneklidou-Stoeter D, Maas Enriquez M. Prophylaxis vs. on-demand treatment with BAY 81-8973, a full-length plasma protein-free recombinant factor VIII product: results from a randomized trial (LEOPOLD II). J Thromb Haemost. 2015 March;13(3):360-9.
Konkle B, Stasyshyn O, Chowdary P, Bevan D H, Mant T, Shima M, et al. Pegylated, full-length, recombinant factor VIII for prophylactic and on-demand treatment of severe hemophilia A. Blood. 2015 August; 126(9):1078-85.
Mahlangu J, Powell J S, Ragni M V, Chowdary P, Josephson N C, Pabinger I, et al. Phase 3 study of recombinant factor VIII Fc fusion protein in severe hemophilia A. Blood. 2014 January;123(3):317-25.
Mahlangu J, Kuliczkowski K, Karim F A, Stasyshyn O, Kosinova M V, Lepatan L M, et al. Efficacy and safety of rVIII-SingleChain: results of a phase 1/3 multicenter clinical trial in severe hemophilia A. Blood. 2016. 2016 August; 128(5):630-7.
Giangrande P, Andreeva T, Chowdary P, Ehrenforth S, Hanabusa H, Leebeek F W, et al. Clinical evaluation of glycoPEGylated recombinant FVIII: Efficacy and safety in severe haemophilia A. Thromb Haemost. 2017 Jan. 26; 117(2):252-261.
Kavakli K, Yang R, Rusen L, Beckmann H, Tseneklidou-Stoeter D, Maas Enriquez M. Prophylaxis vs. on-demand treatment with BAY 81-8973, a full-length plasma protein-free recombinant factor VIII product: results from a randomized trial (LEOPOLD II). J Thromb Haemost. 2015 March;13(3):360-9.
Manco-Johnson M J, Kempton C L, Reding M T, Lissitchkov T, Goranov S, Gercheva L, et al. Randomized, controlled, parallel-group trial of routine prophylaxis vs. on-demand treatment with sucrose-formulated recombinant factor VIII in adults with severe hemophilia A (SPINART). J Thromb Haemost. 2013 June; 11(6):1119-27.
Reding M T, Ng H J, Poulsen L H, Eyster M E, Pabinger I, Shin H J, et al. Safety and efficacy of BAY 94-9027, a prolonged-half-life factor VIII. J Thromb Haemost. 2017 March;15(3):411-419.
Furie B, Furie B C. Molecular basis of hemophilia. Semin Hematol. 1990; 27(3)270-85.
Graw J, Brackmann H H, Oldenburg J, Schneppenheim R, Spannagl M, Schwaab R. Haemophilia A: from mutation analysis to new therapies. Nat Rev Genetic. 2005 June; 6(6): 448.
Amy L and Dunn M D. Hemophilia A, in Transfusion Medicine and Hemostasis (2nd Ed.)2013. Clinical and Laboratory Aspects, Chapter 106, pp 699-704.
Skinner M W. WFH: Closing the global gap-achieving optimal care. Haemophilia. 2012;18(Suppl 4):1-12
World Federation of Hemophilia. World Federation of Hemophilia Report on the Annual Global Survey 2017. Montreal, Quebeck: World Federation of Hemophilia; October 2018. Available at: http://www1.wfh/org/publications/files/pdf-1714.pdf.
Aznar J A, Lucia F, Abad-Franch L, Jimenez-Yuste V, Perez R, Batlle J, et al. Haemophilia in Spain. Haemophilia. 2009 May; 15(3)665-75.
Centers for Disease Control and Prevention (CDC). Summary Report of UDC Activity National, Patient Demographics (Hemophilia) 2017. Available at: https://www2a.cdc.gov/ncbddd/htcweb/UDC_Report/UDC_Report.asp.
United Kingdom Haemophilia Centre Doctors' Organization (UCDO UKHCDO Bleeding Disorder Statistics for 2017 to 2018: a report from the National Haemophila Database. October 2018. Available at: http://www.ukhcdo.org/wp-content/uploads/2019/04/2018_UKHCDO_Annual_Report_2017_18_Data.pdf.
Roosendall G, Lafeber F P. Blood-induced joint damage in hemophilia. Semin Thromb Hemost. 2003; 29(1): 37-42.
Witmer C, Presley R, Kulkarni R, Soucie J, Manno C S, Raffini L. Associations between intracranial haemorrhage and prescribed prophylaxis in a large cohort of haemophilia patients in the United States. Br J Haematol. 2011. January; 152(2):211-6.
Lee S O, Yu S Y. Utilization Patterns of Coagulation Factor Consumption for Patients with Hemophila. J Korean Med Sci. 2016. January 1; 31(1):33-8.
Lusher J M. Hemophilia: From plasma to recombinant factors. 2008. In: 50 Years in Hematology Research That Revolutionized Patient Care. Washington, DC: American Society of Hematology; p. 25-27. Available from: http://www.hematology.org/Publications/50 Years in Hematology/4323.aspx.
Pool J G, Gershgold E J, Pappenhagen A R. High potency antihaemophilic factor concentrate prepared from cryoglobulin precipitate. Nature. 1964; 203:312.
Kingdon H S, Lundblad R L. An adventure in biotechnology: the development of haemophilia A therapeutics from whole blood transfusion to recombinant DNA to gene therapy. Biotechnol Appl Biochem. 2002;35(Pt 2):141-8.
Ramgren O. A clinical and medico-social study of haemophilia in Sweden. Acta Med Scand Suppl. 1962; 379:111-90.
Steen Carlsson K, HojgArd S, Glomstein A, et al. On-demand vs. prophylactic treatment for severe haemophilia in Norway and Sweden: differences in treatment characteristics and outcome. Haemophilia. 2003; 9(5):555-66
Manco-Johnson M J, Abshire T C, Shapiro A D, Riske B, Hacker M R, Kilcoyne R, et al. Prophylaxis versus episodic treatment to prevent joint disease in boys with severe hemophilia. N Engl J Med. 2007; 357:535-544.
Nilsson I M, Berntorp E, Lofqvist T, Pettersson H. Twenty-five years experience of prophylactic treatment in severe hemophilia A and B. J Intern Med. 1992; 232:25-32.
Gringeri A, Lundin B, von Mackensen S, et al. A randomized clinical trial of prophylaxis in children with hemophilia A (the ESPRIT Study). J Thromb Haemost. 2011; 9(4):700-10
Blanchette V S, Shapiro A D, Liesner R J, Navarro F H, Warrier I, Schroth P C, et al. Plasma and albumin-free recombinant factor VIII: pharmacokinetics, efficacy, and safety in previously treated pediatric patients. J Thromb Haemost. 2008; 6:1319-1326.
Valentino L A, Mamonov V, Hellmann A, et al. A randomized comparison of two prophylaxis regimens and a paired comparison of on-demand and prophylaxis treatments in hemophilia A management. J Thromb Haemost. 2012; 10(3):359-67
Mair F S, May C R. Thinking about the burden of treatment. BMJ. 2014; 349.
Lambert T, Benson G, Dolan G, Hermans C, Jimenez-Yuste V, Ljung R, Santagostino E. Practical aspects of extended half-life products for the treatment of haemophilia. Therapeutic Advances in Hematology. 2018; 295-308.
Mazepa M A, Monahan P E, Baker J R, Riske B K, Soucie J M. Men with severe hemophilia in the United States: birth cohort analysis of a large national database. Blood. 2016; 127:30733081.
Collins P W, Blanchette V S, Fischer K, Bjorkman S, Oh M, Fritsch S, et al. Break-through bleeding in relation to predicted factor VIII levels in patients receiving prophylactic treatment for severe hemophilia A. J Thromb Haemost. 2009 March;7(3):413-20.
Manco-Johnson M J, Soucie J M, Gill J C. Prophylaxis usage, bleeding rates, and joint outcomes of hemophilia, 1999 to 2010: a surveillance project. Blood. 2017; 2368-2374.
Soucie J M, Monahan P E, Kulkarni B A, Konkle M A, Mazepa M A and US Hemophilia Treatment Center Network. The frequency of joint hemorrhages and procedures in nonsevere hemophilia A vs B. Blood Adv. 2018; 2:2136-2144.
Oldenburg J. Optimal treatment strategies for hemophilia: achievements and limitations of current prophylactic regimens. Blood. 2015; 125:2038-2044.
Jimenez-Yuste V, Auerswald G, Benson G, et al. Achieving and maintaining an optimal trough level for prophylaxis in haemophilia: the past, the present and the future. Blood Transfus. 2014; 12:314-319.
Pipe S W, Montgomery R R, Pratt K P, Lenting P J, and Lillicrap D. Life in the shadow of a dominant partner: the FVIII-VWF association and its clinical implications for hemophilia A. Blood. 2016;128: 2007-16.
Eloctate [package insert]. Waltham, MA: Bioverativ Therapeutics Inc.;2017
Jazayeri J A, Carroll G. Half-life extension by Fusion to the Fc region. Theraputic Proteins: Strategies to Modulate Their Plasma Half-lives. 2012 Mar. 14; 48.
Benjamin W U, Sun Y N. Pharmacokinetics of peptide-Fc fusion proteins. J Pharm Sci. 2014 Jan. 1; 103(1): 53-64.
Yee A, Gildersleeve R D, Gu S, Kretz C A, McGee B M, Carr K M, et al. A von Willebrand factor fragment containing the D′D3 domains is sufficient to stabilize coagulation factor VIII in mice. Blood. 2014 Jul. 17; 124(3):445-52.
Lenting P J, Muczynski V, Ayme G, Denis C V, Christophe O D. Von Willebrand Factor Interaction with FVIII: Development of Long Acting FVIII Therapies. Blood 2016; 128: SCI-8.
Schellenberger V, Wang C W, Geething N C, Spink B J, Campbell A, To W, et al. A recombinant polypeptide extends the in vivo half-life of peptides and proteins in a tunable manner. Nat Biotechnology. 2009 Nov. 15; 27(12):1186.
European Medicines Agency (EMA) Guideline on the clinical investigation of recombinant and human plasma-derived factor VIII products. 26 Jul. 2018.
WFH. 2012. World Federation of Hemophilia. Guidelines for the management of hemophilia, 2nd edition.
Collins P W, Young G, Knobe K, Abdul Karim F, Angchaisuksiri P, et al. Recombinant long-acting glycoPEGylated factor IX in hemophilia B: a multinational randomized phase 3 trial. Blood. 2014; 124(26): 3880-3886.
Mahlangu J, Oldenburg J, Paz-Priel I, Negrier C, Niggli M, Mancuso M, et al. Emicizumab Prophylaxis in Patients Who Have Hemophilia A without Inhibitors. The New England Journal of Medicine. 2018; 811-822.
Thornburg C D, & Duncan N A. Treatment adherence in hemophilia. Patient Preference and Adherence. 2017; 11:1677-1686.
Oldenburg J, Brackmann H H. Prophylaxis in adult patients with severe haemophilia A. Thrombosis Research. 2018;s33-s37
Lentz S R, Misgav M, Ozelo M, Salek S Z, Veljkovic D, Recht M, et al. Results from a large multinational clinical trial (guardian 1) using prophylactic treatment with turocotocog alfa in adolescent and adult patients with severe haemophilia A: safety and efficacy. Haeomophilia. 2013; 691-697.
den Uijl I E, Mauser Bunschoten E P, Roosendaal G, Schutgens R E, Biesma D H, Grobbee D E, et al. Clinical severity of hemophilia A: does the classification of the 1950s still stand? Hemophilia. 17:849-853.
Oldenburg J, Kulkarni R, Srivastava A, Mahlangu J N, Blanchette V S, Tsao E. Improved joint health in subjects with severe hemophilia A treated prophylactically with recombinant factor VIII Fc fusion protein. Hemophilia. 2018; 24:77-84.
Oldenburg J, Zimmerman R, Katsarou O, Theodossiades G, Zanon E, Niemann B, Kellermann E, Ludin B. Controlled, cross-sectional MRI evaluation of joint status in severe haemophilia A patients treated with prophylaxis vs on demand. Haemophilia. 2015; 21:171-179.
Klamroth R, Windyga J, et al. Results from a phase 3, randomize, multicenter study of rurioctocog alfa pegol P K-guided prophylaxis targeting 2 FVIII trough levels in patients with severe hemophilia A (propel study). Poster P255 presented at: European Association for Haemophilia and Allied Disorders (EAHAD). 2019;Feb. 6-9; Prague, Czech Republic.
Jenkins P V, Rawley O, Smith O P, O'Donnell J S. Elevated factor VIII levels and risk of venous thrombosis. Brit J Haematology. 2012. 157:653-663.
Jivi [package insert]. Whippany, N J: Bayer HealthCare LLC; 2018.
Blanchette V S, Key N S, Ljung L R, Manco-Johnson M J, Van den berg H M, Srivastava A.
For the Subcommittee on Factor VIII, Factor I X, and Rare Coagulation Disorders. Definitions in hemophilia: communication from the SSC of the ISTH. J Thromb Haemost. 2014; 12(11):1935-9.
Cella D, Yount S, Rothrock N, Gershon R, Cook K, Reeve, B, et al. The Patient-Reported Outcomes Measurement Information System (PROMIS): Progress of an NIH Roadmap Cooperative Group During Its First Two Years. Medical Care, 45(5 Suppl 1). 2007; S3-S11
von Mackensen S, Gringeri A& the Haem-A-QoL study Group. Health-related Quality of Life in Adult Patients with Haemophilia—Assessment with a New Disease-specific Questionnaire (Haem-A-QoL). Journal Of Thrombosis and Haemostasis. 2005;3(Sup1):P0813.
Bullinger M, von Mackensen S, Fischer K, Khair K, Petersen C, Ravens-Sieberer U, et al. Pilot testing of the ‘Haemo-QoL’ quality of life questionnaire for haemophiliac children in six European countries. Haemophilia. 2002; March;8 Suppl 2:47-54
Van Genderen F R, Westers P, Heijnen L, De K P, van den Berg H M, Helders P J, et al. Measuring patients' perceptions on their functional abilities: validation of the Haemophilia Activities List. Haemophilia 2006 January; 12(1):36-46
Bharmal M, Payne K, Atkinson M J, Desrosiers M P, Morisky D E, Gemmen E. Validation of an abbreviated Treatment Satisfaction Questionnaire for Medication (TSQM-9) among patients on antihypertensive medications. Health Qual Life Outcomes. 2009; 7:36
Oldenburg J.and Pavlova A. Genetic risk factors for inhibitors to factors VIII and IX. Haemophilia.2006; 12(6):15-22.
Baskin J L, Pui C H, Reiss U, Wilimas J A, Metzger M L, Ribeiro R C, Howard S C. Management of occlusion and thrombosis associated with longterm indwelling central venous catheters. Lancet. 2009. 374(9684): 159-69.
Tagalakis V, Kahn S R, Libman M, Blostein M. The epidemiology of peripheral vein infusion thrombophlebitis: A critical review. Am J Med. 2002. 113(2):146-51.
Astermark J, Oldenburg J, Escobar M, White G C 2nd, Berntorp E, Malmo International Brother Study study group. The Malmo International Brother Study (MIBS). Genetic defects and inhibitor development in siblings with severe Hemophilia A. Haematologica. 2005; 90(7):924-31.
Carpenter S L, Michael Soucie J, Sterner S, Presley R, Hemophilia Treatment Center Network I. Increased prevalence of inhibitors in Hispanic patients with severe haemophilia A enrolled in the Universal Data Collection database. Haemophilia.2012;18(3):e260-5.
Viel K R, Ameri A, Abshire T C, lyer R V, Watts R G, Lutcher C, et al. Inhibitors of factor VIII in black patients with hemophilia. N Engl J Med. 2009; 360(16):1618-27.
Turecek P L, Johnsen J M, Pipe S W, O'Donnell J S. Biological mechanisms underlying inter-individual variation in factor VIII clearance in haemophilia. Haemophilia 2020; 26:575-83.
Lissitchkov T. Evaluation of Octocog Alfa, Rurioctocog Alfa Pegol, and Efanesoctocog Alfa in Severe Hemophilia A. WFH Abstract 2022.
Demers M, Aleman M M, Kistanova E, Peters R, Salas J, Seth Chhabra E. Efanesoctocog alfa elicits functional clot formation that is indistinguishable to that of recombinant factor VIII. J Thromb Haemost 2022.
Cook K F, Jensen S E, Schalet B D, et al. PROMIS measures of pain, fatigue, negative affect, physical function, and social function demonstrated clinical validity across a range of chronic conditions. J Clin Epidemiol 2016; 73:89-102.
Paredes et al. Prevalence and Interference of Chronic Pain Among People With Hemophilia: A Systematic Review and Meta-Analysis. J Pain 2021; 22:1134-4.
Stromer et al. Wien Klin Wochenschr 2021; 133:1042-56.
Van Genderen F R, Westers P, Heijnen L, De K P, van den Berg H M, Helders P J, et al. Measuring patients' perceptions on their functional abilities: validation of the Haemophilia Activities List. Haemophilia 2006 January; 12(1):36-46.
Devlin N J, Brooks R. EQ-5D and the EuroQol Group: Past, present and future. Appl Health Econ Health Policy 2017; 15:127-137.

### SEQUENCES

TABLE A
Exemplary chimeric protein sequences

A-FVIII(ELNN)-Fc: SEQ ID NO: 1

| | |
|---|---|
| ATRRYYLGAV ELSWDYMQSD LGELPVDARF PPRVPKSFPF NTSVVYKKTL FVEFTDHLFN | 60 |
| IAKPRPPWMG LLGPTIQAEV YDTVVITLKN MASHPVSLHA VGVSYWKASE GAEYDDQTSQ | 120 |
| REKEDDKVFP GGSHTYVWQV LKENGPMASD PLCLTYSYLS HVDLVKDLNS GLIGALLVCR | 180 |
| EGSLAKEKTQ TLHKFILLFA VFDEGKSWHS ETKNSLMQDR DAASARAWPK MHTVNGYVNR | 240 |
| SLPGLIGCHR KSVYWHVIGM GTTPEVHSIF LEGHTFLVRN HRQASLEISP ITFLTAQTLL | 300 |
| MDLGQFLLFC HISSHQHDGM EAYVKVDSCP EEPQLRMKNN EEAEDYDDDL TDSEMDVVRF | 360 |
| DDDNSPSFIQ IRSVAKKHPK TWVHYIAAEE EDWDYAPLVL APDDRSYKSQ YLNNGPQRIG | 420 |
| RKYKKVRFMA YTDETFKTRE AIQHESGILG PLLYGEVGDT LLIIFKNQAS RPYNIYPHGI | 480 |
| TDVRPLYSRR LPKGVKHLKD FPILPGEIFK YKWTVTVEDG PTKSDPRCLT RYYSSFVNME | 540 |
| RDLASGLIGP LLICYKESVD QRGNQIMSDK RNVILFSVFD ENRSWYLTEN IQRFLPNPAG | 600 |
| VQLEDPEFQA SNIMHSINGY VFDSLQLSVC LHEVAYWYIL SIGAQTDFLS VFFSGYTFKH | 660 |
| KMVYEDTLTL FPFSGETVFM SMENPGLWIL GCHNSDFRNR GMTALLKVSS CDKNTGDYYE | 720 |
| DSYEDISAYL LSKNNAIEPR SFSQNGTSES ATPESGPGSE PATSGSETPG TSESATPESG | 780 |
| PGSEPATSGS ETPGTSESAT PESGPGTSTE PSEGSAPGSP AGSPTSTEEG TSESATPESG | 840 |
| PGSEPATSGS ETPGTSESAT PESGPGSPAG SPTSTEEGSP AGSPTSTEEG TSTEPSEGSA | 900 |
| PGTSESATPE SGPGTSESAT PESGPGTSES ATPESGPGSE PATSGSETPG SEPATSGSET | 960 |
| PGSPAGSPTS TEEGTSTEPS EGSAPGTSTE PSEGSAPGSE PATSGSETPG TSESATPESG | 1020 |
| PGTSTEPSEG SAPASSEITR TTLQSDQEEI DYDDTISVEM KKEDFDIYDE DENQSPRSFQ | 1080 |
| KKTRHYFIAA VERLWDYGMS SSPHVLRNRA QSGSVPQFKK VVFQEFTDGS FTQPLYRGEL | 1140 |
| NEHLGLLGPY IRAEVEDNIM VTFRNQASRP YSFYSSLISY EEDQRQGAEP RKNFVKPNET | 1200 |
| KTYFWKVQHH MAPTKDEFDC KAWAYFSDVD LEKDVHSGLI GPLLVCHTNT LNPAHGRQVT | 1260 |
| VQEFALFFTI FDETKSWYFT ENMERNCRAP CNIQMEDPTF KENYRFHAIN GYIMDTLPGL | 1320 |
| VMAQDQRIRW YLLSMGSNEN IHSIHFSGHV FTVRKKEEYK MALYNLYPGV FETVEMLPSK | 1380 |
| AGIWRVECLI GEHLHAGMST LFLVYSNKCQ TPLGMASGHI RDFQITASGQ YGQWAPKLAR | 1440 |
| LHYSGSINAW STKEPFSWIK VDLLAPMIIH GIKTQGARQK FSSLYISQFI IMYSLDGKKW | 1500 |
| QTYRGNSTGT LMVFFGNVDS SGIKHNIFNP PIIARYIRLH PTHYSIRSTL RMELMGCDLN | 1560 |
| SCSMPLGMES KAISDAQITA SSYFTNMFAT WSPSKARLHL QGRSNAWRPQ VNNPKEWLQV | 1620 |
| DFQKTMKVTG VTTQGVKSLL TSMYVKEFLI SSSQDGHQWT LFFQNGKVKV FQGNQDSFTP | 1680 |
| WVNSLDPPLL TRYLRIHPQS WVHQIALRME VLGCEAQDLY DKTHTCPPCP APELLGGPSV | 1740 |
| FLFPPKPKDT LMISRTPEVT CVVVDVSHED PEVKFNWYVD GVEVHNAKTK PREEQYNSTY | 1800 |
| RVVSVLTVLH QDWLNGKEYK CKVSNKALPA PIEKTISKAK GQPREPQVYT LPPSRDELTK | 1860 |
| NQVSLTCLVK GFYPSDIAVE WESNGQPENN YKTTPPVLDS DGSFFLYSKL TVDKSRWQQG | 1920 |
| NVFSCSVMHE ALHNHYTQKS LSLSPG | |
| VWF(D′D3)-ELNN-a2 Linker-Fc: SEQ ID NO: 2 | |
| SLSCRPPMVK LVCPADNLRA EGLECTKTCQ NYDLECMSMG CVSGCLCPPG MVRHENRCVA | 60 |
| LERCPCFHQG KEYAPGETVK IGCNTCVCRD RKWNCTDHVC DATCSTIGMA HYLTFDGLKY | 120 |
| LFPGECQYVL VQDYCGSNPG TFRILVGNKG CSHPSVKCKK RVTILVEGGE IELFDGEVNV | 180 |
| KRPMKDETHF EVVESGRYII LLLGKALSVV WDRHLSISVV LKQTYQEKVC GLCGNFDGIQ | 240 |
| NNDLTSSNLQ VEEDPVDFGN SWKVSSQCAD TRKVPLDSSP ATCHNNIMKQ TMVDSSCRIL | 300 |
| TSDVFQDCNK LVDPEPYLDV CIYDTCSCES IGDCAAFCDT IAAYAHVCAQ HGKVVTWRTA | 360 |
| TLCPQSCEER NLRENGYEAE WRYNSCAPAC QVTCQHPEPL ACPVQCVEGC HAHCPPGKIL | 420 |
| DELLQTCVDP EDCPVCEVAG RRFASGKKVT LNPSDPEHCQ ICHCDVVNLT CEACQEPGTS | 480 |
| ESATPESGPG SEPATSGSET PGTSESATPE SGPGSEPATS GSETPGTSES ATPESGPGTS | 540 |
| TEPSEGSAPG SPAGSPTSTE EGTSESATPE SGPGSEPATS GSETPGTSES ATPESGPGSP | 600 |
| AGSPTSTEEG SPAGSPTSTE EGASSDKNTG DYYEDSYEDI SAYLLSKNNA IEPRSFSDKT | 660 |
| HTCPPCPAPE LLGGPSVFLF PPKPKDTLMI SRTPEVTCVV VDVSHEDPEV KFNWYVDGVE | 720 |
| VHNAKTKPRE EQYNSTYRVV SVLTVLHQDW LNGKEYKCKV SNKALPAPIE KTISKAKGQP | 780 |
| REPQVYTLPP SRDELTKNQV SLTCLVKGFY PSDIAVEWES NGQPENNYKT TPPVLDSDGS | 840 |
| FFLYSKLTVD KSRWQQGNVF SCSVMHEALH NHYTQKSLSL SPG | 883 |

Amino acid sequence of FVIII protein (312A)-includes signal peptide: SEQ ID NO: 3

| | |
|---|---|
| 1 | MQIELSTCFF LCLLRFCFSA TRRYYLGAVE LSWDYMQSDL GELPVDARFP |
| 51 | PRVPKSFPFN TSVVYKKTLF VEFTDHLFNI AKPRPPWMGL LGPTIQAEVY |
| 101 | DTVVITLKNM ASHPVSLHAV GVSYWKASEG AEYDDQTSQR EKEDDKVFPG |
| 151 | GSHTYVWQVL KENGPMASDP LCLTYSYLSH VDLVKDLNSG LIGALLVCRE |
| 201 | GSLAKEKTQT LHKFILLFAV FDEGKSWHSE TKNSLMQDRD AASARAWPKM |
| 251 | HTVNGYVNRS LPGLIGCHRK SVYWHVIGMG TTPEVHSIFL EGHTFLVRNH |
| 301 | RQASLEISPI TFLTAQTLLM DLGQFLLFCH ISSHQHDGME AYVKVDSCPE |
| 351 | EPQLRMKNNE EAEDYDDDLT DSEMDVVRFD DDNSPSFIQI RSVAKKHPKT |
| 401 | WVHYIAAEEE DWDYAPLVLA PDDRSYKSQY LNNGPQRIGR KYKKVRFMAY |
| 451 | TDETFKTREA IQHESGILGP LLYGEVGDTL LIIFKNQASR PYNIYPHGIT |
| 501 | DVRPLYSRRL PKGVKHLKDF PILPGEIFKY KWTVTVEDGP TKSDPRCLTR |
| 551 | YYSSFVNMER DLASGLIGPL LICYKESVDQ RGNQIMSDKR NVILFSVFDE |
| 601 | NRSWYLTENI QRFLPNPAGV QLEDPEFQAS NIMHSINGYV FDSLQLSVCL |
| 651 | HEVAYWYILS IGAQTDFLSV FFSGYTFKHK MVYEDTLTLF PFSGETVFMS |
| 701 | MENPGLWILG CHNSDFRNRG MTALLKVSSC DKNTGDYYED SYEDISAYLL |
| 751 | SKNNAIEPRS FSQNGTSESA TPESGPGSEP ATSGSETPGT SESATPESGP |
| 801 | GSEPATSGSE TPGTSESATP ESGPGTSTEP SEGSAPGSPA GSPTSTEEGT |
| 851 | SESATPESGP GSEPATSGSE TPGTSESATP ESGPGSPAGS PTSTEEGSPA |
| 901 | GSPTSTEEGT STEPSEGSAP GTSESATPES GPGTSESATP ESGPGTSESA |
| 951 | TPESGPGSEP ATSGSETPGS EPATSGSETP GSPAGSPTST EEGTSTEPSE |
| 1001 | GSAPGTSTEP SEGSAPGSEP ATSGSETPGT SESATPESGP GTSTEPSEGS |
| 1051 | APASSEITRT TLQSDQEEID YDDTISVEMK KEDFDIYDED ENQSPRSFQK |
| 1101 | KTRHYFIAAV ERLWDYGMSS SPHVLRNRAQ SGSVPQFKKV VFQEFTDGSF |
| 1151 | TQPLYRGELN EHLGLLGPYI RAEVEDNIMV TFRNQASRPY SFYSSLISYE |
| 1201 | EDQRQGAEPR KNFVKPNETK TYFWKVQHHM APTKDEFDCK AWAYFSDVDL |
| 1251 | EKDVHSGLIG PLLVCHTNTL NPAHGRQVTV QEFALFFTIF DETKSWYFTE |
| 1301 | NMERNCRAPC NIQMEDPTFK ENYRFHAING YIMDTLPGLV MAQDQRIRWY |
| 1351 | LLSMGSNENI HSIHFSGHVF TVRKKEEYKM ALYNLYPGVF ETVEMLPSKA |
| 1401 | GIWRVECLIG EHLHAGMSTL FLVYSNKCQT PLGMASGHIR DFQITASGQY |
| 1451 | GQWAPKLARL HYSGSINAWS TKEPFSWIKV DLLAPMIIHG IKTQGARQKF |
| 1501 | SSLYISQFII MYSLDGKKWQ TYRGNSTGTL MVFFGNVDSS GIKHNIFNPP |
| 1551 | IIARYIRLHP THYSIRSTLR MELMGCDLNS CSMPLGMESK AISDAQITAS |
| 1601 | SYFTNMFATW SPSKARLHLQ GRSNAWRPQV NNPKEWLQVD FQKTMKVTGV |
| 1651 | TTQGVKSLLT SMYVKEFLIS SSQDGHQWTL FFQNGKVKVF QGNQDSFTPV |
| 1701 | VNSLDPPLLT RYLRIHPQSW VHQIALRMEV LGCEAQDLYD KTHTCPPCPA |
| 1751 | PELLGGPSVF LFPPKPKDTL MISRTPEVTC VVVDVSHEDP EVKFNWYVDG |
| 1801 | VEVHNAKTKP REEQYNSTYR VVSVLTVLHQ DWLNGKEYKC KVSNKALPAP |
| 1851 | IEKTISKAKG QPREPQVYTL PPSRDELTKN QVSLTCLVKG FYPSDIAVEW |
| 1901 | ESNGQPENNY KTTPPVLDSD GSFFLYSKLT VDKSRWQQGN VFSCSVMHEA |
| 1951 | LHNHYTQKSL SLSPG* |

Nucleotide sequence encoding FVIII 312A: SEQ ID NO: 4

| | |
|---|---|
| 1 | ATGCAAATAG AGCTCTCCAC CTGCTTCTTT CTGTGCCTTT TGCGATTCTG |
| 51 | CTTTAGTGCC ACCAGAAGAT ACTACCTGGG TGCAGTGGAA CTGTCATGGG |
| 101 | ACTATATGCA AAGTGATCTC GGTGAGCTGC CTGTGGACGC AAGATTTCCT |
| 151 | CCTAGAGTGC CAAAATCTTT TCCATTCAAC ACCTCAGTCG TGTACAAAAA |
| 201 | GACTCTGTTT GTAGAATTCA CGGATCACCT TTTCAACATC GCTAAGCCAA |
| 251 | GGCCACCCTG GATGGGTCTG CTAGGTCCTA CCATCCAGGC TGAGGTTTAT |
| 301 | GATACAGTGG TCATTACACT TAAGAACATG GCTTCCCATC CTGTCAGTCT |
| 351 | TCATGCTGTT GGTGTATCCT ACTGGAAAGC TTCTGAGGGA GCTGAATATG |
| 401 | ATGATCAGAC CAGTCAAAGG GAGAAAGAAG ATGATAAAGT CTTCCCTGGT |
| 451 | GGAAGCCATA CATATGTCTG GCAGGTCCTG AAAGAGAATG GTCCAATGGC |
| 501 | CTCTGACCCA CTGTGCCTTA CCTACTCATA TCTTTCTCAT GTGGACCTGG |
| 551 | TAAAAGACTT GAATTCAGGC CTCATTGGAG CCCTACTAGT ATGTAGAGAA |
| 601 | GGGAGTCTGG CCAAGGAAAA GACACAGACC TTGCACAAAT TTATACTACT |
| 651 | TTTTGCTGTA TTTGATGAAG GGAAAAGTTG GCACTCAGAA ACAAAGAACT |
| 701 | CCTTGATGCA GGATAGGGAT GCTGCATCTG CTCGGGCCTG GCCTAAAATG |
| 751 | CACACAGTCA ATGGTTATGT AAACAGGTCT CTGCCAGGTC TGATTGGATG |
| 801 | CCACAGGAAA TCAGTCTATT GGCATGTGAT TGGAATGGGC ACCACTCCTG |
| 851 | AAGTGCACTC AATATTCCTC GAAGGTCACA CATTTCTTGT GAGGAACCAT |
| 901 | CGCCAGGCGT CCTTGGAAAT CTCGCCAATA ACTTTCCTTA CTGCTCAAAC |
| 951 | ACTCTTGATG GACCTTGGAC AGTTTCTACT GTTTTGTCAT ATCTCTTCCC |
| 1001 | ACCAACATGA TGGCATGGAA GCTTATGTCA AAGTAGACAG CTGTCCAGAG |
| 1051 | GAACCCCAAC TACGAATGAA AAATAATGAA GAAGCGGAAG ACTATGATGA |
| 1101 | TGATCTTACT GATTCTGAAA TGGATGTGGT CAGGTTTGAT GATGACAACT |
| 1151 | CTCCTTCCTT TATCCAAATT CGCTCAGTTG CCAAGAAGCA TCCTAAAACT |
| 1201 | TGGGTACATT ACATTGCTGC TGAAGAGGAG GACTGGGACT ATGCTCCCTT |
| 1251 | AGTCCTCGCC CCCGATGACA GAAGTTATAA AAGTCAATAT TTGAACAATG |
| 1301 | GCCCTCAGCG GATTGGTAGG AAGTACAAAA AAGTCCGATT TATGGCATAC |
| 1351 | ACAGATGAAA CCTTTAAGAC TCGTGAAGCT ATTCAGCATG AATCAGGAAT |
| 1401 | CTTGGGACCT TTACTTTATG GGGAAGTTGG AGACACACTG TTGATTATAT |
| 1451 | TTAAGAATCA AGCAAGCAGA CCATATAACA TCTACCCTCA CGGAATCACT |
| 1501 | GATGTCCGTC CTTTGTATTC AAGGAGATTA CCAAAAGGTG TAAAACATTT |
| 1551 | GAAGGATTTT CCAATTCTGC CAGGAGAAAT ATTCAAATAT AAATGGACAG |
| 1601 | TGACTGTAGA AGATGGGCCA ACTAAATCAG ATCCTCGGTG CCTGACCCGC |
| 1651 | TATTACTCTA GTTTCGTTAA TATGGAGAGA GATCTAGCTT CAGGACTCAT |
| 1701 | TGGCCCTCTC CTCATCTGCT ACAAAGAATC TGTAGATCAA AGAGGAAACC |
| 1751 | AGATAATGTC AGACAAGAGG AATGTCATCC TGTTTTCTGT ATTTGATGAG |
| 1801 | AACCGAAGCT GGTACCTCAC AGAGAATATA CAACGCTTTC TCCCCAATCC |
| 1851 | AGCTGGAGTG CAGCTTGAGG ATCCAGAGTT CCAAGCCTCC AACATCATGC |
| 1901 | ACAGCATCAA TGGCTATGTT TTTGATAGTT TGCAGTTGTC AGTTTGTTTG |
| 1951 | CATGAGGTGG CATACTGGTA CATTCTAAGC ATTGGAGCAC AGACTGACTT |
| 2001 | CCTTTCTGTC TTCTTCTCTG GATATACCTT CAAACACAAA ATGGTCTATG |
| 2051 | AAGACACACT CACCCTATTC CCATTCTCAG GAGAAACTGT CTTCATGTCG |
| 2101 | ATGGAAAACC CAGGTCTATG GATTCTGGGG TGCCACAACT CAGACTTTCG |
| 2151 | GAACAGAGGC ATGACCGCCT TACTGAAGGT TTCTAGTTGT GACAAGAACA |
| 2201 | CTGGTGATTA TTACGAGGAC AGTTATGAAG ATATTTCAGC ATACTTGCTG |
| 2251 | AGTAAAAACA ATGCCATTGA ACCAAGAAGC TTCTCTCAAA ACGGTACCTC |
| 2301 | AGAGTCTGCT ACCCCCGAGT CAGGGCCAGG ATCAGAGCCA GCCACCTCCG |
| 2351 | GGTCTGAGAC ACCCGGGACT TCCGAGAGTG CCACCCCTGA GTCCGGACCC |
| 2401 | GGGTCCGAGC CCGCCACTTC CGGCTCCGAA ACTCCCGGCA CAAGCGAGAG |
| 2451 | CGCTACCCCA GAGTCAGGAC CAGGAACATC TACAGAGCCC TCTGAAGGCT |
| 2501 | CCGCTCCAGG GTCCCCAGCC GGCAGTCCCA CTAGCACCGA GGAGGGAACC |
| 2551 | TCTGAAAGCG CCACACCCGA ATCAGGGCCA GGGTCTGAGC CTGCTACCAG |
| 2601 | CGGCAGCGAG ACACCAGGCA CCTCTGAGTC CGCCACACCA GAGTCCGGAC |
| 2651 | CCGGATCTCC CGCTGGGAGC CCCACCTCCA CTGAGGAGGG ATCTCCTGCT |
| 2701 | GGCTCTCCAA CATCTACTGA GGAAGGAACC TCAACCGAGC CATCCGAGGG |
| 2751 | ATCAGCTCCC GGCACCTCAG AGTCGGCAAC CCCGGAGTCT GGACCCGGAA |
| 2801 | CTTCCGAAAG TGCCACACCA GAGTCCGGTC CCGGGACTTC AGAATCAGCA |
| 2851 | ACACCCGAGT CCGGCCCTGG GTCTGAACCC GCCACAAGTG GTAGTGAGAC |
| 2901 | ACCAGGATCA GAACCTGCTA CCTCAGGGTC AGAGACACCC GGATCTCCGG |
| 2951 | CAGGCTCACC AACCTCCACT GAGGAGGGCA CCAGCACAGA ACCAAGCGAG |
| 3001 | GGCTCCGCAC CCGGAACAAG CACTGAACCC AGTGAGGGTT CAGCACCCGG |
| 3051 | CTCTGAGCCG GCCACAAGTG GCAGTGAGAC ACCCGGCACT TCAGAGAGTG |
| 3101 | CCACCCCCGA GAGTGGCCCA GGCACTAGTA CCGAGCCCTC TGAAGGCAGT |
| 3151 | GCGCCAGCCT CGAGCGAAAT AACTCGTACT ACTCTTCAGT CAGATCAAGA |
| 3201 | GGAAATTGAC TATGATGATA CCATATCAGT TGAAATGAAG AAGGAAGATT |
| 3251 | TTGACATTTA TGATGAGGAT GAAAATCAGA GCCCCCGCAG CTTTCAAAAG |
| 3301 | AAAACACGAC ACTATTTTAT TGCTGCAGTG GAGAGGCTCT GGGATTATGG |
| 3351 | GATGAGTAGC TCCCCACATG TTCTAAGAAA CAGGGCTCAG AGTGGCAGTG |
| 3401 | TCCCTCAGTT CAAGAAAGTT GTTTTCCAGG AATTTACTGA TGGCTCCTTT |
| 3451 | ACTCAGCCCT TATACCGTGG AGAACTAAAT GAACATTTGG GACTCCTGGG |
| 3501 | GCCATATATA AGAGCAGAAG TTGAAGATAA TATCATGGTA ACTTTCAGAA |
| 3551 | ATCAGGCCTC TCGTCCCTAT TCCTTCTATT CTAGCCTTAT TTCTTATGAG |
| 3601 | GAAGATCAGA GGCAAGGAGC AGAACCTAGA AAAAACTTTG TCAAGCCTAA |
| 3651 | TGAAACCAAA ACTTACTTTT GGAAAGTGCA ACATCATATG GCACCCACTA |
| 3701 | AAGATGAGTT TGACTGCAAA GCCTGGGCTT ATTTCTCTGA TGTTGACCTG |
| 3751 | GAAAAAGATG TGCACTCAGG CCTGATTGGA CCCCTTCTGG TCTGCCACAC |
| 3801 | TAACACACTG AACCCTGCTC ATGGGAGACA AGTGACAGTA CAGGAATTTG |
| 3851 | CTCTGTTTTT CACCATCTTT GATGAGACCA AAAGCTGGTA CTTCACTGAA |
| 3901 | AATATGGAAA GAAACTGCAG GGCTCCCTGC AATATCCAGA TGGAAGATCC |
| 3951 | CACTTTTAAA GAGAATTATC GCTTCCATGC AATCAATGGC TACATAATGG |
| 4001 | ATACACTACC TGGCTTAGTA ATGGCTCAGG ATCAAAGGAT TCGATGGTAT |
| 4051 | CTGCTCAGCA TGGGCAGCAA TGAAAACATC CATTCTATTC ATTTCAGTGG |
| 4101 | ACATGTGTTC ACTGTACGAA AAAAAGAGGA GTATAAAATG GCACTGTACA |
| 4151 | ATCTCTATCC AGGTGTTTTT GAGACAGTGG AAATGTTACC ATCCAAAGCT |
| 4201 | GGAATTTGGC GGGTGGAATG CCTTATTGGC GAGCATCTAC ATGCTGGGAT |
| 4251 | GAGCACACTT TTTCTGGTGT ACAGCAATAA GTGTCAGACT CCCCTGGGAA |
| 4301 | TGGCTTCTGG ACACATTAGA GATTTTCAGA TTACAGCTTC AGGACAATAT |
| 4351 | GGACAGTGGG CCCCAAAGCT GGCCAGACTT CATTATTCCG GATCAATCAA |
| 4401 | TGCCTGGAGC ACCAAGGAGC CCTTTTCTTG GATCAAGGTG GATCTGTTGG |
| 4451 | CACCAATGAT TATTCACGGC ATCAAGACCC AGGGTGCCCG TCAGAAGTTC |
| 4501 | TCCAGCCTCT ACATCTCTCA GTTTATCATC ATGTATAGTC TTGATGGGAA |
| 4551 | GAAGTGGCAG ACTTATCGAG GAAATTCCAC TGGAACCTTA ATGGTCTTCT |
| 4601 | TTGGCAATGT GGATTCATCT GGGATAAAAC ACAATATTTT TAACCCTCCA |
| 4651 | ATTATTGCTC GATACATCCG TTTGCACCCA ACTCATTATA GCATTCGCAG |
| 4701 | CACTCTTCGC ATGGAGTTGA TGGGCTGTGA TTTAAATAGT TGCAGCATGC |
| 4751 | CATTGGGAAT GGAGAGTAAA GCAATATCAG ATGCACAGAT TACTGCTTCA |
| 4801 | TCCTACTTTA CCAATATGTT TGCCACCTGG TCTCCTTCAA AAGCTCGACT |
| 4851 | TCACCTCCAA GGGAGGAGTA ATGCCTGGAG ACCTCAGGTG AATAATCCAA |
| 4901 | AAGAGTGGCT GCAAGTGGAC TTCCAGAAGA CAATGAAAGT CACAGGAGTA |
| 4951 | ACTACTCAGG GAGTAAAATC TCTGCTTACC AGCATGTATG TGAAGGAGTT |
| 5001 | CCTCATCTCC AGCAGTCAAG ATGGCCATCA GTGGACTCTC TTTTTTCAGA |
| 5051 | ATGGCAAAGT AAAGGTTTTT CAGGGAAATC AAGACTCCTT CACACCTGTG |
| 5101 | GTGAACTCTC TAGACCCACC GTTACTGACT CGCTACCTTC GAATTCACCC |
| 5151 | CCAGAGTTGG GTGCACCAGA TTGCCCTGAG GATGGAGGTT CTGGGCTGCG |
| 5201 | AGGCACAGGA CCTCTACGAC AAAACTCACA CATGCCCACC GTGCCCAGCT |
| 5251 | CCAGAACTCC TGGGCGGACC GTCAGTCTTC CTCTTCCCCC CAAAACCCAA |
| 5301 | GGACACCCTC ATGATCTCCC GGACCCCTGA GGTCACATGC GTGGTGGTGG |
| 5351 | ACGTGAGCCA CGAAGACCCT GAGGTCAAGT TCAACTGGTA TGTGGACGGC |
| 5401 | GTGGAAGTGC ATAATGCCAA GACAAAGCCG CGGGAGGAGC AGTACAACAG |
| 5451 | CACGTACCGT GTGGTCAGCG TCCTCACCGT CCTGCACCAA GACTGGCTGA |
| 5501 | ATGGCAAGGA GTACAAGTGC AAGGTCTCCA ACAAAGCCCT CCCAGCCCCC |
| 5551 | ATCGAGAAAA CCATCTCCAA AGCCAAAGGG CAGCCCCGAG AACCACAGGT |
| 5601 | GTACACCCTG CCCCCATCCC GGGATGAGCT GACCAAGAAC CAAGTTAGCC |
| 5651 | TGACCTGCCT GGTCAAAGGC TTCTATCCCA GCGACATCGC CGTGGAGTGG |
| 5701 | GAGAGCAATG GGCAGCCGGA GAACAACTAC AAGACCACGC CTCCCGTGTT |
| 5751 | GGACTCCGAC GGCTCCTTCT TCCTCTACTC CAAGCTCACC GTGGACAAGA |
| 5801 | GCAGGTGGCA GCAGGGGAAC GTCTTCTCAT GCTCCGTGAT GCATGAGGCT |
| 5851 | CTGCACAACC ACTACACGCA GAAGAGCCTC TCCCTGTCTC CGGGTTGA |

Amino acid sequence of VWF059A protein-includes signal peptide and D1D2 region:
SEQ ID NO: 5

| | |
|---|---|
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP GTSESATPES |
| 1251 | GPGSEPATSG SETPGTSESA TPESGPGSEP ATSGSETPGT SESATPESGP |
| 1301 | GTSTEPSEGS APGSPAGSPT STEEGTSESA TPESGPGSEP ATSGSETPGT |
| 1351 | SESATPESGP GSPAGSPTST EEGSPAGSPT STEEGASSDK NTGDYYEDSY |
| 1401 | EDISAYLLSK NNAIEPRSFS DKTHTCPPCP APELLGGPSV FLFPPKPKDT |
| 1451 | LMISRTPEVT CVVVDVSHED PEVKFNWYVD GVEVHNAKTK PREEQYNSTY |
| 1501 | RVVSVLTVLH QDWLNGKEYK CKVSNKALPA PIEKTISKAK GQPREPQVYT |
| 1551 | LPPSRDELTK NQVSLTCLVK GFYPSDIAVE WESNGQPENN YKTTPPVLDS |
| 1601 | DGSFFLYSKL TVDKSRWQQG NVFSCSVMHE ALHNHYTQKS LSLSPG* |

Nucleotide sequence encoding VWF059A protein: SEQ ID NO: 6

| | |
|---|---|
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 351 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG GGTACATCAG AGAGCGCCAC CCCTGAAAGT |
| 3751 | GGTCCCGGGA GCGAGCCAGC CACATCTGGG TCGGAAACGC CAGGCACATC |
| 3801 | CGAGTCTGCA ACTCCCGAGT CCGGACCTGG CTCCGAGCCT GCCACTAGCG |
| 3851 | GCTCCGAGAC TCCGGGAACT TCCGAGAGCG CTACACCAGA AAGCGGACCC |
| 3901 | GGAACCAGTA CCGAACCTAG CGAGGGCTCT GCTCCGGGCA GCCCAGCCGG |
| 3951 | CTCTCCTACA TCCACGGAGG AGGGCACTTC CGAATCCGCC ACCCCGGAGT |
| 4001 | CAGGGCCAGG ATCTGAACCC GCTACCTCAG GCAGTGAGAC GCCAGGAACG |
| 4051 | AGCGAGTCCG CTACACCGGA GAGTGGGCCA GGGAGCCCTG CTGGATCTCC |
| 4101 | TACGTCCACT GAGGAAGGGT CACCAGCGGG CTCGCCCACC AGCACTGAAG |
| 4151 | AAGGTGCCTC GTCTGACAAG AACACTGGTG ATTATTACGA GGACAGTTAT |
| 4201 | GAAGATATTT CAGCATACTT GCTGAGTAAA AACAATGCCA TTGAACCAAG |
| 4251 | AAGCTTCTCT GACAAAACTC ACACATGCCC ACCGTGCCCA GCTCCAGAAC |
| 4301 | TCCTGGGCGG ACCGTCAGTC TTCCTCTTCC CCCCAAAACC CAAGGACACC |
| 4351 | CTCATGATCT CCCGGACCCC TGAGGTCACA TGCGTGGTGG TGGACGTGAG |
| 4401 | CCACGAAGAC CCTGAGGTCA AGTTCAACTG GTATGTGGAC GGCGTGGAAG |
| 4451 | TGCATAATGC CAAGACAAAG CCGCGGGAGG AGCAGTACAA CAGCACGTAC |
| 4501 | CGTGTGGTCA GCGTCCTCAC CGTCCTGCAC CAAGACTGGC TGAATGGCAA |
| 4551 | GGAGTACAAG TGCAAGGTCT CCAACAAAGC CCTCCCAGCC CCCATCGAGA |
| 4601 | AAACCATCTC CAAAGCCAAA GGGCAGCCCC GAGAACCACA GGTGTACACC |
| 4651 | CTGCCCCCAT CCCGGGATGA GCTGACCAAG AACCAAGTTA GCCTGACCTG |
| 4701 | CCTGGTCAAA GGCTTCTATC CCAGCGACAT CGCCGTGGAG TGGGAGAGCA |
| 4751 | ATGGGCAGCC GGAGAACAAC TACAAGACCA CGCCTCCCGT GTTGGACTCC |
| 4801 | GACGGCTCCT TCTTCCTCTA CTCCAAGCTC ACCGTGGACA AGAGCAGGTG |
| 4851 | GCAGCAGGGG AACGTCTTCT CATGCTCCGT GATGCATGAG GCTCTGCACA |
| 4901 | ACCACTACAC GCAGAAGAGC CTCTCCCTGT CTCCGGGTTG A |

FVIII(ELNN)-Fc: SEQ ID NO: 7

| | |
|---|---|
| TRRYYLGAVE LSWDYMQSDL GELPVDARFP PRVPKSFPFN TSVVYKKTLF VEFTDHLFNI | 60 |
| AKPRPPWMGL LGPTIQAEVY DTVVITLKNM ASHPVSLHAV GVSYWKASEG AEYDDQTSQR | 120 |
| EKEDDKVFPG GSHTYVWQVL KENGPMASDP LCLTYSYLSH VDLVKDLNSG LIGALLVCRE | 180 |
| GSLAKEKTQT LHKFILLFAV FDEGKSWHSE TKNSLMQDRD AASARAWPKM HTVNGYVNRS | 240 |
| LPGLIGCHRK SVYWHVIGMG TTPEVHSIFL EGHTFLVRNH RQASLEISPI TFLTAQTLLM | 300 |
| DLGQFLLFCH ISSHQHDGME AYVKVDSCPE EPQLRMKNNE EAEDYDDDLT DSEMDVVRFD | 360 |
| DDNSPSFIQI RSVAKKHPKT WVHYIAAEEE DWDYAPLVLA PDDRSYKSQY LNNGPQRIGR | 420 |
| KYKKVRFMAY TDETFKTREA IQHESGILGP LLYGEVGDTL LIIFKNQASR PYNIYPHGIT | 480 |
| DVRPLYSRRL PKGVKHLKDF PILPGEIFKY KWTVTVEDGP TKSDPRCLTR YYSSFVNMER | 540 |
| DLASGLIGPL LICYKESVDQ RGNQIMSDKR NVILFSVFDE NRSWYLTENI QRFLPNPAGV | 600 |
| QLEDPEFQAS NIMHSINGYV FDSLQLSVCL HEVAYWYILS IGAQTDFLSV FFSGYTFKHK | 660 |
| MVYEDTLTLF PFSGETVFMS MENPGLWILG CHNSDFRNRG MTALLKVSSC DKNTGDYYED | 720 |
| SYEDISAYLL SKNNAIEPRS FSQNGTSESA TPESGPGSEP ATSGSETPGT SESATPESGP | 780 |
| GSEPATSGSE TPGTSESATP ESGPGTSTEP SEGSAPGSPA GSPTSTEEGT SESATPESGP | 840 |
| GSEPATSGSE TPGTSESATP ESGPGSPAGS PTSTEEGSPA GSPTSTEEGT STEPSEGSAP | 900 |
| GTSESATPES GPGTSESATP ESGPGTSESA TPESGPGSEP ATSGSETPGS EPATSGSETP | 960 |
| GSPAGSPTST EEGTSTEPSE GSAPGTSTEP SEGSAPGSEP ATSGSETPGT SESATPESGP | 1020 |
| GTSTEPSEGS APASSEITRT TLQSDQEEID YDDTISVEMK KEDFDIYDED ENQSPRSFQK | 1080 |
| KTRHYFIAAV ERLWDYGMSS SPHVLRNRAQ SGSVPQFKKV VFQEFTDGSF TQPLYRGELN | 1140 |
| EHLGLLGPYI RAEVEDNIMV TERNQASRPY SFYSSLISYE EDQRQGAEPR KNFVKPNETK | 1200 |
| TYFWKVQHHM APTKDEFDCK AWAYFSDVDL EKDVHSGLIG PLLVCHTNTL NPAHGRQVTV | 1260 |
| QEFALFFTIF DETKSWYFTE NMERNCRAPC NIQMEDPTFK ENYRFHAING YIMDTLPGLV | 1320 |
| MAQDQRIRWY LLSMGSNENI HSIHFSGHVF TVRKKEEYKM ALYNLYPGVF ETVEMLPSKA | 1380 |
| GIWRVECLIG EHLHAGMSTL FLVYSNKCQT PLGMASGHIR DFQITASGQY GQWAPKLARL | 1440 |
| HYSGSINAWS TKEPFSWIKV DLLAPMIIHG IKTQGARQKF SSLYISQFII MYSLDGKKWQ | 1500 |
| TYRGNSTGTL MVFFGNVDSS GIKHNIFNPP IIARYIRLHP THYSIRSTLR MELMGCDLNS | 1560 |
| CSMPLGMESK AISDAQITAS SYFTNMFATW SPSKARLHLQ GRSNAWRPQV NNPKEWLQVD | 1620 |
| FQKTMKVTGV TTQGVKSLLT SMYVKEFLIS SSQDGHQWTL FFQNGKVKVF QGNQDSFTPV | 1680 |
| VNSLDPPLLT RYLRIHPQSW VHQIALRMEV LGCEAQDLYD KTHTCPPCPA PELLGGPSVF | 1740 |
| LFPPKPKDTL MISRTPEVTC VVVDVSHEDP EVKFNWYVDG VEVHNAKTKP REEQYNSTYR | 1800 |
| VVSVLTVLHQ DWLNGKEYKC KVSNKALPAP IEKTISKAKG QPREPQVYTL PPSRDELTKN | 1860 |
| QVSLTCLVKG FYPSDIAVEW ESNGQPENNY KTTPPVLDSD GSFFLYSKLT VDKSRWQQGN | 1920 |
| VFSCSVMHEA LHNHYTQKSL SLSPG | 1945 |

TABLE B
Additional chimeric protein sequences

| Description/ | | |
|---|---|---|
| SEQ ID NO. | Sequence | |
| Mature native human | ATRRYYLGAV ELSWDYMQSD LGELPVDARF PPRVPKSFPF NTSVVYKKTL FVEFTDHLFN | 60 |
| FVIII | IAKPRPPWMG LLGPTIQAEV YDTVVITLKN MASHPVSLHA VGVSYWKASE GAEYDDQTSQ | 120 |
| SEQ ID NO: 8 | REKEDDKVFP GGSHTYVWQV LKENGPMASD PLCLTYSYLS HVDLVKDLNS GLIGALLVCR | 180 |
| | EGSLAKEKTQ TLHKFILLFA VFDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVFD ENRSWYLTEN IQRFLPNPAG | 600 |
| | VQLEDPEFQA SNIMHSINGY VFDSLQLSVC LHEVAYWYIL SIGAQTDFLS VFFSGYTFKH | 660 |
| | KMVYEDTLTL FPFSGETVFM SMENPGLWIL GCHNSDFRNR GMTALLKVSS CDKNTGDYYE | 720 |
| | DSYEDISAYL LSKNNAIEPR SFSQNSRHPS TRQKQFNATT IPENDIEKTD PWFAHRTPMP | 780 |
| | KIQNVSSSDL LMLLRQSPTP HGLSLSDLQE AKYETFSDDP SPGAIDSNNS LSEMTHFRPQ | 840 |
| | LHHSGDMVFT PESGLQLRLN EKLGTTAATE LKKLDFKVSS TSNNLISTIP SDNLAAGTDN | 900 |
| | TSSLGPPSMP VHYDSQLDTT LFGKKSSPLT ESGGPLSLSE ENNDSKLLES GLMNSQESSW | 960 |
| | GKNVSSTESG RLFKGKRAHG PALLTKDNAL FKVSISLLKT NKTSNNSATN RKTHIDGPSL | 1020 |
| | LIENSPSVWQ NILESDTEFK KVTPLIHDRM LMDKNATALR LNHMSNKTTS SKNMEMVQQK | 1080 |
| | KEGPIPPDAQ NPDMSFFKML FLPESARWIQ RTHGKNSLNS GQGPSPKQLV SLGPEKSVEG | 1140 |
| | QNFLSEKNKV VVGKGEFTKD VGLKEMVFPS SRNLFLTNLD NLHENNTHNQ EKKIQEEIEK | 1200 |
| | KETLIQENVV LPQIHTVTGT KNFMKNLFLL STRQNVEGSY DGAYAPVLQD FRSLNDSTNR | 1260 |
| | TKKHTAHFSK KGEEENLEGL GNQTKQIVEK YACTTRISPN TSQQNFVTQR SKRALKQFRL | 1320 |
| | PLEETELEKR IIVDDTSTQW SKNMKHLTPS TLTQIDYNEK EKGAITQSPL SDCLTRSHSI | 1380 |
| | PQANRSPLPI AKVSSFPSIR PIYLTRVLFQ DNSSHLPAAS YRKKDSGVQE SSHFLQGAKK | 1440 |
| | NNLSLAILTL EMTGDQREVG SLGTSATNSV TYKKVENTVL PKPDLPKTSG KVELLPKVHI | 1500 |
| | YQKDLFPTET SNGSPGHLDL VEGSLLQGTE GAIKWNEANR PGKVPFLRVA TESSAKTPSK | 1560 |
| | LLDPLAWDNH YGTQIPKEEW KSQEKSPEKT AFKKKDTILS LNACESNHAI AAINEGQNKP | 1620 |
| | EIEVTWAKQG RTERLCSQNP PVLKRHQREI TRTTLQSDQE EIDYDDTISV EMKKEDFDIY | 1680 |
| | DEDENQSPRS FQKKTRHYFI AAVERLWDYG MSSSPHVLRN RAQSGSVPQF KKVVFQEFTD | 1740 |
| | GSFTQPLYRG ELNEHLGLLG PYIRAEVEDN IMVTFRNQAS RPYSFYSSLI SYEEDQRQGA | 1800 |
| | EPRKNFVKPN ETKTYFWKVQ HHMAPTKDEF DCKAWAYFSD VDLEKDVHSG LIGPLLVCHT | 1860 |
| | NTLNPAHGRQ VTVQEFALFF TIFDETKSWY FTENMERNCR APCNIQMEDP TFKENYRFHA | 1920 |
| | INGYIMDTLP GLVMAQDQRI RWYLLSMGSN ENIHSIHFSG HVFTVRKKEE YKMALYNLYP | 1980 |
| | GVFETVEMLP SKAGIWRVEC LIGEHLHAGM STLFLVYSNK CQTPLGMASG HIRDFQITAS | 2040 |
| | GQYGQWAPKL ARLHYSGSIN AWSTKEPFSW IKVDLLAPMI IHGIKTQGAR QKFSSLYISQ | 2100 |
| | FIIMYSLDGK KWQTYRGNST GTLMVFFGNV DSSGIKHNIF NPPIIARYIR LHPTHYSIRS | 2160 |
| | TLRMELMGCD LNSCSMPLGM ESKAISDAQI TASSYFTNMF ATWSPSKARL HLQGRSNAWR | 2220 |
| | PQVNNPKEWL QVDFQKTMKV TGVTTQGVKS LLTSMYVKEF LISSSQDGHQ WTLFFQNGKV | 2280 |
| | KVFQGNQDSF TPVVNSLDPP LLTRYLRIHP QSWVHQIALR MEVLGCEAQD LY | 2332 |
| ELNN polypeptide | GTSESATPES GPGSEPATSG SETPGTSESA TPESGPGSEP ATSGSETPGT SESATPESGP | 60 |
| AE288 | GTSTEPSEGS APGSPAGSPT STEEGTSESA TPESGPGSEP ATSGSETPGT SESATPESGP | 120 |
| SEQ ID NO: 9 | GSPAGSPTST EEGSPAGSPT STEEGTSTEP SEGSAPGTSE SATPESGPGT SESATPESGP | 180 |
| | GTSESATPES GPGSEPATSG SETPGSEPAT SGSETPGSPA GSPTSTEEGT STEPSEGSAP | 240 |
| | GTSTEPSEGS APGSEPATSG SETPGTSESA TPESGPGTST EPSEGSAP | 288 |
| pSYN VWF059 | TSTEEGASIS DKNTGDYYED SYEDISAYLL SKNNAIEPRS FSDKTH | |
| SEQ ID NO: 10 | | |
| pSYN VWF059A | TSTEEGASSD KNTGDYYEDS YEDISAYLLS KNNAIEPRSF SDKTH | |
| SEQ ID NO: 11 | | |
| pSYN FVIII 312 | ATRRYYLGAV ELSWDYMQSD LGELPVDARF PPRVPKSFPF NTSVVYKKTL FVEFTDHLFN | 60 |
| SEQ ID NO: 12 | IAKPRPPWMG LLGPTIQAEV YDTVVITLKN MASHPVSLHA VGVSYWKASE GAEYDDQTSQ | 120 |
| | REKEDDKVFP GGSHTYVWQV LKENGPMASD PLCLTYSYLS HVDLVKDLNS GLIGALLVCR | 180 |
| | EGSLAKEKTQ TLHKFILLFA VFDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVFD ENRSWYLTEN IQRFLPNPAG | 600 |
| | VQLEDPEFQA SNIMHSINGY VFDSLQLSVC LHEVAYWYIL SIGAQTDFLS VFFSGYTFKH | 660 |
| | KMVYEDTLTL FPFSGETVFM SMENPGLWIL GCHNSDFRNR GMTALLKVSS CDKNTGDYYE | 720 |
| | DSYEDISAYL LSKNNAIEPR SFSQNGTSES ATPESGPGSE PATSGSETPG TSESATPESG | 780 |
| | PGSEPATSGS ETPGTSESAT PESGPGTSTE PSEGSAPGSP AGSPTSTEEG TSESATPESG | 840 |
| | PGSEPATSGS ETPGTSESAT PESGPGSPAG SPTSTEEGSP AGSPTSTEEG TSTEPSEGSA | 900 |
| | PGTSESATPE SGPGTSESAT PESGPGTSES ATPESGPGSE PATSGSETPG SEPATSGSET | 960 |
| | PGSPAGSPTS TEEGTSTEPS EGSAPGTSTE PSEGSAPGSE PATSGSETPG TSESATPESG | 1020 |
| | PGTSTEPSEG SAPASSEITR TTLQSDQEEI DYDDTISVEM KKEDFDIYDE DENQSPRSFQ | 1080 |
| | KKTRHYFIAA VERLWDYGMS SSPHVLRNRA QSGSVPQFKK VVFQEFTDGS FTQPLYRGEL | 1140 |
| | NEHLGLLGPY IRAEVEDNIM VTFRNQASRP YSFYSSLISY EEDQRQGAEP RKNFVKPNET | 1200 |
| | KTYFWKVQHH MAPTKDEFDC KAWAYFSDVD LEKDVHSGLI GPLLVCHTNT LNPAHGRQVT | 1260 |
| | VQEFALFFTI FDETKSWYFT ENMERNCRAP CNIQMEDPTF KENYRFHAIN GYIMDTLPGL | 1320 |
| | VMAQDQRIRW YLLSMGSNEN IHSIHFSGHV FTVRKKEEYK MALYNLYPGV FETVEMLPSK | 1380 |
| | AGIWRVECLI GEHLHAGMST LFLVYSNKCQ TPLGMASGHI RDFQITASGQ YGQWAPKLAR | 1440 |
| | LHYSGSINAW STKEPFSWIK VDLLAPMIIH GIKTQGARQK FSSLYISQFI IMYSLDGKKW | 1500 |
| | QTYRGNSTGT LMVFFGNVDS SGIKHNIFNP PIIARYIRLH PTHYSIRSTL RMELMGCDLN | 1560 |
| | SCSMPLGMES KAISDAQITA SSYFTNMFAT WSPSKARLHL QGRSNAWRPQ VNNPKEWLQV | 1620 |
| | DFQKTMKVTG VTTQGVKSLL TSMYVKEFLI SSSQDGHQWT LFFQNGKVKV FQGNQDSFTP | 1680 |
| | VVNSLDPPLL TRYLRIHPQS WVHQIALRME VLGCEAQDLY DKTHTCPPCP APELLGGPSV | 1740 |
| | FLFPPKPKDT LMISRTPEVT CVVVDVSHED PEVKFNWYVD GVEVHNAKTK PREEQYNSTY | 1800 |
| | RVVSVLTVLH QDWLNGKEYK CKVSNKALPA PIEKTISKAK GQPREPQVYT LPPSRDELTK | 1860 |
| | NQVSLTCLVK GFYPSDIAVE WESNGQPENN YKTTPPVLDS DGSFFLYSKL TVDKSRWQQG | 1920 |
| | NVFSCSVMHE ALHNHYTQKS LSLSPGK | |
| ELNN polypeptide | GSPAGSPTST EEGTSESATP ESGPGSEPAT SGSETPGTSE SATPESGPGT STEPSEGSAP | 60 |
| AE288_2 | GTSTEPSEGS APGTSTEPSE GSAPGTSTEP SEGSAPGTST EPSEGSAPGT STEPSEGSAP | 120 |
| SEQ ID NO: 13 | GSPAGSPTST EEGTSTEPSE GSAPGTSESA TPESGPGSEP ATSGSETPGT SESATPESGP | 180 |
| | GSEPATSGSE TPGTSESATP ESGPGTSTEP SEGSAPGTSE SATPESGPGS PAGSPTSTEE | 240 |
| | GSPAGSPTST EEGSPAGSPT STEEGTSESA TPESGPGTST EPSEGSAP | 288 |
| ELNN polypeptide | TSESATPESG PGSEPATSGS ETPGTSESAT PESGPGSEPA TSGSETPGTS ESATPESGPG | 60 |
| AE144_5A | TSTEPSEGSA PGSPAGSPTS TEEGTSESAT PESGPGSEPA TSGSETPGTS ESATPESGPG | 120 |
| SEQ ID NO: 14 | SPAGSPTSTE EGSPAGSPTS TEEG | |
| a2 Linker of chimeric | DKNTGDYYED SYEDISAYLL SKNNAIEPRS FS | 32 |
| protein | | |
| SEQ ID NO: 15 | | |
| Signal Peptide of | MQIELSTCFFLCLLRFCFS | |
| FVIII | | |
| SEQ ID NO: 16 | | |
| FVIII fragment 1 of | ATRRYYLGAV ELSWDYMQSD LGELPVDARF PPRVPKSFPF NTSVVYKKTL FVEFTDHLFN | 60 |
| chimeric protein | IAKPRPPWMG LLGPTIQAEV YDTVVITLKN MASHPVSLHA VGVSYWKASE GAEYDDQTSQ | 120 |
| SEQ ID NO. 17 | REKEDDKVFP GGSHTYVWQV LKENGPMASD PLCLTYSYLS HVDLVKDLNS GLIGALLVCR | 180 |
| | EGSLAKEKTQ TLHKFILLFA VFDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVFD ENRSWYLTEN IQRFLPNPAG | 600 |
| | VQLEDPEFQA SNIMHSINGY VFDSLQLSVC LHEVAYWYIL SIGAQTDFLS VFFSGYTFKH | 660 |
| | KMVYEDTLTL FPFSGETVFM SMENPGLWIL GCHNSDFRNR GMTALLKVSS CDKNTGDYYE | 720 |
| | DSYEDISAYL LSKNNAIEPR SFSQN | |
| FVIII fragment 2 | EITRTTLQSD QEEIDYDDTI SVEMKKEDFD IYDEDENQSP RSFQKKTRHY FIAAVERLWD | 60 |
| of chimeric protein | YGMSSSPHVL RNRAQSGSVP QFKKVVFQEF TDGSFTQPLY RGELNEHLGL LGPYIRAEVE | 120 |
| SEQ ID NO. 18 | DNIMVTFRNQ ASRPYSFYSS LISYEEDQRQ GAEPRKNFVK PNETKTYFWK VQHHMAPTKD | 180 |
| | EFDCKAWAYF SDVDLEKDVH SGLIGPLLVC HTNTLNPAHG RQVTVQEFAL FFTIFDETKS | 240 |
| | WYFTENMERN CRAPCNIQME DPTFKENYRF HAINGYIMDT LPGLVMAQDQ RIRWYLLSMG | 300 |
| | SNENIHSIHF SGHVFTVRKK EEYKMALYNL YPGVFETVEM LPSKAGIWRV ECLIGEHLHA | 360 |
| | GMSTLFLVYS NKCQTPLGMA SGHIRDFQIT ASGQYGQWAP KLARLHYSGS INAWSTKEPF | 420 |
| | SWIKVDLLAP MIIHGIKTQG ARQKFSSLYI SQFIIMYSLD GKKWQTYRGN STGTLMVFFG | 480 |
| | NVDSSGIKHN IFNPPIIARY IRLHPTHYSI RSTLRMELMG CDLNSCSMPL GMESKAISDA | 540 |
| | QITASSYFTN MFATWSPSKA RLHLQGRSNA WRPQVNNPKE WLQVDFQKTM KVTGVTTQGV | 600 |
| | KSLLTSMYVK EFLISSSQDG HQWTLFFQNG KVKVFQGNQD SFTPVVNSLD PPLLTRYLRI | 660 |
| | HPQSWVHQIA LRMEVLGCEA QDLY | |
| VWF Signal Peptide | MIPARFAGVL LALALILPGT LC | |
| SEQ ID NO: 19 | | |
| VWF D1D2 domain | AEGTRGRSST ARCSLFGSDF VNTFDGSMYS FAGYCSYLLA GGCQKRSFSI IGDFQNGKRV | 60 |
| of chimeric protein | SLSVYLGEFF DIHLFVNGTV TQGDQRVSMP YASKGLYLET EAGYYKLSGE AYGFVARIDG | 120 |
| SEQ ID NO: 20 | SGNFQVLLSD RYFNKTCGLC GNFNIFAEDD FMTQEGTLTS DPYDFANSWA LSSGEQWCER | 180 |
| | ASPPSSSCNI SSGEMQKGLW EQCQLLKSTS VFARCHPLVD PEPFVALCEK TLCECAGGLE | 240 |
| | CACPALLEYA RTCAQEGMVL YGWTDHSACS PVCPAGMEYR QCVSPCARTC QSLHINEMCQ | 300 |
| | ERCVDGCSCP EGQLLDEGLC VESTECPCVH SGKRYPPGTS LSRDCNTCIC RNSQWICSNE | 360 |
| | ECPGECLVTG QSHFKSFDNR YFTFSGICQY LLARDCQDHS FSIVIETVQC ADDRDAVCTR | 420 |
| | SVTVRLPGLH NSLVKLKHGA GVAMDGQDIQ LPLLKGDLRI QHTVTASVRL SYGEDLQMDW | 480 |
| | DGRGRLLVKL SPVYAGKTCG LCGNYNGNQG DDFLTPSGLA EPRVEDFGNA WKLHGDCQDL | 540 |
| | QKQHSDPCAL NPRMTRFSEE ACAVLTSPTF EACHRAVSPL PYLRNCRYDV CSCSDGRECL | 600 |
| | CGALASYAAA CAGRGVRVAW REPGRCELNC PKGQVYLQCG TPCNLTCRSL SYPDEECNEA | 660 |
| | CLEGCFCPPG LYMDERGDCV PKAQCPCYYD GEIFQPEDIF SDHHTMCYCE DGFMHCTMSG | 720 |
| | VPGSLLPDAV LSSPLSHRSK R | |
| VWF D′ domain of | SLSCRPPMVK LVCPADNLRA EGLECTKTCQ NYDLECMSMG CVSGCLCPPG MVRHENRCVA | 60 |
| chimeric protein | LERCPCFHQG KEYAPGETVK IGCNTCVCRD RKWNCTDHVC DAT | 103 |
| SEQ ID NO: 21 | | |
| VWVF D3 domain of | CSTIGMAHYL TFDGLKYLFP GECQYVLVQD YCGSNPGTFR ILVGNKGCSH PSVKCKKRVT | 60 |
| chimeric protein | ILVEGGEIEL FDGEVNVKRP MKDETHFEVV ESGRYIILLL GKALSVVWDR HLSISVVLKQ | 120 |
| SEQ ID NO: 22 | TYQEKVCGLC GNFDGIQNND LTSSNLQVEE DPVDFGNSWK VSSQCADTRK VPLDSSPATC | 180 |
| | HNNIMKQTMV DSSCRILTSD VFQDCNKLVD PEPYLDVCIY DTCSCESIGD CAAFCDTIAA | 240 |
| | YAHVCAQHGK VVTWRTATLC PQSCEERNLR ENGYEAEWRY NSCAPACQVT CQHPEPLACP | 300 |
| | VQCVEGCHAH CPPGKILDEL LQTCVDPEDC PVCEVAGRRF ASGKKVTLNP SDPEHCQICH | 360 |
| | CDVVNLTCEA CQEP | 374 |
| Fc region | DKTHTCPPCP APELLGGPSV FLFPPKPKDT LMISRTPEVT CVVVDVSHED PEVKFNWYVD | 60 |
| SEQ ID NO: 23 | GVEVHNAKTK PREEQYNSTY RVVSVLTVLH QDWLNGKEYK CKVSNKALPA PIEKTISKAK | 120 |
| | GQPREPQVYT LPPSRDELTK NQVSLTCLVK GFYPSDIAVE WESNGQPENN YKTTPPVLDS | 180 |
| | DGSFFLYSKL TVDKSRWQQG NVFSCSVMHE ALHNHYTQKS LSLSPG | |
| ELNN polypeptide | GTSESATPES GPGSEPATSG SETPGTSESA TPESGPGSEP ATSGSETPGT SESATPESGP | 60 |
| AE288_3 | GTSTEPSEGS APGSPAGSPT STEEGTSESA TPESGPGSEP ATSGSETPGT SESATPESGP | 120 |
| SEQ ID NO: 24 | GSPAGSPTST EEGSPAGSPT STEEGTSTEP SEGSAPGTSE SATPESGPGT SESATPESGP | 180 |
| | GTSESATPES GPGSEPATSG SETPGSEPAT SGSETPGSPA GSPTSTEEGT STEPSEGSAP | 240 |
| | GTSTEPSEGS APGSEPATSG SETPGTSESA TPESGPGTST EPSEGSAPAS S | 291 |

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. A method of reducing an amount of pain medication needed to decrease or manage pain associated with hemophilia A, comprising administering to a human subject who has hemophilia A in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond, and
wherein the amount of pain medication needed decreases when the subject is treated with the chimeric protein compared to the amount needed when the subject is treated for hemophilia A with (i) a FVIII replacement protein that is capable of being bound by endogenous von Willebrand Factor (VWF); (ii) a complex, wherein the complex comprises a FVIII protein and a VWF protein or a portion thereof, wherein the FVIII protein and the VWF protein or portion thereof are not directly or indirectly covalently attached to each other; or (iii) emicizumab.

2. The method of claim 1, wherein the amount of pain medication needed decreases when the subject is treated with the chimeric protein compared to before the chimeric protein was administered to the subject for the first time.

3. The method of claim 1, wherein the dose of the pain medication is reduced and/or the number of doses of the pain medication over the course of a day or a week is reduced.

4. (canceled)

5. A method of improving joint health in a human subject during the prophylactic treatment of hemophilia A, wherein the prophylactic treatment of hemophilia A comprises administering to the human subject in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and
wherein the first Fc region and the second Fc region are covalently attached to each other by at least one disulfide bond,
wherein the improvement in joint health comprises an improvement in cartilage in the subject.

6. A method of prophylactically treating hemophilia A, comprising administering to a human subject in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and
wherein the subject is not administered any on-demand treatment for hemophilia A before engaging in strenuous activity.

7. (canceled)

8. A method of improving quality of life with the prophylactic treatment of hemophilia A, comprising administering to a human subject in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond, and
wherein the subject's quality of life improves compared to (i) a corresponding subject who receives prophylactic treatment with emicizumab, (ii) a corresponding subject who receives prophylactic treatment for hemophilia A with a FVIII replacement protein other than the chimeric protein, wherein the FVIII replacement protein is capable of being bound by endogenous VWF, or (iii) a corresponding subject who receives prophylactic treatment for hemophilia A with a complex, wherein the complex comprises a FVIII protein and a VWF protein or a portion thereof, wherein the FVIII protein and the VWF protein or portion thereof are not directly or indirectly covalently attached to each other.

9. (canceled)

10. (canceled)

11. The method of claim 1, wherein the subject has (i) less pain from swelling, (ii) less joint pain, (iii) improved mobility, (iv) less disruption to school activities, (v) less disruption to work activities, (vi) less difficulty walking, (vii) less fatigue, and/or (viii) increased fitness,
when being treated with the chimeric protein compared to (i) a corresponding subject who receives prophylactic treatment with emicizumab, (ii) a corresponding subject who receives prophylactic treatment for hemophilia A with a FVIII replacement protein other than the chimeric protein, wherein the FVIII replacement protein is capable of being bound by endogenous VWF, or (iii) a corresponding subject who receives prophylactic treatment for hemophilia A with a complex, wherein the complex comprises a FVIII protein and a VWF protein or a portion thereof, wherein the FVIII protein and the VWF protein or portion thereof are not directly or indirectly covalently attached to each other.

12. A method of achieving an annualized bleeding rate (ABR) of 2 or less, comprising administering to a human subject who has hemophilia A in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and
wherein a total of from about 2600 IU/kg to about 2650 IU/kg of the chimeric protein is administered to the subject per year.

13. A method of reducing an annualized bleeding rate (ABR) compared to treatment with a Factor VIII (FVIII) replacement protein that is capable of being bound by endogenous von Willebrand Factor (VWF), a complex, or emicizumab comprising administering to a human subject who has hemophilia A in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a VWF fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and
wherein the FVIII replacement protein that is capable of being bound by endogenous VWF is lonoctocog alfa, octocog alfa, rurioctocog alfa pegol, damoctocog alfa pegol, efmoroctocog alfa, simoctocog alfa, moroctocog alfa, beroctocog alfa, or turoctocog alfa, or wherein the complex comprises a FVIII protein and a wild-type VWF protein or a portion thereof, wherein the FVIII protein and the VWF protein or portion thereof are not directly or indirectly covalently attached to each other.

14. (canceled)

15. A method of reducing the risk of a bleeding episode that requires more than one supplemental on-demand administration of a FVIII replacement protein during the prophylactic treatment of hemophilia A, wherein the prophylactic treatment of hemophilia A comprises administering to a human subject in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond,
wherein the risk is decreased by at least 90% compared to the risk of a corresponding subject who receives prophylactic treatment for hemophilia A that comprises administration of an FVIII replacement protein other than the chimeric protein, and
wherein the FVIII replacement protein other than the chimeric protein is capable of being bound by endogenous VWF, and wherein the supplemental on-demand dose is 45 IU/kg to 70 IU/kg.

16. The method of claim 12, wherein the therapeutically effective amount is a dose of the chimeric protein of about 50 IU/kg once about every 7 days.

17. A method of resolving a bleeding episode, comprising administering to a human subject in need thereof a single on-demand dose of a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond,
wherein the single on-demand dose is about 50 IU/kg, and
wherein the bleeding episode is in a joint.

18. A method of on-demand treatment of a major bleed, comprising administering to a human subject who has hemophilia A in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, wherein the first Fc region and the second Fc region are covalently attached to each other by at least one disulfide bond, and
wherein the major bleed is an intracranial bleed, a retroperitoneal bleed, an iliopsoas bleed, a neck bleed, a muscle bleed with compartment syndrome, and/or a bleed associated with a significant decrease in hemoglobin level.

19. A method for the perioperative management of bleeding, comprising administering to a human subject who has hemophilia A in need thereof a therapeutically effective amount of a chimeric protein,
wherein the chimeric protein comprises a first polypeptide and a second polypeptide,
wherein the first polypeptide comprises (a) a FVIII polypeptide with a full or partial B domain deletion, wherein a first ELNN polypeptide is inserted within the FVIII polypeptide, and (b) a first Fc region; and the second polypeptide comprises (a) a von Willebrand Factor (VWF) fragment, (b) a second ELNN polypeptide, (c) a thrombin-cleavable linker comprising at least a portion of the a2 region of FVIII, and (d) a second Fc region, and wherein the first Fc region and the second Fc region are covalently attached to each other by at least one disulfide bond.

20-42. (canceled)

43. The method of claim 12, wherein the first polypeptide comprises the amino acid sequence set forth as SEQ ID NO: 1 and the second polypeptide comprises the amino acid sequence set forth as SEQ ID NO: 2, wherein the first polypeptide and the second polypeptide are covalently linked by two disulfide bonds between the first Fc region and the second Fc region.

44. The method of claim 12, wherein the chimeric protein is efanesoctocog alfa.

45. The method of a claim 12, wherein the subject has severe hemophilia A.

46. The method of claim 12, wherein the chimeric protein is administered intravenously.

47. The method of claim 12, wherein the subject is not administered any on-demand treatment for hemophilia A before engaging in strenuous activity.

48. The method of claim 47, wherein the strenuous activity comprises basketball, football, hockey, rugby, boxing, wrestling, or a martial art.
